(19) United States
(12) Patent Application Publication
(10) Pub. No.: US 2024/0294519 A1
(43) Pub. Date: Sep. 5, 2024

(54) PYRROLO[2,3-b]PYRIDINE PGDH INHIBITORS AND METHODS OF MAKING AND USING

(71) Applicant: EPIRIUM BIO INC., San Diego, CA (US)

(72) Inventors: Sundeep DUGAR, San Jose, CA (US); Bruce FAHR, San Diego, CA (US); Roopa RAI, San Carlos, CA (US); Michael J. GREEN, Half Moon Bay, CA (US)

(21) Appl. No.: 18/292,856

(22) PCT Filed: Jul. 27, 2022
(86) PCT No.: PCT/US2022/038548

§ 371 Date: Jan. 26, 2024

Related U.S. Application Data
(60) Provisional application No. 63/226,670, filed on Jul. 28, 2021.

(51) Int. Cl. C07D 471/04; A61K 31/437; A61K 31/438; A61K 31/444; A61K 31/4545; A61K 31/5377; A61K 31/5386; A61K 31/541; A61K 31/553; C07D 519/00

(52) U.S. Cl. CPC C07D 471/04; A61K 31/437; A61K 31/438; A61K 31/444; A61K 31/4545; A61K 31/5377; A61K 31/5386; A61K 31/541; A61K 31/553; C07D 519/00

(57) ABSTRACT

Disclosed herein are 15-hydroxyprostaglandin dehydrogenase inhibitor compounds, including variations of pyrrolopy-rimidines. Such compounds may be administered to subjects that may benefit from modulation of prostaglandin levels.

### CROSS REFERENCE TO RELATED APPLICATIONS

This application claims the benefit of U.S. Provisional Application No. 63/226,670, filed Jul. 28, 2021, which is hereby incorporated by reference in its entirety.

### BACKGROUND OF THE INVENTION

Prostaglandins are a group of physiologically active lipid compounds with diverse biological effects including vasodilation, inhibition of platelet aggregation, bronchodilation, bronchoconstriction, immune responses, contraction and relaxation of gastrointestinal smooth muscles, gastric acid secretion, gastric mucus secretion, uterus contraction, lipolysis inhibition, neurotransmission, clotting, hyperalgesia, and pyrexia.

Treatment of diseases or disorders may require activation of prostaglandins, or inhibition of inactivation of prostaglandins. Hydroxyprostaglandin dehydrogenases, such as 15-hydroxyprostaglandin dehydrogenase (15-PGDH) are involved in the inactivation of prostaglandins. As such, diseases/disorders associated with prostaglandins can be prevented, treated and/or managed using inhibitors of hydroxyprostaglandin dehydrogenase such as inhibitors of 15-PGDH.

### SUMMARY OF THE INVENTION

In an aspect, provided herein is a compound having the structure of Formula (N), or a pharmaceutically acceptable salt or solvate thereof:

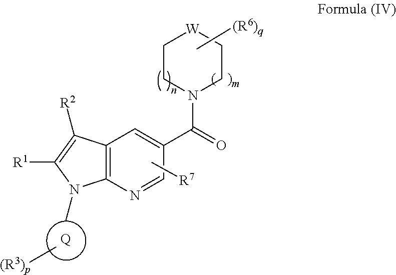

wherein,
ring Q is C₆ aryl or 5- to 10-membered heteroaryl;
W is —CR⁶R⁶—, —O—, —S—, —NR⁵—, —S(O)₂—, or —C(O)—;
R¹ and R² are each independently H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, —CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —OC(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁵ is H, C₁-C₆ alkyl, or
each R⁶ is independently H, halogen, CN, —NR⁸R⁹, —OR¹⁰, —C(O)R¹⁰, —C(O)OR¹⁰, —C(O)NR⁸R⁹, —SOR¹¹, —SO₂R¹¹, substituted or unsubstituted C₁-C₆ alkyl;
or two R⁶ can join together with the atom(s) to which they are attached to form a C₃-C₆ cycloalkyl or C₃-C₈ heterocycloalkyl ring;
each R⁷ is independently H, halogen, —CN, —NR¹⁰R¹⁰, —OR¹⁰, —C(O)R¹⁰, —C(O)OR¹⁰, or substituted or unsubstituted C₁-C₆ alkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
n and m are each independently 0, 1, 2, or 3;
q is 0, 1, 2, or 3; and
p is 1,2,3, or 4.

In some embodiments, the compounds has the structure of Formula (V), or a pharmaceutically acceptable salt or solvate thereof:

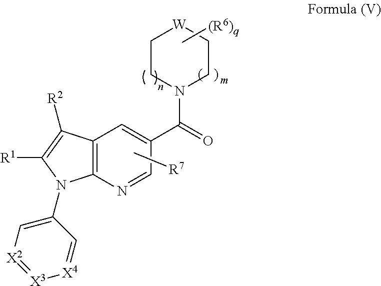

wherein,
X² is N, NR^{3A}, or CR^{3A};
X³ is N or CR^{3B};
X⁴ isN, NR^{3C}, or CR^{3C}; and
R^{3A}, R^{3B}, and R^{3C} are each independently H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R^{3A} and R^{3B} together with the atoms to which they are attached form a substituted or unsubstituted 5 to 6-membered aryl or heteroaryl; or
R^{3B} and R^{3C} together with the atoms to which they are attached form a substituted or unsubstituted 5 to 6-membered aryl or heteroaryl; and
wherein CR^{3A}, CR^{3B}, and CR^{3C} are not all H at the same time.

In some embodiments, the compound has the structure of Formula (Va), or a pharmaceutically acceptable salt or solvate thereof:

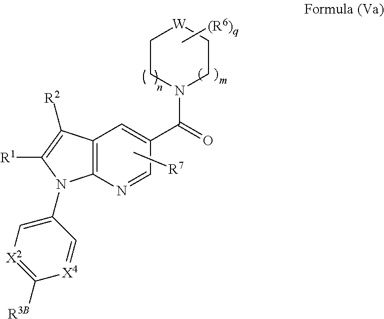

wherein,
X² is N or CR^{3A}; and
X⁴ is N or CR^{3C}; and
R^{3A}, R^{3B} and R^{3C} are each independently H, halogen, —CN, —NR⁸R⁹, —OR¹⁰, CN, —C(O)R¹⁰, —C(O)OR¹⁰, —C(O)NR⁸R⁹, —NR¹²C(O)R¹⁰, —NR¹²C(O)OR¹⁰, —NR¹²C(O)NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl; and
wherein R^{3A}, R^{3B}, and R^{3C} are not each H.

In some embodiments, X² is N and X⁴ is CR^{3C}. In some embodiments, R^{3B} is H, and R^{3C} is —C(O)R¹⁰, —C(O)OR¹⁰, —C(O)NR⁸R⁹, —NR¹²C(O)R¹⁰, substituted or unsubstituted C₃-C₈ heterocycloalkyl, or substituted or unsubstituted 5-membered heteroaryl. In some embodiments, R^{3C} is H, and R^{3B} is —C(O)R¹⁰, —C(O)OR¹⁰, —C(O)NR⁸R⁹, —NR¹²C(O)R¹⁰, substituted or unsubstituted C₃-C₈ heterocycloalkyl, or substituted or unsubstituted 5-membered heteroaryl. In some embodiments, X² is C^{3A} and X⁴ is N. In some embodiments, X² is CR³ and X⁴ is CR^{3C}. In some embodiments, R^{3A} is H, and R^{3B} is —C(O)R¹⁰, —C(O)OR¹⁰, —C(O)NR⁸R⁹, —NR¹²C(O)R¹⁰, substituted or unsubstituted C₃-C₈ heterocycloalkyl, or substituted or unsubstituted 5-membered heteroaryl. In some embodiments, R^{3B} is H, and R^{3A} is —C(O)R¹⁰, —C(O)OR¹⁰, —C(O)NR⁸R⁹, —NR¹²C(O)R¹⁰, substituted or unsubstituted C₃-C₈ heterocycloalkyl, or substituted or unsubstituted 5-membered heteroaryl. In some embodiments, X² is CR^{3A} and X⁴ is CR. In some embodiments, R^{3A} and R^{3B} are each H; and R is —C(O)R¹⁰, —NR¹²C(O)R¹⁰, substituted or unsubstituted C₃-C₈ heterocycloalkyl, or substituted or unsubstituted 5-membered heteroaryl. In some embodiments, R^{3A} and R^{3C} are each H; and R^{3B} is —C(O)R¹⁰, —NR¹²C(O)R¹⁰, substituted or unsubstituted C₃-C₈ heterocycloalkyl, or substituted or unsubstituted 5-membered heteroaryl. In some embodiments, R^{3B} and R^{3C} are each H; and R^{3A} is —C(O)R¹⁰, —NR¹²C(O)R¹⁰, substituted or unsubstituted C₃-C₈ heterocycloalkyl, or substituted or unsubstituted 5-membered heteroaryl.

In some embodiments, one of R^{3A}, R^{3B}, or R^{3C} is a substituted or unsubstituted 5-membered heteroaryl. In some embodiments, the 5-membered heteroaryl is triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl.

In some embodiments, the compound has the structure of Formula (VIIa) or (VIIb), or a pharmaceutically acceptable salt or solvate thereof:

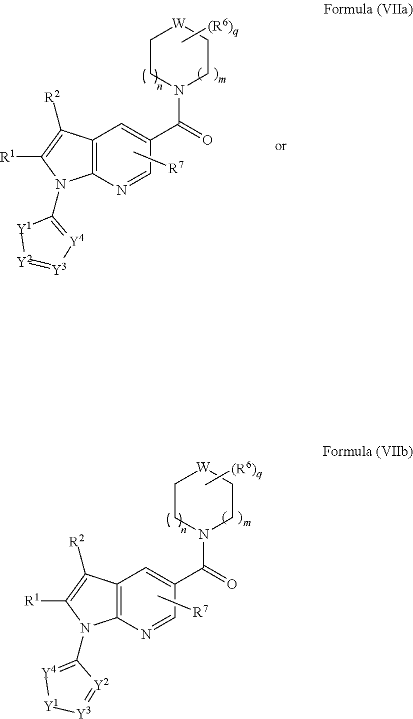

wherein,
Y¹ is O, S, or NR^{3D};
Y² is N or CR^{3A};
Y³ and Y⁴ are each independently N or CR^{3B};
R^{3A} and R^{3B} are each independently selected from H, halogen, —NR⁸R⁹, —OR¹⁰, —C(O)R¹⁰, —C(O)OR¹⁰, —C(O)NR⁸R⁹,-substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, or substituted or unsubstituted 5-membered heteroaryl; and
R^{3D} is H or C₁-C₆ alkyl.

In some embodiments, the compound has the structure of Formula (VIII), or a pharmaceutically acceptable salt or solvate thereof:

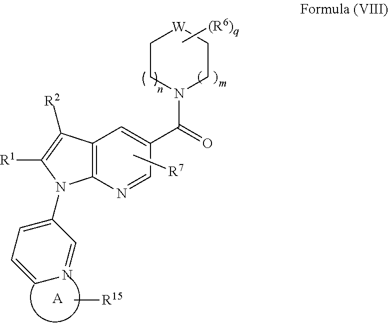

wherein,
ring A is a 5-membered heteroaryl optionally comprising 1 or 2 N atoms;
X⁶ is C or N; and
R¹⁵ is H, halogen, —NR⁸R⁹,-substituted or unsubstituted C₁-C₆ alkyl, substituted or unsubstituted C₁-C₆ haloalkyl, substituted or unsubstituted C₃-C₈ cycloalkyl, or substituted or unsubstituted C₃-C₈ heterocycloalkyl.

In another aspect, provided herein is a compound having the structure of Formula (Ia), or a pharmaceutically acceptable salt or solvate thereof:

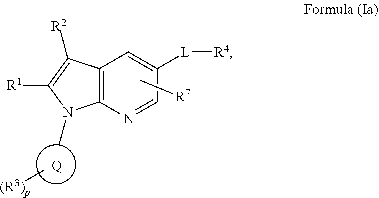

wherein,
ring Q is C₆ aryl or 5- to 10-membered heteroaryl;
L is —CR^{13A}R^{13H}—, —C(O)—, —S—, —S(O)—, or
R¹ and R² are each independently H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5-to 10-membered heteroaryl;
   R⁴ is substituted or unsubstituted C₁, —C₈ alkyl, substituted or unsubstituted C₂-C₈ alkenyl, substituted or unsubstituted C₁-C₈ heteroalkyl; or R⁴ is

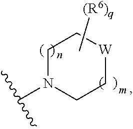

wherein
W is —CR⁶R⁶—, —O—, —S—, —NR⁵—, —S(O)₂—, or —C(O)—;
   R⁵ is H or substituted or unsubstituted C₁-C₆ alkyl, substituted or unsubstituted cycloalkyl, or substituted or unsubstituted heterocycloalkyl;
   each R⁶ is independently H, halogen, CN, —NR⁸R⁹, —OR¹⁰, —C(O)R¹⁰, —C(O)OR¹⁰, —C(O)NR⁸R⁹, —SOR¹¹, —SO₂R¹¹, substituted or unsubstituted C₁-C₆ alkyl;
   or two R⁶ can join together with the atom(s) to which they are attached to form a C₃-C₆ cycloalkyl or C₃-C₈ heterocycloalkyl ring;
   n and m are each independently 0, 1, 2, or 3; and
   g is 0, 1, 2, 3, 4, 5, or 6;
R⁷ is H, halogen, —OR¹⁰, —C(O)R¹⁰, —C(O)OR¹⁰, or substituted or unsubstituted C₁-C₆ alkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆haloalkyl, and C₃-C₁₀cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
R^{13A} and R^{13B} are each independently H, CF₃, halogen, or C₁-C₆ alkyl; and
p is 1,2,3, or 4.

In some embodiments, the compound has the structure of Formula (II), or a pharmaceutically acceptable salt or solvate thereof:

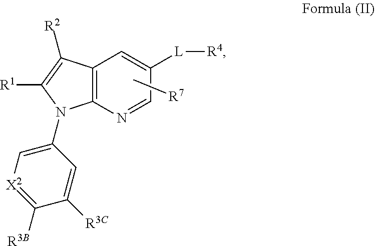

wherein,
X² is N or CR^{3A}; and
R^{3A}, R^{3B}, and R^{3C} are independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, and substituted or unsubstituted 5-to 10-membered heteroaryl;
provided that R^{3A}, R^{3B}, and R^{3C} are not all H at the same time.

In some embodiments, the compound has the structure of Formula (IIIa), or a pharmaceutically acceptable salt or solvate thereof:

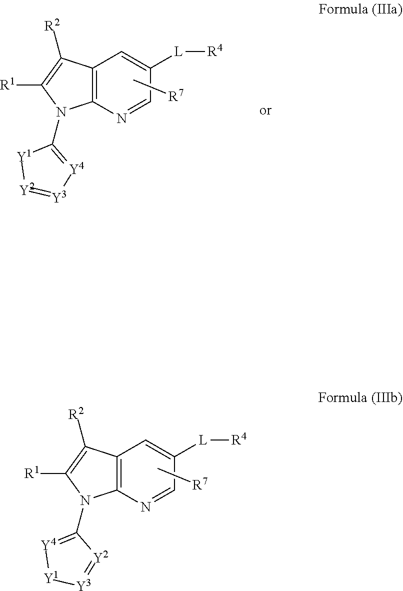

wherein,
Y¹ is O, S, or NR^{3D};
Y² is N or CR^{3A};
Y³ and Y⁴ are each independently N or CR^{3B};
R^{3A} and R^{3B} are each independently selected from H, halogen, —NR⁸R⁹, —OR¹⁰, —C(O)R¹⁰, —C(O)OR¹⁰, —C(O)NR⁸R⁹,-substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, or substituted or unsubstituted 5-membered heteroaryl; and
R^{3D} is H or C₁-C₆ alkyl.

In another aspect, provided herein is a pharmaceutical composition comprising a compound described herein, or a pharmaceutically acceptable salt or solvate thereof; and a pharmaceutically acceptable excipient.

In another aspect, provided herein is a method of promoting and/or stimulation skin pigmentation, comprising administering one or more of the compositions described herein to a subject in need thereof.

In another aspect, provided herein is a method of inhibiting hair loss, comprising administering one or more of the compositions described herein to a subject in need thereof.

method of preventing and/or treating skin inflammation and/or damage, comprising administering one or more of the compositions described herein to a subject in need thereof.

In another aspect, provided herein is a method of preventing and/or treating vascular insufficiency, comprising administering one or more of the compositions described herein to a subject in need thereof.

In another aspect, provided herein is a method of preventing, treating, minimizing and/or reversing congestive heart failure, cardiomyopathy, comprising administering one or more of the compositions described herein to a subject in need thereof.

In another aspect, provided herein is a method of reducing cardiac ejection fraction, comprising administering one or more of the compositions described herein to a subject in need thereof.

In another aspect, provided herein is a method of preventing and/or treating a gastrointestinal disease, comprising administering one or more of the compositions described herein to a subject in need thereof.

In another aspect, provided herein is a method of preventing and/or treating renal dysfunction, comprising administering one or more of the compositions described herein to a subject in need thereof.

In another aspect, provided herein is a method of stimulation bone resorption and bone formation, comprising administering one or more of the compositions described herein to a subject in need thereof.

In another aspect, provided herein is a method of stimulating tissue regeneration by stimulating, comprising administering one or more of the compositions described herein to a subject in need thereof.

In another aspect, provided herein is a method of modulating cervical ripening, comprising administering one or more of the compositions described herein to a subject in need thereof.

In another aspect, provided herein is a method of promoting neuroprotection and/or stimulating neuronal regeneration, comprising administering one or more of the compositions described herein to a subject in need thereof.

In another aspect, provided herein is a method of treating and/or preventing a neurological disorder, a neuropsychiatric disorder, a neural injury, a neural toxicity disorder, a neuropathic pain, or a neural degenerative disorder, comprising administering one or more of the compositions described herein to a subject in need thereof.

In another aspect, provided herein is a method of treating and/or preventing fibrotic or adhesion disease, disorder or condition, comprising administering one or more of the compositions described herein to a subject in need thereof.

In another aspect, provided herein is a method of reducing and/or preventing scar formation, comprising administering one or more of the compositions described herein to a subject in need thereof.

In another aspect, provided herein is a method of treating and/or preventing muscle disorder, muscle injury and/or muscle atrophy, comprising administering one or more of the compositions described herein to a subject in need thereof.

In another aspect, provided herein is a method of treating and/or preventing fibrosis, comprising administering one or more of the compositions described herein to a subject in need thereof.

In another aspect, provided herein is a method of treating and/or preventing idiopathic pulmonary fibrosis, comprising administering one or more of the compositions described herein to a subject in need thereof.

In another aspect, provided herein is a method of treating and/or preventing kidney fibrosis, comprising administering one or more of the compositions described herein to a subject in need thereof.

In another aspect, provided herein is a method of stimulating muscle regeneration, comprising administering one or more of said compositions described herein to a subject in need thereof.

In another aspect, provided herein is a method of promoting organ fitness, comprising administering one or more of said compositions described herein to a subject in need thereof.

In another aspect, provided herein is a method of promoting wound healing, comprising administering one or more of said compositions described herein to a subject in need thereof.

In another aspect, provided herein is a method of treating acute kidney injury, comprising administering one or more of said compositions described herein to a subject in need thereof.

In another aspect, provided herein is a method of treating sarcopenia, comprising administering one or more of said compositions described herein to a subject in need thereof.

In another aspect, provided herein is a method of treating a neuromuscular disease, comprising administering one or more of said compositions of any of the preceding claims to a subject in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

### Definitions

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Unless defined otherwise, all technical and scientific terms used herein have the same meaning as is commonly understood by one of skill in the art to which this invention belongs. All patents and publications referred to herein are incorporated by reference.

Unless the context requires otherwise, throughout the specification and claims which follow, the word “comprise” and variations thereof, such as, “comprises” and “comprising” are to be construed in an open, inclusive sense, that is, as “including, but not limited to” Further, headings provided herein are for convenience only and do not interpret the scope or meaning of the claimed invention.

Reference throughout this specification to “some embodiments” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to =O.

“C₈ rboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁-₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COON, COOMe, —OH, —OMe, —NH₂, or —NO₂.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂.

“Alkoxy” refers to a radical of the formula —OR where R is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2)π-electron system in accordance with the Hückel theory. The ring system from which aryl groups are derived include, but are not limited to, groups such as benzene, fluorene, indane, indene, tetralin and naphthalene. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂.

“C₈ rbocycle” refers to a saturated, unsaturated or aromatic rings in which each atom of the ring is carbon. C₈ rbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle may be optionally substituted.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-05 fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3-to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbomyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃—OH, —OMe, —NH₂, or —NO₂.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Halo” or “halogen” refers to bromo, chloro, fluoro or iodo. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc). When an alkyl group is substituted with more than one halogen radicals, each halogen may be independently selected e.g., 1-chloro, 2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₇ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂.

“Heteroaryl” or “aromatic heterocycle” refers to a radical derived from a heteroaromatic ring radical that comprises one to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2)π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, pyridine, pyrimidine, oxazole, furan, thiophene, benzthiazole, and imdazopyridine. An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, l-oxidopyrimidinyl, l-oxidopyrazinyl, l-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be unsubstituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc).

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—N₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)R^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜ N(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O) R^{a} (where t is 1 or 2), —R^{b}— S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, and heterocycle, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)O R^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O) R^{a} (where t is 1 or 2), —R^{b}—S(O)ₜR^{a} (where t is 1 or 2), —R^{b}—S(O)ₜOR^{a} (where t is 1 or 2) and —R^{b}—S(O)₁N(R^{a})₂ (where t is 1 or 2); and wherein each R^{b} is independently selected from a direct bond or a straight or branched alkylene, alkenylene, or alkynylene chain, and each R^{C} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Where substituent groups are specified by their conventional chemical formulae, written from left to right, they equally encompass the chemically identical substituents that would result from writing the structure from right to left, e.g., —CH₂O— is equivalent to —OCH₂—.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the C₈ hn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)—. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z— or E- form (or cis- or trans- form). Furthermore, some chemical entities may exist in various tautomeric forms. Unless otherwise specified, chemical entities described herein are intended to include all Z—, E- and tautomeric forms as well.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z— and E- forms (or cis- and trans- forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that may induce a particular response in target cells, e.g., reduction of platelet adhesion and/or cell migration. The specific dose may vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

The terms “antagonist” and “inhibitor” are used interchangeably, and they refer to a compound having the ability to inhibit a biological function (e.g., activity, expression, binding, protein-protein interaction) of a target protein or enzyme. Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

Whenever a protein is referred to herein, it will be understood that a single protein can be referred to by different names. For example, “15-PGDH”, “PGDH”, and “hPGDH” all refer to the same protein, 15-hydroxyprostaglandin dehydrogenase.

### Compounds

Provided herein are compounds and methods of inhibiting 15-hydroxyprostaglandin dehydrogenase (15-PGDH).

In an aspect, provided herein is a compound having the structure of Formula (Ia), or a pharmaceutically acceptable salt or solvate thereof:

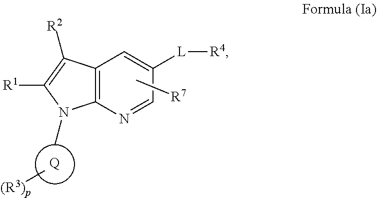

wherein,
ring Q is C₆-C₁₀ aryl or 5- to 10-membered heteroaryl;
L is —CR⁵R⁵—, —C(O)—, —S—, —S(O)—, or
R¹ and R² are each independently H, halogen, —CN, —OR¹⁰, —C(O)R¹⁰, —C(O)OR¹⁰, —NR⁸R⁹, —C(O)NR⁸R⁹, —NR¹⁰C(O)R¹⁰, substituted or unsubstituted C₁-C₆ alkyl, or substituted or unsubstituted C₃-C₈ cycloalkyl, substituted or unsubstituted alkenyl, or substituted or unsubstituted alkynyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —OC(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5-to 10-membered heteroaryl, wherein each or which is substituted or unsubstituted with one, two, or three R¹⁴;
R⁴ is substituted or unsubstituted C₁, —C₈ alkyl, substituted or unsubstituted C₂-C₈ alkenyl, substituted or unsubstituted C₁, —C₈ heteroalkyl, or substituted or unsubstituted C₁, —C₈ heteroalkyl, wherein each is substituted or unsubstituted with one, two, or three R¹⁴; or R⁴ is

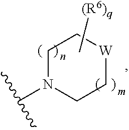

wherein
W is —CR⁶R⁶—, —C(O)R¹⁰—, —O—, —S—, —NR⁵—, —S(O)₂—, or —C(O)—;
   R⁵ is H or substituted or unsubstituted C₁-C₆ alkyl, substituted or unsubstituted cycloalkyl, or substituted or unsubstituted heterocycloalkyl;
   each R⁶ is independently H, halogen, CN, —NR⁸R⁹, —OR¹⁰, —C(O)R¹⁰, —C(O)OR¹⁰, —C(O)NR⁸R⁹, —SOR¹¹, —SO₂R¹¹, —NR⁸C(O)R⁹, —SR⁸, substituted or unsubstituted C₁-C₆ alkyl, or substituted or unsubstituted C₃-C₈ cycloalkyl;
   or two R⁶ can join together with the atom(s) to which they are attached to form a C₃-C₆ cycloalkyl or C₃-C₈ heterocycloalkyl ring;
   n and m are each independently 0, 1, 2, or 3; and
   g is 0, 1, 2, 3, 4, 5, or 6;
each R⁷ is independently H, halogen, —CN, —NR¹⁰R¹⁰, —OR¹⁰, —C(O)R¹⁰, —C(O)OR¹⁰, or substituted or unsubstituted C₁-C₆ alkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
each R¹¹ is independently selected from halogen, —CN, —NR⁸R⁹, —OR¹⁰, —C(O)R¹⁰, —C(O)OR¹⁰, —C(O)NR⁸R⁹, C₁-C₆ alkyl, C₂-C₆ alkenyl, C₂-C₆ alkynyl, C₁-C₆ heteroalkyl, C₁-C₆ haloalkyl, C₃-C₁₀ cycloalkyl, or C₃-C₁₀ heterocycloalkyl; and
p is 1,2,3, or 4.

In some embodiments, ring Q is an aryl or heteroaryl. In some embodiments, ring Q is aryl. In some embodiments, ring Q is a bicyclic or monocyclic heteroaryl. In some embodiments, ring Q is a bicyclic heteroaryl. In some embodiments, ring Q is a monocyclic heteroaryl. In some embodiments, ring Q is a 5- to 6-membered heteroaryl.

In some embodiments, ring Q is C₆ aryl. In some embodiments, ring Q is a 6-membered monocyclic heteroaryl. In some embodiments, ring Q is phenyl or a 6-membered monocyclic heteroaryl. In some embodiments, ring Q is phenyl, pyridinyl, pyrazinyl, pyrimidinyl, or pyridazinyl. In some embodiments, ring Q is phenyl or pyridinyl. In some embodiments, ring Q is phenyl. In some embodiments, ring Q is pyridinyl. In some embodiments, ring Q is pyrazinyl. In some embodiments, ring Q is pyrimidinyl. In some embodiments, ring Q is pyridazinyl.

In some embodiments, ring Q is:

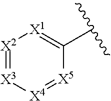

wherein,
X¹, X², X³, X⁴, and X⁵ are each independently N or CR³; and wherein at least two of X¹—X⁵ is CR³.

In some embodiments, X² is N; and X¹, X³, X⁴, and X⁵ are each CR³. In some embodiments, X³ is N; and X¹, X², X⁴, and X⁵ are each CR³. In some embodiments, X¹ is N; and X², X³, X⁴, and X⁵ are each CR³. In some embodiments, X² and X⁴ are each N; and X¹, X³ and X⁵ are each CR³. In some embodiments, X² and X³ are each N; and X¹, X⁴ and X⁵ are each CR³. In some embodiments, X¹ and X⁴ are each N; and X², X³ and X⁵ are each CR³. In some embodiments, X¹, X², and X⁴ are N; and X³ and X⁵ are each CR³.

In some embodiments, ring Q is:

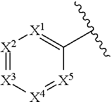

wherein,
X¹ and X⁵ are each independently N or CH;
X² is N or CR^{3A};
X³ is N or CR^{3B};
X⁴ is N, NR^{3C}, or CR^{3C}; and
R^{3A}, R^{3B}, and R^{3C} are each independently selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, —OC(O)NR⁸R⁹, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl.

In some embodiments, the compound has the structure of Formula (II), or a pharmaceutically acceptable salt or solvate thereof:

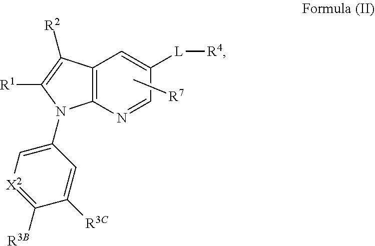

wherein, R^{3C}X² is N or CR^{3A}; and Formula (II),
R^{3A}, R^{3B}, and R^{3C} are each independently selected from H, halogen, —CN, —NR⁸R⁹, —OR¹⁰, CN, —C(O)R¹⁰,
C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5-to 10-membered heteroaryl;
provided that R^{3A}, R^{3B}, and R^{3C} are not all H at the same time.

In some embodiments, X² is N. In some embodiments, X² is CR^{3A}.

In some embodiments, X² is N; and R^{3B} and R^{3C} are each independently selected from H, halogen, —CN, —NR⁸R⁹, —OR¹⁰, —C(O)R¹⁰, —C(O)OR¹⁰, —C(O)NR⁸R⁹, —NR¹²C(O)OR¹⁰,-substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, or substituted or unsubstituted 5-membered heteroaryl.

In some embodiments, R^{3B} is H or halogen; and R^{C} is substituted or unsubstituted 5-membered heteroaryl. In some embodiments, R^{3B} is H, Br, C₁, or F; and R^{C} is substituted or unsubstituted 5-membered heteroaryl.

In some embodiments, ring Q is a 5-membered heteroaryl. In some embodiments, ring Q is triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl. In some embodiments, ring Q is imidazolyl, pyrazolyl, oxazolyl, isoxazolyl, thiophenyl, thiazolyl, isothiazolyl, triazolyl, oxadiazolyl, thiadiazolyl, or tetrazolyl. In some embodiments, ring Q is imidazolyl or pyrazolyl. In some embodiments, ring Q is imidazolyl. In some embodiments, ring Q is pyrazolyl. In some embodiments, ring Q is thiophenyl or thiazolyl. In some embodiments, ring Q is thiophenyl. In some embodiments, ring Q is thiazolyl.

In some embodiments, ring Q is:

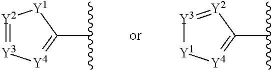

wherein,
Y¹ is O, S, or NR^{3D};
Y² is N or CR^{3A};
Y³ and Y³ are each independently N or CR^{3B};
R^{3A} and R^{3B} are each independently selected from H, halogen, —NR⁸R⁹, —OR¹⁰, —C(O)R¹⁰, —C(O)OR¹⁰, —C(O)NR⁸R⁹,-substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, or substituted or unsubstituted 5-membered heteroaryl; and
R^{3D} is H or C₁-C₆ alkyl.

In some embodiments, Y³ is 0 or S; Y² is CR^{3A}; and Y³ and Y⁴ are each independently N or CR^{3B}. In some embodiments, Y¹ is O; Y² is CR^{3A}; and Y³ and Y³ are each independently N or CR^{3B}. In some embodiments, Y³ is S; Y² is CR^{3A}; and Y³ and Y³ are each independently N or CR^{3B}.

In some embodiments, Y³ is 0 or S; Y² is N; and Y³ and Y⁴ are each independently N or CR^{3B}. In some embodiments, Y¹ is O; Y² is N; and Y³ and Y⁴ are each independently N or CR^{3B}. In some embodiments, Y³ is S; Y² is N; and Y³ and Y⁴ are each independently N or CR^{3B}.

In some embodiments, R^{3A}, R^{3B}, and R^{3C} are each independently selected from H, halogen, —NR⁸R⁹, —OR¹⁰, —C(O)R¹⁰, —C(O)OR¹⁰, —C(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, or substituted or unsubstituted 5-membered heteroaryl. In some embodiments, R^{3A}, R^{3B}, and R^{3C} are each independently selected from —NR⁸R⁹, —OR¹⁰, —C(O)R¹⁰, —C(O)OR¹⁰, —C(O)NR⁸R⁹. In some embodiments, R^{3A}, R^{3B}, and R^{3C} are each independently selected from substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl. In some embodiments, R^{3A}, R^{3B}, and R^{3C} are each independently selected from substituted or unsubstituted 5-membered heteroaryl. In some embodiments, R^{3A}, R^{3B}, and R^{3C} are each independently selected from H or halogen.

In some embodiments, R^{3D} is H. In some embodiments, R^{3D} is C₁-C₆ alkyl.

In some embodiments, the compound has the structure of Formula (IIIa) or (IIIb), or a salt or solvate thereof:

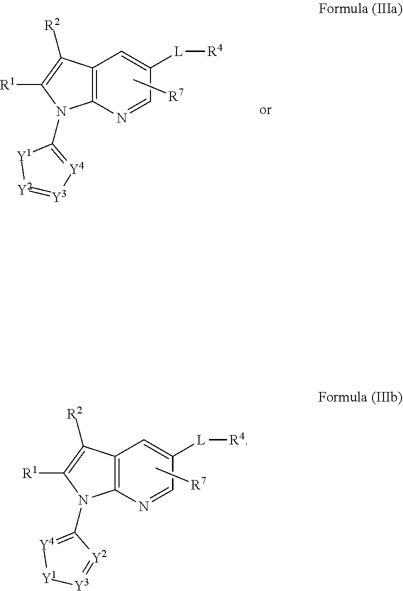

In some embodiments, the compound has the structure of Formula (IIIa), or a pharmaceutically acceptable salt or solvate thereof. In some embodiments, the compound has the structure of Formula (IIIb), or a pharmaceutically acceptable salt or solvate thereof.

In some embodiments, ring Q is a bicyclic heteroaryl. In some embodiments, ring Q is a bicyclic heteroaryl comprising 1-3 heteroatoms selected from N, O, and S atoms. In some embodiments, ring Q is a bicyclic heteroaryl comprising 1, 2, or 3 N atoms. In some embodiments, ring Q is [1,2,4]triazolo[1,5-a]pyridine.

In some embodiments, ring Q is

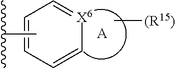

wherein,
ring A is a 5-membered heteroaryl optionally comprising 1 or 2 N atoms;
X⁶ is C or N; and
R¹⁵ is H, halogen, —NR⁸R⁹,-substituted or unsubstituted C₁-C₆ alkyl, substituted or unsubstituted C₁-C₆ haloalkyl, substituted or unsubstituted C₃-C₈ cycloalkyl, or substituted or unsubstituted C₃-C₈ heterocycloalkyl.

In some embodiments, ring A is imidazolyl, pyrazolyl, oxazolyl, isoxazolyl, thiophenyl, thiazolyl, isothiazolyl, triazolyl, oxadiazolyl, thiadiazolyl, or tetrazolyl. In some embodiments, ring Q is pyrazolyl. In some embodiments, ring Q is imidazolyl. In some embodiments, ring Q is triazolyl.

In some embodiments, X⁶ is C. In some embodiments, X⁶ is N.

In some embodiments, each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)NR⁸R⁹, —NR¹²C(O)OR¹⁰, —NR¹²SO₂R^{a}, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5-to 10-membered heteroaryl, substituted or unsubstituted C₂-C₈ alkenyl, or C₂-C₈ substituted or unsubstituted alkynyl. In some embodiment, each R³ is independently selected from H, halogen, —NR⁸R⁹, —OR¹⁰, CN, —C(O)R¹⁰, —C(O)OR¹⁰, —C(O)NR⁸R⁹, —NR¹²C(O)OR¹⁰, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5-membered heteroaryl.

In some embodiments, each R³ is independently selected from H, C₁, F, substituted or unsubstituted C₃-C₈ heterocycloalkyl, or substituted or unsubstituted 5-membered heteroaryl.

In some embodiments, each R³ is independently selected from substituted or unsubstituted C₃-C₈ heterocycloalkyl or substituted or unsubstituted 5-membered heteroaryl., substituted or unsubstituted with one or two —NH₂, CF₃, C₁-C₆ alkyl, or C₃-C₈ cycloalkyl. In some embodiments, each R³ is independently a substituted or unsubstituted 5-membered heteroaryl. In some embodiments, each R³ is independently triazinyl, pyrrolyl, imidazolyl, pyrazolyl, oxazolyl, isoxazolyl, thiophenyl, thiazolyl, isothiazolyl, triazolyl, oxadiazolyl, thiadiazolyl, or tetrazolyl, each of which is substituted or unsubstituted with one or two halogen, —NH₂, CF₃, C₁-C₆ alkyl, or C₃-C₈ cycloalkyl.

In some embodiments, each R³ is independently selected from the group consisting of

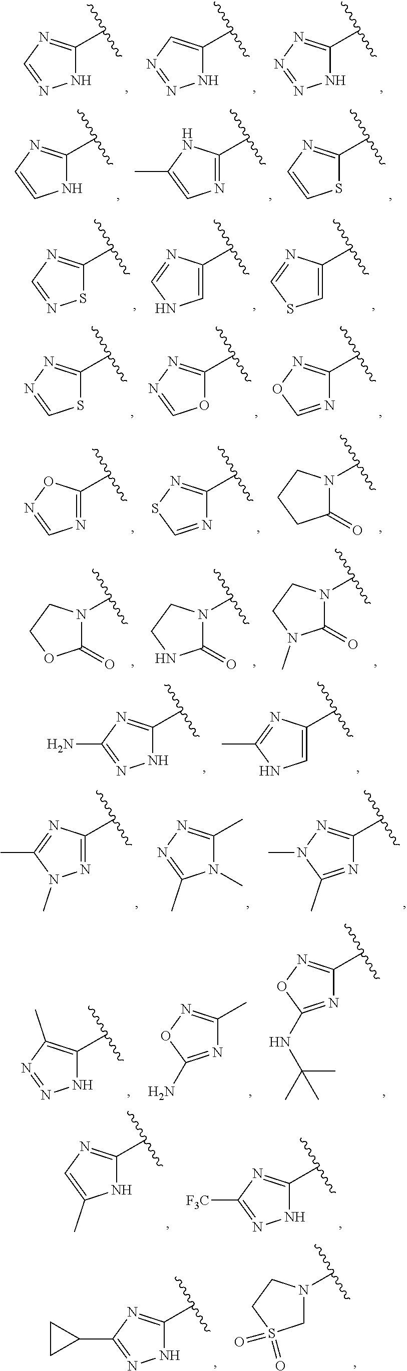

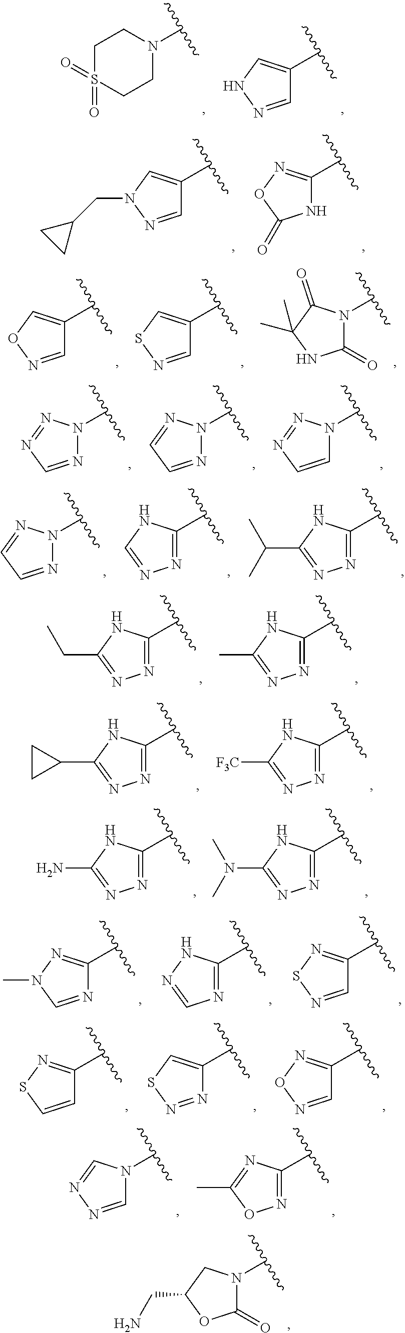

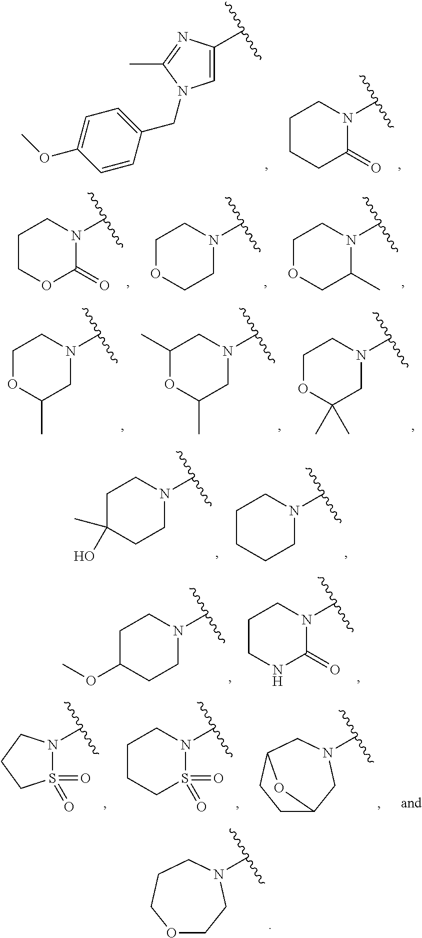

In some embodiments, each R³ is independently selected from the group consisting or

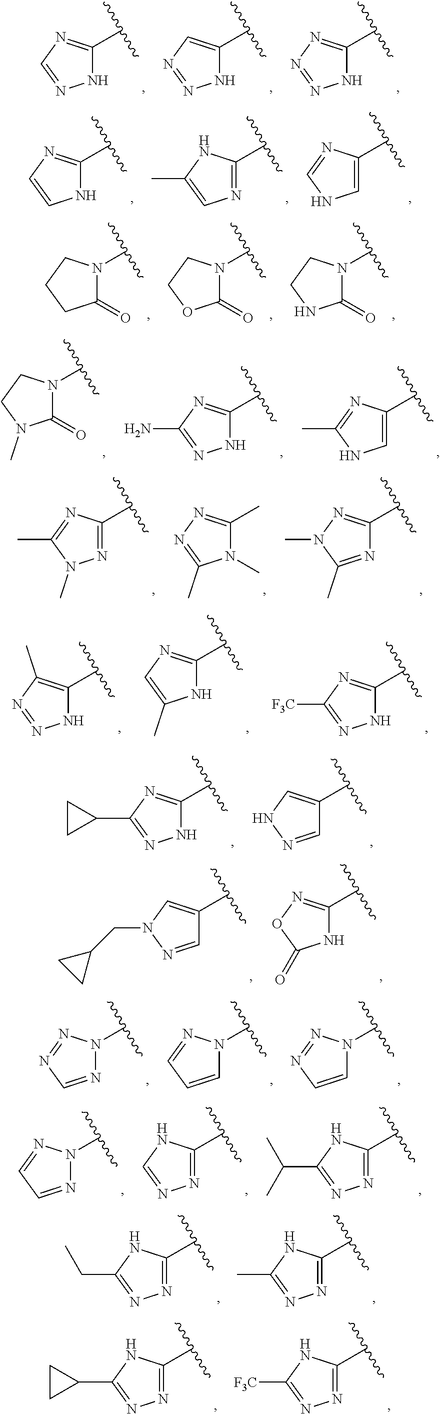

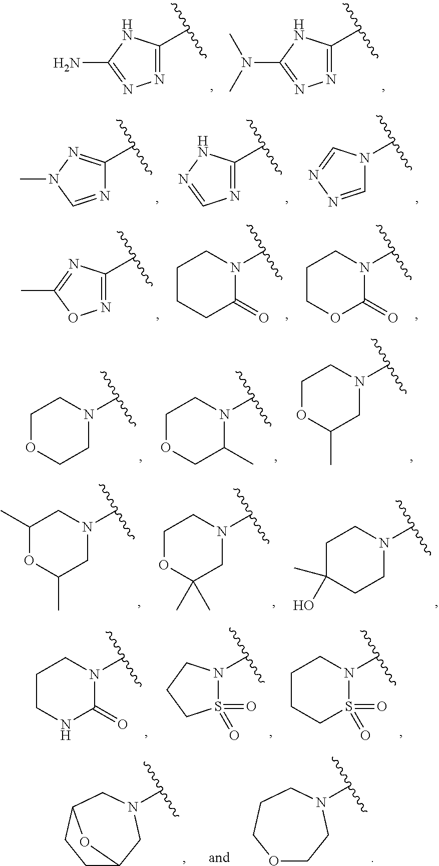

In some embodiments, each R³ is independently —C(O)R¹⁰, —C(O)OR¹⁰, —C(O)NR⁸R⁹, or —NR¹²C(O)OR¹⁰. In some embodiments, each R³ is independently —C(O)R¹⁰, —C(O)NR⁸R⁹, or —NR¹²C(O)OR¹⁰. In some embodiments, each R³ is independently —C(O)R¹⁰. In some embodiments, each R³ is independently —C(O)OR¹⁰. In some embodiments, each R³ is independently —C(O)NR⁸R⁹. In some embodiments, each R³ is independently —NR¹²C(O)OR¹⁰—.

In some embodiments, each R⁷ is independently selected from

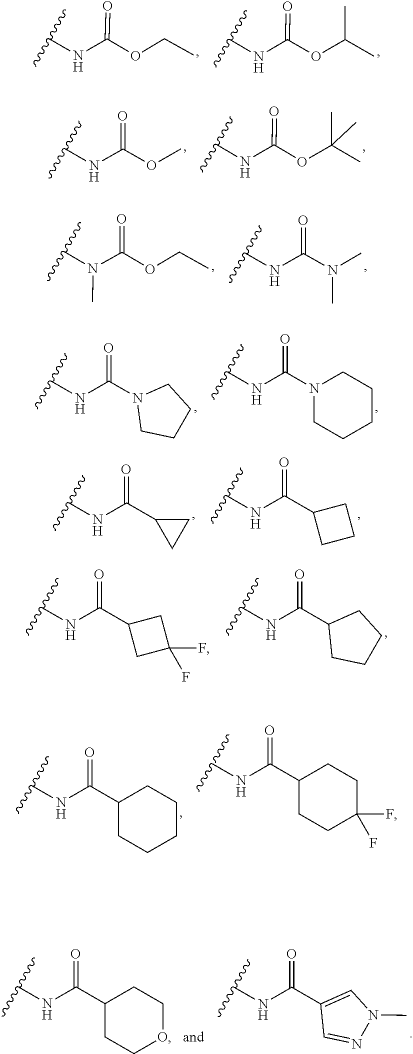

In some embodiments, L is —S—, —S(O)—, or —S(O)₂—. In some embodiments, L is —S—. In some embodiments, L is —S(O)—. In some embodiments, L is —S(O)₂—. In some embodiments, L is —C(O)—. In some embodiments, L is —O—. In some embodiments, L is —CR⁶R⁶—. In some embodiments, L is —C(O)R¹⁰—.

In some embodiments, Lis —S—, —S(O)—, or —S(O)₂—; and R⁴ is substituted or unsubstituted C₁-C₈ alkyl, substituted or unsubstituted C₂-C₈ alkenyl, substituted or unsubstituted C₁-C₈ heteroalkyl.

In some embodiments, Lis —S—, —S(O)—, or —S(O)₂—; and R⁴ is substituted or unsubstituted C₁-C₆ alkyl.

In some embodiments, R⁷ is substituted or unsubstituted C₁-C₈ alkyl, substituted or unsubstituted C₂-C₈ alkenyl, substituted or unsubstituted C₁-C₈ heteroalkyl. In some embodiments, R⁷ is substituted or unsubstituted C₁-C₈ alkyl. In some embodiments, R⁴ is substituted or unsubstituted C₂-C₈ alkenyl. In some embodiments, R⁴ is substituted or unsubstituted C₁-C₈ heteroalkyl.

In some embodiments, L is C(O); and R⁴

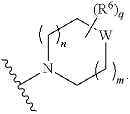

In another aspect, provided herein is a compound having the structure of Formula (N), or a pharmaceutically acceptable salt thereof:

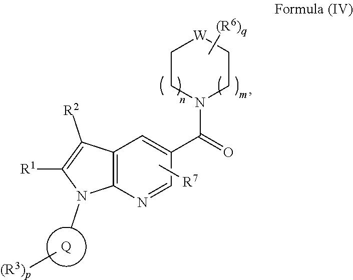

wherein,
ring Q is C₆ aryl or 5-to 10-membered heteroaryl;
W is —CR⁶R⁶—, —O—, —S—, —NR⁵—, —S(O)₂—, or —C(O)—;
R¹ and R² are each independently H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO²R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁵ is H or C₁-C₆ alkyl;
each R⁶ is independently H, halogen, CN, —NR⁸R⁹, —OR¹⁰, —C(O)R¹⁰, —C(O)OR¹⁰, —C(O)NR⁸R⁹, —SOR¹¹, —SO₂R¹¹, substituted or unsubstituted C₁-C₆ alkyl;
or two R⁶ can join together with the atom(s) to which they are attached to form a C₃-C₆ cycloalkyl or C₃-C₈ heterocycloalkyl ring;
R⁷ is H, halogen, —CN, —NR¹⁰R¹⁰, —OR¹⁰, —C(O)R¹⁰, —C(O)OR¹⁰, or substituted or unsubstituted C₁-C₆ alkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
n and m are each independently 0, 1, 2, or 3;
q is 0, 1, 2, or 3; and
p is 1,2,3, or 4.

In some embodiments, the compound has the structure of Formula (V-1), or a pharmaceutically acceptable salt or solvate thereof:

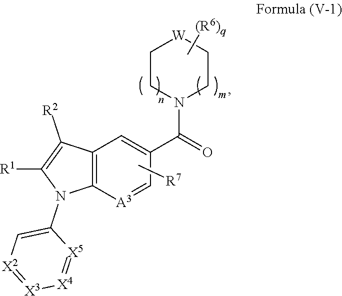

wherein,
X² is CH; Formula (V-1),
X³ is N or CR^{3B};
X⁴ is N or CR^{3C};
X⁵ is N or CR^{3F}, provided that one of one of X²—X⁵ must be N; and
R^{3B}, R^{3C}I, and R^{3F} are each independently H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5-to 10-membered heteroaryl.

In some embodiments, X² and X⁵ are each N; X³ is CR^{3B} and X⁴ is CR^{3C}. In some embodiments, X² and X⁴ are each N; X³ is CR^{3B} and X⁵ is CH. In some embodiments, X² is N; X³ is CR^{3B}; X⁴ is CR^{3C}; and X⁵ is CH. In some embodiments, X² and X⁵ are each CH; X³ is N; and X⁴ is CR^{3C}.

In some embodiments, the compound has the structure of Formula (V), or a pharmaceutically acceptable salt or solvate thereof:

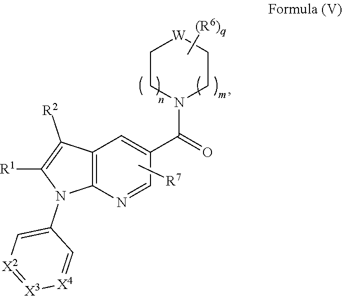

wherein,
X² is N, NR^{3A}, or CR^{3A};
X³ is N or CR^{3B};
X⁴ is N, NR^{3C}, or CR^{3C}; and
R^{3A}, R^{3B} and R^{3C} are each independently H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5-to 10-membered heteroaryl;
R^{3A} and R^{3B} together with the atoms to which they are attached form a substituted or unsubstituted 5 to 6-membered aryl or heteroaryl; or
R^{3B} and R^{3C} together with the atoms to which they are attached form a substituted or unsubstituted 5 to 6-membered aryl or heteroaryl;
wherein R^{3A}, R^{3B}, and R^{3C} are not all H at the same time.

In some embodiments, X² is N or CR^{3A}; X³ is N or CR^{3B}; and X⁴ is N or CR^{3C}.

In some embodiments, one of X², X³, or X⁴ is N. In some embodiments, two of X², X³, or X⁴ is N.

In some embodiments, X² is N; X³ is CR^{3B}; and X⁴ is CR^{3C}. In some embodiments, X² is CR^{3A}; X⁴ is N; X³ is CR^{3B}. In some embodiments, X² is CR^{3A}; X³ is CR^{3B}; and X⁴ is N.

In some embodiments, R^{3A} and R^{3B} together with the atoms to which they are attached form a substituted or unsubstituted 5 to 6-membered aryl or heteroaryl optionally comprising 1, 2, or 3 heteroatoms selected from O, S, and N. In some embodiments, R^{3A} and R^{3B} together with the atoms to which they are attached form a substituted or unsubstituted 5-membered heteroaryl comprising 1, 2, or 3 heteroatoms selected from N.

In some embodiments, R^{3B} and R^{3C} together with the atoms to which they are attached form a substituted or unsubstituted 5 to 6-membered aryl or heteroaryl optionally comprising 1, 2, or 3 heteroatoms selected from O, S, and N. In some embodiments, R^{3B} and R^{3C} together with the atoms to which they are attached form a substituted or unsubstituted 5-membered heteroaryl comprising 1, 2, or 3 heteroatoms selected from N.

In some embodiments, the compound has the structure of Formula (Va), or a pharmaceutically acceptable salt or solvate thereof:

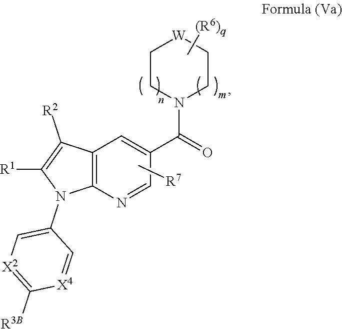

wherein,
X² is N or CR^{3A}; and
X⁴ is Nor CR^{3C}; and
R^{3A}, R^{3B} and R^{3C} are each independently H, halogen, —CN, —NR⁸R⁹, —OR¹⁰, CN, —C(O)R¹⁰, —C(O)OR¹⁰, —C(O)NR⁸R⁹, —NR¹²C(O)R¹⁰, —NR¹²C(O)OR¹⁰, —NR¹²C(O)NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
wherein R^{3A}, R^{3B}, and R^{3C} are not each H.

In some embodiments, X² is N and X⁴ is CR^{3C}. In some embodiments, R^{3B} is H, and R^{3C} is —C(O)R¹⁰, —C(O)OR¹⁰, —C(O)NR⁸R⁹, —NR¹²C(O)R¹⁰, —NR¹²C(O)OR¹⁰, substituted or unsubstituted C₃-C₈ heterocycloalkyl, or substituted or unsubstituted 5-membered heteroaryl. In some embodiments, R^{3C} is H, and R^{3B} is —C(O)R¹⁰, —C(O)OR¹⁰, —C(O)NR⁸R⁹, —NR¹²C(O)R¹⁰, —NR¹²C(O)OR¹⁰, substituted or unsubstituted C₃-C₈ heterocycloalkyl, or substituted or unsubstituted 5-membered heteroaryl.

In some embodiments, X² is C^{3A} and X⁴ is N. In some embodiments, R^{3A} is H, and R^{3B} is —C(O)R¹⁰, —NR¹²C(O)R¹⁰, —NR¹²C(O)OR¹⁰, substituted or unsubstituted C₃-C₈ heterocycloalkyl, or substituted or unsubstituted 5-membered heteroaryl. In some embodiments, R^{3B} is H, and R^{3A} is —C(O)R¹⁰, —C(O)OR¹⁰, —C(O)NR⁸R⁹, —NR¹²C(O)R¹⁰, —NR¹²C(O)OR¹⁰, substituted or unsubstituted C₃-C₈ heterocycloalkyl, or substituted or unsubstituted 5-membered heteroaryl.

In some embodiments, X² is CR^{3A} and X⁴ is CR^{3C}. In some embodiments, R^{3A} and R^{3B} are each H; and R^{3C} is —C(O)R¹⁰, —NR¹²C(O)R¹⁰, —NR¹²C(O)OR¹⁰, substituted or unsubstituted C₃-C₈ heterocycloalkyl, or substituted or unsubstituted 5-membered heteroaryl. In some embodiments, R^{3A} and R^{3C} are each H; and R^{3B} is —C(O)R¹⁰, —NR¹²C(O)R¹⁰, —NR¹²C(O)OR¹⁰, substituted or unsubstituted C₃-C₈ heterocycloalkyl, or substituted or unsubstituted 5-membered heteroaryl. In some embodiments, R^{3B} and R^{3C} are each H; and R^{3A} is —C(O)R¹⁰, —NR¹²C(O)R¹⁰, —NR¹²C(O)OR¹⁰, substituted or unsubstituted C₃-C₈ heterocycloalkyl, or substituted or unsubstituted 5-membered heteroaryl.

In some embodiments, the compound has the structure of Formula (Vb), or a pharmaceutically acceptable salt or solvate thereof:

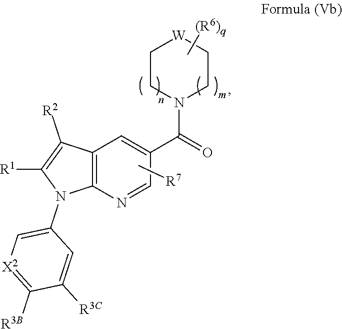

wherein,
X² is N; and
R^{3B} is H or halogen and R^{3C} is —C(O)R¹⁰, —C(O)OR¹⁰, —C(O)NR⁸R⁹, —NR¹²C(O)R¹⁰, —NR¹²C(O)OR¹⁰, —NR¹²C(O)NR⁸R⁹, substituted or unsubstituted C₃-C₈ heterocycloalkyl, or substituted or unsubstituted 5-membered heteroaryl; or
R^{3C} is H or halogen and R^{3B} is —C(O)R¹⁰, —C(O)OR¹⁰, —C(O)NR⁸R⁹, —NR¹²C(O)R¹⁰, —NR¹²C(O)OR¹⁰, —NR¹²C(O)NR⁸R⁹, substituted or unsubstituted C₃-C₈ heterocycloalkyl, or substituted or unsubstituted 5-membered heteroaryl.

In some embodiments, R³¹ is H, and R^{3B} is substituted or unsubstituted C₃-C₈ heterocycloalkyl, or substituted or unsubstituted 5-to 10-membered heteroaryl. In some embodiments, R^{3A} is substituted or unsubstituted C₃-C₈ heterocycloalkyl, or substituted or unsubstituted 5-to 10-membered heteroaryl, and R^{3B} is H.

In some embodiments, one of R^{3A}, R^{3B}, or R^{3C} is a substituted or unsubstituted 5-membered heteroaryl. In some embodiments, the 5-membered heteroaryl is triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl.

In some embodiments, one of R^{3A}, R^{3B} or R^{3C} is represented by moiety:

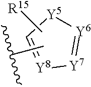

wherein,
Y⁵ is NR^{15A}, S, or O;
Y⁶, Y⁷, and Y⁵ are each independently N or CR¹⁵;
R¹⁵ is H, halogen, —NR⁸R⁹, —C₁-C₆ alkyl, C₁-C₆ haloalkyl, substituted or unsubstituted C₃-C₈ cycloalkyl, or substituted or unsubstituted C₃-C₈ heterocycloalkyl; and
R^{15A} is H or C₁-C₆ alkyl.

In some embodiments, one of R^{3A}, R^{3B}, or R^{3C} is represented by moiety:

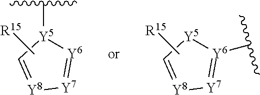

wherein

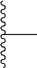

represents the connection point to R^{3A}, R^{3B}, or R^{3C}.

In some embodiments, the compound has the structure of Formula (VI), or a pharmaceutically acceptable salt or solvate thereof:

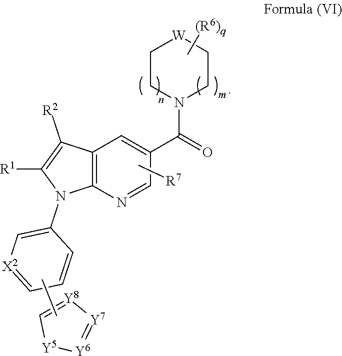

In some embodiments, the compound has the structure of Formula (VIa) or (VIb), or a pharmaceutically acceptable salt or solvate thereof:

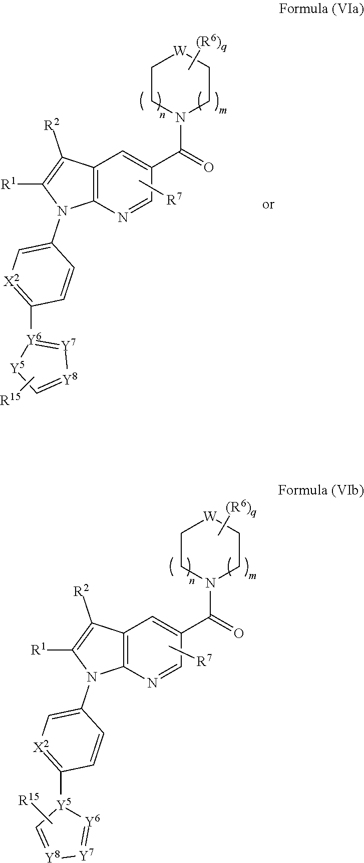

In some embodiments, the compound has the structure of Formula (VIc) or (VId), or a pharmaceutically acceptable salt or solvate thereof:

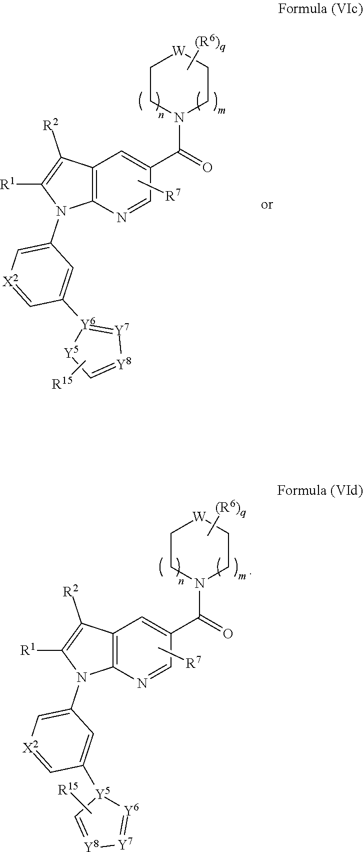

In some embodiments, the compound has the structure of Formula (VIa), or a pharmaceutically acceptable salt or solvate thereof. In some embodiments, the compound has the structure of Formula (VIb), or a pharmaceutically acceptable salt or solvate thereof. In some embodiments, the compound has the structure of Formula (VIc), or a pharmaceutically acceptable salt or solvate thereof. In some embodiments, the compound has the structure of Formula (VId), or a pharmaceutically acceptable salt or solvate thereof:
In some embodiments, Y⁵ is S, or O. In some embodiments, Y⁵ is NR^{15A} In some embodiments, Y⁵ is NH. In some embodiments, Y⁵ is NCH₃.

In some embodiments, Y⁷, and Y⁸ are each N. In some embodiments, Y⁶ is N. In some embodiments, Y⁶ is CR¹⁵. In some embodiments, Y⁷ is N. In some embodiments, Y⁷ is CR¹⁵. In some embodiments, Y⁵ is N. In some embodiments, Y⁸ is CR¹⁵.

In some embodiments, the compound has the structure of Formula (VIIa), or a pharmaceutically acceptable salt or solvate thereof:

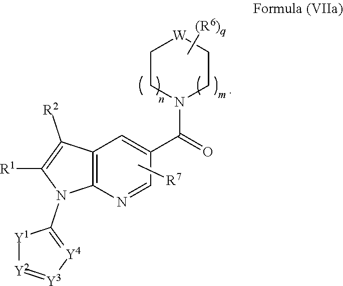

In some embodiments, the compound has the structure of Formula (VIIb), or a pharmaceutically acceptable salt or solvate thereof:

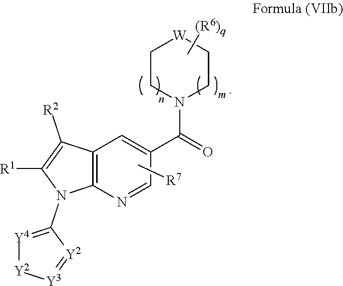

In some embodiments, the compound has the structure of Formula (VIII), or a pharmaceutically acceptable salt or solvate thereof.

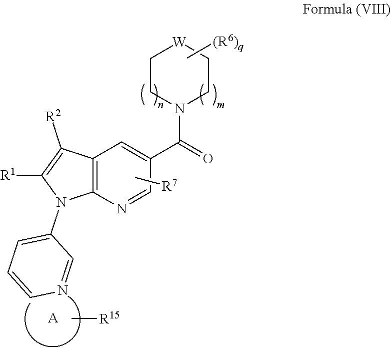

wherein,
ring A is a 5-membered heteroaryl optionally comprising 1 or 2 N atoms; and
R¹⁵ is H, halogen, —NR⁸R⁹,-substituted or unsubstituted C₁-C₆ alkyl, substituted or unsubstituted C₁-C₆ haloalkyl, substituted or unsubstituted C₃-C₈ cycloalkyl, or substituted or unsubstituted C₃-C₈ heterocycloalkyl.

In some embodiments, R¹⁵ is H, halogen, —NR⁸R⁹, —C₁-C₆ alkyl, or C₁-C₆ haloalkyl. In some embodiments, R¹⁵ is H. In some embodiments, R¹⁵ is —NR⁸R⁹. In some embodiments, R¹⁵ is —NH₂, —NHCH₃, or —N(CH₃)₂. In some embodiments, R¹⁵ is —C₁-C₆ alkyl or C₁-C₆ haloalkyl. In some embodiments, R¹⁵ is —CH₃, —CH₂CH₃, —CH(CH₃)₂, —C(CH₃)₃, —CF₃, or CHF₂.

In some embodiments, R¹⁵ is substituted or unsubstituted C₃-C₈ cycloalkyl. In some embodiments, R¹⁵ is substituted or unsubstituted C₃-C₈ hetercycloalkyl. In some embodiments, R¹⁵ is

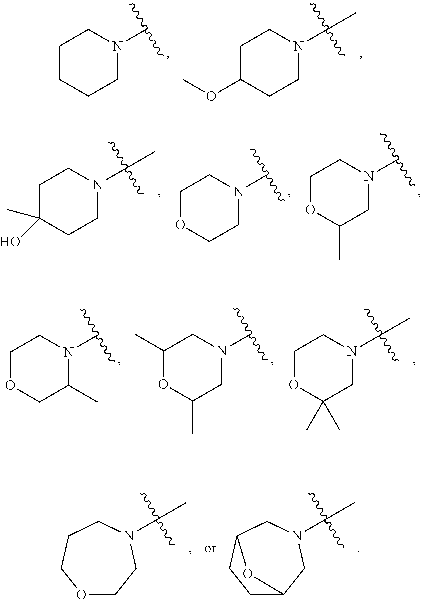

In some embodiments, the compound has the structure of Formula (IX), or a pharmaceutically acceptable salt or solvate thereof:

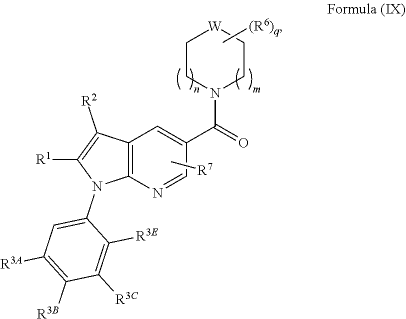

wherein,
R^{3A}, R^{3B}, R^{3C} and R^{3E} are each independently H, halogen, —C(O)R¹⁰, —C(O)OR¹⁰, —C(O)NR⁸R⁹, —NR¹²C(O)OR¹⁰, —NR¹²C(O)NR⁸R⁹, substituted or unsubstituted C₃-C₈ heterocycloalkyl, or substituted or unsubstituted 5-membered heteroaryl;
provided that one of R^{3A}, R^{3B}, R^{3C}, or R^{3D} is not H.

In some embodiments, R^{3A}, R^{3B}, R^{3C} and R^{3E} are each independently H, halogen, —C(O)R¹⁰, —C(O)NR⁸R⁹, —NR¹²C(O)OR¹⁰, substituted or unsubstituted C₃-C₈ heterocycloalkyl, or substituted or unsubstituted 5-membered heteroaryl.

In some embodiments, W is —CR⁶R⁶—, —O—, —S—, —NR⁵—, or —S(O)₂—. In some embodiments, W is —O—, —S— or —S(O)₂—. In some embodiments, W is —O—. In some embodiments, W is —S—. In some embodiments, W is —NR⁵—. In some embodiments, W is —S(O)₂—.

In some embodiments, W is —CR⁶R⁶—. In some embodiments, W is —CH₂—. In some embodiments, W is —CF₂—. In some embodiments, W is —CHF—.

In some embodiments, each R⁶ is independently H, halogen, CN, —NR⁸R⁹, —OR¹⁰, —C(O)R¹⁰, —C(O)OR¹⁰, —C(O)NR⁸R⁹, —NR⁸C(O)R¹¹—SOR¹¹, —SO₂R¹¹, —SR¹¹, substituted or unsubstituted C₁-C₆ alkyl, or C₃-C₈ cycloalkyl.

In some embodiments, each R⁶ is independently H, halogen, CN, —NR⁸R⁹, —OR¹⁰, —C(O)R¹⁰, —C(O)OR¹⁰, —C(O)NR⁸R⁹, or substituted or unsubstituted C₁-C₆ alkyl. In some embodiments, each R⁶ is halogen.

In some embodiments, each R⁶ is independently F, —NH₂, —OH, —OCH₃, or —CH₃. In some embodiments, each R⁶ is independently F. In some embodiments, each R⁶ is independently —CH₃.

In some embodiments, two R⁶ can join together with the atom(s) to which they are attached to form a C₃-C₆ cycloalkyl ring. In some embodiments, two R⁶ on the same carbon atom can join together to form a cycloalkyl ring. In some embodiments, two R⁶ on different carbon atoms can join together to form a cycloalkyl ring. In some embodiments, the ring is a spirocycle. In some embodiments, two R⁶ join together to form a cyclopropyl, cyclobutyl, cyclopentyl or cyclohexyl. In some embodiments, two R⁶ join together to form a cyclopropyl.

In some embodiments, R⁷ is H, halogen, —OR¹⁰, —C(O)R¹⁰, —C(O)OR¹⁰, or substituted or unsubstituted C₁-C₆alkyl. In some embodiments, R⁷ is H.

In some embodiments,

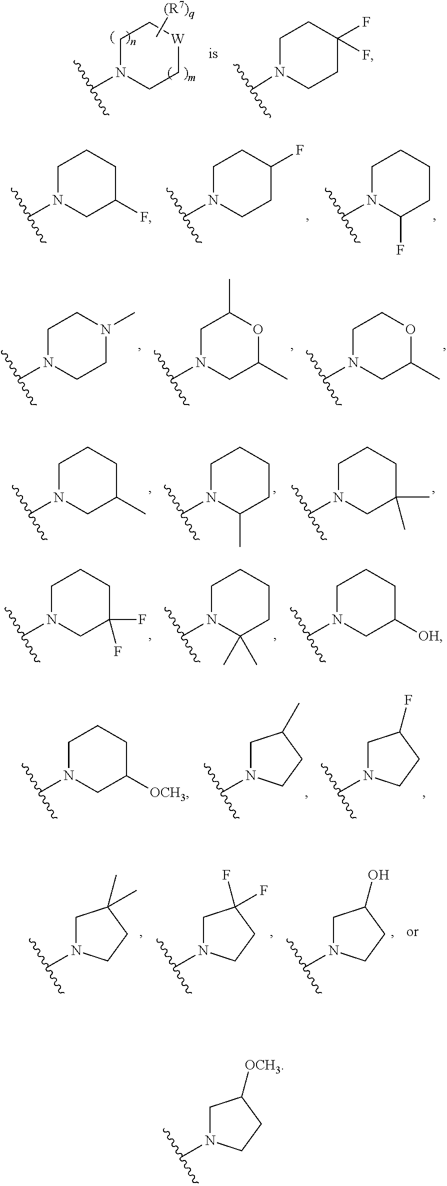

In some embodiments,

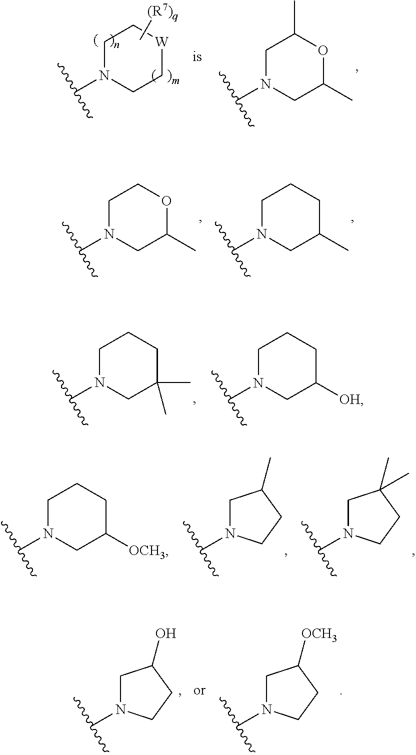

In some embodiments,

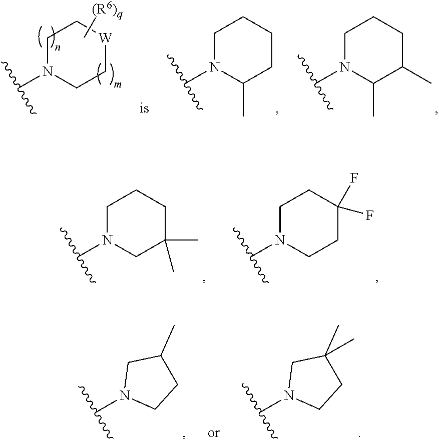

In some embodiments,

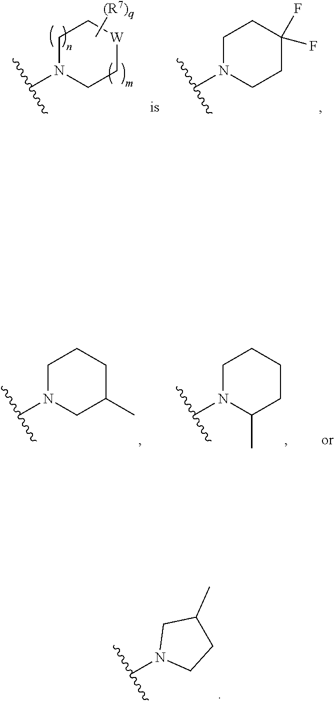

In some embodiments,

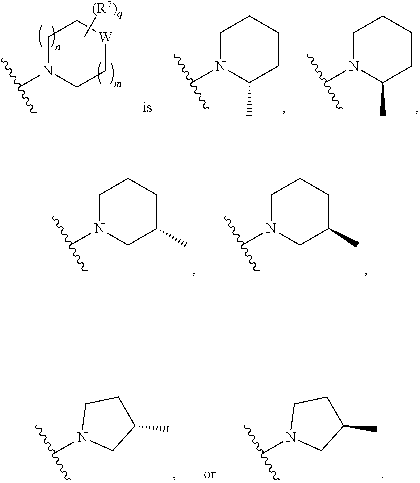

In some embodiments

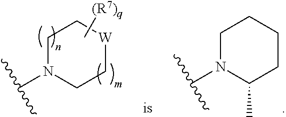

In some embodiments,

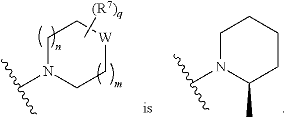

In some embodiments,

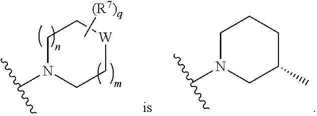

some embodiments,

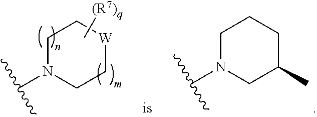

In some embodiments,

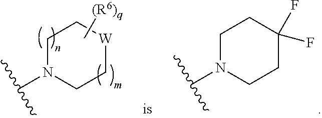

In some embodiments,

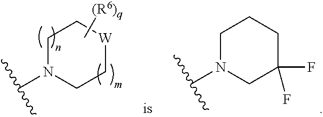

In some embodiments,

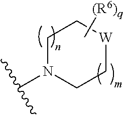

is not

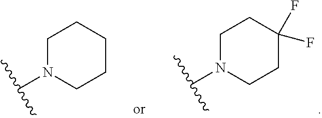 [SMILES: *N1CCCCC1 ; *N1CCC(F)(F)CC1]

In some embodiments, R¹ and R² are each independently H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl. In some embodiments, R¹ and R² are each independently H, halogen, —CN, —OR¹⁰, or —NR⁸R⁹. In some embodiments, R¹ and R² are each independently —C(O)R¹⁰, —C(O)OR¹⁰, or —C(O)NR⁸R⁹. In some embodiments, R¹ and R² are each independently substituted or unsubstituted C₁-C₆ alkyl, or substituted or unsubstituted C₃-C₈ cycloalkyl.

In some embodiments, R¹ is H; and R² is H.

In some embodiments, each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆haloalkyl, and C₃-C₁₀cycloalkyl. In some embodiments, each R⁸ and R⁹ are independently selected at each occurrence from H or C₁-C₆ alkyl. In some embodiments, each R⁸ and R⁹ are independently selected at each occurrence from H. In some embodiments, each R⁸ and R⁹ are independently selected at each occurrence from C₁-C₆ alkyl.

In some embodiments, each R¹⁰ is independently selected from H, C₁-C₆alkyl, C₂-C₆ alkenyl, C₁-C₆ heteroalkyl, C₁-C₆ haloalkyl, C₃-C₈ cycloalkyl, C₆-C₁₀ aryl, and 5-to 10-membered heteroaryl. In some embodiments, each R¹⁰ is independently selected from H or C₁-C₆ alkyl. In some embodiments, each R^{a}is independently selected from C₁-C₆haloalkyl. In some embodiments, each R¹⁰ is independently selected from C₃-C₈ cycloalkyl. In some embodiments, each R¹⁰ is independently selected from C₆-C₁₀ aryl and 5-to 10-membered heteroaryl. In some embodiments, each R¹⁰ is independently 5-membered heteroaryl.

In some embodiments, each R¹¹ is independently selected from C₁-C₆alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl. In some embodiments, each R¹¹ is independently selected from C₁-C₆alkyl. In some embodiments, each R¹¹ is selected from C₁-C₆ haloalkyl. In some embodiments, each R¹¹ is selected from C₃-C₈ cycloalkyl. In some embodiments, each R¹¹ is selected from C₆-C₁₀ aryl, and 5- to 10-membered heteroaryl.

In some embodiments, each R¹² is independently selected from H, C₁-C₆ alkyl, C₂-C₆ alkenyl, C₁-C₆ haloalkyl, and C₃-C₈ cycloalkyl. In some embodiments, each R¹² is independently selected from H or C₁-C₆alkyl. In some embodiments, each R¹² is independently selected from C₁-C₆haloalkyl. In some embodiments, each R¹² is independently selected from C₃-C₈ cycloalkyl.

In some embodiments, each R¹¹ is independently selected from —CN, —NR⁸R⁹, —OR¹⁰, —C(O)R¹⁰, —C(O)OR¹⁰, —C(O)NR⁸R⁹, C₁-C₆ alkyl, C₂-C₆ alkenyl, C₂-C₆ alkynyl, C₁-C₆ heteroalkyl, C₁-C₆ haloalkyl, C₃-C₁₀ cycloalkyl, or C₃-C₁₀ heterocycloalkyl. In some embodiments, each R¹¹ is independently selected from —NR⁸R⁹ or —OR¹⁰. In some embodiments, each R¹¹ is independently selected from —C(O)R¹⁰, —C(O)OR¹⁰, or —C(O)NR⁸R⁹. In some embodiments, each R¹¹ is independently selected from C₁-C₆ alkyl, C₂-C₆ alkenyl, C₂-C₆ alkynyl, C₁-C₆ heteroalkyl, or C₁-C₆ haloalkyl. In some embodiments, each R¹¹ is independently selected from C₃-C₁₀ cycloalkyl or C₃-C₁₀ heterocycloalkyl.

In some embodiments, p is 1, 2, 3, or 4. In some embodiments, p is 2 or 3. I some embodiments, p is 3. In some embodiments, p is 5. In some embodiments, p is 4. In some embodiments, p is 3. In some embodiments, p is 2. In some embodiments, p is 1.

In some embodiments, q is 0, 1, 2, 3, 4, 5, or 6. In some embodiments, q is 1 or 2. In some embodiments, q is 6. In some embodiments, q is 5. In some embodiments, q is 4. In some embodiments, q is 3. In some embodiments, q is 2. In some embodiments, q is 1. In some embodiments, q is 0.

In some embodiments, n and m are independently 0, 1, or 2. In some embodiments, n and m are independently 0. In some embodiments, n and m are independently 1. In some embodiments, n and m are independently 2. In some embodiments, n is 0, 1, or 2; and m is 1 or 2. In some embodiments, n is 0; and m is 1 or 2. In some embodiments, n is 1; and m is 1 or 2. In some embodiments, n is 2; and m is 1 or 2.

In some embodiments, the PDGH inhibitor is a compound presented in Table 1, or pharmaceutically acceptable salt or solvate thereof.

TABLE 1
Representative PGDH inhibitors.

| Compound | |
|---|---|
| No. | Structure |
| B-1 | 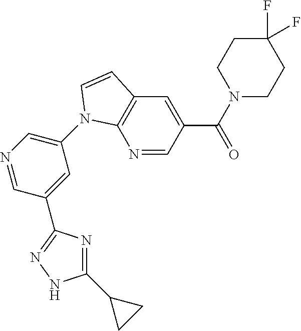 [SMILES: O=C(c1cnc2c(ccn2-c2cncc(-c3n[nH]c(C4CC4)n3)c2)c1)N1CCC(F)(F)CC1] |
| B-2 | 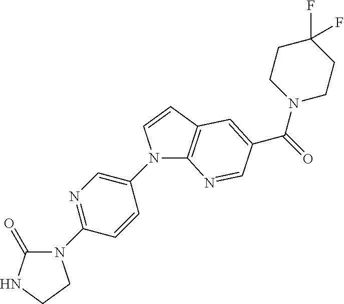 [SMILES: O=C(c1cnc2c(ccn2-c2ccc(N3CCNC3=O)nc2)c1)N1CCC(F)(F)CC1] |
| B-3 | 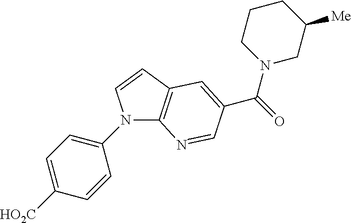 [SMILES: C[C@@H]1CCCN(C(=O)c2cnc3c(ccn3-c3ccc(C(=O)O)cc3)c2)C1] |
| B-4 | 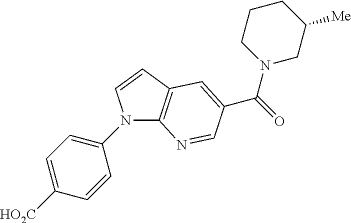 [SMILES: C[C@H]1CCCN(C(=O)c2cnc3c(ccn3-c3ccc(C(=O)O)cc3)c2)C1] |
| B-5 | 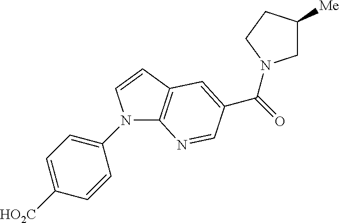 [SMILES: C[C@@H]1CCN(C(=O)c2cnc3c(ccn3-c3ccc(C(=O)O)cc3)c2)C1] |
| B-6 | 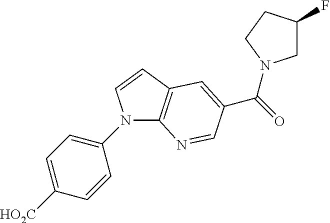 [SMILES: O=C(O)c1ccc(-n2ccc3cc(C(=O)N4CC[C@@H](F)C4)cnc32)cc1] |
| B-7 | 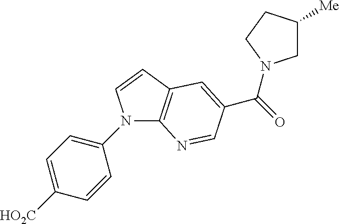 [SMILES: C[C@H]1CCN(C(=O)c2cnc3c(ccn3-c3ccc(C(=O)O)cc3)c2)C1] |
| B-8 | 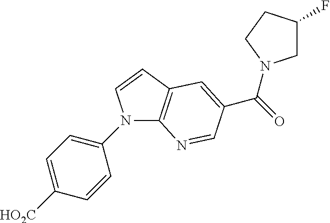 [SMILES: O=C(O)c1ccc(-n2ccc3cc(C(=O)N4CC[C@H](F)C4)cnc32)cc1] |
| B-9 | 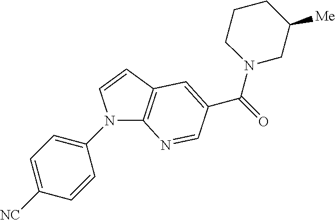 [SMILES: C[C@@H]1CCCN(C(=O)c2cnc3c(ccn3-c3ccc(C#N)cc3)c2)C1] |
| B-10 | 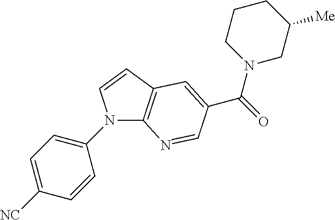 [SMILES: C[C@H]1CCCN(C(=O)c2cnc3c(ccn3-c3ccc(C#N)cc3)c2)C1] |
| B-11 | 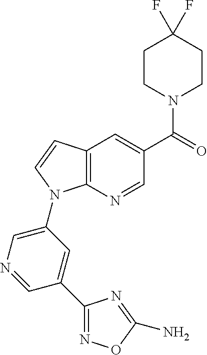 [SMILES: Nc1nc(-c2cncc(-n3ccc4cc(C(=O)N5CCC(F)(F)CC5)cnc43)c2)no1] |
| B-12 | 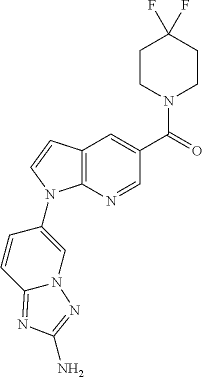 [SMILES: Nc1nc2ccc(-n3ccc4cc(C(=O)N5CCC(F)(F)CC5)cnc43)cn2n1] |
| B-13 | 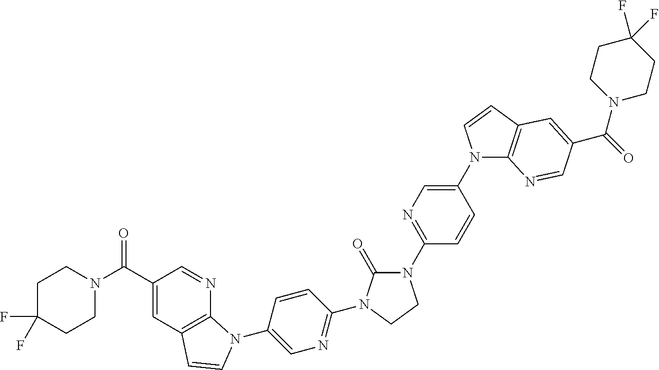 [SMILES: O=C(c1cnc2c(ccn2-c2ccc(N3CCN(c4ccc(-n5ccc6cc(C(=O)N7CCC(F)(F)CC7)cnc65)cn4)C3=O)nc2)c1)N1CCC(F)(F)CC1] |
| B-14 | 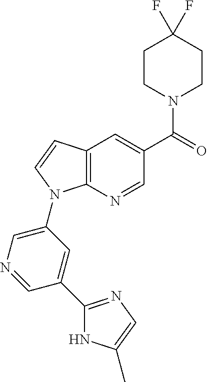 [SMILES: Cc1cnc(-c2cncc(-n3ccc4cc(C(=O)N5CCC(F)(F)CC5)cnc43)c2)[nH]1] |
| B-15 | 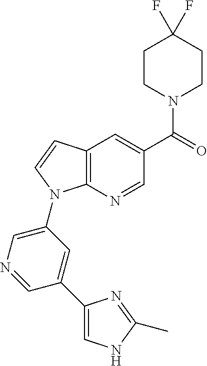 [SMILES: Cc1nc(-c2cncc(-n3ccc4cc(C(=O)N5CCC(F)(F)CC5)cnc43)c2)c[nH]1] |
| B-16 | 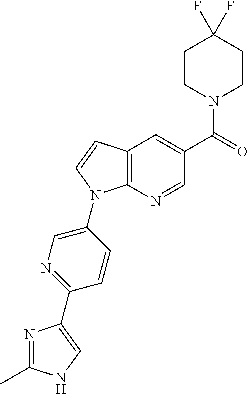 [SMILES: Cc1nc(-c2ccc(-n3ccc4cc(C(=O)N5CCC(F)(F)CC5)cnc43)cn2)c[nH]1] |
| B-17 | 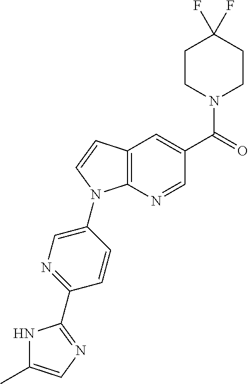 [SMILES: Cc1cnc(-c2ccc(-n3ccc4cc(C(=O)N5CCC(F)(F)CC5)cnc43)cn2)[nH]1] |
| B-18 | 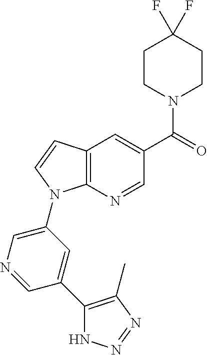 [SMILES: Cc1nn[nH]c1-c1cncc(-n2ccc3cc(C(=O)N4CCC(F)(F)CC4)cnc32)c1] |
| B-19 | 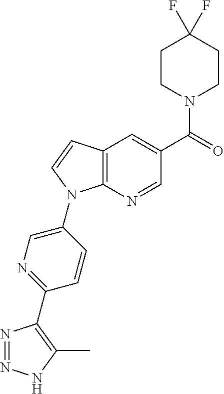 [SMILES: Cc1[nH]nnc1-c1ccc(-n2ccc3cc(C(=O)N4CCC(F)(F)CC4)cnc32)cn1] |
| B-20 | 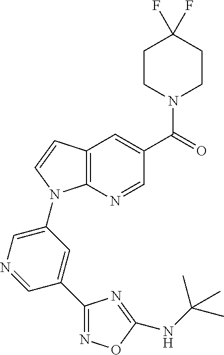 [SMILES: CC(C)(C)Nc1nc(-c2cncc(-n3ccc4cc(C(=O)N5CCC(F)(F)CC5)cnc43)c2)no1] |
| B-21 | 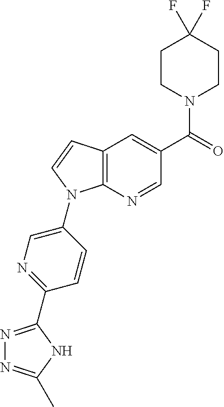 [SMILES: Cc1nnc(-c2ccc(-n3ccc4cc(C(=O)N5CCC(F)(F)CC5)cnc43)cn2)[nH]1] |
| B-22 | 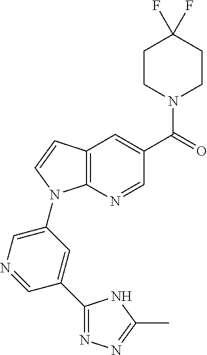 [SMILES: Cc1nnc(-c2cncc(-n3ccc4cc(C(=O)N5CCC(F)(F)CC5)cnc43)c2)[nH]1] |
| B-23 | 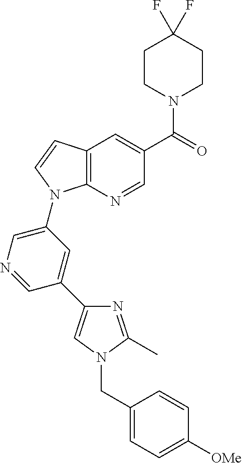 [SMILES: COc1ccc(Cn2cc(-c3cncc(-n4ccc5cc(C(=O)N6CCC(F)(F)CC6)cnc54)c3)nc2C)cc1] |
| B-24 | 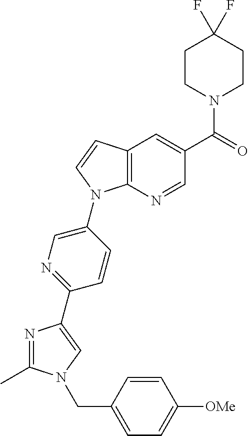 [SMILES: COc1ccc(Cn2cc(-c3ccc(-n4ccc5cc(C(=O)N6CCC(F)(F)CC6)cnc54)cn3)nc2C)cc1] |
| B-25 | 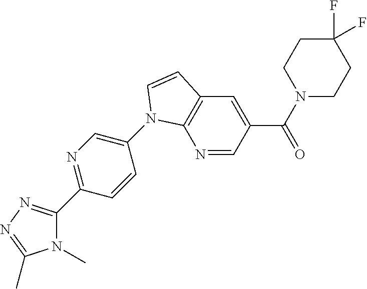 [SMILES: Cc1nnc(-c2ccc(-n3ccc4cc(C(=O)N5CCC(F)(F)CC5)cnc43)cn2)n1C] |
| B-26 | 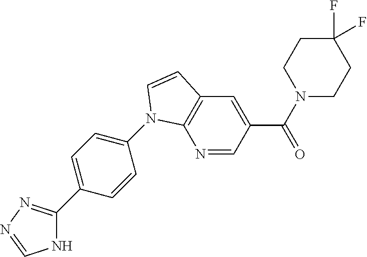 [SMILES: O=C(c1cnc2c(ccn2-c2ccc(-c3nnc[nH]3)cc2)c1)N1CCC(F)(F)CC1] |
| B-27 | 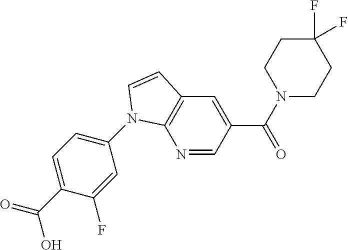 [SMILES: O=C(O)c1ccc(-n2ccc3cc(C(=O)N4CCC(F)(F)CC4)cnc32)cc1F] |
| B-28 | 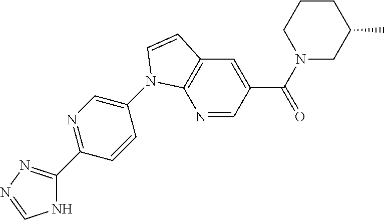 [SMILES: C[C@H]1CCCN(C(=O)c2cnc3c(ccn3-c3ccc(-c4nnc[nH]4)nc3)c2)C1] |
| B-29 | 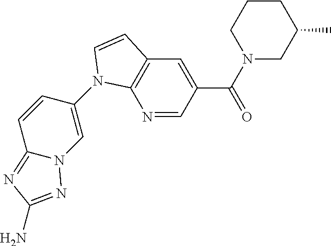 [SMILES: C[C@H]1CCCN(C(=O)c2cnc3c(ccn3-c3ccc4nc(N)nn4c3)c2)C1] |
| B-30 | 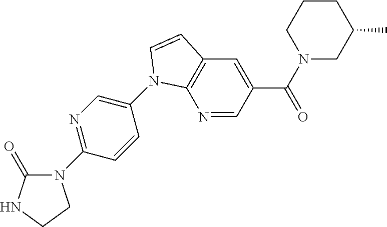 [SMILES: C[C@H]1CCCN(C(=O)c2cnc3c(ccn3-c3ccc(N4CCNC4=O)nc3)c2)C1] |
| B-31 | 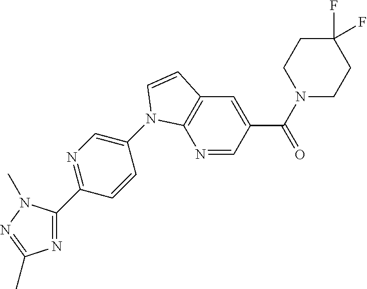 [SMILES: Cc1nc(-c2ccc(-n3ccc4cc(C(=O)N5CCC(F)(F)CC5)cnc43)cn2)n(C)n1] |
| B-32 | 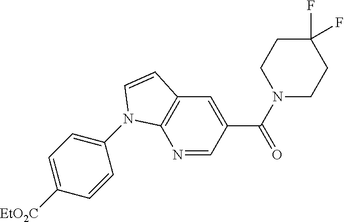 [SMILES: CCOC(=O)c1ccc(-n2ccc3cc(C(=O)N4CCC(F)(F)CC4)cnc32)cc1] |
| B-33 | 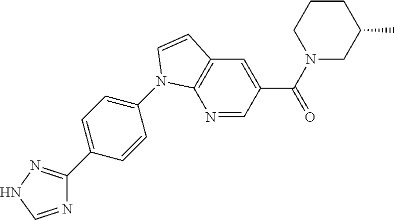 [SMILES: C[C@H]1CCCN(C(=O)c2cnc3c(ccn3-c3ccc(-c4nc[nH]n4)cc3)c2)C1] |
| B-34 | 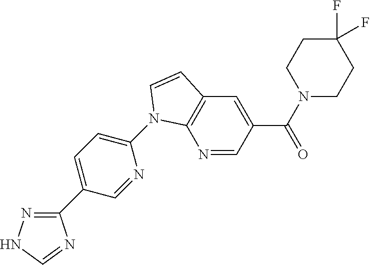 [SMILES: O=C(c1cnc2c(ccn2-c2ccc(-c3nc[nH]n3)cn2)c1)N1CCC(F)(F)CC1] |
| B-35 | 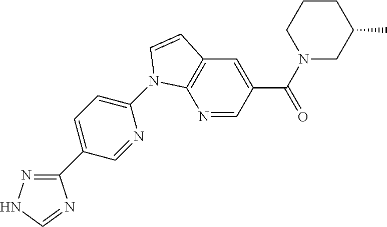 [SMILES: C[C@H]1CCCN(C(=O)c2cnc3c(ccn3-c3ccc(-c4nc[nH]n4)cn3)c2)C1] |
| B-36 | 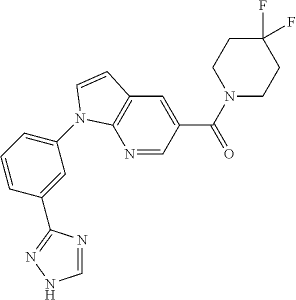 [SMILES: O=C(c1cnc2c(ccn2-c2cccc(-c3nc[nH]n3)c2)c1)N1CCC(F)(F)CC1] |
| B-37 | 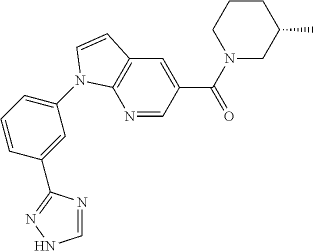 [SMILES: C[C@H]1CCCN(C(=O)c2cnc3c(ccn3-c3cccc(-c4nc[nH]n4)c3)c2)C1] |
| B-38 | 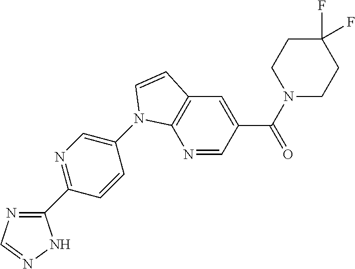 [SMILES: O=C(c1cnc2c(ccn2-c2ccc(-c3ncn[nH]3)nc2)c1)N1CCC(F)(F)CC1] |
| B-39 | 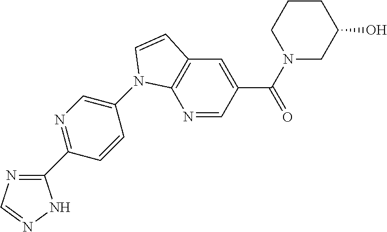 [SMILES: O=C(c1cnc2c(ccn2-c2ccc(-c3ncn[nH]3)nc2)c1)N1CCC[C@H](O)C1] |
| B-40 | 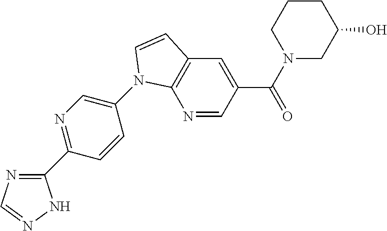 [SMILES: O=C(c1cnc2c(ccn2-c2ccc(-c3ncn[nH]3)nc2)c1)N1CCC[C@H](O)C1] |
| B-41 | 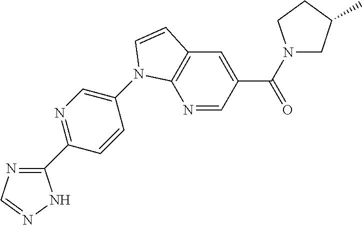 [SMILES: C[C@H]1CCN(C(=O)c2cnc3c(ccn3-c3ccc(-c4ncn[nH]4)nc3)c2)C1] |
| B-42 | 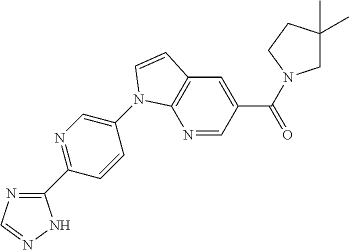 [SMILES: CC1(C)CCN(C(=O)c2cnc3c(ccn3-c3ccc(-c4ncn[nH]4)nc3)c2)C1] |
| B-43 | 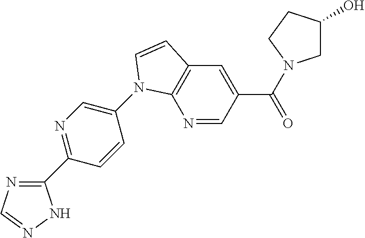 [SMILES: O=C(c1cnc2c(ccn2-c2ccc(-c3ncn[nH]3)nc2)c1)N1CC[C@H](O)C1] |
| B-44 | 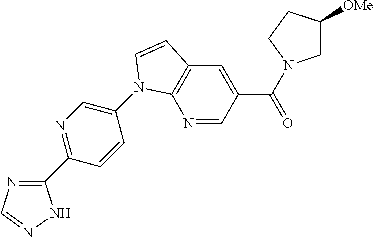 [SMILES: CO[C@@H]1CCN(C(=O)c2cnc3c(ccn3-c3ccc(-c4ncn[nH]4)nc3)c2)C1] |
| B-45 | 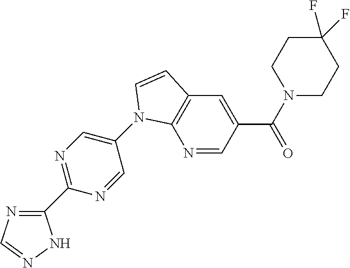 [SMILES: O=C(c1cnc2c(ccn2-c2cnc(-c3ncn[nH]3)nc2)c1)N1CCC(F)(F)CC1] |
| B-46 | 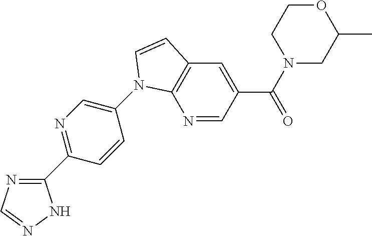 [SMILES: CC1CN(C(=O)c2cnc3c(ccn3-c3ccc(-c4ncn[nH]4)nc3)c2)CCO1] |
| B-47 | 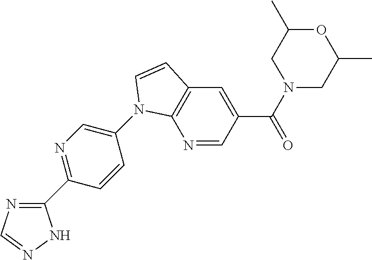 [SMILES: CC1CN(C(=O)c2cnc3c(ccn3-c3ccc(-c4ncn[nH]4)nc3)c2)CC(C)O1] |
| B-48 | 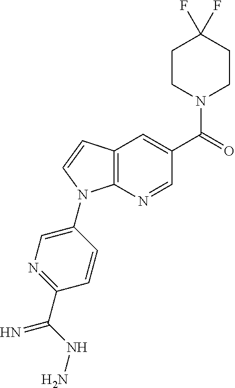 [SMILES: N=C(NN)c1ccc(-n2ccc3cc(C(=O)N4CCC(F)(F)CC4)cnc32)cn1] |
| B-49 | 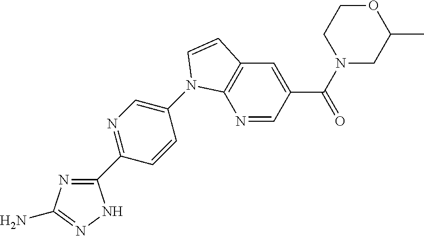 [SMILES: CC1CN(C(=O)c2cnc3c(ccn3-c3ccc(-c4nc(N)n[nH]4)nc3)c2)CCO1] |
| B-50 | 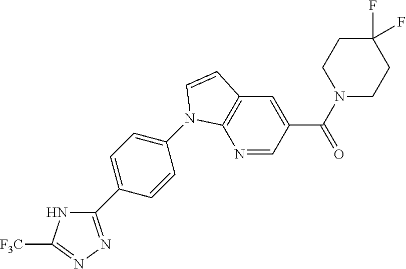 [SMILES: O=C(c1cnc2c(ccn2-c2ccc(-c3nnc(C(F)(F)F)[nH]3)cc2)c1)N1CCC(F)(F)CC1] |
| B-51 | 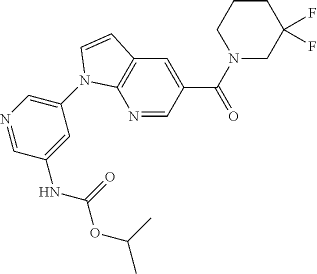 [SMILES: CC(C)OC(=O)Nc1cncc(-n2ccc3cc(C(=O)N4CCCC(F)(F)C4)cnc32)c1] |
| B-52 | 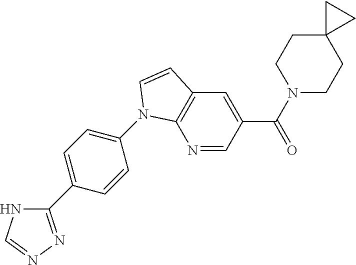 [SMILES: O=C(c1cnc2c(ccn2-c2ccc(-c3nnc[nH]3)cc2)c1)N1CCC2(CC1)CC2] |
| B-53 | 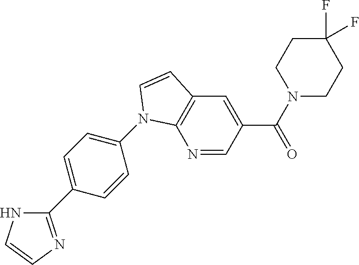 [SMILES: O=C(c1cnc2c(ccn2-c2ccc(-c3ncc[nH]3)cc2)c1)N1CCC(F)(F)CC1] |
| B-54 | 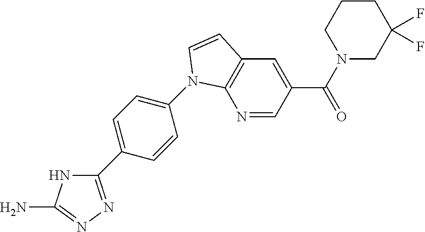 [SMILES: Nc1nnc(-c2ccc(-n3ccc4cc(C(=O)N5CCCC(F)(F)C5)cnc43)cc2)[nH]1] |
| B-55 | 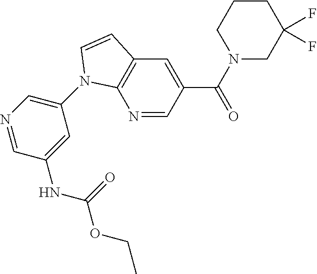 [SMILES: CCOC(=O)Nc1cncc(-n2ccc3cc(C(=O)N4CCCC(F)(F)C4)cnc32)c1] |
| B-56 | 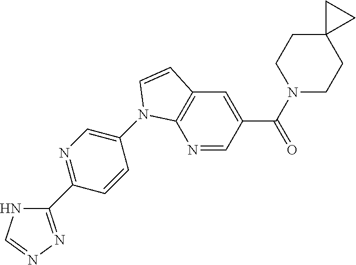 [SMILES: O=C(c1cnc2c(ccn2-c2ccc(-c3nnc[nH]3)nc2)c1)N1CCC2(CC1)CC2] |
| B-57 | 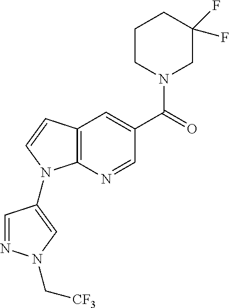 [SMILES: O=C(c1cnc2c(ccn2-c2cnn(CC(F)(F)F)c2)c1)N1CCCC(F)(F)C1] |
| B-58 | 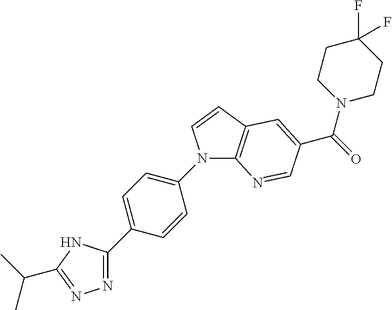 [SMILES: CC(C)c1nnc(-c2ccc(-n3ccc4cc(C(=O)N5CCC(F)(F)CC5)cnc43)cc2)[nH]1] |
| B-59 | 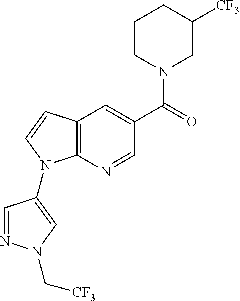 [SMILES: O=C(c1cnc2c(ccn2-c2cnn(CC(F)(F)F)c2)c1)N1CCCC(C(F)(F)F)C1] |
| B-60 | 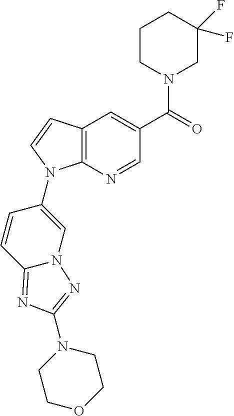 [SMILES: O=C(c1cnc2c(ccn2-c2ccc3nc(N4CCOCC4)nn3c2)c1)N1CCCC(F)(F)C1] |
| B-61 | 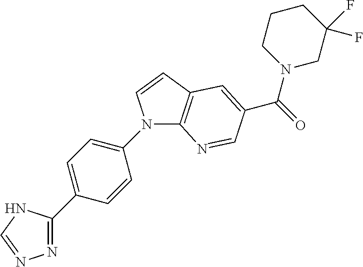 [SMILES: O=C(c1cnc2c(ccn2-c2ccc(-c3nnc[nH]3)cc2)c1)N1CCCC(F)(F)C1] |
| B-62 | 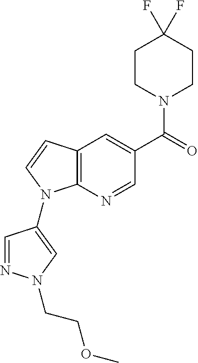 [SMILES: COCCn1cc(-n2ccc3cc(C(=O)N4CCC(F)(F)CC4)cnc32)cn1] |
| B-63 | 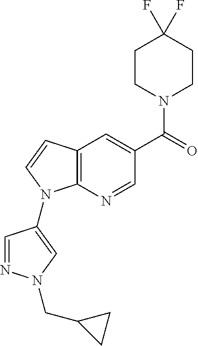 [SMILES: O=C(c1cnc2c(ccn2-c2cnn(CC3CC3)c2)c1)N1CCC(F)(F)CC1] |
| B-64 | 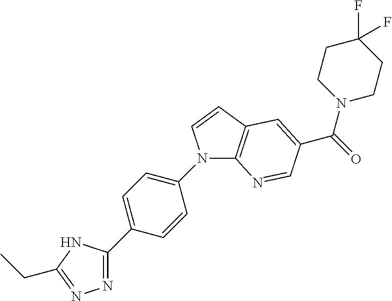 [SMILES: CCc1nnc(-c2ccc(-n3ccc4cc(C(=O)N5CCC(F)(F)CC5)cnc43)cc2)[nH]1] |
| B-65 | 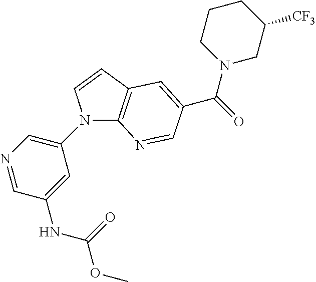 [SMILES: COC(=O)Nc1cncc(-n2ccc3cc(C(=O)N4CCC[C@H](C(F)(F)F)C4)cnc32)c1] |
| B-66 | 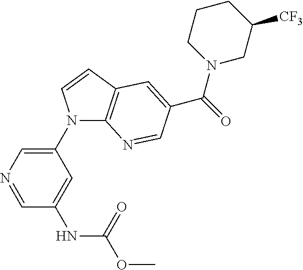 [SMILES: COC(=O)Nc1cncc(-n2ccc3cc(C(=O)N4CCC[C@@H](C(F)(F)F)C4)cnc32)c1] |
| B-67 | 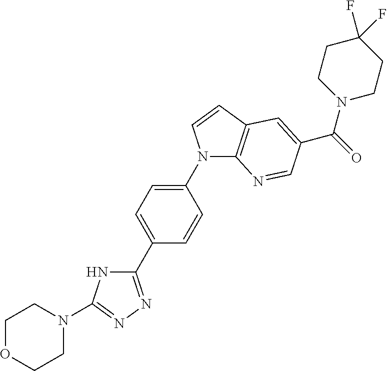 [SMILES: O=C(c1cnc2c(ccn2-c2ccc(-c3nnc(N4CCOCC4)[nH]3)cc2)c1)N1CCC(F)(F)CC1] |
| B-69 | 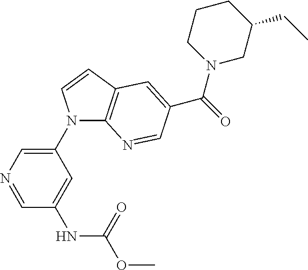 [SMILES: CC[C@H]1CCCN(C(=O)c2cnc3c(ccn3-c3cncc(NC(=O)OC)c3)c2)C1] |
| B-70 | 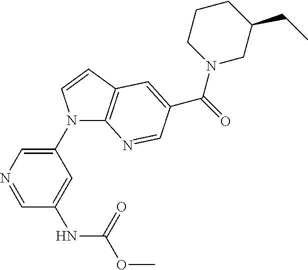 [SMILES: CC[C@@H]1CCCN(C(=O)c2cnc3c(ccn3-c3cncc(NC(=O)OC)c3)c2)C1] |
| B-71 | 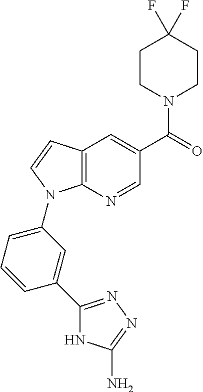 [SMILES: Nc1nnc(-c2cccc(-n3ccc4cc(C(=O)N5CCC(F)(F)CC5)cnc43)c2)[nH]1] |
| B-72 | 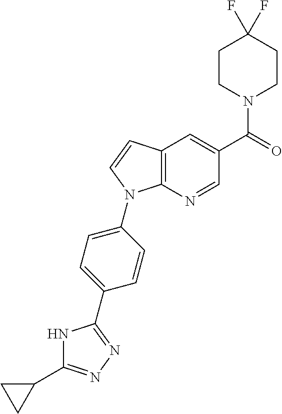 [SMILES: O=C(c1cnc2c(ccn2-c2ccc(-c3nnc(C4CC4)[nH]3)cc2)c1)N1CCC(F)(F)CC1] |
| B-73 | 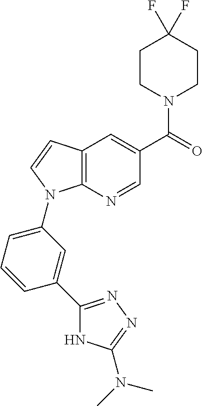 [SMILES: CN(C)c1nnc(-c2cccc(-n3ccc4cc(C(=O)N5CCC(F)(F)CC5)cnc43)c2)[nH]1] |
| B-74 | 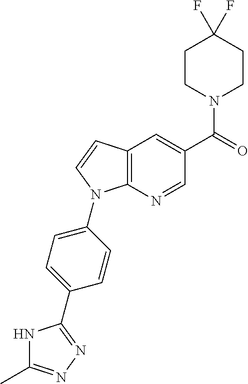 [SMILES: Cc1nnc(-c2ccc(-n3ccc4cc(C(=O)N5CCC(F)(F)CC5)cnc43)cc2)[nH]1] |
| B-75 | 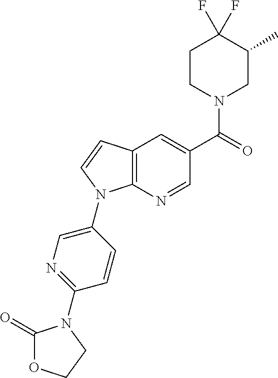 [SMILES: C[C@@H]1CN(C(=O)c2cnc3c(ccn3-c3ccc(N4CCOC4=O)nc3)c2)CCC1(F)F] |
| B-76 | 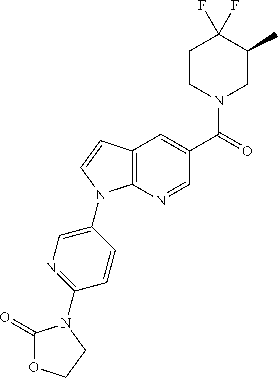 [SMILES: C[C@H]1CN(C(=O)c2cnc3c(ccn3-c3ccc(N4CCOC4=O)nc3)c2)CCC1(F)F] |
| B-77 | 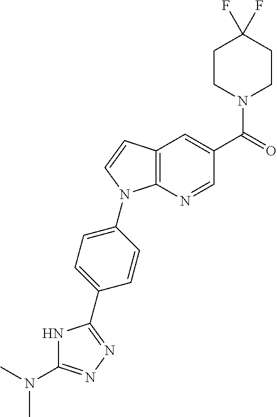 [SMILES: CN(C)c1nnc(-c2ccc(-n3ccc4cc(C(=O)N5CCC(F)(F)CC5)cnc43)cc2)[nH]1] |
| B-78 | 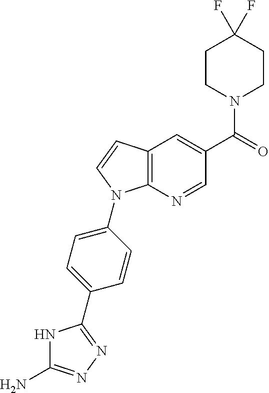 [SMILES: Nc1nnc(-c2ccc(-n3ccc4cc(C(=O)N5CCC(F)(F)CC5)cnc43)cc2)[nH]1] |
| B-79 | 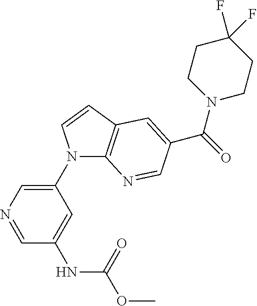 [SMILES: COC(=O)Nc1cncc(-n2ccc3cc(C(=O)N4CCC(F)(F)CC4)cnc32)c1] |
| B-80 | 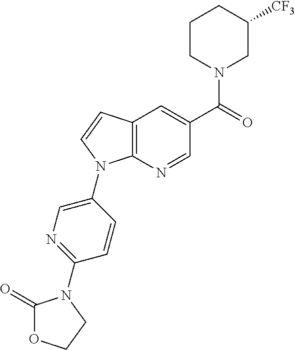 [SMILES: O=C(c1cnc2c(ccn2-c2ccc(N3CCOC3=O)nc2)c1)N1CCC[C@H](C(F)(F)F)C1] |
| B-81 | 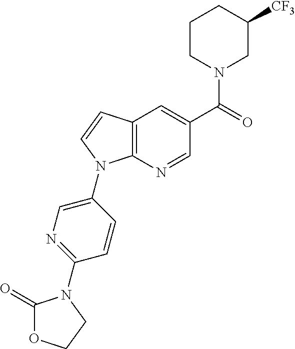 [SMILES: O=C(c1cnc2c(ccn2-c2ccc(N3CCOC3=O)nc2)c1)N1CCC[C@@H](C(F)(F)F)C1] |
| B-82 | 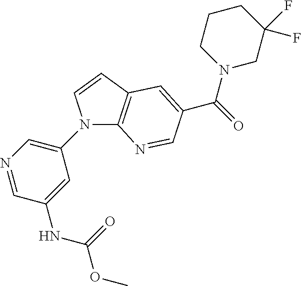 [SMILES: COC(=O)Nc1cncc(-n2ccc3cc(C(=O)N4CCCC(F)(F)C4)cnc32)c1] |
| B-83 | 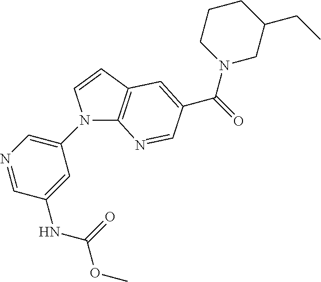 [SMILES: CCC1CCCN(C(=O)c2cnc3c(ccn3-c3cncc(NC(=O)OC)c3)c2)C1] |
| B-84 | 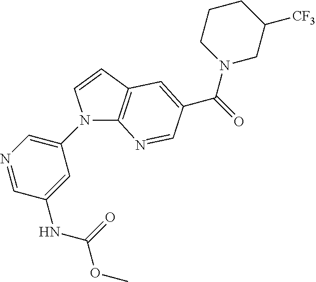 [SMILES: COC(=O)Nc1cncc(-n2ccc3cc(C(=O)N4CCCC(C(F)(F)F)C4)cnc32)c1] |
| B-85 | 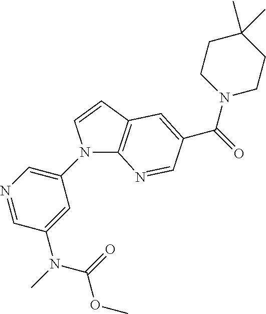 [SMILES: COC(=O)N(C)c1cncc(-n2ccc3cc(C(=O)N4CCC(C)(C)CC4)cnc32)c1] |
| B-86 | 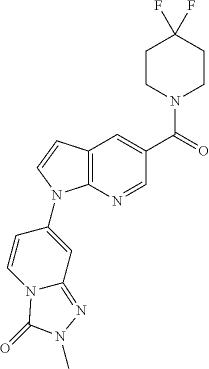 [SMILES: Cn1nc2cc(-n3ccc4cc(C(=O)N5CCC(F)(F)CC5)cnc43)ccn2c1=O] |
| B-87 | 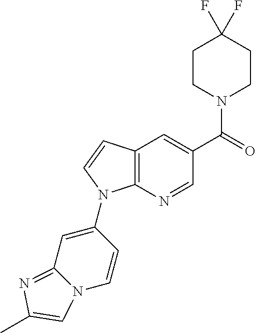 [SMILES: Cc1cn2ccc(-n3ccc4cc(C(=O)N5CCC(F)(F)CC5)cnc43)cc2n1] |
| B-88 | 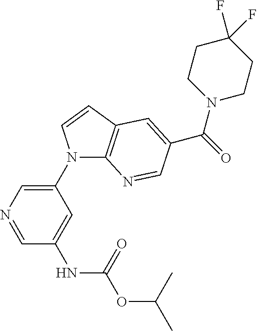 [SMILES: CC(C)OC(=O)Nc1cncc(-n2ccc3cc(C(=O)N4CCC(F)(F)CC4)cnc32)c1] |
| MB-89 | 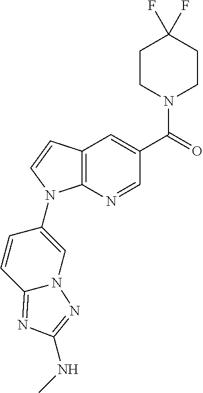 [SMILES: CNc1nc2ccc(-n3ccc4cc(C(=O)N5CCC(F)(F)CC5)cnc43)cn2n1] |
| B-90 | 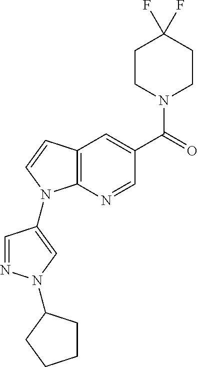 [SMILES: O=C(c1cnc2c(ccn2-c2cnn(C3CCCC3)c2)c1)N1CCC(F)(F)CC1] |
| B-91 | 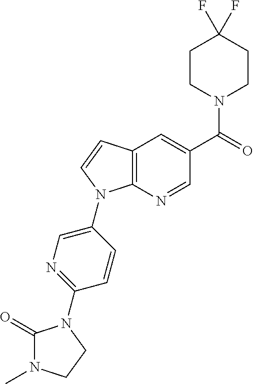 [SMILES: CN1CCN(c2ccc(-n3ccc4cc(C(=O)N5CCC(F)(F)CC5)cnc43)cn2)C1=O] |
| B-92 | 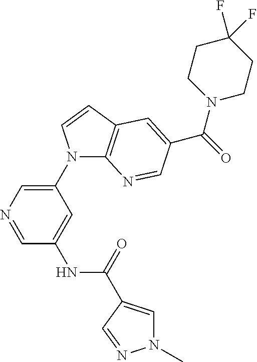 [SMILES: Cn1cc(C(=O)Nc2cncc(-n3ccc4cc(C(=O)N5CCC(F)(F)CC5)cnc43)c2)cn1] |
| B-93 | 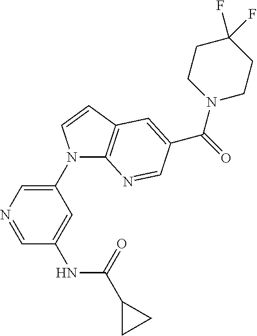 [SMILES: O=C(Nc1cncc(-n2ccc3cc(C(=O)N4CCC(F)(F)CC4)cnc32)c1)C1CC1] |
| B-94 | 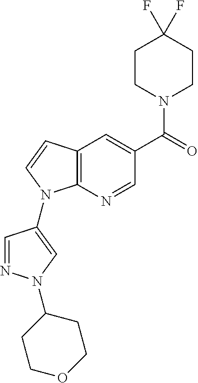 [SMILES: O=C(c1cnc2c(ccn2-c2cnn(C3CCOCC3)c2)c1)N1CCC(F)(F)CC1] |
| B-95 | 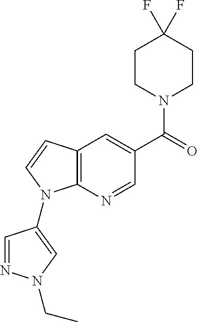 [SMILES: CCn1cc(-n2ccc3cc(C(=O)N4CCC(F)(F)CC4)cnc32)cn1] |
| B-96 | 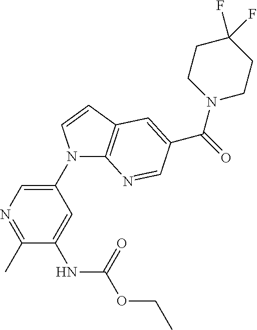 [SMILES: CCOC(=O)Nc1cc(-n2ccc3cc(C(=O)N4CCC(F)(F)CC4)cnc32)cnc1C] |
| B-97 | 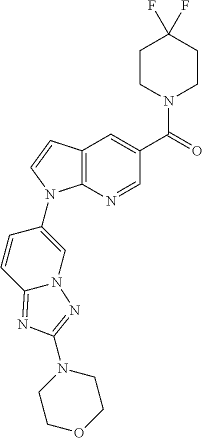 [SMILES: O=C(c1cnc2c(ccn2-c2ccc3nc(N4CCOCC4)nn3c2)c1)N1CCC(F)(F)CC1] |
| B-98 | 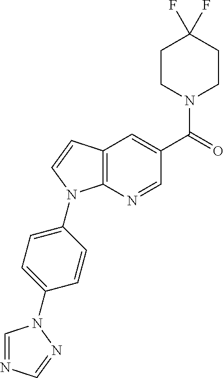 [SMILES: O=C(c1cnc2c(ccn2-c2ccc(-n3cncn3)cc2)c1)N1CCC(F)(F)CC1] |
| B-99 | 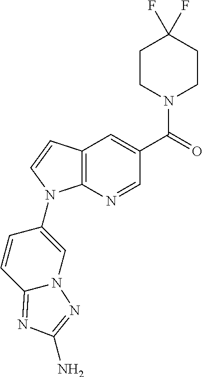 [SMILES: Nc1nc2ccc(-n3ccc4cc(C(=O)N5CCC(F)(F)CC5)cnc43)cn2n1] |
| B-100 | 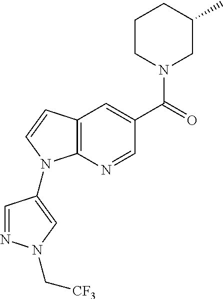 [SMILES: C[C@H]1CCCN(C(=O)c2cnc3c(ccn3-c3cnn(CC(F)(F)F)c3)c2)C1] |
| B-101 | 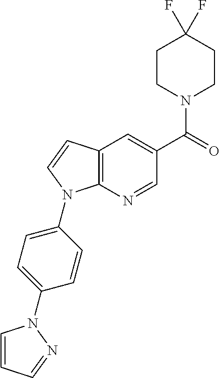 [SMILES: O=C(c1cnc2c(ccn2-c2ccc(-n3cccn3)cc2)c1)N1CCC(F)(F)CC1] |
| B-102 | 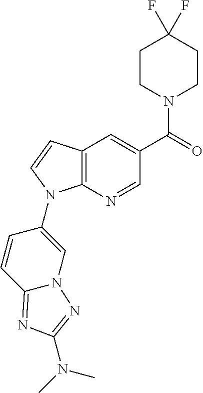 [SMILES: CN(C)c1nc2ccc(-n3ccc4cc(C(=O)N5CCC(F)(F)CC5)cnc43)cn2n1] |
| B-103 | 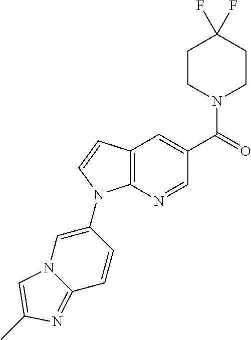 [SMILES: Cc1cn2cc(-n3ccc4cc(C(=O)N5CCC(F)(F)CC5)cnc43)ccc2n1] |
| B-104 | 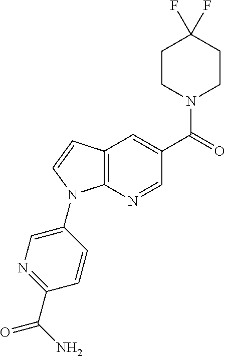 [SMILES: NC(=O)c1ccc(-n2ccc3cc(C(=O)N4CCC(F)(F)CC4)cnc32)cn1] |
| B-105 | 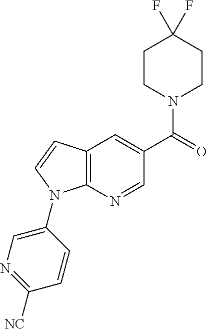 [SMILES: N#Cc1ccc(-n2ccc3cc(C(=O)N4CCC(F)(F)CC4)cnc32)cn1] |
| B-106 | 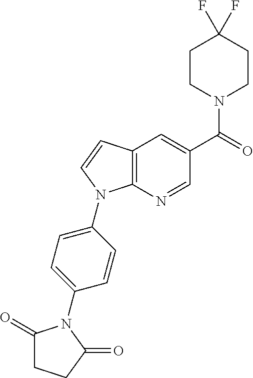 [SMILES: O=C(c1cnc2c(ccn2-c2ccc(N3C(=O)CCC3=O)cc2)c1)N1CCC(F)(F)CC1] |
| B-107 | 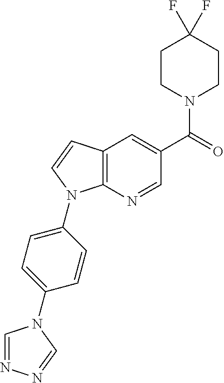 [SMILES: O=C(c1cnc2c(ccn2-c2ccc(-n3cnnc3)cc2)c1)N1CCC(F)(F)CC1] |
| B-108 | 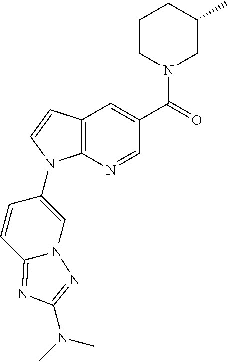 [SMILES: C[C@H]1CCCN(C(=O)c2cnc3c(ccn3-c3ccc4nc(N(C)C)nn4c3)c2)C1] |
| B-109 | 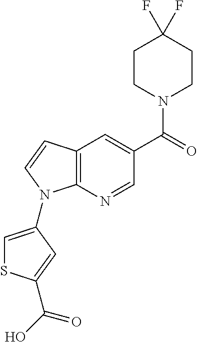 [SMILES: O=C(O)c1cc(-n2ccc3cc(C(=O)N4CCC(F)(F)CC4)cnc32)cs1] |
| B-110 | 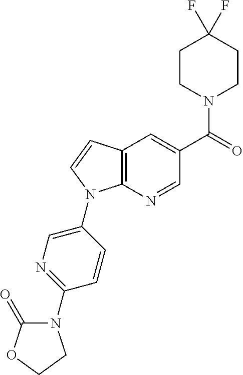 [SMILES: O=C(c1cnc2c(ccn2-c2ccc(N3CCOC3=O)nc2)c1)N1CCC(F)(F)CC1] |
| B-112 | 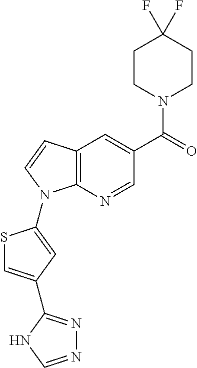 [SMILES: O=C(c1cnc2c(ccn2-c2cc(-c3nnc[nH]3)cs2)c1)N1CCC(F)(F)CC1] |
| B-113 | 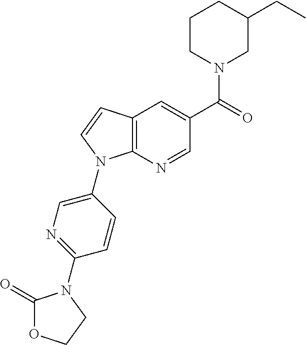 [SMILES: CCC1CCCN(C(=O)c2cnc3c(ccn3-c3ccc(N4CCOC4=O)nc3)c2)C1] |
| B-114 | 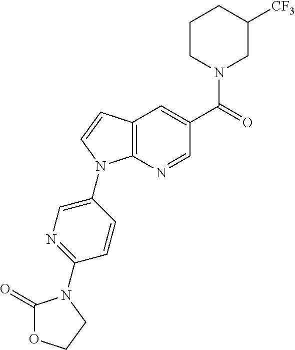 [SMILES: O=C(c1cnc2c(ccn2-c2ccc(N3CCOC3=O)nc2)c1)N1CCCC(C(F)(F)F)C1] |
| B-115 | 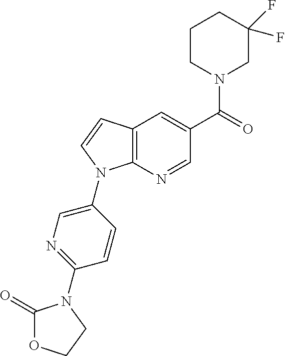 [SMILES: O=C(c1cnc2c(ccn2-c2ccc(N3CCOC3=O)nc2)c1)N1CCCC(F)(F)C1] |
| B-116 | 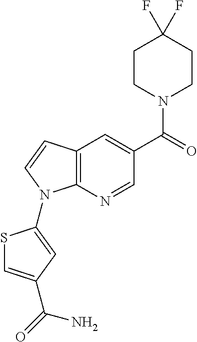 [SMILES: NC(=O)c1csc(-n2ccc3cc(C(=O)N4CCC(F)(F)CC4)cnc32)c1] |
| B-117 | 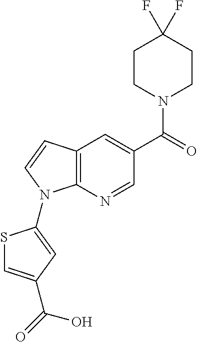 [SMILES: O=C(O)c1csc(-n2ccc3cc(C(=O)N4CCC(F)(F)CC4)cnc32)c1] |
| B-118 | 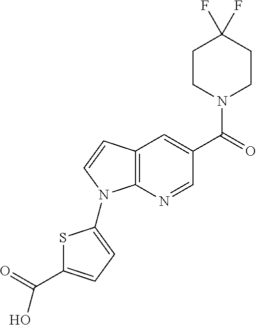 [SMILES: O=C(O)c1ccc(-n2ccc3cc(C(=O)N4CCC(F)(F)CC4)cnc32)s1] |
| B-119 | 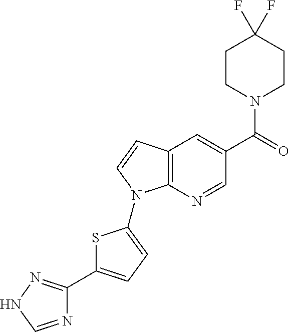 [SMILES: O=C(c1cnc2c(ccn2-c2ccc(-c3nc[nH]n3)s2)c1)N1CCC(F)(F)CC1] |
| B-120 | 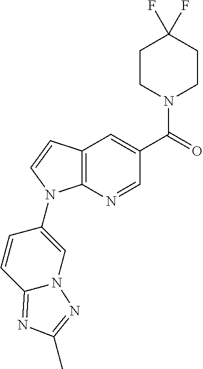 [SMILES: Cc1nc2ccc(-n3ccc4cc(C(=O)N5CCC(F)(F)CC5)cnc43)cn2n1] |
| B-121 | 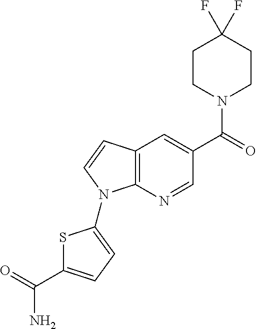 [SMILES: NC(=O)c1ccc(-n2ccc3cc(C(=O)N4CCC(F)(F)CC4)cnc32)s1] |
| B-122 | 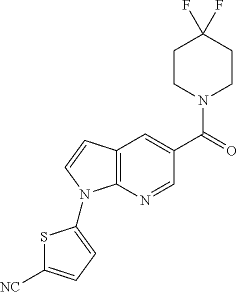 [SMILES: N#Cc1ccc(-n2ccc3cc(C(=O)N4CCC(F)(F)CC4)cnc32)s1] |
| B-123 | 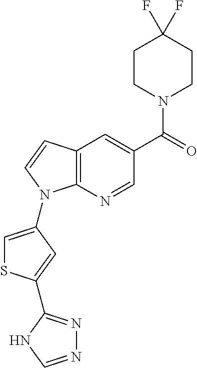 [SMILES: O=C(c1cnc2c(ccn2-c2csc(-c3nnc[nH]3)c2)c1)N1CCC(F)(F)CC1] |
| B-124 | 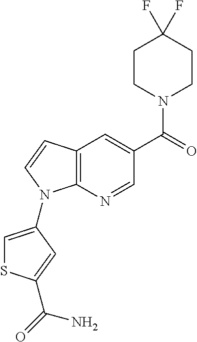 [SMILES: NC(=O)c1cc(-n2ccc3cc(C(=O)N4CCC(F)(F)CC4)cnc32)cs1] |
| B-125 | 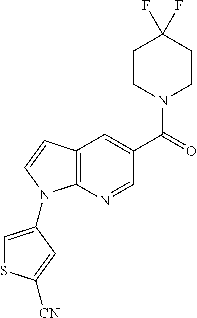 [SMILES: N#Cc1cc(-n2ccc3cc(C(=O)N4CCC(F)(F)CC4)cnc32)cs1] |
| B-126 | 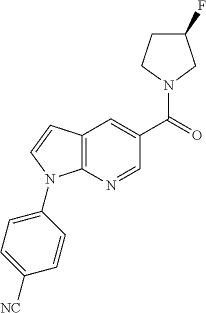 [SMILES: N#Cc1ccc(-n2ccc3cc(C(=O)N4CC[C@@H](F)C4)cnc32)cc1] |
| B-127 | 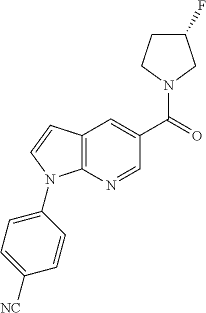 [SMILES: N#Cc1ccc(-n2ccc3cc(C(=O)N4CC[C@H](F)C4)cnc32)cc1] |
| B-128 | 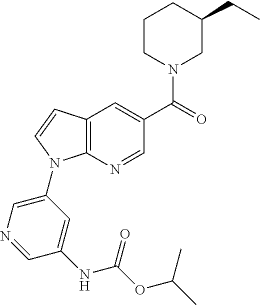 [SMILES: CC[C@@H]1CCCN(C(=O)c2cnc3c(ccn3-c3cncc(NC(=O)OC(C)C)c3)c2)C1] |
| B-129 | 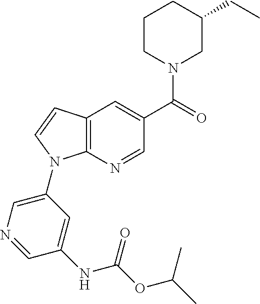 [SMILES: CC[C@H]1CCCN(C(=O)c2cnc3c(ccn3-c3cncc(NC(=O)OC(C)C)c3)c2)C1] |
| B-130 | 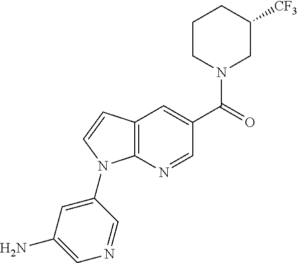 [SMILES: Nc1cncc(-n2ccc3cc(C(=O)N4CCC[C@H](C(F)(F)F)C4)cnc32)c1] |
| B-131 | 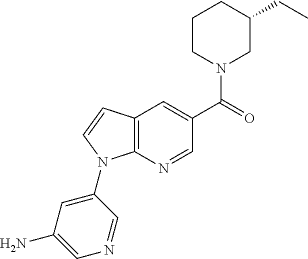 [SMILES: CC[C@H]1CCCN(C(=O)c2cnc3c(ccn3-c3cncc(N)c3)c2)C1] |
| B-132 | 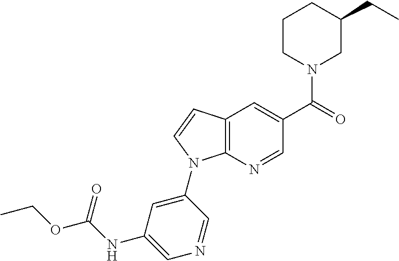 [SMILES: CCOC(=O)Nc1cncc(-n2ccc3cc(C(=O)N4CCC[C@@H](CC)C4)cnc32)c1] |
| B-133 | 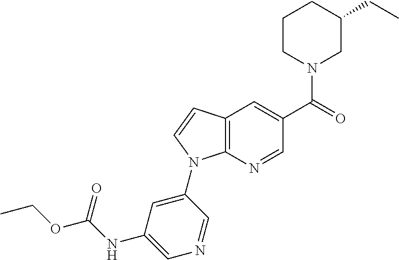 [SMILES: CCOC(=O)Nc1cncc(-n2ccc3cc(C(=O)N4CCC[C@H](CC)C4)cnc32)c1] |
| B-134 | 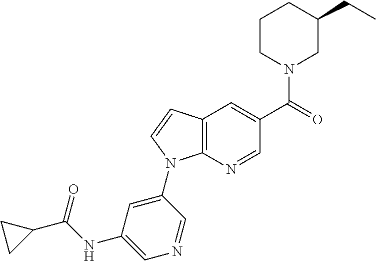 [SMILES: CC[C@@H]1CCCN(C(=O)c2cnc3c(ccn3-c3cncc(NC(=O)C4CC4)c3)c2)C1] |
| B-135 | 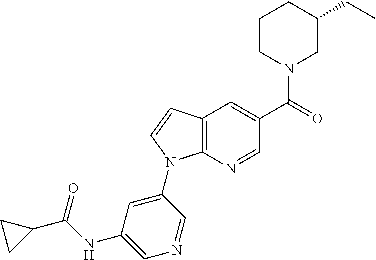 [SMILES: CC[C@H]1CCCN(C(=O)c2cnc3c(ccn3-c3cncc(NC(=O)C4CC4)c3)c2)C1] |
| B-136 | 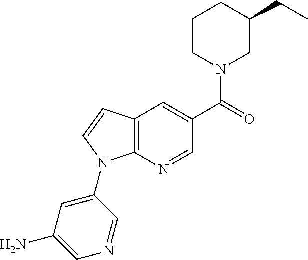 [SMILES: CC[C@@H]1CCCN(C(=O)c2cnc3c(ccn3-c3cncc(N)c3)c2)C1] |
| B-137 | 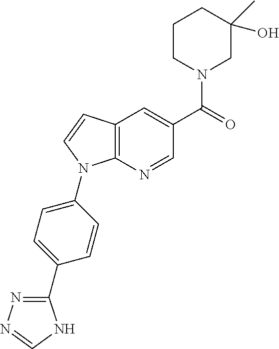 [SMILES: CC1(O)CCCN(C(=O)c2cnc3c(ccn3-c3ccc(-c4nnc[nH]4)cc3)c2)C1] |
| B-138 | 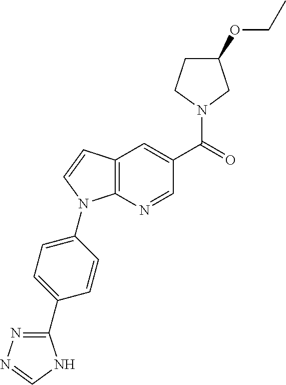 [SMILES: CCO[C@@H]1CCN(C(=O)c2cnc3c(ccn3-c3ccc(-c4nnc[nH]4)cc3)c2)C1] |
| B-139 | 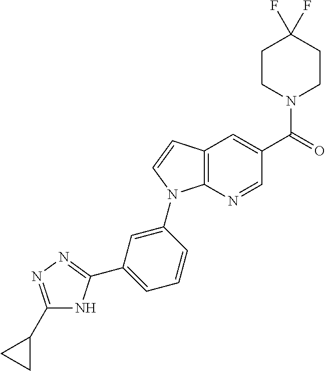 [SMILES: O=C(c1cnc2c(ccn2-c2cccc(-c3nnc(C4CC4)[nH]3)c2)c1)N1CCC(F)(F)CC1] |
| B-140 | 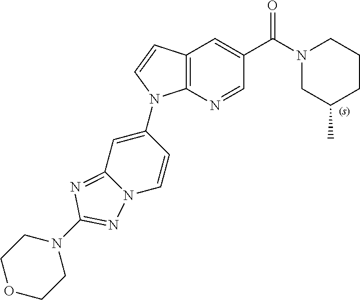 [SMILES: C[C@H]1CCCN(C(=O)c2cnc3c(ccn3-c3ccn4nc(N5CCOCC5)nc4c3)c2)C1] |
| B-141 | 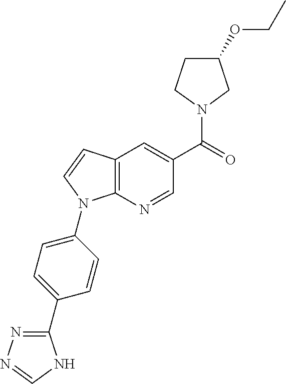 [SMILES: CCO[C@H]1CCN(C(=O)c2cnc3c(ccn3-c3ccc(-c4nnc[nH]4)cc3)c2)C1] |
| B-142 | 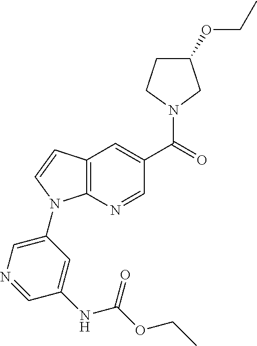 [SMILES: CCOC(=O)Nc1cncc(-n2ccc3cc(C(=O)N4CC[C@H](OCC)C4)cnc32)c1] |
| B-143 | 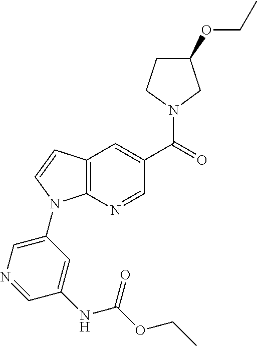 [SMILES: CCOC(=O)Nc1cncc(-n2ccc3cc(C(=O)N4CC[C@@H](OCC)C4)cnc32)c1] |
| B-144 | 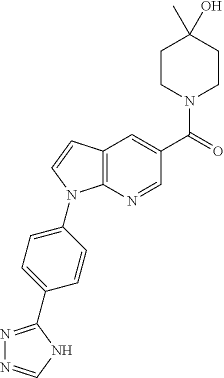 [SMILES: CC1(O)CCN(C(=O)c2cnc3c(ccn3-c3ccc(-c4nnc[nH]4)cc3)c2)CC1] |
| B-145 | 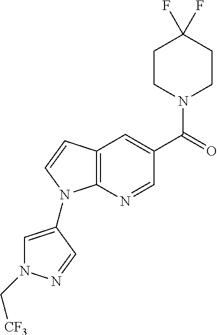 [SMILES: O=C(c1cnc2c(ccn2-c2cnn(CC(F)(F)F)c2)c1)N1CCC(F)(F)CC1] |
| B-146 | 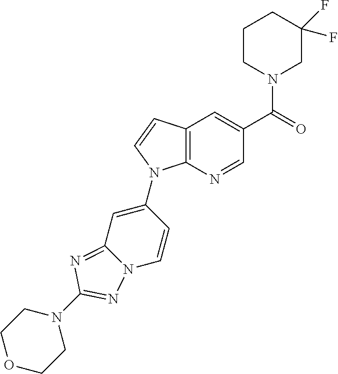 [SMILES: O=C(c1cnc2c(ccn2-c2ccn3nc(N4CCOCC4)nc3c2)c1)N1CCCC(F)(F)C1] |
| B-147 | 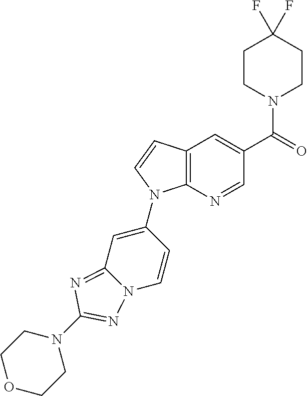 [SMILES: O=C(c1cnc2c(ccn2-c2ccn3nc(N4CCOCC4)nc3c2)c1)N1CCC(F)(F)CC1] |
| B-148 | 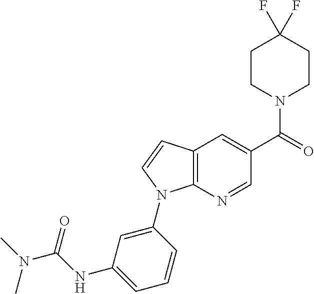 [SMILES: CN(C)C(=O)Nc1cccc(-n2ccc3cc(C(=O)N4CCC(F)(F)CC4)cnc32)c1] |
| B-149 | 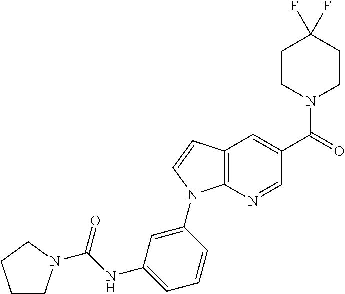 [SMILES: O=C(Nc1cccc(-n2ccc3cc(C(=O)N4CCC(F)(F)CC4)cnc32)c1)N1CCCC1] |
| B-150 | 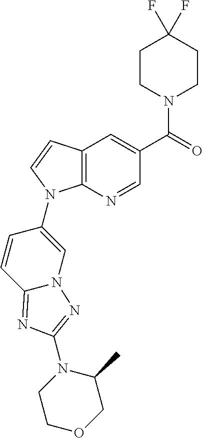 [SMILES: C[C@H]1COCCN1c1nc2ccc(-n3ccc4cc(C(=O)N5CCC(F)(F)CC5)cnc43)cn2n1] |
| B-151 | 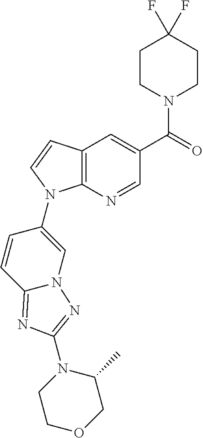 [SMILES: C[C@@H]1COCCN1c1nc2ccc(-n3ccc4cc(C(=O)N5CCC(F)(F)CC5)cnc43)cn2n1] |
| B-152 | 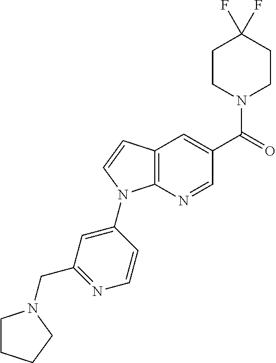 [SMILES: O=C(c1cnc2c(ccn2-c2ccnc(CN3CCCC3)c2)c1)N1CCC(F)(F)CC1] |
| B-153 | 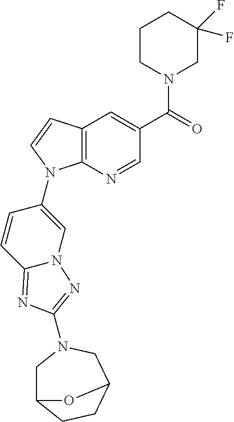 [SMILES: O=C(c1cnc2c(ccn2-c2ccc3nc(N4CC5CCC(C4)O5)nn3c2)c1)N1CCCC(F)(F)C1] |
| B-154 | 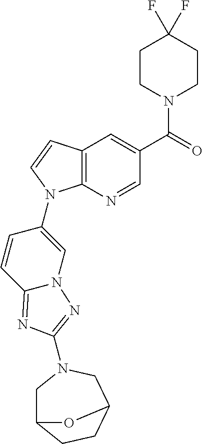 [SMILES: O=C(c1cnc2c(ccn2-c2ccc3nc(N4CC5CCC(C4)O5)nn3c2)c1)N1CCC(F)(F)CC1] |
| B-155 | 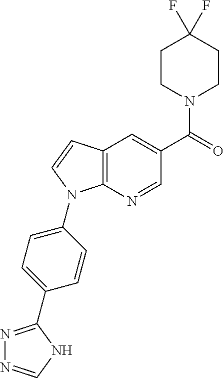 [SMILES: O=C(c1cnc2c(ccn2-c2ccc(-c3nnc[nH]3)cc2)c1)N1CCC(F)(F)CC1] |
| B-156 | 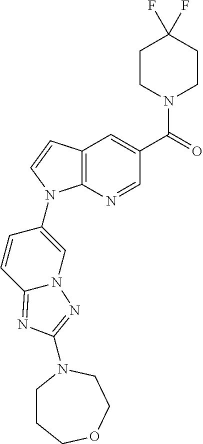 [SMILES: O=C(c1cnc2c(ccn2-c2ccc3nc(N4CCCOCC4)nn3c2)c1)N1CCC(F)(F)CC1] |
| B-157 | 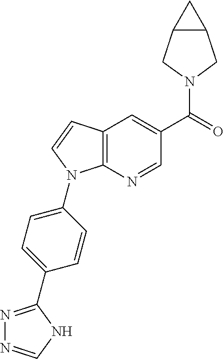 [SMILES: O=C(c1cnc2c(ccn2-c2ccc(-c3nnc[nH]3)cc2)c1)N1CC2CC2C1] |
| B-158 | 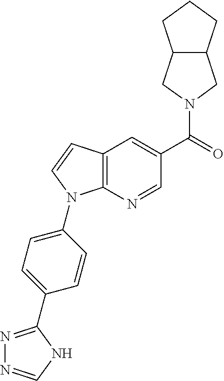 [SMILES: O=C(c1cnc2c(ccn2-c2ccc(-c3nnc[nH]3)cc2)c1)N1CC2CCCC2C1] |
| B-159 | 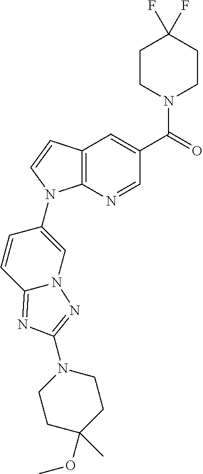 [SMILES: COC1(C)CCN(c2nc3ccc(-n4ccc5cc(C(=O)N6CCC(F)(F)CC6)cnc54)cn3n2)CC1] |
| B-160 | 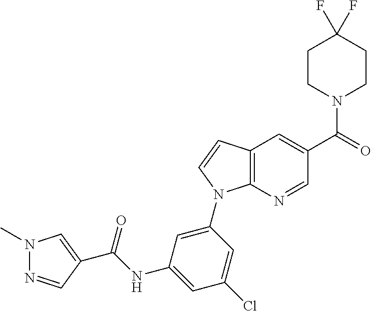 [SMILES: Cn1cc(C(=O)Nc2cc(Cl)cc(-n3ccc4cc(C(=O)N5CCC(F)(F)CC5)cnc43)c2)cn1] |
| B-161 | 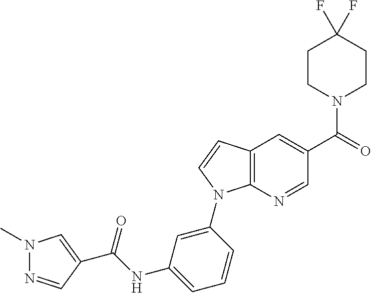 [SMILES: Cn1cc(C(=O)Nc2cccc(-n3ccc4cc(C(=O)N5CCC(F)(F)CC5)cnc43)c2)cn1] |
| B-162 | 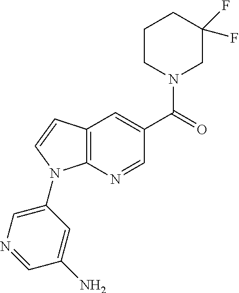 [SMILES: Nc1cncc(-n2ccc3cc(C(=O)N4CCCC(F)(F)C4)cnc32)c1] |
| B-163 | 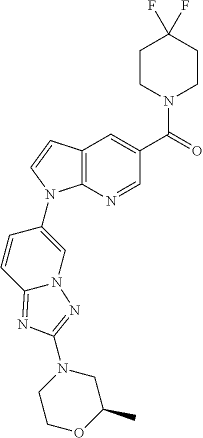 [SMILES: C[C@@H]1CN(c2nc3ccc(-n4ccc5cc(C(=O)N6CCC(F)(F)CC6)cnc54)cn3n2)CCO1] |
| B-164 | 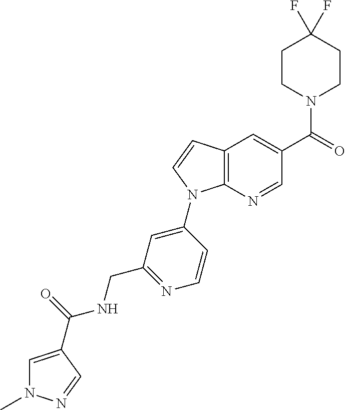 [SMILES: Cn1cc(C(=O)NCc2cc(-n3ccc4cc(C(=O)N5CCC(F)(F)CC5)cnc43)ccn2)cn1] |
| B-165 | 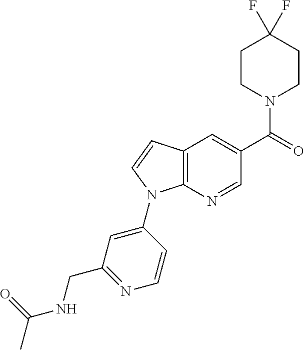 [SMILES: CC(=O)NCc1cc(-n2ccc3cc(C(=O)N4CCC(F)(F)CC4)cnc32)ccn1] |
| B-166 | 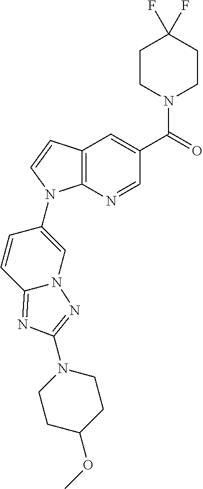 [SMILES: COC1CCN(c2nc3ccc(-n4ccc5cc(C(=O)N6CCC(F)(F)CC6)cnc54)cn3n2)CC1] |
| B-167 | 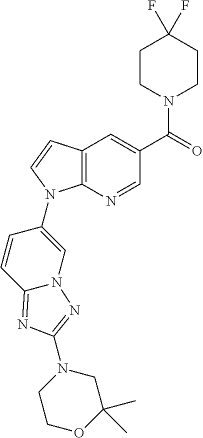 [SMILES: CC1(C)CN(c2nc3ccc(-n4ccc5cc(C(=O)N6CCC(F)(F)CC6)cnc54)cn3n2)CCO1] |
| B-168 | 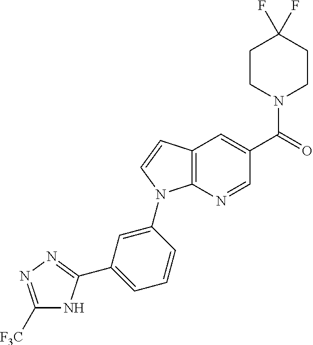 [SMILES: O=C(c1cnc2c(ccn2-c2cccc(-c3nnc(C(F)(F)F)[nH]3)c2)c1)N1CCC(F)(F)CC1] |
| B-169 | 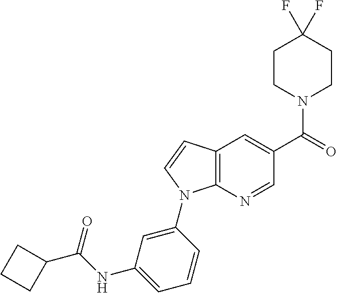 [SMILES: O=C(Nc1cccc(-n2ccc3cc(C(=O)N4CCC(F)(F)CC4)cnc32)c1)C1CCC1] |
| B-170 | 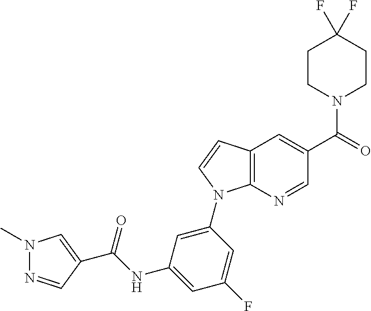 [SMILES: Cn1cc(C(=O)Nc2cc(F)cc(-n3ccc4cc(C(=O)N5CCC(F)(F)CC5)cnc43)c2)cn1] |
| B-171 | 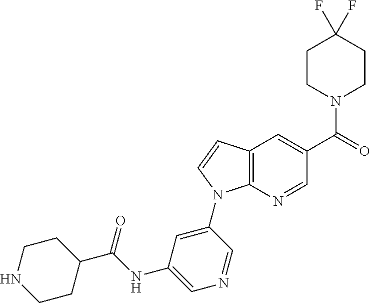 [SMILES: O=C(Nc1cncc(-n2ccc3cc(C(=O)N4CCC(F)(F)CC4)cnc32)c1)C1CCNCC1] |
| B-172 | 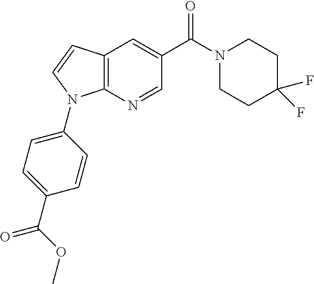 [SMILES: COC(=O)c1ccc(-n2ccc3cc(C(=O)N4CCC(F)(F)CC4)cnc32)cc1] |
| B-173 | 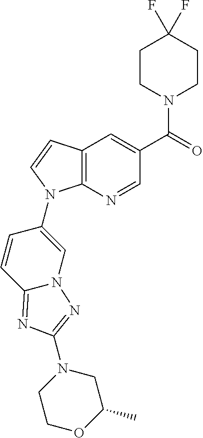 [SMILES: C[C@H]1CN(c2nc3ccc(-n4ccc5cc(C(=O)N6CCC(F)(F)CC6)cnc54)cn3n2)CCO1] |
| B-174 | 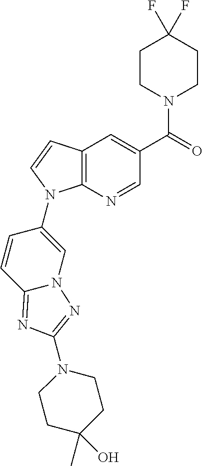 [SMILES: CC1(O)CCN(c2nc3ccc(-n4ccc5cc(C(=O)N6CCC(F)(F)CC6)cnc54)cn3n2)CC1] |
| B-175 | 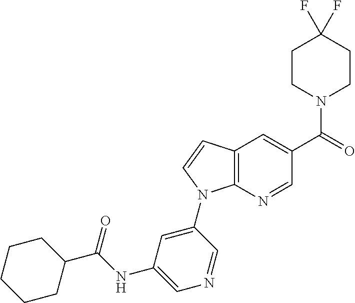 [SMILES: O=C(Nc1cncc(-n2ccc3cc(C(=O)N4CCC(F)(F)CC4)cnc32)c1)C1CCCCC1] |
| B-176 | 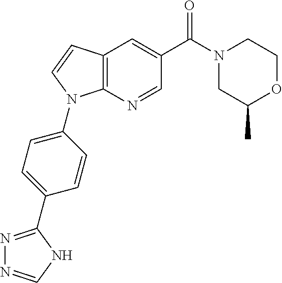 [SMILES: C[C@H]1CN(C(=O)c2cnc3c(ccn3-c3ccc(-c4nnc[nH]4)cc3)c2)CCO1] |
| B-177 | 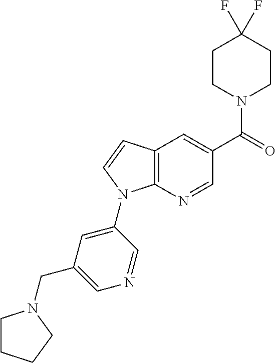 [SMILES: O=C(c1cnc2c(ccn2-c2cncc(CN3CCCC3)c2)c1)N1CCC(F)(F)CC1] |
| B-178 | 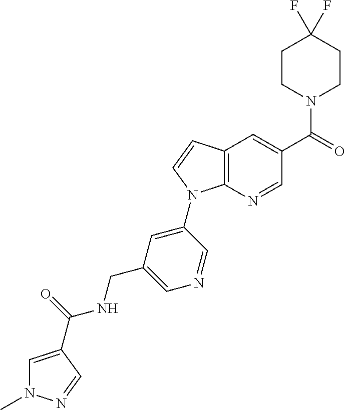 [SMILES: Cn1cc(C(=O)NCc2cncc(-n3ccc4cc(C(=O)N5CCC(F)(F)CC5)cnc43)c2)cn1] |
| B-179 | 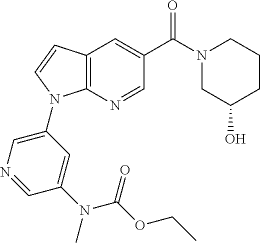 [SMILES: CCOC(=O)N(C)c1cncc(-n2ccc3cc(C(=O)N4CCC[C@H](O)C4)cnc32)c1] |
| B-180 | 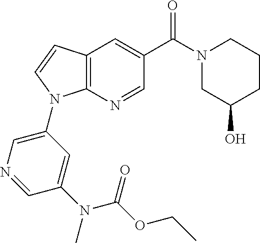 [SMILES: CCOC(=O)N(C)c1cncc(-n2ccc3cc(C(=O)N4CCC[C@@H](O)C4)cnc32)c1] |
| B-181 | 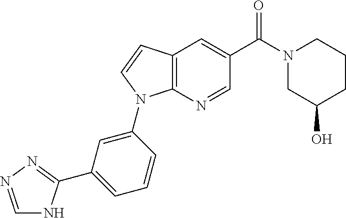 [SMILES: O=C(c1cnc2c(ccn2-c2cccc(-c3nnc[nH]3)c2)c1)N1CCC[C@@H](O)C1] |
| B-182 | 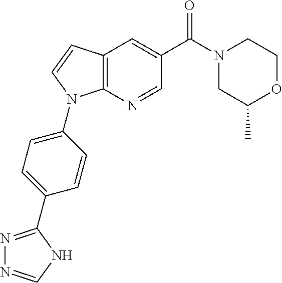 [SMILES: C[C@@H]1CN(C(=O)c2cnc3c(ccn3-c3ccc(-c4nnc[nH]4)cc3)c2)CCO1] |
| B-183 | 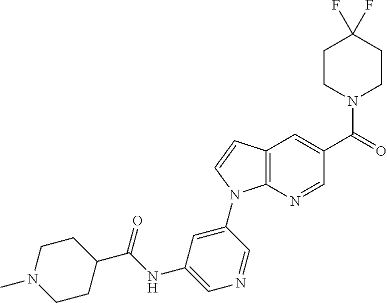 [SMILES: CN1CCC(C(=O)Nc2cncc(-n3ccc4cc(C(=O)N5CCC(F)(F)CC5)cnc43)c2)CC1] |
| B-184 | 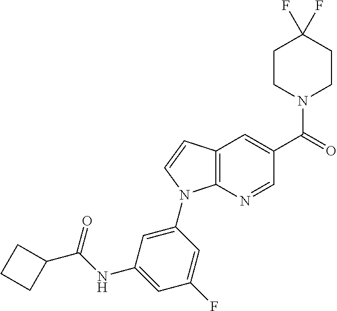 [SMILES: O=C(Nc1cc(F)cc(-n2ccc3cc(C(=O)N4CCC(F)(F)CC4)cnc32)c1)C1CCC1] |
| B-185 | 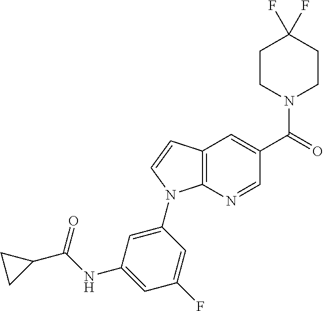 [SMILES: O=C(Nc1cc(F)cc(-n2ccc3cc(C(=O)N4CCC(F)(F)CC4)cnc32)c1)C1CC1] |
| B-186 | 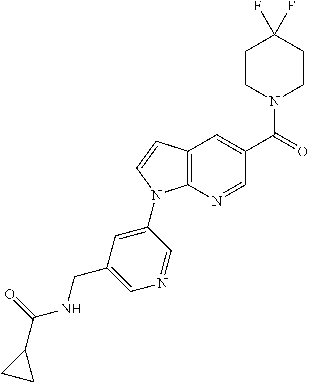 [SMILES: O=C(NCc1cncc(-n2ccc3cc(C(=O)N4CCC(F)(F)CC4)cnc32)c1)C1CC1] |
| B-187 | 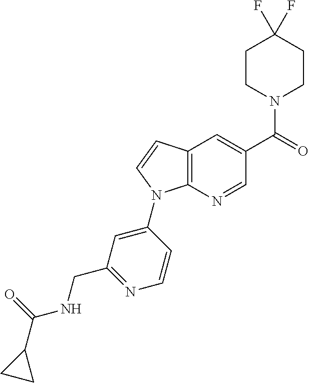 [SMILES: O=C(NCc1cc(-n2ccc3cc(C(=O)N4CCC(F)(F)CC4)cnc32)ccn1)C1CC1] |
| B-188 | 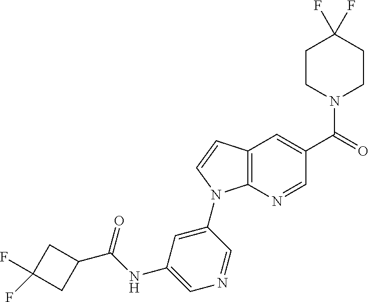 [SMILES: O=C(Nc1cncc(-n2ccc3cc(C(=O)N4CCC(F)(F)CC4)cnc32)c1)C1CC(F)(F)C1] |
| B-189 | 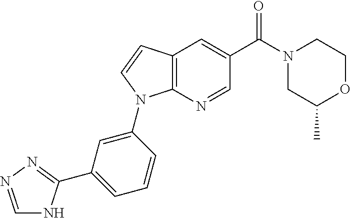 [SMILES: C[C@@H]1CN(C(=O)c2cnc3c(ccn3-c3cccc(-c4nnc[nH]4)c3)c2)CCO1] |
| B-190 | 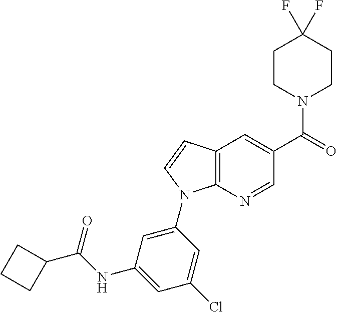 [SMILES: O=C(Nc1cc(Cl)cc(-n2ccc3cc(C(=O)N4CCC(F)(F)CC4)cnc32)c1)C1CCC1] |
| B-191 | 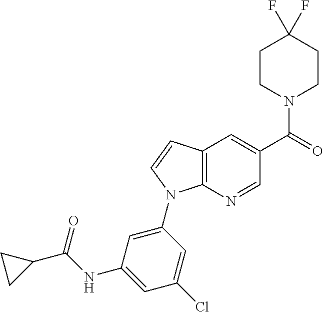 [SMILES: O=C(Nc1cc(Cl)cc(-n2ccc3cc(C(=O)N4CCC(F)(F)CC4)cnc32)c1)C1CC1] |
| B-192 | 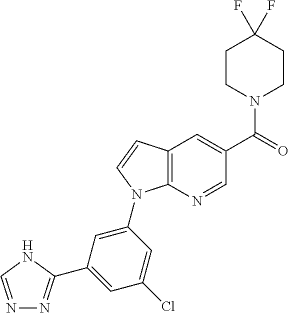 [SMILES: O=C(c1cnc2c(ccn2-c2cc(Cl)cc(-c3nnc[nH]3)c2)c1)N1CCC(F)(F)CC1] |
| B-193 | 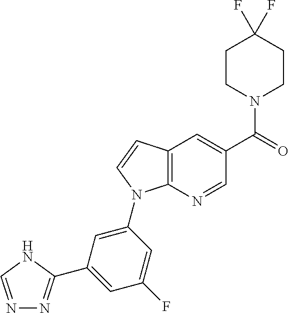 [SMILES: O=C(c1cnc2c(ccn2-c2cc(F)cc(-c3nnc[nH]3)c2)c1)N1CCC(F)(F)CC1] |
| B-194 | 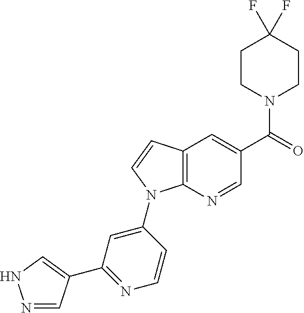 [SMILES: O=C(c1cnc2c(ccn2-c2ccnc(-c3cn[nH]c3)c2)c1)N1CCC(F)(F)CC1] |
| B-195 | 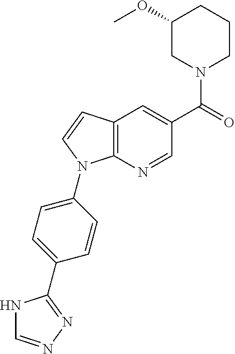 [SMILES: CO[C@@H]1CCCN(C(=O)c2cnc3c(ccn3-c3ccc(-c4nnc[nH]4)cc3)c2)C1] |
| B-196 | 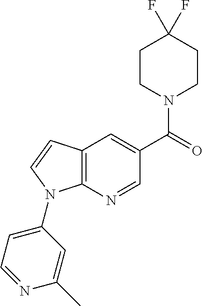 [SMILES: Cc1cc(-n2ccc3cc(C(=O)N4CCC(F)(F)CC4)cnc32)ccn1] |
| B-197 | 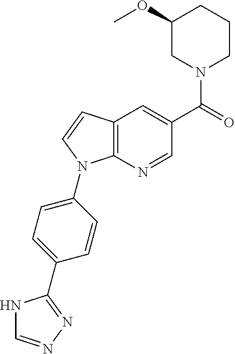 [SMILES: CO[C@H]1CCCN(C(=O)c2cnc3c(ccn3-c3ccc(-c4nnc[nH]4)cc3)c2)C1] |
| B-198 | 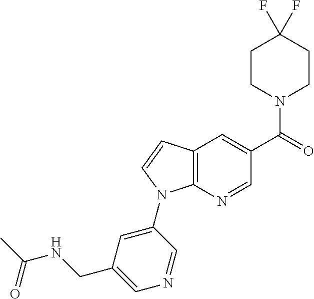 [SMILES: CC(=O)NCc1cncc(-n2ccc3cc(C(=O)N4CCC(F)(F)CC4)cnc32)c1] |
| B-199 | 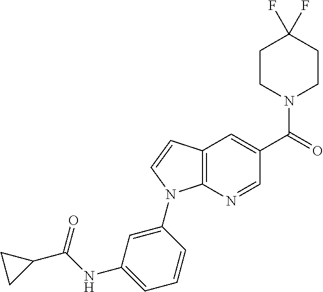 [SMILES: O=C(Nc1cccc(-n2ccc3cc(C(=O)N4CCC(F)(F)CC4)cnc32)c1)C1CC1] |
| B-200 | 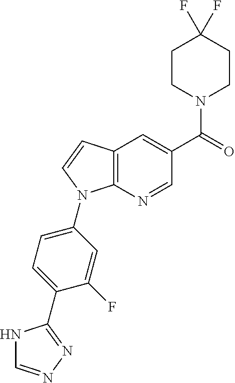 [SMILES: O=C(c1cnc2c(ccn2-c2ccc(-c3nnc[nH]3)c(F)c2)c1)N1CCC(F)(F)CC1] |
| B-201 | 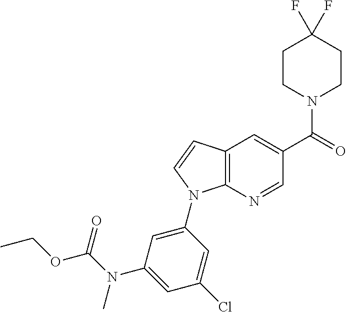 [SMILES: CCOC(=O)N(C)c1cc(Cl)cc(-n2ccc3cc(C(=O)N4CCC(F)(F)CC4)cnc32)c1] |
| B-202 | 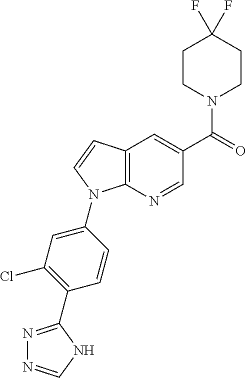 [SMILES: O=C(c1cnc2c(ccn2-c2ccc(-c3nnc[nH]3)c(Cl)c2)c1)N1CCC(F)(F)CC1] |
| B-203 | 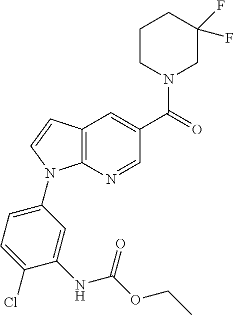 [SMILES: CCOC(=O)Nc1cc(-n2ccc3cc(C(=O)N4CCCC(F)(F)C4)cnc32)ccc1Cl] |
| B-204 | 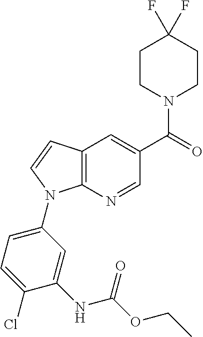 [SMILES: CCOC(=O)Nc1cc(-n2ccc3cc(C(=O)N4CCC(F)(F)CC4)cnc32)ccc1Cl] |
| B-205 | 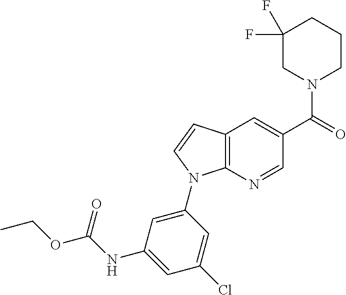 [SMILES: CCOC(=O)Nc1cc(Cl)cc(-n2ccc3cc(C(=O)N4CCCC(F)(F)C4)cnc32)c1] |
| B-206 | 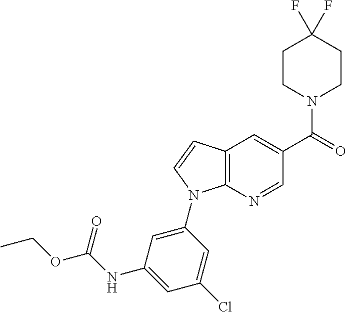 [SMILES: CCOC(=O)Nc1cc(Cl)cc(-n2ccc3cc(C(=O)N4CCC(F)(F)CC4)cnc32)c1] |
| B-207 | 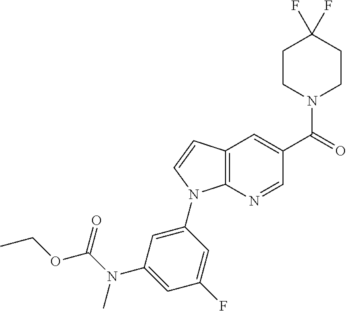 [SMILES: CCOC(=O)N(C)c1cc(F)cc(-n2ccc3cc(C(=O)N4CCC(F)(F)CC4)cnc32)c1] |
| B-208 | 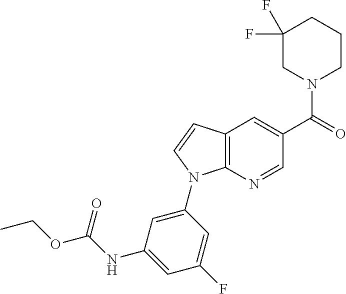 [SMILES: CCOC(=O)Nc1cc(F)cc(-n2ccc3cc(C(=O)N4CCCC(F)(F)C4)cnc32)c1] |
| B-209 | 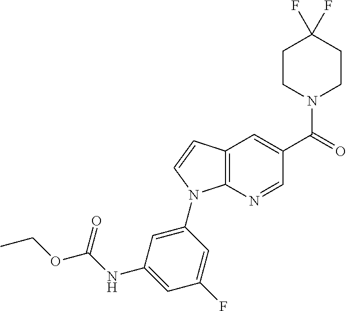 [SMILES: CCOC(=O)Nc1cc(F)cc(-n2ccc3cc(C(=O)N4CCC(F)(F)CC4)cnc32)c1] |
| B-210 | 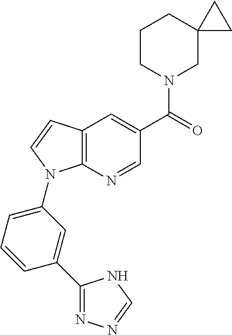 [SMILES: O=C(c1cnc2c(ccn2-c2cccc(-c3nnc[nH]3)c2)c1)N1CCCC2(CC2)C1] |
| B-211 | 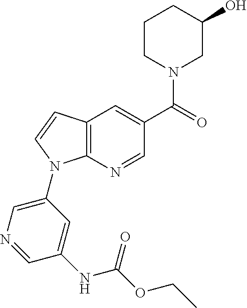 [SMILES: CCOC(=O)Nc1cncc(-n2ccc3cc(C(=O)N4CCC[C@@H](O)C4)cnc32)c1] |
| B-212 | 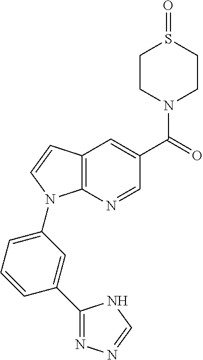 [SMILES: O=C(c1cnc2c(ccn2-c2cccc(-c3nnc[nH]3)c2)c1)N1CCS(=O)CC1] |
| B-213 | 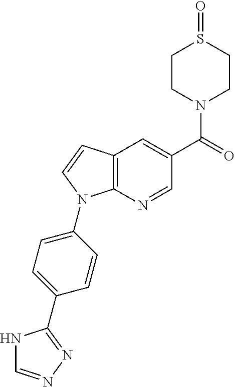 [SMILES: O=C(c1cnc2c(ccn2-c2ccc(-c3nnc[nH]3)cc2)c1)N1CCS(=O)CC1] |
| B-214 | 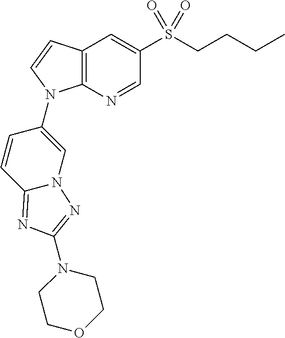 [SMILES: CCCCS(=O)(=O)c1cnc2c(ccn2-c2ccc3nc(N4CCOCC4)nn3c2)c1] |
| B-215 | 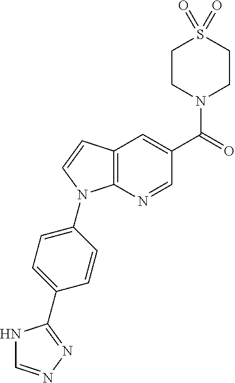 [SMILES: O=C(c1cnc2c(ccn2-c2ccc(-c3nnc[nH]3)cc2)c1)N1CCS(=O)(=O)CC1] |
| B-216 | 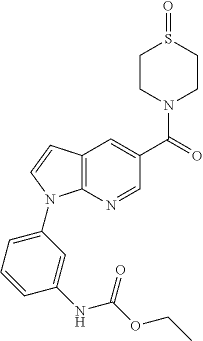 [SMILES: CCOC(=O)Nc1cccc(-n2ccc3cc(C(=O)N4CCS(=O)CC4)cnc32)c1] |
| B-217 | 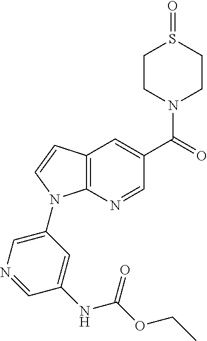 [SMILES: CCOC(=O)Nc1cncc(-n2ccc3cc(C(=O)N4CCS(=O)CC4)cnc32)c1] |
| B-218 | 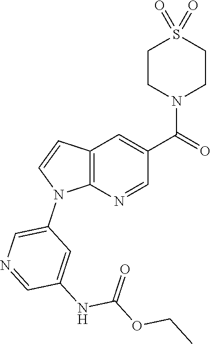 [SMILES: CCOC(=O)Nc1cncc(-n2ccc3cc(C(=O)N4CCS(=O)(=O)CC4)cnc32)c1] |
| B-219 | 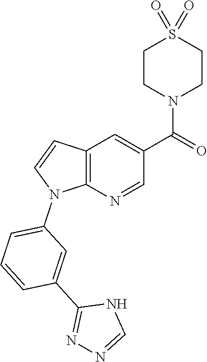 [SMILES: O=C(c1cnc2c(ccn2-c2cccc(-c3nnc[nH]3)c2)c1)N1CCS(=O)(=O)CC1] |
| B-220 | 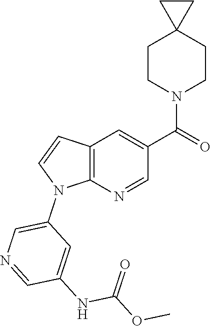 [SMILES: COC(=O)Nc1cncc(-n2ccc3cc(C(=O)N4CCC5(CC4)CC5)cnc32)c1] |
| B-221 | 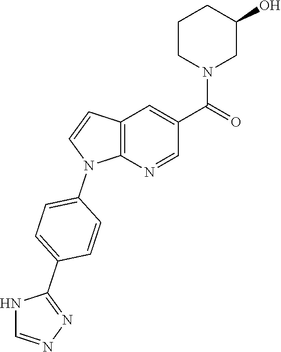 [SMILES: O=C(c1cnc2c(ccn2-c2ccc(-c3nnc[nH]3)cc2)c1)N1CCC[C@@H](O)C1] |
| B-222 | 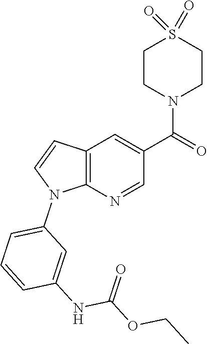 [SMILES: CCOC(=O)Nc1cccc(-n2ccc3cc(C(=O)N4CCS(=O)(=O)CC4)cnc32)c1] |
| B-223 | 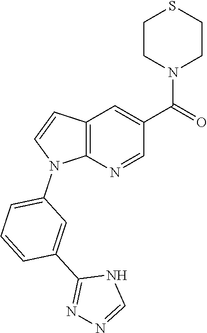 [SMILES: O=C(c1cnc2c(ccn2-c2cccc(-c3nnc[nH]3)c2)c1)N1CCSCC1] |
| B-224 | 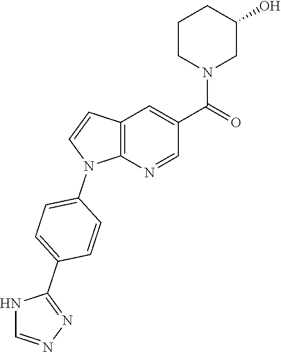 [SMILES: O=C(c1cnc2c(ccn2-c2ccc(-c3nnc[nH]3)cc2)c1)N1CCC[C@H](O)C1] |
| B-225 | 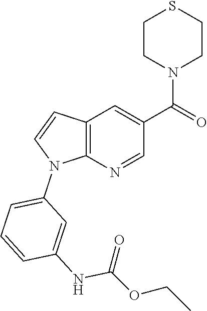 [SMILES: CCOC(=O)Nc1cccc(-n2ccc3cc(C(=O)N4CCSCC4)cnc32)c1] |
| B-226 | 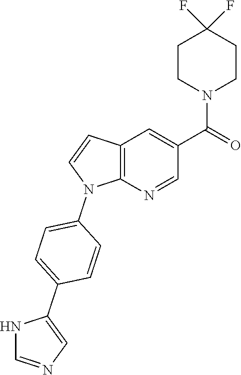 [SMILES: O=C(c1cnc2c(ccn2-c2ccc(-c3cnc[nH]3)cc2)c1)N1CCC(F)(F)CC1] |
| B-227 | 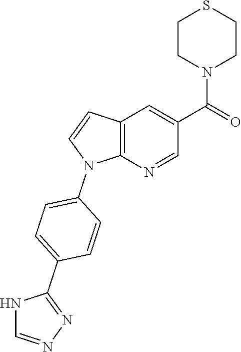 [SMILES: O=C(c1cnc2c(ccn2-c2ccc(-c3nnc[nH]3)cc2)c1)N1CCSCC1] |
| B-228 | 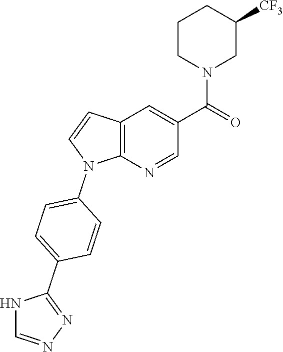 [SMILES: O=C(c1cnc2c(ccn2-c2ccc(-c3nnc[nH]3)cc2)c1)N1CCC[C@@H](C(F)(F)F)C1] |
| B-229 | 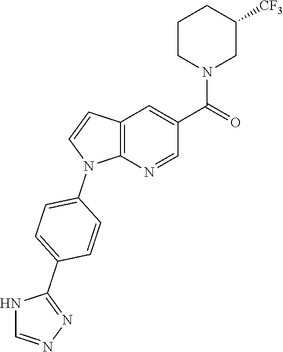 [SMILES: O=C(c1cnc2c(ccn2-c2ccc(-c3nnc[nH]3)cc2)c1)N1CCC[C@H](C(F)(F)F)C1] |
| B-230 | 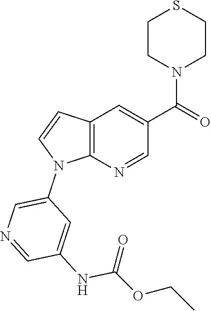 [SMILES: CCOC(=O)Nc1cncc(-n2ccc3cc(C(=O)N4CCSCC4)cnc32)c1] |
| B-231 | 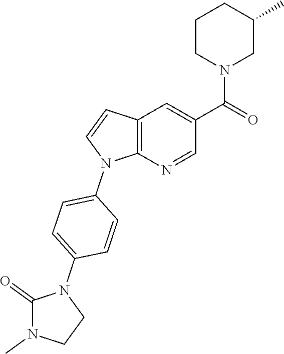 [SMILES: C[C@H]1CCCN(C(=O)c2cnc3c(ccn3-c3ccc(N4CCN(C)C4=O)cc3)c2)C1] |
| B-232 | 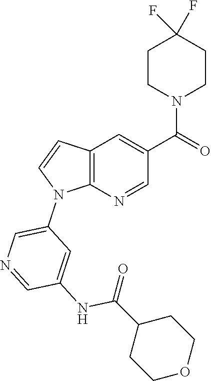 [SMILES: O=C(Nc1cncc(-n2ccc3cc(C(=O)N4CCC(F)(F)CC4)cnc32)c1)C1CCOCC1] |
| B-233 | 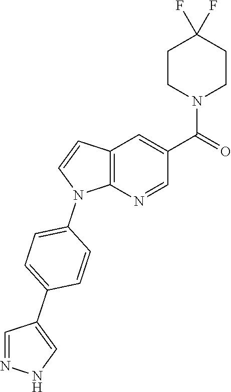 [SMILES: O=C(c1cnc2c(ccn2-c2ccc(-c3cn[nH]c3)cc2)c1)N1CCC(F)(F)CC1] |
| B-234 | 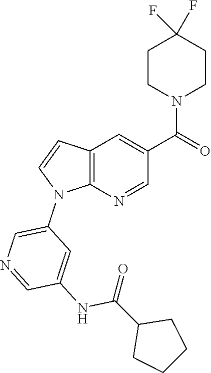 [SMILES: O=C(Nc1cncc(-n2ccc3cc(C(=O)N4CCC(F)(F)CC4)cnc32)c1)C1CCCC1] |
| B-235 | 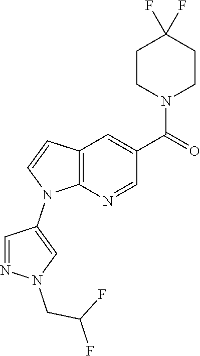 [SMILES: O=C(c1cnc2c(ccn2-c2cnn(CC(F)F)c2)c1)N1CCC(F)(F)CC1] |
| B-236 | 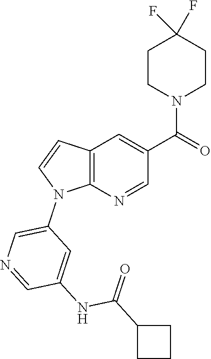 [SMILES: O=C(Nc1cncc(-n2ccc3cc(C(=O)N4CCC(F)(F)CC4)cnc32)c1)C1CCC1] |
| B-237 | 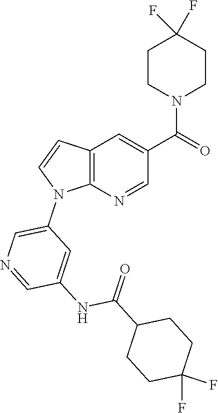 [SMILES: O=C(Nc1cncc(-n2ccc3cc(C(=O)N4CCC(F)(F)CC4)cnc32)c1)C1CCC(F)(F)CC1] |
| B-238 | 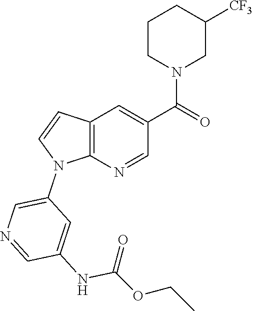 [SMILES: CCOC(=O)Nc1cncc(-n2ccc3cc(C(=O)N4CCCC(C(F)(F)F)C4)cnc32)c1] |
| B-239 | 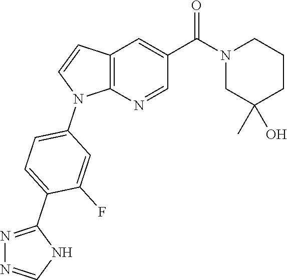 [SMILES: CC1(O)CCCN(C(=O)c2cnc3c(ccn3-c3ccc(-c4nnc[nH]4)c(F)c3)c2)C1] |
| B-240 | 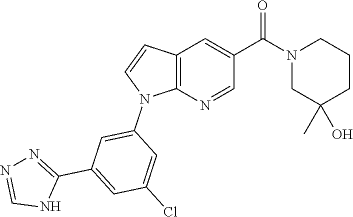 [SMILES: CC1(O)CCCN(C(=O)c2cnc3c(ccn3-c3cc(Cl)cc(-c4nnc[nH]4)c3)c2)C1] |
| B-241 | 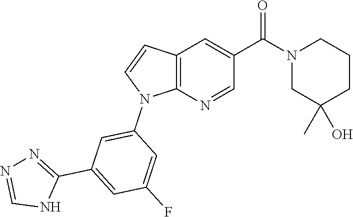 [SMILES: CC1(O)CCCN(C(=O)c2cnc3c(ccn3-c3cc(F)cc(-c4nnc[nH]4)c3)c2)C1] |
| B-242 852 | 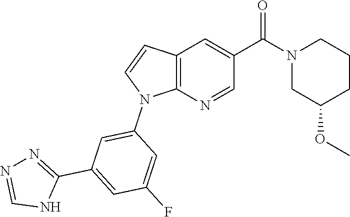 [SMILES: CO[C@H]1CCCN(C(=O)c2cnc3c(ccn3-c3cc(F)cc(-c4nnc[nH]4)c3)c2)C1] |
| B-243 854 | 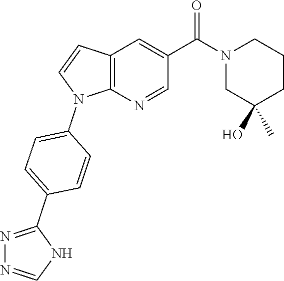 [SMILES: C[C@@]1(O)CCCN(C(=O)c2cnc3c(ccn3-c3ccc(-c4nnc[nH]4)cc3)c2)C1] |
| B-244 855 | 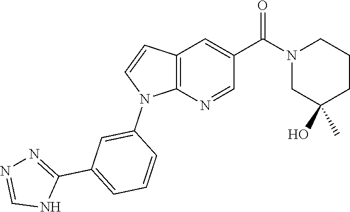 [SMILES: C[C@@]1(O)CCCN(C(=O)c2cnc3c(ccn3-c3cccc(-c4nnc[nH]4)c3)c2)C1] |
| B-245 | 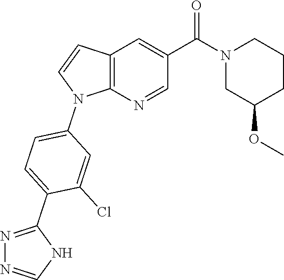 [SMILES: CO[C@@H]1CCCN(C(=O)c2cnc3c(ccn3-c3ccc(-c4nnc[nH]4)c(Cl)c3)c2)C1] |
| B-246 | 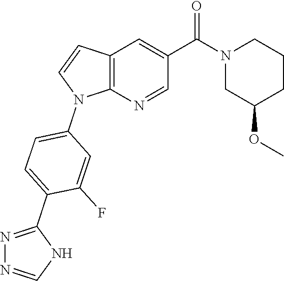 [SMILES: CO[C@@H]1CCCN(C(=O)c2cnc3c(ccn3-c3ccc(-c4nnc[nH]4)c(F)c3)c2)C1] |
| B-247 | 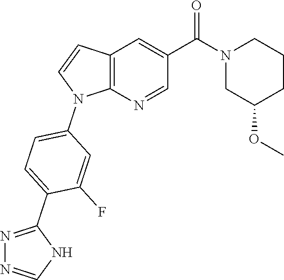 [SMILES: CO[C@H]1CCCN(C(=O)c2cnc3c(ccn3-c3ccc(-c4nnc[nH]4)c(F)c3)c2)C1] |
| B-248 | 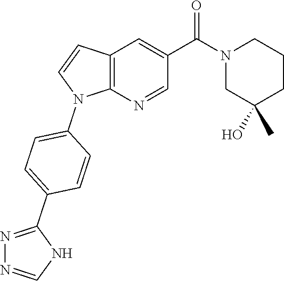 [SMILES: C[C@]1(O)CCCN(C(=O)c2cnc3c(ccn3-c3ccc(-c4nnc[nH]4)cc3)c2)C1] |
| B-249 | 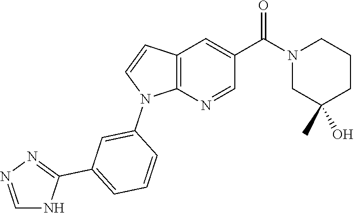 [SMILES: C[C@]1(O)CCCN(C(=O)c2cnc3c(ccn3-c3cccc(-c4nnc[nH]4)c3)c2)C1] |
| B-250 | 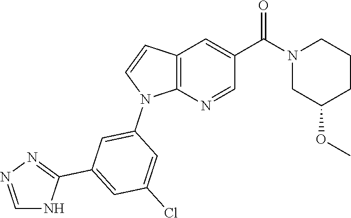 [SMILES: CO[C@H]1CCCN(C(=O)c2cnc3c(ccn3-c3cc(Cl)cc(-c4nnc[nH]4)c3)c2)C1] |
| B-251 | 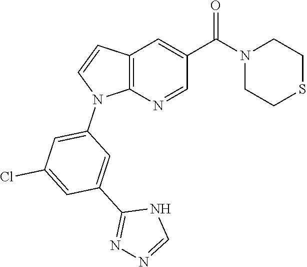 [SMILES: O=C(c1cnc2c(ccn2-c2cc(Cl)cc(-c3nnc[nH]3)c2)c1)N1CCSCC1] |
| B-252 | 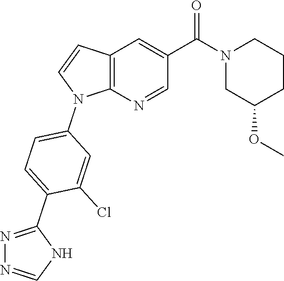 [SMILES: CO[C@H]1CCCN(C(=O)c2cnc3c(ccn3-c3ccc(-c4nnc[nH]4)c(Cl)c3)c2)C1] |
| B-253 | 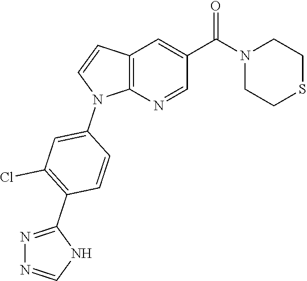 [SMILES: O=C(c1cnc2c(ccn2-c2ccc(-c3nnc[nH]3)c(Cl)c2)c1)N1CCSCC1] |
| B-254 | 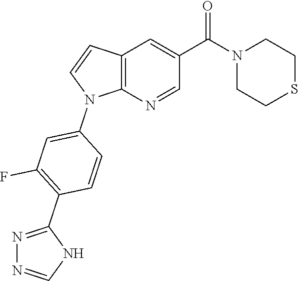 [SMILES: O=C(c1cnc2c(ccn2-c2ccc(-c3nnc[nH]3)c(F)c2)c1)N1CCSCC1] |
| B-255 | 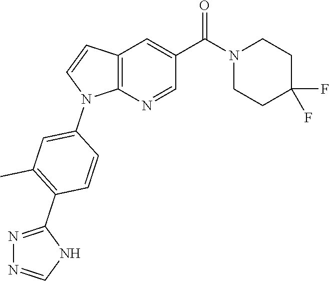 [SMILES: Cc1cc(-n2ccc3cc(C(=O)N4CCC(F)(F)CC4)cnc32)ccc1-c1nnc[nH]1] |
| B-256 | 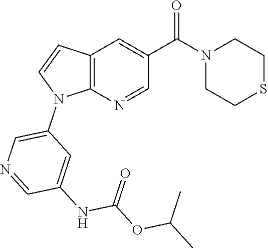 [SMILES: CC(C)OC(=O)Nc1cncc(-n2ccc3cc(C(=O)N4CCSCC4)cnc32)c1] |
| B-257 | 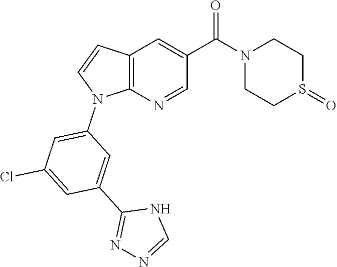 [SMILES: O=C(c1cnc2c(ccn2-c2cc(Cl)cc(-c3nnc[nH]3)c2)c1)N1CCS(=O)CC1] |
| B-258 | 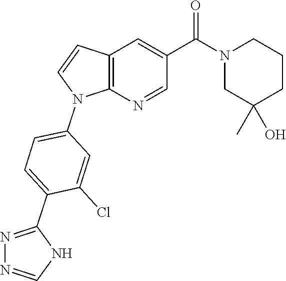 [SMILES: CC1(O)CCCN(C(=O)c2cnc3c(ccn3-c3ccc(-c4nnc[nH]4)c(Cl)c3)c2)C1] |
| B-259 | 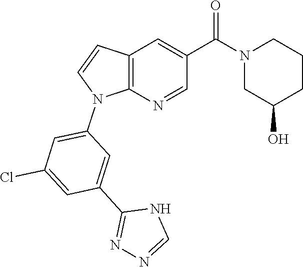 [SMILES: O=C(c1cnc2c(ccn2-c2cc(Cl)cc(-c3nnc[nH]3)c2)c1)N1CCC[C@@H](O)C1] |
| B-260 | 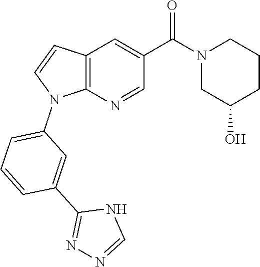 [SMILES: O=C(c1cnc2c(ccn2-c2cccc(-c3nnc[nH]3)c2)c1)N1CCC[C@H](O)C1] |
| B-261 | 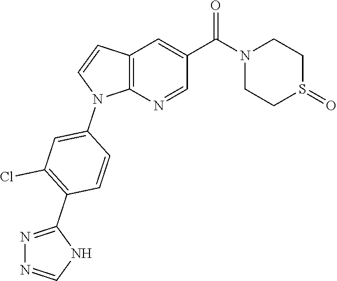 [SMILES: O=C(c1cnc2c(ccn2-c2ccc(-c3nnc[nH]3)c(Cl)c2)c1)N1CCS(=O)CC1] |
| B-262 | 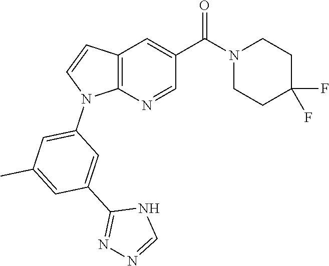 [SMILES: Cc1cc(-c2nnc[nH]2)cc(-n2ccc3cc(C(=O)N4CCC(F)(F)CC4)cnc32)c1] |
| B-263 | 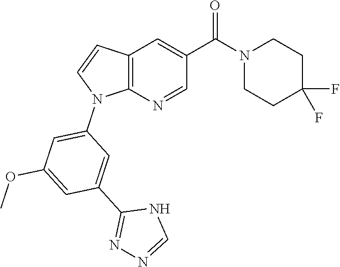 [SMILES: COc1cc(-c2nnc[nH]2)cc(-n2ccc3cc(C(=O)N4CCC(F)(F)CC4)cnc32)c1] |
| B-264 | 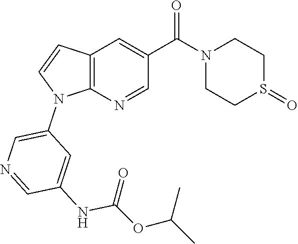 [SMILES: CC(C)OC(=O)Nc1cncc(-n2ccc3cc(C(=O)N4CCS(=O)CC4)cnc32)c1] |
| B-265 | 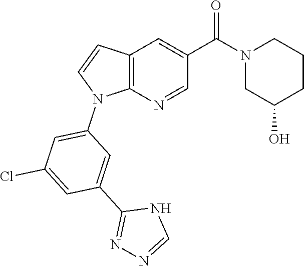 [SMILES: O=C(c1cnc2c(ccn2-c2cc(Cl)cc(-c3nnc[nH]3)c2)c1)N1CCC[C@H](O)C1] |
| B-266 | 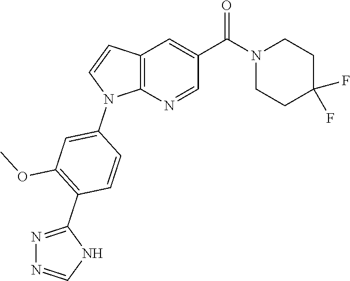 [SMILES: COc1cc(-n2ccc3cc(C(=O)N4CCC(F)(F)CC4)cnc32)ccc1-c1nnc[nH]1] |
| B-267 | 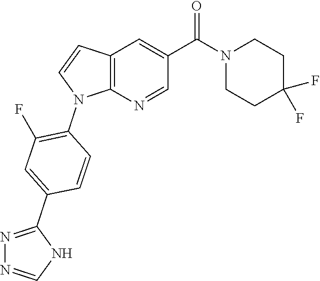 [SMILES: O=C(c1cnc2c(ccn2-c2ccc(-c3nnc[nH]3)cc2F)c1)N1CCC(F)(F)CC1] |
| B-268 | 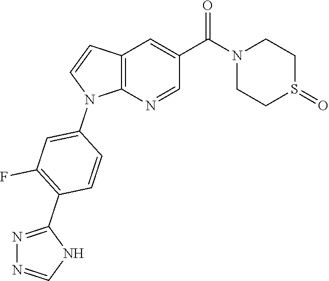 [SMILES: O=C(c1cnc2c(ccn2-c2ccc(-c3nnc[nH]3)c(F)c2)c1)N1CCS(=O)CC1] |
| B-269 | 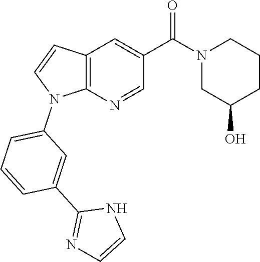 [SMILES: O=C(c1cnc2c(ccn2-c2cccc(-c3ncc[nH]3)c2)c1)N1CCC[C@@H](O)C1] |
| B-270 | 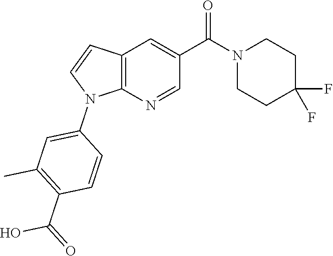 [SMILES: Cc1cc(-n2ccc3cc(C(=O)N4CCC(F)(F)CC4)cnc32)ccc1C(=O)O] |
| B-271 | 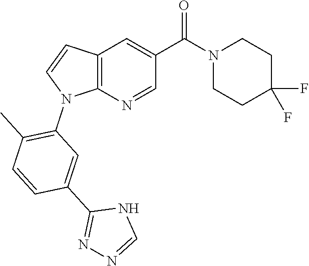 [SMILES: Cc1ccc(-c2nnc[nH]2)cc1-n1ccc2cc(C(=O)N3CCC(F)(F)CC3)cnc21] |
| B-272 | 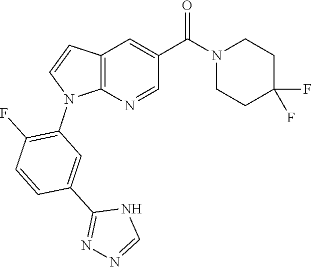 [SMILES: O=C(c1cnc2c(ccn2-c2cc(-c3nnc[nH]3)ccc2F)c1)N1CCC(F)(F)CC1] |
| B-273 | 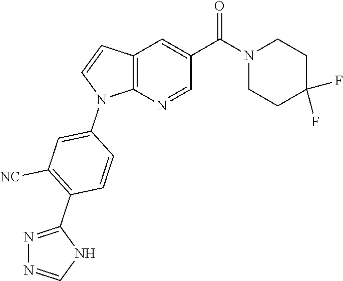 [SMILES: N#Cc1cc(-n2ccc3cc(C(=O)N4CCC(F)(F)CC4)cnc32)ccc1-c1nnc[nH]1] |
| B-274 | 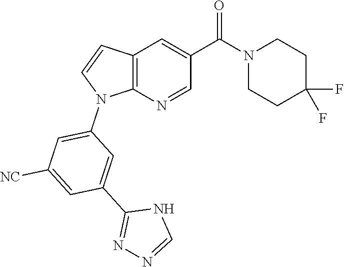 [SMILES: N#Cc1cc(-c2nnc[nH]2)cc(-n2ccc3cc(C(=O)N4CCC(F)(F)CC4)cnc32)c1] |
| B-275 | 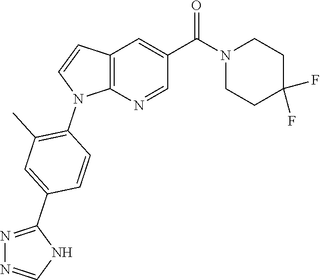 [SMILES: Cc1cc(-c2nnc[nH]2)ccc1-n1ccc2cc(C(=O)N3CCC(F)(F)CC3)cnc21] |
| B-276 | 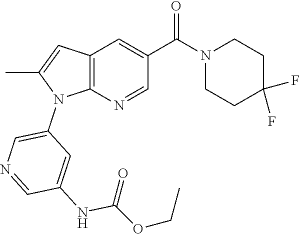 [SMILES: CCOC(=O)Nc1cncc(-n2c(C)cc3cc(C(=O)N4CCC(F)(F)CC4)cnc32)c1] |
| B-277 | 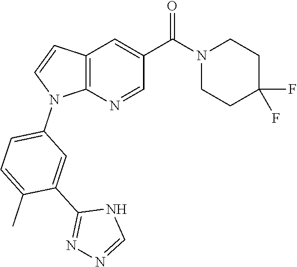 [SMILES: Cc1ccc(-n2ccc3cc(C(=O)N4CCC(F)(F)CC4)cnc32)cc1-c1nnc[nH]1] |
| B-278 | 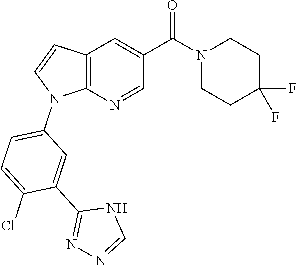 [SMILES: O=C(c1cnc2c(ccn2-c2ccc(Cl)c(-c3nnc[nH]3)c2)c1)N1CCC(F)(F)CC1] |
| B-279 | 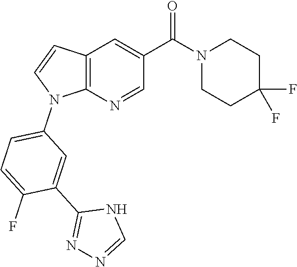 [SMILES: O=C(c1cnc2c(ccn2-c2ccc(F)c(-c3nnc[nH]3)c2)c1)N1CCC(F)(F)CC1] |
| B-280 | 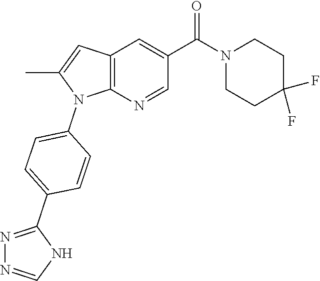 [SMILES: Cc1cc2cc(C(=O)N3CCC(F)(F)CC3)cnc2n1-c1ccc(-c2nnc[nH]2)cc1] |
| B-281 | 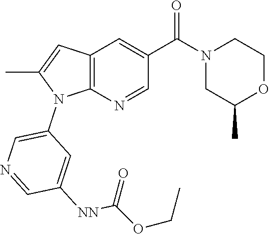 |
| B-282 | 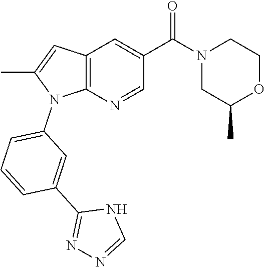 [SMILES: Cc1cc2cc(C(=O)N3CCO[C@@H](C)C3)cnc2n1-c1cccc(-c2nnc[nH]2)c1] |
| B-283 | 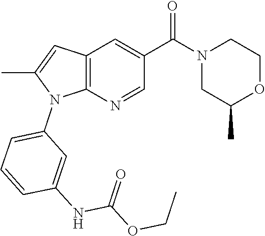 [SMILES: CCOC(=O)Nc1cccc(-n2c(C)cc3cc(C(=O)N4CCO[C@@H](C)C4)cnc32)c1] |
| B-284 | 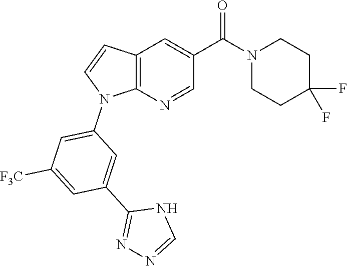 [SMILES: O=C(c1cnc2c(ccn2-c2cc(-c3nnc[nH]3)cc(C(F)(F)F)c2)c1)N1CCC(F)(F)CC1] |
| B-285 | 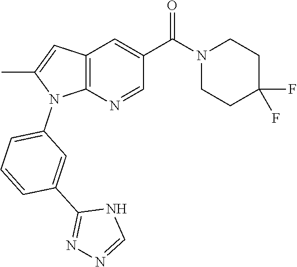 [SMILES: Cc1cc2cc(C(=O)N3CCC(F)(F)CC3)cnc2n1-c1cccc(-c2nnc[nH]2)c1] |
| B-286 | 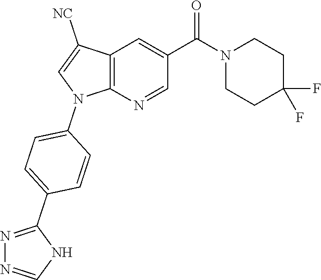 [SMILES: N#Cc1cn(-c2ccc(-c3nnc[nH]3)cc2)c2ncc(C(=O)N3CCC(F)(F)CC3)cc12] |
| B-287 | 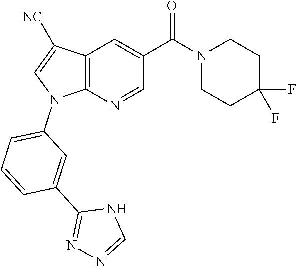 [SMILES: N#Cc1cn(-c2cccc(-c3nnc[nH]3)c2)c2ncc(C(=O)N3CCC(F)(F)CC3)cc12] |
| B-288 | 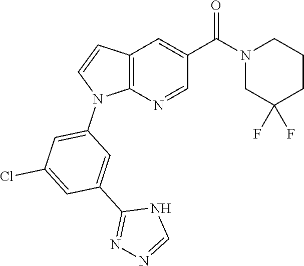 [SMILES: O=C(c1cnc2c(ccn2-c2cc(Cl)cc(-c3nnc[nH]3)c2)c1)N1CCCC(F)(F)C1] |
| B-289 | 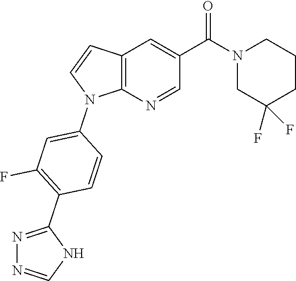 [SMILES: O=C(c1cnc2c(ccn2-c2ccc(-c3nnc[nH]3)c(F)c2)c1)N1CCCC(F)(F)C1] |
| B-290 | 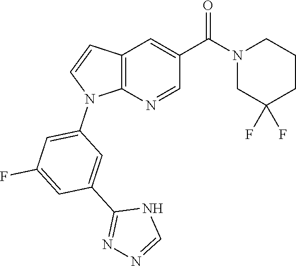 [SMILES: O=C(c1cnc2c(ccn2-c2cc(F)cc(-c3nnc[nH]3)c2)c1)N1CCCC(F)(F)C1] |
| B-291 | 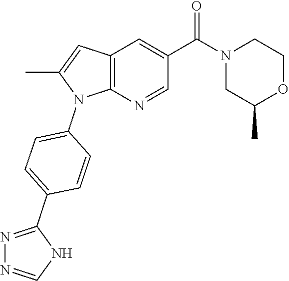 [SMILES: Cc1cc2cc(C(=O)N3CCO[C@@H](C)C3)cnc2n1-c1ccc(-c2nnc[nH]2)cc1] |
| B-292 | 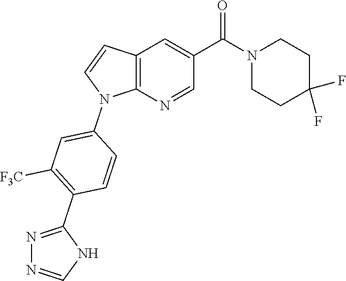 [SMILES: O=C(c1cnc2c(ccn2-c2ccc(-c3nnc[nH]3)c(C(F)(F)F)c2)c1)N1CCC(F)(F)CC1] |

In some embodiments, the PGDH inhibitor is a compound provided in Table 2, or a pharmaceutically acceptable salt or solvate thereof.

TABLE 2
Representative sulfoxide PGDH inhibitors.

| Compound | |
|---|---|
| No | Structure |
| B-293 | 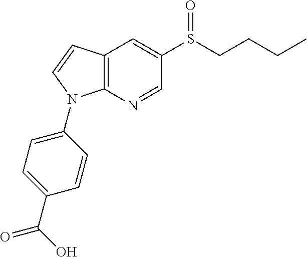 [SMILES: CCCCS(=O)c1cnc2c(ccn2-c2ccc(C(=O)O)cc2)c1] |
| B-294 | 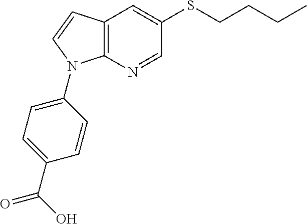 [SMILES: CCCCSc1cnc2c(ccn2-c2ccc(C(=O)O)cc2)c1] |
| B-295 | 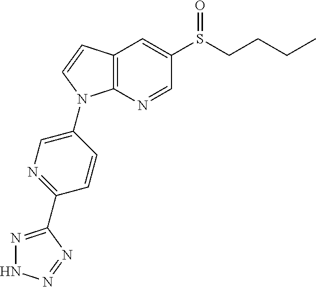 [SMILES: CCCCS(=O)c1cnc2c(ccn2-c2ccc(-c3nn[nH]n3)nc2)c1] |
| B-296 | 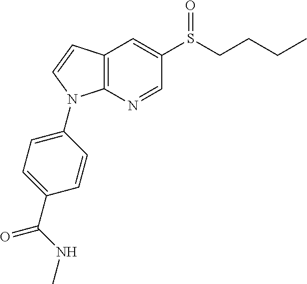 [SMILES: CCCCS(=O)c1cnc2c(ccn2-c2ccc(C(=O)NC)cc2)c1] |
| B-297 | 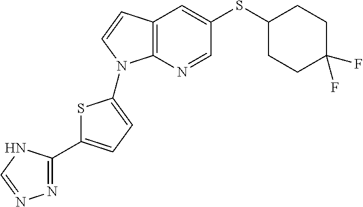 [SMILES: FC1(F)CCC(Sc2cnc3c(ccn3-c3ccc(-c4nnc[nH]4)s3)c2)CC1] |
| B-298 | 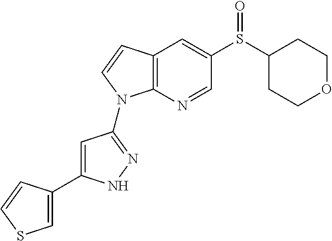 [SMILES: O=S(c1cnc2c(ccn2-c2cc(-c3ccsc3)[nH]n2)c1)C1CCOCC1] |
| B-299 | 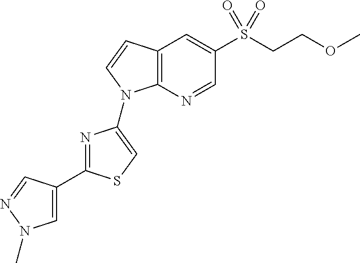 [SMILES: COCCS(=O)(=O)c1cnc2c(ccn2-c2csc(-c3cnn(C)c3)n2)c1] |
| B-300 | 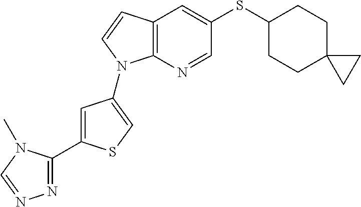 [SMILES: Cn1cnnc1-c1cc(-n2ccc3cc(SC4CCC5(CC4)CC5)cnc32)cs1] |
| B-301 | 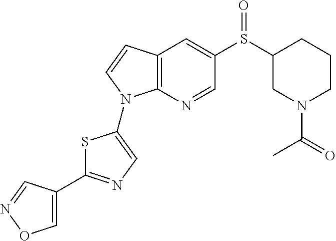 [SMILES: CC(=O)N1CCCC(S(=O)c2cnc3c(ccn3-c3cnc(-c4cnoc4)s3)c2)C1] |
| B-302 | 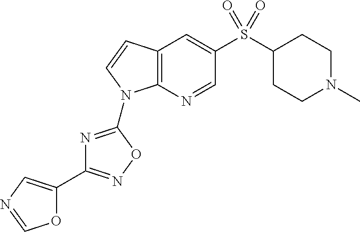 [SMILES: CN1CCC(S(=O)(=O)c2cnc3c(ccn3-c3nc(-c4cnco4)no3)c2)CC1] |
| B-303 | 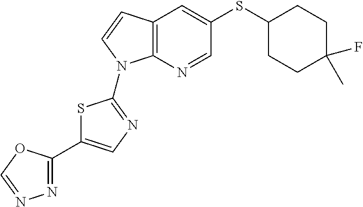 [SMILES: CC1(F)CCC(Sc2cnc3c(ccn3-c3ncc(-c4nnco4)s3)c2)CC1] |
| B-304 | 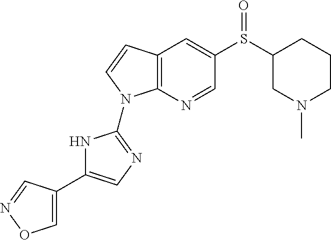 [SMILES: CN1CCCC(S(=O)c2cnc3c(ccn3-c3ncc(-c4cnoc4)[nH]3)c2)C1] |
| B-305 | 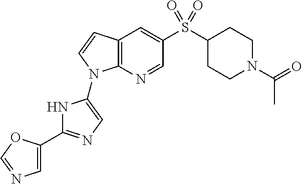 [SMILES: CC(=O)N1CCC(S(=O)(=O)c2cnc3c(ccn3-c3cnc(-c4cnco4)[nH]3)c2)CC1] |
| B-306 | 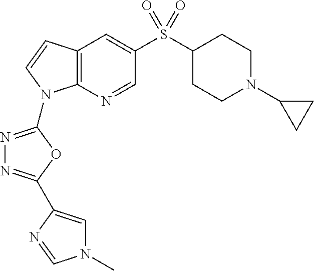 [SMILES: Cn1cnc(-c2nnc(-n3ccc4cc(S(=O)(=O)C5CCN(C6CC6)CC5)cnc43)o2)c1] |
| B-307 | 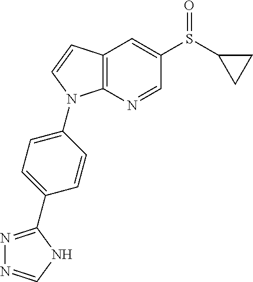 [SMILES: O=S(c1cnc2c(ccn2-c2ccc(-c3nnc[nH]3)cc2)c1)C1CC1] |
| B-308 | 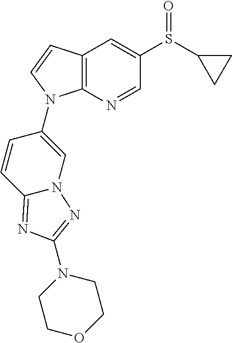 [SMILES: O=S(c1cnc2c(ccn2-c2ccc3nc(N4CCOCC4)nn3c2)c1)C1CC1] |
| B-309 | 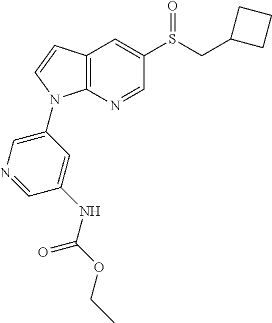 [SMILES: CCOC(=O)Nc1cncc(-n2ccc3cc(S(=O)CC4CCC4)cnc32)c1] |
| B-310 | 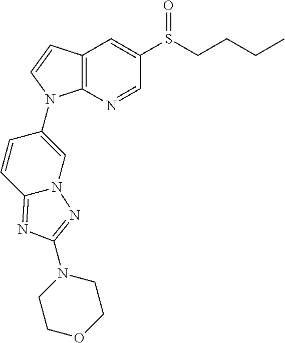 [SMILES: CCCCS(=O)c1cnc2c(ccn2-c2ccc3nc(N4CCOCC4)nn3c2)c1] |
| B-311 | 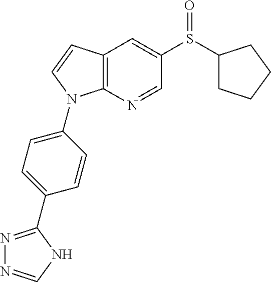 [SMILES: O=S(c1cnc2c(ccn2-c2ccc(-c3nnc[nH]3)cc2)c1)C1CCCC1] |
| B-312 | 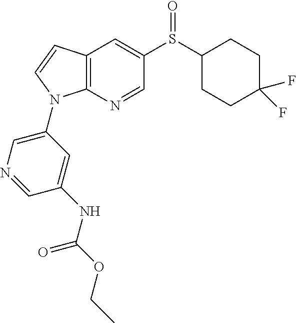 [SMILES: CCOC(=O)Nc1cncc(-n2ccc3cc(S(=O)C4CCC(F)(F)CC4)cnc32)c1] |
| B-313 | 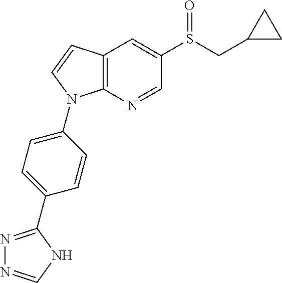 [SMILES: O=S(CC1CC1)c1cnc2c(ccn2-c2ccc(-c3nnc[nH]3)cc2)c1] |
| B-314 | 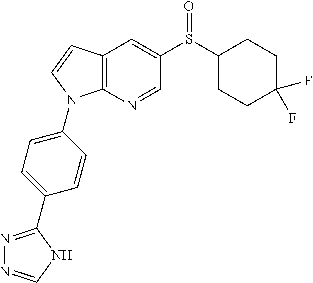 [SMILES: O=S(c1cnc2c(ccn2-c2ccc(-c3nnc[nH]3)cc2)c1)C1CCC(F)(F)CC1] |
| B-315 | 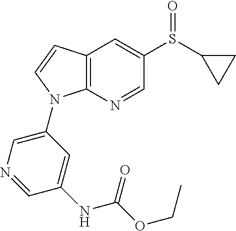 [SMILES: CCOC(=O)Nc1cncc(-n2ccc3cc(S(=O)C4CC4)cnc32)c1] |
| B-316 | 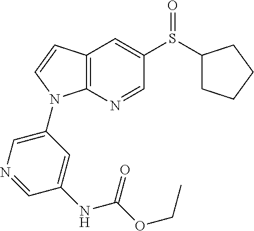 [SMILES: CCOC(=O)Nc1cncc(-n2ccc3cc(S(=O)C4CCCC4)cnc32)c1] |
| B-317 | 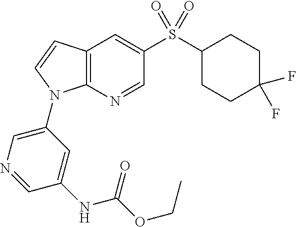 [SMILES: CCOC(=O)Nc1cncc(-n2ccc3cc(S(=O)(=O)C4CCC(F)(F)CC4)cnc32)c1] |
| B-318 | 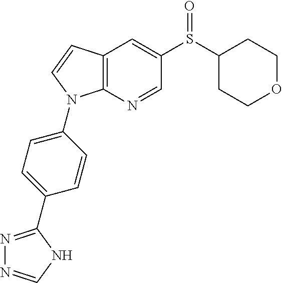 [SMILES: O=S(c1cnc2c(ccn2-c2ccc(-c3nnc[nH]3)cc2)c1)C1CCOCC1] |
| B-319 | 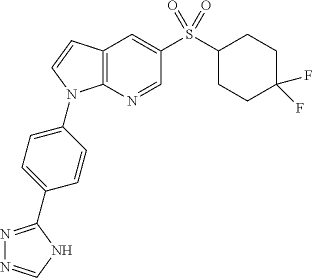 [SMILES: O=S(=O)(c1cnc2c(ccn2-c2ccc(-c3nnc[nH]3)cc2)c1)C1CCC(F)(F)CC1] |

### Methods of Use

In one aspect, provided herein are methods for treating various disorders in a subject in need thereof, comprising administering to said subject a compound described herein. In some embodiments, the inhibitors of hydroxyprostaglandin dehydrogenase provided herein may be used for the prevention or treatment of a disease or a disorder that is associated with hydroxyprostaglandin dehydrogenase (such as 15-PGDH) and/or decreased levels of prostaglandin. In some embodiments, the inhibitors of hydroxyprostaglandin dehydrogenase provided herein may be used for the prevention or treatment of a disease or a disorder in which it is desirable to increase prostaglandin levels in the subject having said disease or disorder.

In some embodiments, the methods for treating the disorders comprises administering to said subject a 15-PGDH inhibitor. In some embodiments, a compound described herein is the 15-PGDH inhibitor (e.g. a compound of Formula (N) or a pharmaceutically acceptable salt or solvate thereof). In some embodiments, the methods comprise administering a therapeutically effective amount of a compound described herein. In some embodiments, the methods comprise administering a therapeutically effective amount of a compound described herein or a pharmaceutically acceptable salt or solvate thereof (e.g. a compound of Formula (N) or a pharmaceutically acceptable salt or solvate thereof). In some embodiments, the compound described herein is a 15-PGDH inhibitor (e.g. a compound of Formula (N), or a pharmaceutically acceptable salt or solvate thereof). In some embodiments, the administration takes place in vitro. In other embodiments, the administration takes place in vivo.

As used herein, a therapeutically effective amount of a 15-PGDH inhibitor refers to an amount sufficient to effect the intended application, including but not limited to, disease treatment, as defined herein. Also contemplated in the subject methods is the use of a sub-therapeutic amount of a 15-PGDH inhibitor for treating an intended disease condition.

The amount of the 15-PGDH inhibitor administered may vary depending upon the intended application (in vitro or in vivo), or the subject and disease condition being treated, e.g., the weight and age of the subject, the severity of the disease condition, the manner of administration and the like, which can readily be determined by one of ordinary skill in the art.

Measuring inhibition of biological effects of 15-PGDH can comprise performing an assay on a biological sample, such as a sample from a subject. Any of a variety of samples may be selected, depending on the assay. Examples of samples include, but are not limited to, blood samples (e.g. blood plasma or serum), exhaled breath condensate samples, bronchoalveolar lavage fluid, sputum samples, urine samples, and tissue samples.

A subject being treated with a 15-PGDH inhibitor may be monitored to determine the effectiveness of treatment, and the treatment regimen may be adjusted based on the subject's physiological response to treatment. For example, if inhibition of a biological effect of 15-PGDH is above or below a threshold, the dosing amount or frequency may be decreased or increased, respectively. The methods can further comprise continuing the therapy if the therapy is determined to be efficacious. The methods can comprise maintaining, tapering, reducing, or stopping the administered amount of a compound in the therapy if the therapy is determined to be efficacious. The methods can comprise increasing the administered amount of a compound in the therapy if it is determined not to be efficacious. Alternatively, the methods can comprise stopping therapy if it is determined not to be efficacious. In some embodiments, treatment with a 15-PGDH inhibitor is discontinued if inhibition of the biological effect is above or below a threshold, such as in a lack of response or an adverse reaction. The biological effect may be a change in any of a variety of physiological indicators.

In general, a 15-PGDH inhibitor is a compound that inhibits one or more biological effects of 15-PGDH. Such biological effects may be inhibited by about or more than about 10%, 20%, 30%, 40%, 50%, 60%, 70%, 80%, 90%, 95%, or more.

In some other embodiments, the subject methods are useful for treating a disease condition associated with 15-PGDH. Any disease condition that results directly or indirectly from an abnormal activity or expression level of 15-PGDH can be an intended disease condition.

In one aspect, provided herein is a method of promoting and/or stimulation skin pigmentation, comprising administering one or more of the compositions described herein to a subject in need thereof. Inhibitors of 15-PGDH are known to promote skin pigmentation (Markowitz et. al., WO 2015/065716). The hydroxyprostaglandin dehydrogenase inhibitors described herein can be used for promoting and/or inducing and/or stimulating pigmentation of the skin and/or skin appendages, and/or as an agent for preventing and/or limiting depigmentation and/or whitening of the skin and/or skin appendages, in particular as an agent for preventing and/or limiting canities. In some embodiments, the 15-PGDH inhibitors provided herein can be applied to skin of a subject, e.g., in a topical application, to promote and/or stimulate pigmentation of the skin and/or hair growth, inhibit hair loss, and/or treat skin damage or inflammation, such as skin damage caused by physical or chemical irritants and/or UV-exposure.

In another aspect, provided herein is a method of inhibiting hair loss, comprising administering one or more of the compositions described herein to a subject in need thereof. It is known that prostaglandins play an important role in hair growth. Prostaglandins such as prostaglandin A1, F2a and E2 are stored in hair follicles or adjacent skin environments and have been shown to be essential in maintaining and increasing hair density (Colombe L et. al, 2007, Exp. Dermatol, 16(9), 762-9). It has been reported that 15-PGDH, which is involved in the degradation of prostaglandins is present in the hair follicle dermal papillae, inactivates prostaglandins, especially, PGF2a and PGE2, to cause scalp damage and alopecia (Michelet J F et. al., 2008, Exp. Dermatol, 17(10), 821-8). Thus, the hydroxyprostaglandin dehydrogenase inhibitors described herein that have a suppressive or inhibitory activity against 15-PGDH can improve scalp damage, prevent alopecia and promote hair growth and be used in a pharmaceutical composition for the prevention of alopecia and the promotion of hair growth.

In another aspect, provided herein is a method of preventing and/or treating skin inflammation and/or damage, comprising administering one or more of the compositions described herein to a subject in need thereof.

In another aspect, provided herein is a method of preventing and/or treating vascular insufficiency, comprising administering one or more of the compositions described herein to a subject in need thereof. Prostaglandins including prostaglandin homologues produced in the body have been known to maintain the proper action of the blood vessel wall, especially to contribute to vasodilation for blood flow, preventing platelet aggregation and modulating the proliferation of smooth muscle that surrounds blood vessel walls (Yan. Cheng et. al., 2006, J. Clin., Invest). In addition, the inhibition of prostaglandins production or the loss of their activity causes the degeneration of the endothelium in the blood vessel walls, platelet aggregation and the dysfunction of cellular mechanism in the smooth muscle. Among others, the production of prostaglandins in blood vessels was shown to be decreased in hypertension patients, including pulmonary artery hypertension. the 15-PGDH inhibitors described herein can be used in a pharmaceutical composition for the prevention or the treatment of cardiovascular disease and/or diseases of vascular insufficiency, such as Raynaud's disease, Buerger's disease, diabetic neuropathy, and pulmonary artery hypertension.

In another aspect, provided herein is a method of preventing, treating, minimizing and/or reversing congestive heart failure, cardiomyopathy, comprising administering one or more of the compositions described herein to a subject in need thereof. In another aspect, provided herein is a method of reducing cardiac ejection fraction, comprising administering one or more of the compositions described herein to a subject in need thereof. It has been shown that administration of a 15-PGDH inhibitor can be used to treat, prevent, minimize, and/or reverse congestive heart failure, cardiomyopathy, and/or reduction of cardiac ejection fraction (Markowitz et. al., WO2018/187810). As such, the hydroxyprostaglandin dehydrogenase inhibitors described herein can be administered to a subject in need to treat, prevent, minimize and/or reverse congestive heart failure, cardiomyopathy, and/or reduction of cardiac ejection fraction.

In another aspect, provided herein is a method of preventing and/or treating a gastrointestinal disease, comprising administering one or more of the compositions described herein to a subject in need thereof. Prostaglandins are essential for maintaining the mechanism for protecting and defending gastric mucus membrane (Wallace J L., 2008, Physiol Rev., 88(4), 1547-65, S. J. Konturek et al., 2005, Journal of Physiology and Pharmacology, 56 (5)). The inhibitors of hydroxyprostaglandin dehydrogenase described herein show a suppressive or inhibitory activity against 15-PGDH, which degrades prostaglandins that protect gastric mucus membranes. As such, the hydroxyprostaglandin dehydrogenase inhibitors can be effective for the prevention or the treatment of gastrointestinal diseases, inter alia, gastritis and gastric ulcer. In addition, the hydroxyprostaglandin dehydrogenase inhibitors provided herein may be used to prevent and/or treat other forms of intestinal injury including toxicity from radiation and/or chemotherapy, and chemotherapy-induced mucositis.

Additionally, it has been shown that administration of 15-PGDH inhibitors, alone or in combination with corticosteroids and/or TNF inhibitors can treat intestinal, gastrointestinal, or bowel disorders such as oral ulcers, gum disease, gastritis, colitis, ulcerative colitis, gastric ulcers, inflammatory bowel disease, and Crohn's disease (Markowitz et. al., WO 2018/102552). As such, the hydroxyprostaglandin dehydrogenase inhibitors provided herein can be used to treat and/or prevent treat intestinal, gastrointestinal, or bowel disorders such as oral ulcers, gum disease, gastritis, colitis, ulcerative colitis, gastric ulcers, inflammatory bowel disease, and Crohn's disease.

In another aspect, provided herein is a method of preventing and/or treating renal dysfunction, comprising administering one or more of the compositions described herein to a subject in need thereof. In the kidney, prostaglandins modulate renal blood flow and may serve to regulate urine formation by both renovascular and tubular effects. In clinical studies, inhibitors of prostaglandin have been used to improve creatinine clearance in patients with chronic renal disease, to prevent graft rejection and cyclosporine toxicity in renal transplant patients, to reduce the urinary albumin excretion rate and N-acetyl-beta-D-glucosaminidase levels in patients with diabetic nephropathy (Porter, A m., 1989, J. C₈ rdiol., 64: 22E-26E). Furthermore, it has been reported that prostaglandins serve as vasodilators in the kidney, and, thus, the inhibition of prostaglandin production in the kidney results in renal dysfunction (Hao. C M, 2008, Annu Rev Physiol, 70, 357.about.77). The hydroxyprostaglandin dehydrogenase inhibitors described herein have a suppressive or inhibitory activity against 15-PGDH that degrades prostaglandins and can be used for the prevention and/or treatment of renal diseases that are associated with renal dysfunction.

In another aspect, provided herein is a method of stimulation bone resorption and bone formation, comprising administering one or more of the compositions described herein to a subject in need thereof. Prostaglandins have been shown to stimulate bone resorption and bone formation to increase the volume and the strength of the bone (H. Kawaguchi et. al., Clinical Orthop. Rel. Res., 313, 1995; J. Keller et al., Eur. Jr. Exp. Musculoskeletal Res., 1, 1992, 8692). Furthermore, inhibition of 15-PGDH increases callus size and mineralization after bone fracture (Collier et. al., OR⁸ 2017 Annual Meeting Paper No. 0190). Considering that 15-PGDH inhibits the activities of prostaglandins as mentioned in the above, the inhibition of 15-PGDH activity may lead to the promotion of bone resorption and bone formation that are inhibited by 15-PGDH. Thus, the inhibitors of hydroxyprostaglandin dehydrogenase described herein can be effective for the promotion of bone resorption and bone formation by inhibiting 15-PGDH activity. The hydroxyprostaglandin dehydrogenase inhibitors provided herein can also be used to increase bone density, treat osteoporosis, promote healing of fractures, promote healing after bone surgery or joint replacement, and/or to promote healing of bone to bone implants, bone to artificial implants, dental implants, and bone grafts.

In another aspect, provided herein is a method of stimulating tissue regeneration by stimulating, comprising administering one or more of the compositions described herein to a subject in need thereof. Prostaglandin PGE2 supports expansion of several types of tissue stem cells. Inhibition of 15-hydroxyprostaglandin dehydrogenase (15-PGDH), a prostaglandin-degrading enzyme, potentiates tissue regeneration in multiple organs. Studies show that inhibition of 15-PGDH increases prostaglandin PGE2 levels in bone marrow and other tissues; accelerates hematopoietic recovery following a bone marrow transplant; promotes tissue regeneration of colon and liver injury (Zhang, Y. et. al. Science 2015, 348 (6240)). The hydroxyprostaglandin dehydrogenase inhibitors provided herein can be used for tissue regeneration by supporting the expansion of tissue stem cells.

In another aspect, provided herein is a method of modulating cervical ripening, comprising administering one or more of the compositions described herein to a subject in need thereof. Prostaglandin E2 (PGE2) is a known cervical ripening agent that mediates EP2-receptor- signaling pathways in human cervical stromal cells; targets its own synthesis by increasing COX-2 and PTGES expression; and decreases its metabolism by loss of its degradative enzyme 15-PGDH (Word et. Al., WO2019010482) Downregulation of 15-PGDH was also found to be crucial for PGE2-induced cervical ripening and preterm birth. Modulation of 15-PDGH activity can be used to modulate cervical ripening; and induce or prevent preterm labor. The hydroxyprostaglandin dehydrogenase inhibitors provided herein can be used to induce cervical ripening and labor, alone or in combination with another labor inducing agent.

In another aspect, provided herein is a method of promoting neuroprotection and/or stimulating neuronal regeneration, comprising administering one or more of the compositions described herein to a subject in need thereof. Prostaglandins, via their specific G protein coupled receptors, have a variety of physiological functions in the central nervous system. The major prostaglandin, prostaglandin E2 (PGE2) can activate receptor types EP1, 2, 3, and 4. Activation of EP2 and EP4 receptors can regulate adenylate cyclase and the generation of 3,5′-cyclic adenosine monophosphate (cAMP), whereas the activation of EP1 and EP3 receptors can regulate C₈ 2+ signaling. Studies show that the EP1 and EP2 receptors are expressed in neurons and microglia as well as neurons of the cerebral cortex, striatum, and hippocampus. In addition, activation of the EP2 receptor by PGE2 is involved in long-term synaptic plasticity and cognitive function (Chemtob et al. Semin Perinatol. 1994 February; 18(1):23-9; Yang et al., J Neurochem.2009 Jan; 108(1):295-304). Studies also show that following activation, different PGE2 receptors can contribute or protect against N-methyl-D-aspartate (NMDA) neurotoxicity and ischemic stroke (Ahmad et al., Exp Transl Stroke Med.2010 Jul 8; 2(1):12). Other studies show that activation of the EP2 receptors protected neurons from amyloid β-peptide neurotoxicity in vitro (Echeverria et al., Eur J Neurosci.2005 Nov; 22(9):2199-206). Several studies suggest that the mechanism by which PGE2 affords neuroprotection is through EP2 or EP4 receptors, as they both increases cAMP, followed by a protein kinase A (PKA)-dependent pathway (Echeverria et al. Eur J Neurosci.2005 Nov; 22(9):2199-206; McCullough et al., J Neurosci.2004 Jan 7; 24(1):257-68). Stimulation of these receptors with PGE2 by administration of a compound that inhibits, reduces, and/or antagonizes 15-PGDH activity, such as the hydroxyprostaglandin dehydrogenase inhibitors that can inhibit 15-PGDH described herein, can promote neuroprotection in a subject from axonal degeneration, neuronal cell death, and/or glia cell damage after injury, augment neuronal signaling underlying learning and memory, stimulate neuronal regeneration after injury, and/or treat diseases, disorders, and/or conditions of the nervous system.

In another aspect, provided herein is a method of treating and/or preventing a neurological disorder, a neuropsychiatric disorder, a neural injury, a neural toxicity disorder, a neuropathic pain, or a neural degenerative disorder, comprising administering one or more of the compositions described herein to a subject in need thereof. In some embodiments, the disease, disorder, and/or condition of the nervous system, which can be treated with hydroxyprostaglandin dehydrogenase inhibitors provided herein, can include at least one of a neurological disorder, a neuropsychiatric disorder, a neural injury, a neural toxicity disorder, a neuropathic pain, or a neural degenerative disorder. For example, the neurological disorder can include at least one of traumatic or toxic injuries to peripheral or cranial nerves, spinal cord or brain, such as traumatic brain injury, stroke, cerebral aneurism, and spinal cord injury. The neurological disorder can also include at least one of Alzheimer's disease, dementias related to Alzheimer's disease, Parkinson's, Lewy diffuse body diseases, senile dementia, Huntington's disease, Gilles de Ia Tourette's syndrome, multiple sclerosis, amyotrophic lateral sclerosis, hereditary motor and sensory neuropathy, diabetic neuropathy, progressive supranuclear palsy, epilepsy, or Jakob- Creutzfieldt disease.

In some embodiments, the neural injury can be caused by or associated with at least one of epilepsy, cerebrovascular diseases, autoimmune diseases, sleep disorders, autonomic disorders, urinary bladder disorders, abnormal metabolic states, disorders of the muscular system, infectious and parasitic diseases, neoplasms, endocrine diseases, nutritional and metabolic diseases, immunological diseases, diseases of the blood and blood-forming organs, mental disorders, diseases of the nervous system, diseases of the sense organs, diseases of the circulatory system, diseases of the respiratory system, diseases of the digestive system, diseases of the genitourinary system, diseases of the skin and subcutaneous tissue, diseases of the musculoskeletal system and connective tissue, congenital anomalies, or conditions originating in the perinatal period.

In certain embodiments, the hydroxyprostaglandin dehydrogenase inhibitors can be administered to a subject or neurons of the subject to promote the survival, growth, development and/or function of the neurons, particularly, the central nervous system (CNS), brain, cerebral, and hippocampal neurons. In certain embodiments, the hydroxyprostaglandin dehydrogenase inhibitors can be used stimulate hippocampal neurogenesis, for the treatment of neuropsychiatric and neurodegenerative diseases, including (but not limited to) schizophrenia, major depression, bipolar disorder, normal aging, epilepsy, traumatic brain injury, post-traumatic stress disorder, Parkinson's disease, Alzheimer's disease, Down syndrome, spinocerebellar ataxia, amyotrophic lateral sclerosis, Huntington's disease, stroke, radiation therapy, chronic stress, and abuse of neuro-active drugs, such as alcohol, opiates, methamphetamine, phencyclidine, and cocaine.

In another aspect, provided herein is a method of treating and/or preventing fibrotic or adhesion disease, disorder or condition, comprising administering one or more of the compositions described herein to a subject in need thereof. It has been shown that inhibitors of short-chain dehydrogenase activity, such as 15-PGDH inhibitors, can be administered to a subject in need thereof to decrease fibrotic symptoms, such as collagen deposition, collagen accumulation, collagen fiber formation, inflammatory cytokine expression, and inflammatory cell infiltration, and treat and/or prevent various fibrotic diseases, disorders, and conditions characterized, in whole or in part, by the excess production of fibrous material, including excess production of fibrotic material within the extracellular matrix, or the replacement of normal tissue elements by abnormal, non-functional, and/or excessive accumulation of matrix-associated components (Markowitz et. al., WO2016/144958).

Fibrotic diseases, disorders and conditions characterized, in whole or in part, by excess production of fibrotic material can include systemic sclerosis, multifocal fibrosclerosis, nephrogenic systemic fibrosis, scleroderma(including morphea, generalized morphea, or linear scleroderma), sclerodermatous graft- vs-host-disease, kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g. pulmonary fibrosis, glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fasciitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, myelodysplastic syndrome, myeloproliferative syndrome, collagenous colitis, acute fibrosis, organ specific fibrosis, and the like. The hydroxyprostaglandin dehydrogenase inhibitors provided herein can be used to treat or prevent a fibrotic disease, disorder or condition.

The hydroxyprostaglandin dehydrogenase inhibitors provided herein can be used to treat or prevent kidney fibrosis, including kidney fibrosis resulting from dialysis following kidney failure, catheter placement, a nephropathy, glomerulosclerosis, glomerulonephritis, chronic renal insufficiency, acute kidney injury, end stage renal disease or renal failure, or combinations thereof.

The hydroxyprostaglandin dehydrogenase inhibitors provided herein can be used to treat or prevent liver fibrosis, including liver fibrosis resulting from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, alpha- 1-antitrypsin deficiency, primary biliary cirrhosis, drug reaction and exposure to toxins, or combinations thereof.

The hydroxyprostaglandin dehydrogenase inhibitors provided herein can be used to treat or prevent heart fibrosis such as cardiac fibrosis, endomyocardial fibrosis, idiopathic pulmonary fibrosis, and kidney fibrosis.

The hydroxyprostaglandin dehydrogenase inhibitors provided herein can be used to treat or prevent systemic sclerosis.

The hydroxyprostaglandin dehydrogenase inhibitors provided herein can be used to treat or prevent fibrotic diseases, disorders or conditions caused by post-surgical adhesion formation.

The hydroxyprostaglandin dehydrogenase inhibitors provided herein can be used to reduce in intensity, severity, or frequency, and/or delay onset of one or more symptoms or features of a fibrotic disease, disorder or condition, or other related diseases, disorders or conditions.

The hydroxyprostaglandin dehydrogenase inhibitors provided herein can be used to decrease or reduce collagen secretion, or collagen deposition, or collagen fiber accumulation, in a tissue or organ, such as the lung, the liver, the intestines, the colon, the skin or the heart, or a combination thereof.

Studies have shown that 15-PGDH inhibition ameliorates inflammatory pathology and fibrosis in pulmonary fibrosis (Smith et. al., bioRxiv 2019.12.16.878215; Barnthaler et. al., J. Allergy Clin. Immunol. 2019, 145 (3), 818-833). In some embodiments, the hydroxyprostaglandin dehydrogenase inhibitors described herein can be used to treat or prevent lung fibrosis, including pulmonary fibrosis, pulmonary hypertension, chronic obstructive pulmonary disease (COPD), asthma, idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension, and combinations thereof.

In another aspect, provided herein is a method of reducing and/or preventing scar formation, comprising administering one or more of the compositions described herein to a subject in need thereof. The hydroxyprostaglandin dehydrogenase inhibitors provided herein can used for reducing or preventing scar formation in a subject. The hydroxyprostaglandin dehydrogenase inhibitors provided herein can be used to reduce or prevent scar formation on skin or scleroderma.

In another aspect, provided herein is a method of treating and/or preventing muscle disorder, muscle injury and/or muscle atrophy, comprising administering one or more of the compositions described herein to a subject in need thereof. Studies have shown that inhibition of PGE2 degrading enzymes such as 15-PGDH, enable muscle regeneration and muscle repair after injury (Ho et al., PNAS 2017; Dong et al., Stem cell research and therapy 2020). The inhibitors of hydroxyprostaglandin dehydrogenase provided herein can be used to treat muscle disorder, muscle injury and/or muscle atrophy in a subject. In some cases, said subject suffering from a muscle disorder, muscle injury and/or muscle atrophy may have Duchenne muscular dystrophy (DMD), Becker muscular dystrophy, Fukuyama congenital muscular dystrophy (FCMD), limb girdle muscular dystrophy, congenital muscular dystrophy, facioscapulohumeral muscular dystrophy (FHMD), amyotrophic lateral sclerosis (ALS), distal muscular dystrophy (DD), an inherited myopathy, myotonic muscular dystrophy (MDD), oculopharyngeal muscular dystrophy, distal muscular dystrophy, Emery-Dreifuss muscular dystrophy, myotonia congenita, mitochondrial myopathy (DD), myotubular myopathy (MM), myasthenia gravis (MG), periodic paralysis, polymyositis, rhabdomyolysis, dermatomyositis, cancer cachexia, AIDS cachexia, stress induced urinary incontinence, urethral sphincter deficiency, sarcopenia, or a combination thereof.

In some embodiments, the inhibitors of hydroxyprostaglandin dehydrogenase provided herein can be used to treat sarcopenia. In another embodiment, the inhibitors of hydroxyprostaglandin dehydrogenase provided herein can be used to treat diaphragmatic atrophy or limb muscle atrophy due to the use of a mechanical ventilator. In some embodiments, the inhibitors of hydroxyprostaglandin dehydrogenase provided herein can be used to treat genetic disorders or neuromuscular disorders such as Spinal Muscular Atrophy (SMA). In some embodiments, the inhibitors of hydroxyprostaglandin dehydrogenase provided herein can be used to treat ptosis, rotator cuff muscle atrophy, immobilization related muscle atrophy, surgical procedure related muscle atrophy, sarcopenia, or a combination thereof.

### Pharmaceutical Compositions

The inhibitors of hydroxyprostaglandin dehydrogenase can be formulated into pharmaceutical compositions to treat diseases and disorders described herein. In some embodiments, a pharmaceutical composition may comprise a therapeutically effective amount of one or more inhibitors of hydroxyprostaglandin dehydrogenase provided herein.

The pharmaceutical composition described herein may be administered in such oral dosage forms as tablets, capsules (each of which includes sustained release or timed release formulations), pills, powders, micronized compositions, granules, elixirs, tinctures, suspensions, ointments, vapors, liposomal particles, nanoparticles, syrups and emulsions. In some embodiments, the pharmaceutical composition may also be administered in intravenous (bolus or infusion), subcutaneous injection, suppository, intraperitoneal, topical (e.g., dermal epidermal, transdermal), ophthalmically such as ocular eyedrop, intranasally, subcutaneous, inhalation, intramuscular or transdermal (e.g., patch) form, all using forms well known to those of ordinary skill in the pharmaceutical arts.

In some embodiments, a compound provided herein can be administered as part of a therapeutic regimen that comprises administering one or more second agents (e.g. 1, 2, 3, 4, 5, or more second agents), either simultaneously or sequentially with the compound provided herein. When administered sequentially, the compound provided herein may be administered before or after the one or more second agents. When administered simultaneously, the compound provided herein and the one or more second agents may be administered by the same route (e.g. injections to the same location; tablets taken orally at the same time), by a different route (e.g. a tablet taken orally while receiving an intravenous infusion), or as part of the same combination (e.g. a solution comprising a compound provided herein and one or more second agents).

A combination treatment according to the disclosure may be effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. The exact dosage will depend upon the agent selected, the route of administration, the form in which the compound is administered, the subject to be treated, the body weight of the subject to be treated, and the preference and experience of the attending physician.

### Examples

### Synthesis and Characterization of Compounds

In another aspect, methods of making the inhibitors described herein are provided herein. In some cases, the inhibitors are isolated or extracted from one or more plants. In some cases, the inhibitors derived from the one or more plants may be further modified. In some cases, the inhibitors are further purified after isolation from the one or more plants.

Exemplary synthesis schemes for the inhibitors with phenyl core as described herein include:

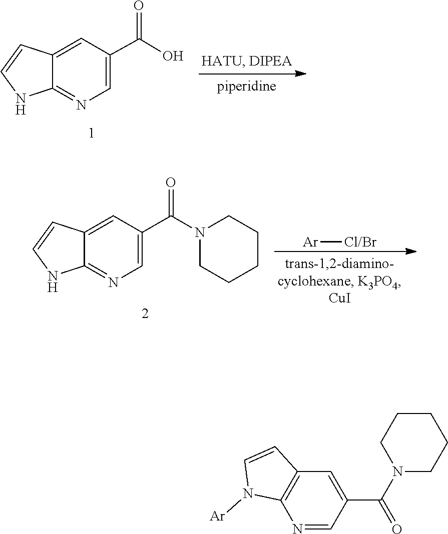

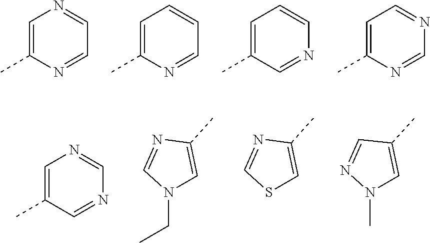

In some cases, synthesis schemes may be entire synthesis schemes for producing the inhibitors provided herein. In other cases, synthesis schemes may be partial schemes for producing inhibitors provided herein.

Described herein are exemplary synthesis schemes that can be used to synthesize the inhibitors described herein. The following abbreviations are used:

| Abbreviation | Description |
|---|---|
| AIBN | azobisisobutyronitrile |
| DCM | dichloromethane |
| DIAD | diisopropyl azodicarboxylate |
| DIPEA | N,N′-diisopropylethylamine |
| DMAP | 4-dimethylaminopyridine |
| DMF | dimethylformamide |
| EDCI | 1-ethyl-3-(3-dimethylaminopropyl)carbodiimide |
| HATU | 1-[Bis(dimethylamino)methylene]-1H-1,2,3-triazolo[4,5- |
| | b]pyridinium 3-oxide hexafluorophosphate |
| HOBt | hydroxybenzotriazole |
| m-CPBA | Meta-chloroperoxybenzoic acid |
| NBS | N-bromosuccinimide |
| NCS | N-chlorosuccinimide |
| NIS | N-iodosuccinimide |
| p-TSA | para-toluenesulfonic acid |
| TEA | triethylamine |
| TFA | trifluoroacetic acid |
| THF | tetrahydrofuran |
| TPP | triphenylphosphine |
| mmol | Milli molar |
| vol | Volume |
| g | Gram |
| kg | Kilogram |
| L | Liter |
| mL | Milli liter |
| ° C. | Degree Celsius |
| TLC | Thin Layer Chromatography |
| HPLC | High-performance liquid chromatography |
| LCMS | Liquid chromatography-mass spectrometry |
| min | Minutes |
| h | Hour |
| eq | Equivalents |
| RT | Room temperature |
| Rf | Retention factor |
| RP | Reversed phase |
| NMR | Nuclear magnetic resonance |
| Ppm | Parts per million |

### Example 1. Synthesis of B-3. B-4. B-5. B-6. B-7 and B-8

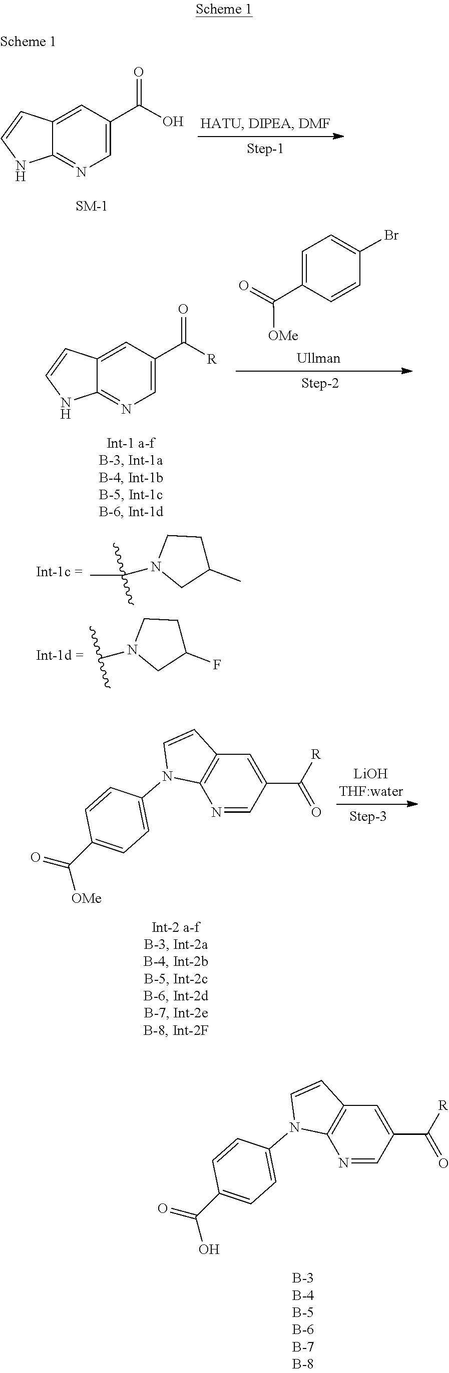

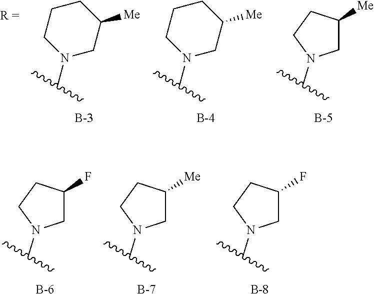

General procedure for acid-amine coupling using HATU (Step-1): To the stirred solution of 1H-pyrrolo[2,3-b]pyridine-5-carboxylic acid (SM-1) (1.0 eq) in DMF (10 V) at 0° C., HATU (1.2 eq), amine (1.2 eq) were added. To this stirred solution N, N-diisopropylethylamine (3.0 eq) was added at 0° C. and then continued for stirring at RT for 16 h. The progress of the reaction was monitored with TLC and LCMS. After consumption of starting material, mixture was diluted with ice cold water (10 mL) and extracted with EtOAc (3×10 mL). The combined extracts were washed with water and brine, dried over sodium sulphate, filtered and concentrated under reduced pressure. The crude compound was purified by silica gel column chromatography using 50% EtOAc/heptane to afford Int-1a-d.

Int-1a: Yield=66.22% MS: m/z=244.1 [M+H]⁺.

Int-1b: Yield=66.23% MS: m/z=244.2 [M+H]⁺.

Int-1c: Yield=99.21% MS: m/z=230.1 [M+H]⁺.

Int-1d: Yield=43% MS: m/z=234.1 [M+H]⁺.

General procedure for Ullmann coupling (Step-2): To a stirred solution of amide (Int-1a-e) (1 eq.) in dioxane (10 mmol), 4-bromobenzoate (1.2 eq), K₃PO₄ (1 eq.) were added in a sealed tube under inert atmosphere. Argon gas was purged for 15 min then CuI (0.2 eq) and trans-dimethylcyclohexane-1,2-diamine (0.2 eq) were added at room temperature The resultant sealed reaction mixture was heated to 100° C. for 16 h. The reaction was monitored by crude LCMS/TLC; after completion of the reaction, the mixture was quenched with saturated NH₄Cl, filtered through celite bed, washed with EtOAc (twice). The EtOAc extract was washed with brine (10 mL), dried over sodium sulphate, filtered and concentrated in vacuo to obtain the crude. The crude compound was purified by silica gel column chromatography to afford Int-2a/Int-2b as well as the enantiomeric mixtures Int-2c-f. The racemic product (Int-2c-f) was separated via Chiral Prep-HPLC purification to get both the enantiomers separately; the stereochemistry assignment is arbitrary.

Int-2a: Yield=45.16% MS: m/z=378.2 [M+H]⁺.

Int-2b: Yield=61.29% MS: m/z=378.2 [M+H]⁺.

Int-2c: Yield=99.21% MS: m/z=364.1 [M+H]⁺.

Int-2d: Yield=15.5% MS: m/z=368.1 [M+H]⁺.

Int-2e: Yield=99.71% MS: m/z=364.1 [M+H]⁺.

Int-2f: Yield=11.5% MS: m/z=368.1 [M+H]⁺.

General procedure for ester hydrolysis with LiOH (Step-3): To a stirred solution of ester (Int-2a-f) (1.0 eq) in THF/water (1:1), LiOH (3.0 eq) was added at room temperature and the resulting reaction mixture was stirred at room temperature for 16 h. The reaction was monitored by crude LCMS/TLC; upon completion, the reaction mixture was concentrated and neutralized with 1N HCl. The resulting solids were filtered, washed with Et₂O and dried in vacuo to afford B-3, B-4, B-5, B-6, B-7 and B-8.

B-3: Yield=45.16% MS: m/z=364.1 [M+H]⁺

B-4: Yield=61.29% MS: m/z=364.1 [M+H]⁺

B-5: Yield=78% MS: m/z=350.1 [M+H]⁺

B-6: Yield=80.1% MS: m/z=354.2 [M+H]⁺

B-7: Yield=84.5% MS: m/z=350.2 [M+H]⁺

B-8: Yield=88.54% MS: m/z=354.2 [M+H]⁺

### Example 2. Synthesis of (R)-4-(5-(3-methylpiperidine-1-carbonyl)-1H-pyrrolo12,3-b1 pyridin-1-yl)benzonitrile (B-9) and synthesis of (R)-4-(5-(3-methylpiperidine-1-carbonyl)-1H-pyrrolo[2,3-b1 pyridin-1-yl)benzonitrile (B-10)

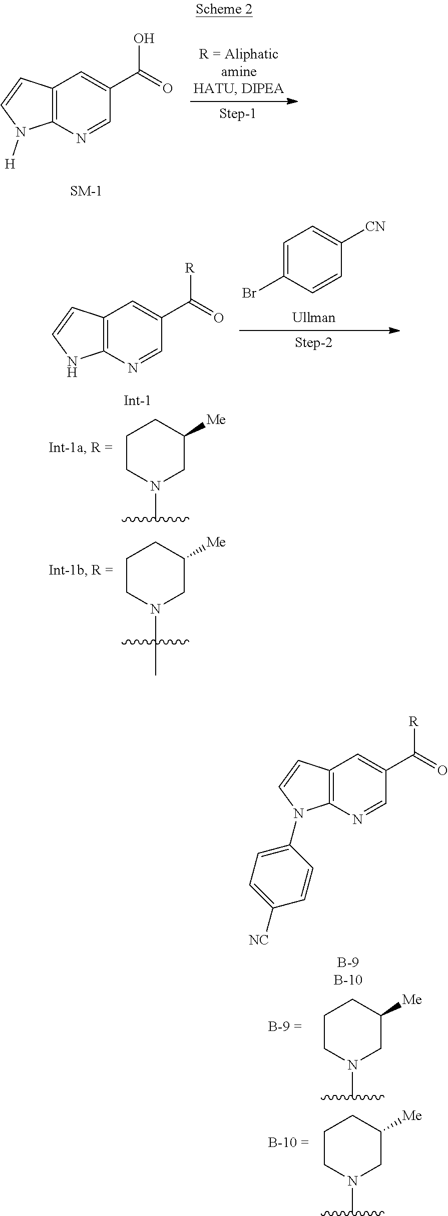

Step-1: Synthesis of Int-1a and Int-1b: Using the general procedure for acid-amine coupling with HATU, SM-1 was converted to Int-1a (Yield=39.79%, MS: m/z=244.1 [M+H]⁺) and Int-1b (Yield=66.37%, MS: m/z=244.2 [M+H]⁺).

Step-2: Synthesis of B-9 and B-10: Using the general procedure for Ullman coupling Int-1a/Int-1b was converted to B-9 (Yield=23.23%, MS: m/z=345.2[M+H]⁺) and B-10 (Yield=35.57%, MS: m/z=345.1 [M+H]⁺.)

### Example 3. General Synthesis of B-2. B-13 and B-30

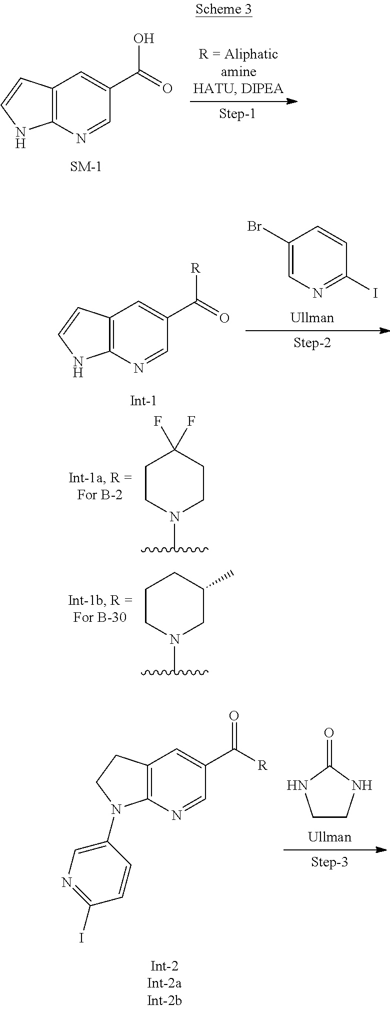

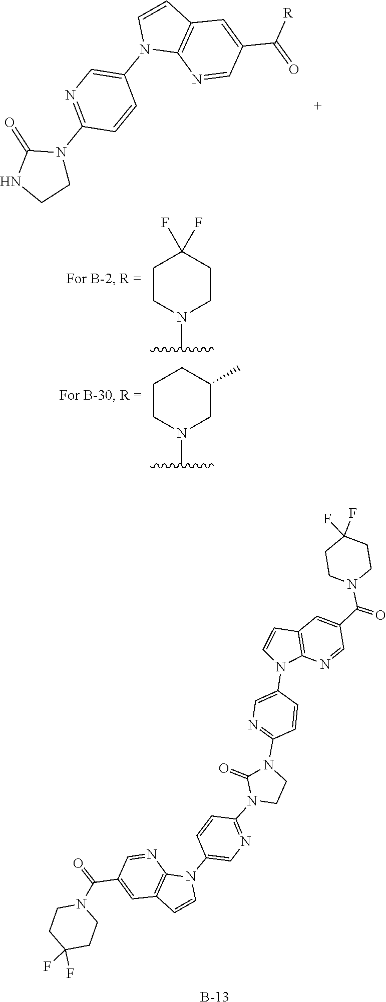

Step-1: Synthesis of Int-1: To the stirred solution of 1H-pyrrolo [2,3-b]pyridine-5-carboxylic acid (SM-1) (1.0 eq) in DMF (10 V) at 0° C., HATU (1.2 eq), amine (1.2 eq) were added. To this stirred solution N, N-diisopropylethylamine (3 eq) was added at 0° C. and then continued for stirring at RT for 16 h. The progress of the reaction was monitored with TLC and LCMS. After consumption of starting material, the mixture was diluted with ice cold water (10 mL) and extracted with EtOAc (3×10 mL). The combined extracts were washed with water and brine, dried over sodium sulphate, filtered and concentrated under reduced pressure. The crude compound was purified by silica gel column chromatography using 50% EtOAc/heptane to afford Int-1a (Yield=53.1% MS: m/z=266.1 [M+H]⁺) and hit-1b (Yield=53.50%, MS: m/z=244.1 [M+H]⁺.)

Step-2: Synthesis of Int-2 using the general procedure for Ullmann coupling: To a stirred solution of amine (Int-1) (1 eq.) in dioxane (10 V), aryl halide (1.2 eq), K₃PO₄ (1 eq.) were added in a sealed tube under inert atmosphere. Argon gas was purged for 15 min then CuI (0.2 eq), trans-dimethylcyclohexane-1,2-diamine (0.2 eq) were added at room temperature. The resultant sealed reaction mixture was heated to 100° C. for 16 h. The reaction was monitored by crude LCMS/TLC; after completion of the reaction, the mixture was quenched with saturated NH₄C₁, filtered through celite bed, washed with EtOAc (twice). The EtOAc extract was washed with brine (10 mL), dried over sodium sulphate, filtered and concentrated in vacuo to obtain the crude. Crude was purified by combi-flash column chromatography using 50% EtOAc/heptane to afford Int-2a (Yield=56% MS: m/z=427.1 [M+H]⁺) and Int-2b (Yield=88%, MS: m/z447.1 [M+H]⁺.)

Step-3: Synthesis of B-2, B-13, B-30: Using the general procedure for Ullmann coupling Int-2a was converted to B-2 and B-13, and Int-2b was converted to B-30. The crude was purified by prep-HPLC purification to afford B-2 (Yield=33% MS: m/z=427.1 [M+H]⁺), B-13 (Yield=15.3% MS: m/z=767.1 [M+H]⁺) as an off white solid and combiflash column chromatography using 50% EtOAc/heptane to afford B-30 (48 mg, 88%), as an off white solid.

### Example 4. Synthesis of B-12 and B-29

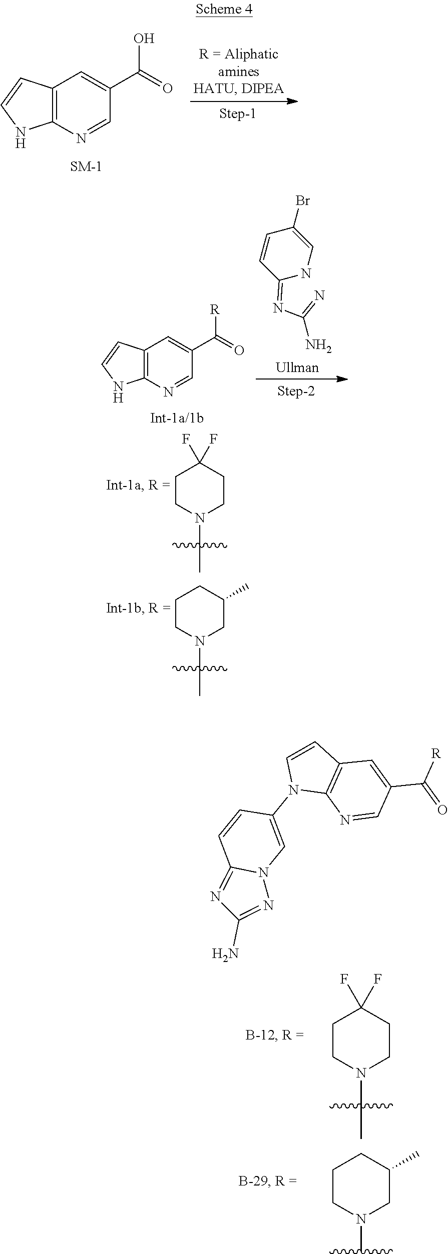

Step-1: Synthesis of Int-1a/1b: Using general procedure for acid-amine coupling using HATU, SM-1 was converted to Int-1a (Yield=68%, MS: m/z=266.1 [M+H]⁺) and Int-1b (Yield=53.50%, MS: m/z=244.1 [M+H]⁺).

Step-2: Synthesis of B-12 and B-29: Using the general procedure for Ullman reaction Int-1a/Int-1b, was converted to B-12 (Yield=26.70%, MS: m/z=398.1 [M+H]⁺) and B-29 (Yield=5.7%, MS: m/z=376.2 [M+H]⁺).

### Example 5. Synthesis of B-28

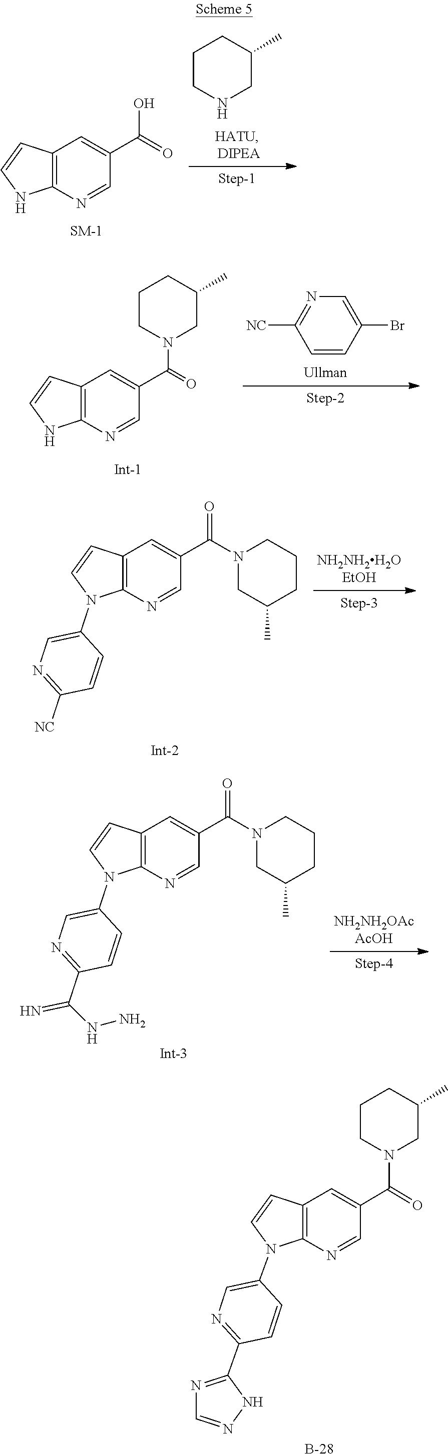

Step-1: Int-1 is described above in the synthesis of B-12.

Step-22: Using the general procedure for Ullman reaction Int-1 was converted to Int-2. (Yield=30.20% MS: m/z=346.2 [M+H]⁺.)

Step-3: Synthesis of (S)-5-(5-(3-methylpiperidine-1-carbonyl)-1H-pyrrolo[2,3-b]pyridin-1-yl)picolinimidohydrazide: To a stirred solution of (S)-5-(5-(3-methylpiperidine-1-carbonyl)-1H-pyrrolo[2,3-b]pyridin-1-yl)picolinonitrile Int-2 (250 mg, 0.724 mmol, 1.0 eq) in ethanol (3 mL) was added hydrazine monohydrate (6 mL). The mixture was stirred at 60° C. for 1 h. The progress of the reaction was monitored with TLC. The solid obtained was filtered and dried, triturated with Et₂O afforded (S)-5-(5-(3-methylpiperidine-1-carbonyl)-1H-pyrrolo[2,3-b]pyridin-1-yl)picolinimidohydrazide Int-3 (220 mg) as yellow solid. (MS: m/z=378.2 [M+H]⁺). Crude obtained was used as such without purification for next step.

Step-4: Synthesis of B-28: Int-3 (200 mg, 0.529 mmol) was converted to B-28 (12.20%, 25 mg) using general procedure for 1,3,4-triazol formation using hydrazine acetate as described above for B-28.

### Example 6. Synthesis of (4,4-difluoropiperidin-1-yl)1-(5-(2-methyl-1H-imidazol-4-yl)pyridin-3-yl)-1H-pyrrolo[2,3-b]pyridin-5-yl)methanone. (B-15) and (4,4-difluoropiperidin-1-yl)(1-(5-(1-(4-methoxybenzyl)-2-methyl-1H-imidazol-4-yl)pyridin-3-yl)-1H-pyrrolo [2,3-b]pyridin-5-yl)methanone. (B-23)

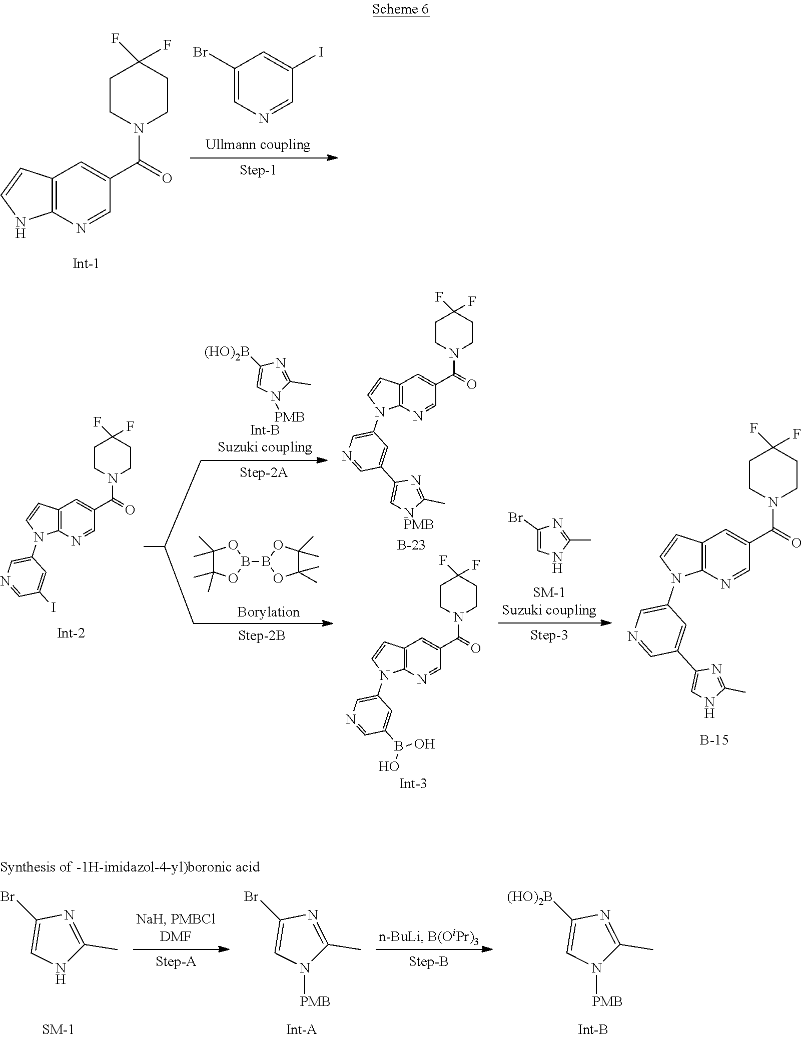

Int-1 is described above in the synthesis of B-12.

Step-1: (4,4-difluoropiperidin-1-yl(1-(5-iodopyridin-3-yl)-1H-pyrrolo[2,3-b]pyridin-5-yl) methanone, Int-2: Using the general procedure for Ullmann coupling Int-1 and 3-bromo-5-iodopyridine were coupled to afford Int-2 (84.4%) as an off white solid. TLC: 50% EtOAc/Heptane (Rf: 0.40) MS: m/z=469.05 [M+H]⁺.

Step-2A: Synthesis of (4,4-difluoropiperidin-1-yl(1-(5-(1-(4-methozybenzyl)-2-methyl-1H-imidazol-4-yl)pyridin-3-yl)-1H-pyrrolo[2,3-b]pyridin-5-yl)methanone, B-23, general procedure for Suzuki coupling: To a stirred solution of (4,4-difluoropiperidin-1-yl)(1-(5-iodopyridin-3-yl)-1H-pyrrolo[2,3-b]pyridin-5-yl) methanone, Int-2 (210 mg, 0.448 mmol, 1 eq.) and (1-(4-methoxybenzyl)-2-methyl-1H-imidazol-4-yl) boronic acid (165 mg, 0.672 mmol, 1.5 eq) in 1,4-dioxane:water (3:1, 10 mL), Na₂CO₃ (118 mg, 1.120 mmol, 2.5 eq) was added and then the mixture was purged with Argon for 15 min. To this solution, PdCl₂ (dppf).DCM (36 mg, 0.044 mmol, 0.1 eq) was added under argon. The resulting reaction mixture was stirred at 100° C. for 16 h. The progress of the reaction was monitored by TLC. After completion of the reaction, the reaction mixture was filtered through celite and evaporated to dryness. The residue was diluted with ethyl acetate (2×10 mL), washed with brine (10 mL) and the organic phase dried over sodium sulfate, filtered and concentrated under reduced pressure. The crude product was purified by preparative HPLC to obtain (4,4-difluoropiperidin-1-yl) (1-(5-(1-(4-methoxybenzyl)-2-methyl-1H-imidazol-4-yl)pyridin-3-yl)-1H-pyrrolo[2,3-b]pyridin-5-yl) methanone, B-23 (13.63 mg, 5.6%) as an off white solid, TLC: 10% MeOH/DCM; MS: m/z=543.2 [M+H]⁺.

Step-2B: Synthesis of (5-(5-(4,4-difluoropiperidine-1-carbonyl)₁H-pyrrolo[2,3-b]pyridin-1-yl)pyridin-2-yl) boronic acid, Int-3 general procedure for boronic acid formation: To a stirred solution of (4,4-difluoropiperidin-1-yl)(1-(5-iodopyridin-3-yl)-1H-pyrrolo[2,3-b]pyridin-5-yl) methanone, Int-2 (310 mg, 0.662 mol, 1 eq) and Bis(pinacolato)diboron (252 mg, 0.993 mol, 1.5 eq.) in 1,4-dioxane (10 mL), KOAc (129.9 mg, 1.324 mmol, 2 eq.) was added and purged with argon for 15 min. To this solution, PdCl₂ (dppf).DCM (5.40 mg, 0.06 mmol, 0.1 eq.) was added and purged with Argon for another 10 min. The resulting reaction mixture was stirred at 100° C. for 16 h. The progress of the reaction was monitored by TLC. After completion of the reaction, the reaction mixture was filtered through Celite and evaporated to dryness. The crude was triturated with n-pentane and dried in vacuo to afford (5-(5-(4,4-difluoropiperidine-1-carbonyl)-1H-pyrrolo[2,3-b]pyridin-1-yl)pyridin-2-yl) boronic acid, Int-3 (210 mg, 82.14%) as brown liquid. TLC: 10% MeOH/DCM; MS: m/z=387.4 [M+H]⁺.

Step-3: Synthesis of (4,4-difluoropiperidin-1-yl)(1-(6-(2-methyl-1H-imidazol-4-yl)pyridin-3-yl)-1H-pyrrolo[2,3-b]pyridin-5-yl)methanone B-15: (5-(5-(4,4-difluoropiperidine-1-carbonyl)-1H-pyrrolo[2,3-b]pyridin-1-yl)pyridin-2-yl) boronic acid, Int-3 was converted to B-15 (4.7%) using the general procedure for Suzuki coupling. TLC: 10% MeOH/DCM; MS: m/z=423.25 [M+H]⁺.

Step-A: Synthesis of 4-bromo-1-(4-methozybenzyl)-2-methyl-1H-imidazole (Int-A): To a stirred solution of 4-bromo-2-methyl-1H-imidazole (1 g, 6.21 mmol, 1 eq) in DMF (15 mL), NaH (60% in mineral oil) (0.298 mg, 7.45 mmol, 1.2 eq) was added at 0° C. to room temperature for 1 h. To this stirred suspension of PMBCI (1.46 g, 9.32 mmol, 1.5 eq) was added and then the resulting reaction mixture was stirred for 4 h. The reaction was monitored by crude LCMS/TLC; after complete consumption of the starting material, the reaction mixture was quenched with sat. NH₄Cl (10 ml) and extracted with EtOAc (2×50 mL). Combined organic extracts were washed with brine (20 mL), dried over sodium sulfate, filtered and concentrated in vacuo to obtain 4-bromo-1-(4-methoxybenzyl)-2-methyl-1H-imidazole, Int-A (800 mg). The crude was used in the next step without further purification. TLC: 10% MeOH/DCM MS: m/z=281.1 [M+H]⁺.

Step-B: Synthesis of (1-(4-methozybenzyl)-2-methyl-1H-imidazol-4-yl)boronic acid (Int-B): To a stirred solution of 4-bromo-1-(4-methoxybenzyl)-2-methyl-1H-imidazole, Int-A (800 mg, 2.85 mmol, 1 eq) in THE (10 mL) was added triisopropyl borate (1.97 mL, 8.54 mmol). The reaction mixture was cooled to −78° C. and n-BuLi (1.6M, 2.67 mL, 4.27 mmol, 1.5 mmol) was added over a period of 45 min. The reaction mixture was stirred at same temperature for 30 min and further stirred at room temperature for 3 h. Progress of the reaction was monitored by TLC. After completion, the reaction mixture was quenched with 2N HCl (10 mL) and stirred at room temperature for 3 h. Solvent was removed in vacuo. Crude obtained was dissolved in ethyl acetate (20 mL) washed with brine (20 mL), dried over sodium sulfate, filtered and concentrated to afford (1-(4-methoxybenzyl)-2-methyl-1H-imidazol-4-yl)boronic acid, Int-B (500 mg) as brown liquid. TLC: 10% MeOH/DCM; MS: m/z=247.04 [M+H]⁺.

### Example 7. Synthesis of (4,4-difluoropiperidin-1-yl)(1-(6-(2-methyl-1H-imidazol-4-yl)pyridin-3-yl)-1H-pyrrolo[2,3-b]pyridin-5-yl)methanone, (B-16) and (44-difluoropiperidin-1-yl) (1-(6-(1-(4-methoxybenzyl)-2-methyl-1H-imidazol-4-yl)pyridin-3-yl)-1H-pyrrolo2,3-b1 pyridin-5-vi) methanone, (B-24)

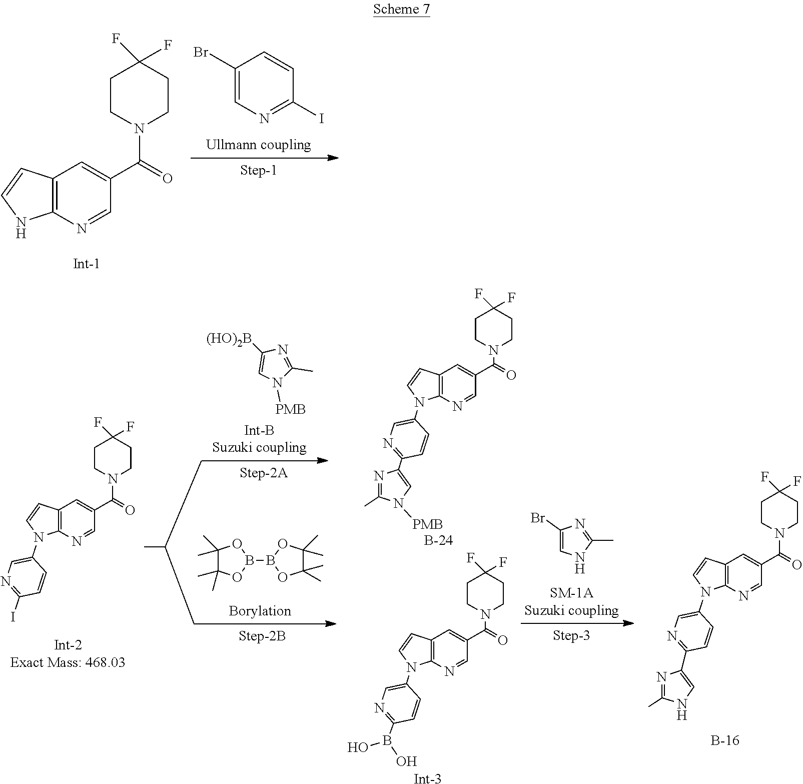

Int-1 is described above in the synthesis of B-12.

Step-1: Synthesis of (4,4-difluoropiperidin-1-yl)(1-(6-iodopyridin-3-yl)-1H-pyrrolo [2,3-b]pyridin-5-yl)methanone, Int-2: Using general procedure for Ullmann coupling Int-1 was converted to (4,4-difluoropiperidin-1-yl)(1-(6-iodopyridin-3-yl)-1H-pyrrolo [2,3-b]pyridin-5-yl) methanone, Int-2 (80%) as an off white solid. TLC: 50% EtOAc/Heptane (Rf: 0.40); MS: m/z=469.05 [M+H]⁺.

Step-2A: Synthesis of (4,4-difluoropiperidin-1-yl) (1-(6-(1-(4-methozybenzyl)-2-methyl-1H-imidazol-4-yl)pyridin-3-yl)-1H-pyrrolo[2,3-b]pyridin-5-yl)methanone B-24: Using the general procedure for Suzuki coupling (4,4-difluoropiperidin-1-yl)(1-(6-iodopyridin-3-yl)-1H-pyrrolo [2,3-b]pyridin-5-yl) methanone, Int-2 was converted to B-24, using Int-B (described above for the synthesis of B-23). The crude was purified by silica gel column chromatography using 5% MeOH:DCM followed by prep-HPLC purification to obtain B-24 (41.36 mg, 19.29%) as an off-white solid.

Step-2B: Synthesis of (5-(5-(4,4-difluoropiperidine-1-carbonyl)₁H-pyrrolo[2,3-b]pyridin-1-yl)pyridin-2-yl) boronic acid, Int-3: Using general procedure for boronic acid formation (4,4-difluoropiperidin-1-yl)(1-(6-iodopyridin-3-yl)-1H-pyrrolo[2,3-b]pyridin-5-yl) methanone, Int-2 was converted to (5-(5-(4,4-difluoropiperidine-1-carbonyl)-1H-pyrrolo[2,3-b]pyridin-1-yl)pyridin-2-yl) boronic acid, Int-3. The crude was used in the next step without further purification. TLC: 5% MeOH/DCM; MS: m/z=387.1 [M+H]⁺.

Step-3: Synthesis of (4,4-difluoropiperidin-1-yl)(1-(6-(2-methyl-1H-imidazol-4-yl)pyridin-3-yl)-1H-pyrrolo[2,3-b]pyridin-5-yl)methanone, B-16: Using general procedure for Suzuki coupling (5-(5-(4,4-difluoropiperidine-1-carbonyl)-1H-pyrrolo[2,3-b]pyridin-1-yl)pyridin-2-yl) boronic acid, Int-3 (250 mg, 0.647 mmol, 1 eq) was converted to (4,4-difluoropiperidin-1-yl) 1-(6-(2-methyl-1H-imidazol-4-yl)pyridin-3-yl)-1H-pyrrolo[2,3-b]pyridin-5-yl) methanone B-16 (10.57 mg, 3.86%) as an off white solid prep-HPLC purification. TLC: 5% MeOH/DCM; MS: m/z=423.15 [M+H]⁺.

### Example 8. Synthesis of (4,4-difluoropiperidin-1-yl)(1-(5-(4-methyl-1H-1,2,3-triazol-5-yl) pyridin-3-yl)-1H-pyrrolo[2,3-b]pyridin-5-yl) methanone (B-18) and (4,4-difluoropiperidin-1-yl) (1-(6-(5-methyl-1H-1,2,3-triazol-4-yl)pyridin-3-yl)-1H-pyrrolo[2,3-b]pyridin-5-yl) methanone (B-19)

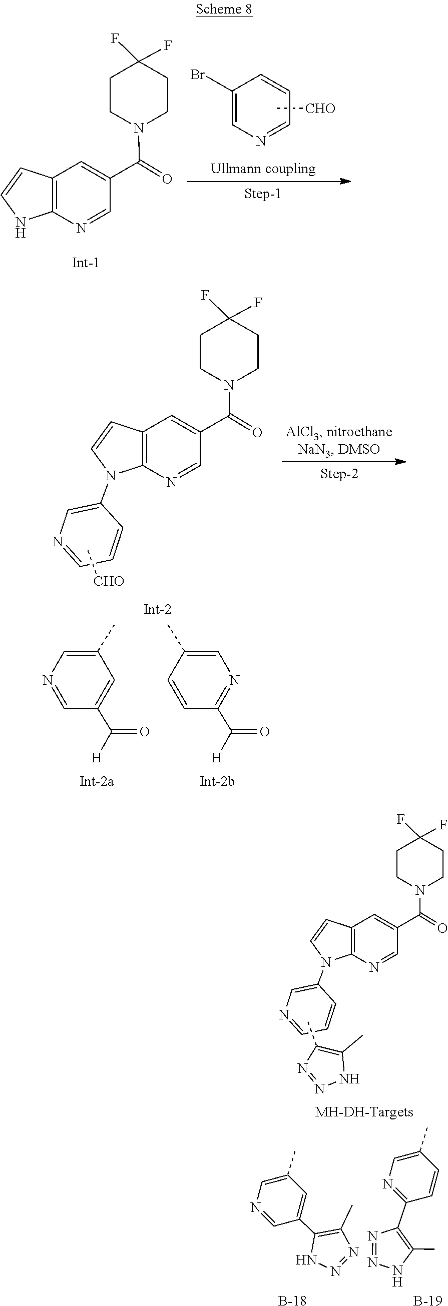

Int-1 is described above in the synthesis of B-12.

Step-1: Synthesis of Int-2: Using general procedure for Ullmann coupling, (4,4-difluoropiperidin-1-yl) (1H-pyrrolo[2,3-b]pyridin-5-yl)methanone Int-1 (described previously in the synthesis of B-12, 1 g, 3.77 mmol, 1 eq) was converted to 5-(5-(4,4-difluoropiperidine-1-carbonyl)-1H-pyrrolo[2,3-b]pyridin-1-yl) nicotinaldehyde, Int-2a (400 mg, 28.7%), isolated as a yellow gummy liquid, TLC: 100% EtOAc/heptane, MS: m/z=369.1 [M−H]⁻and 5-(5-(4,4-difluoropiperidine-1-carbonyl)-1H-pyrrolo[2,3-b]pyridin-1-yl) picolinaldehyde, Int-2b (420 mg, 30.2%). TLC: 100% EtOAc/heptane, MS: m/z=371.1 [M+H]⁺370.36

Step-2: Synthesis of (4,4-difluoropiperidin-1-yl) (1-(5-(4-methyl-1H-1,2,3-triazol-5-yl)pyridin-3-yl)-1H-pyrrolo[2,3-b]pyridin-5-yl) methanone, B-18 (General procedure for 1,2,3-triazol formation): To stirred solution of 5-(5-(4,4-difluoropiperidine-1-carbonyl)-1H-pyrrolo[2,3-b]pyridin-1-yl) nicotinaldehyde, Int-2a (400 mg, 1.08 mmol, 1 eq), nitroethane (0.1 mL, 1.62 mmol, 1.5 eq), NaN₃ (77 mg, 1.1 mmol, 1.1 eq), and AlCl₃ (20 mg, 0.129 mmol, 0.12 eq) were stirred in 8 mL DMSO at 80° C. for 16 h. The reaction was monitored by crude LCMS/TLC; after complete consumption of the starting material, the reaction mixture was quenched with water (10 mL) and extracted with EtOAc (3×20 mL). The combined organic layers were dried over anhydrous sodium sulfate, and the solvent was evaporated in vacuo. The crude was purified by combi-flash column chromatography using 70% EtOAc/heptane to afford (4,4-difluoropiperidin-1-yl) (1-(5-(4-methyl-1H-1,2,3-triazol-5-yl)pyridin-3-yl)-1H-pyrrolo[2,3-b]pyridin-5-yl)methanone, B-18 (85 mg, 18.5%) as an off white solid. TLC: 100% EtOAc/heptane, MS: m/z=424.5 [M+H]⁺.

Step-2: Synthesis of (4,4-difluoropiperidin-1-yl)(1-(6-(5-methyl-1H-1,2,3-triazol-4-yl)pyridin-3-yl)-1H-pyrrolo[2,3-b]pyridin-5-yl)methanone, B-19: Using general procedure for 1,2,3-triazol formation Int-2b (420 mg, 1.13 mmol, 1 eq) was converted to (4,4-difluoropiperidin-1-yl)(1-(6-(5-methyl-1H-1,2,3-triazol-4-yl)pyridin-3-yl)-1H-pyrrolo[2,3-b]pyridine-5-yl)methanone, B-19 (105 mg, 21.87%) as an off white solid. TLC: 100% EtOAc/heptane, MS: m/z=424.1 [M+H]⁺.

### Example 9, Synthesis of (4,4-difluoropiperidin-1-yl)(1-(6-(5-methyl-4H-1,2,4-triazol-3-yl)pyridin-3-yl)-1H-pyrrolo[2,3-b]pyridin-5-yl)methanone (b-21) and (4,4-difluoropiperidin-1-YL)(1-(5-(5-methyl-4H-1,2,4-triazol-3-yl)pyridin-3-yl)-1H-pyrrolo[2,3-b]pyridine-5-yl) methanone (b-22) and (1-(5-(5-cyclopropyl- 1H-1,2,4-triazol-3-yl)pyridin-3-yl)-1H-pyrrolo [2,3-b]pyridin-5-yl(4,4-difluoropiperidin-1-yl)methanone (B-1)

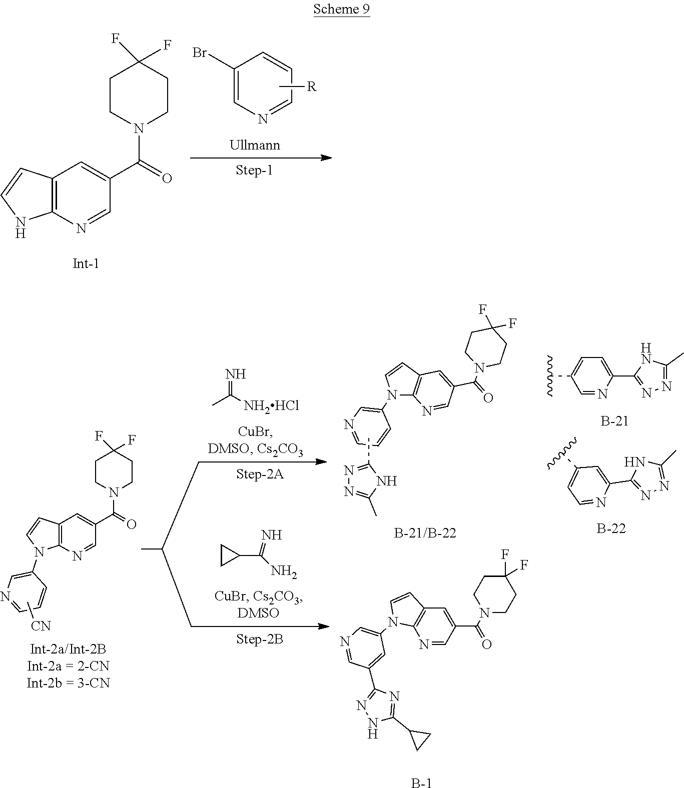

Int-1 is described above in the synthesis of B-12.

Step-1: Synthesis of Int-2a/Int-2b: Using general procedure for Ullmann coupling, (4,4-difluoropiperidin-1-yl)(1H-pyrrolo[2,3-b]pyridin-5-yl)methanone Int-1 (5 g, 18.55 mmol, 1 eq) was converted to 5-(5-(4,4-difluoropiperidine-1-carbonyl)-1H-pyrrolo[2,3-b]pyridin-1-yl) picolinonitrile, Int-2a (2 g, 30%), TLC: 100% EtOAc/heptane, MS: m/z=366.1 [M−H]⁻and 5-(5-(4,4-difluoropiperidine-1-carbonyl)-1H-pyrrolo[2,3-b]pyridin-1-yl)nicotinonitrile, Int-2b (3.5 g, 51%). TLC: 100% EtOAc/heptane, MS: m/z=366.1[M−H].

Step-2A: Synthesis of (4,4-difluoropiperidin-1-yl(1-(6-(5-methyl-4H-1,2,4-triazol-3-yl)pyridin-3-yl)-1H-pyrrolo[2,3-b]pyridin-5-yl)methanone (B-21) and (4,4-difluoropiperidin-1-yl) (1-(5-(5-methyl-4H-1,2,4-triazol-3-yl)pyridin-3-yl-1H-pyrrolo[2,3-b]pyridine-5-yl) methanone (B-22) (general procedure for triazole formation): To a stirred solution of 5-(5-(4,4-difluoropiperidine-1-carbonyl)-1H-pyrrolo[2,3-b]pyridin-1-yl) picolinonitrile, Int-2a (200 mg, 0.544 mmol, 1 eq) in DMSO (5 mL) acetamidine hydrochloride (77 mg, 0.816 mmol, 1.5 eq), Cs₂CO₃ (531 mg, 1.63 mmol, 3 eq), CuBr (12 mg, 0.054 mmol, 0.1 eq) was added. The reaction mixture was stirred at 120° C. for 14 h. The reaction was monitored by TLC; after complete consumption of the starting material, the reaction mixture was quenched with saturated soln. of NaHCO₃ (10 mL) and extracted with EtOAc (3×10 mL). The combined organic extracts were washed with brine (10 mL); dried over sodium sulphate, and concentrated in vacuo to obtain the crude. The crude was purified by combiflash column chromatography using 5% MeOH: DCM to afford (4,4-difluoropiperidin-1-yl)(1-(6-(5-methyl-4H-1,2,4-triazol-3-yl)pyridin-3-yl)-1H-pyrrolo[2,3-b]pyridin-5-yl)methanone, B-21 (70 mg, 30%) as an off white solid. TLC: 100% EtOAc/heptane, MS: m/z=424.2 [M+H]⁺. 5-(5-(4,4-difluoropiperidine-1-carbonyl)-1H-pyrrolo[2,3-b]pyridin-1-yl)nicotinonitrile, Int-2b (700 mg, 1.91 mmol, 1 eq) was converted to (4,4-difluoropiperidin-1-yl) (1-(5-(5-methyl-4H-1,2,4-triazol-3-yl)pyridin-3-yl)-1H-pyrrolo[2,3-b]pyridine-5-yl) methanone, B-22, using similar protocol as described above to afford (4,4-difluoropiperidin-1-yl) (1 (5-(5-methyl-4H-1,2,4-triazol-3-yl)pyridin-3-yl)-1H-pyrrolo[2,3-b]pyridine-5-yl) methanone, B-22 (100 mg, 12%) as an off white solid. TLC: 100% EtOAc/heptane, MS: m/z=424.2 [M+H]⁺.

Step-2B: Synthesis of (1-(5-(5-cyclopropyl-1H-1,2,4-triazol-3-yl)pyridin-3-yl)-1H-pyrrolo [2,3-b]pyridin-5-yl)(4,4-difluoropiperidin-1-yl)methanone, (B-1): 5-(5-(4,4-difluoropiperidine-1-carbonyl)-1H-pyrrolo[2,3-b]pyridin-1-yl)nicotinonitrile, Int-2b (100 mg, 1.91 mmol, 1 eq) was converted to (1-(5-(5-cyclopropyl-1H-1,2,4-triazol-3-yl)pyridin-3-yl)-1H-pyrrolo[2,3-b]pyridin-5-yl)(4,4-difluoropiperidin-1-yl)methanone, using the general procedure for triazole formation to afford crude which was purified by prep-HPLC to obtain (1-(5-(5-cyclopropyl-1H-1,2,4-triazol-3-yl)pyridin-3-yl)-1H-pyrrolo[2,3-b]pyridin-5-yl) (4,4-difluoropiperidin-1-yl)methanone, B-1 (10 mg, 8.19%) as an off white solid. TLC: 10% MeOH:DCM (Rf 0.23) MS: m/z=450.1 [M+H]⁺.

### Example 10. Synthesis of (4,4-difluoropiperidin-1-yl)(1-(6-(1,5-dimethyl-1H-1,2,4-triazol-3-yl) pyridin-3-yl)-1H-pyrrolo[23-b]pyridin-5-yl)methanone (B-25) and (4,4-difluoropiperidin-1-yl) (1-(6-(4,5-dimethyl-4H-1,2,4-triazol-3-yl)pyridin-3-yl)-1H-pyrrolo 1,2,3-b1 pyridin-5-yl) methanone (B-31)

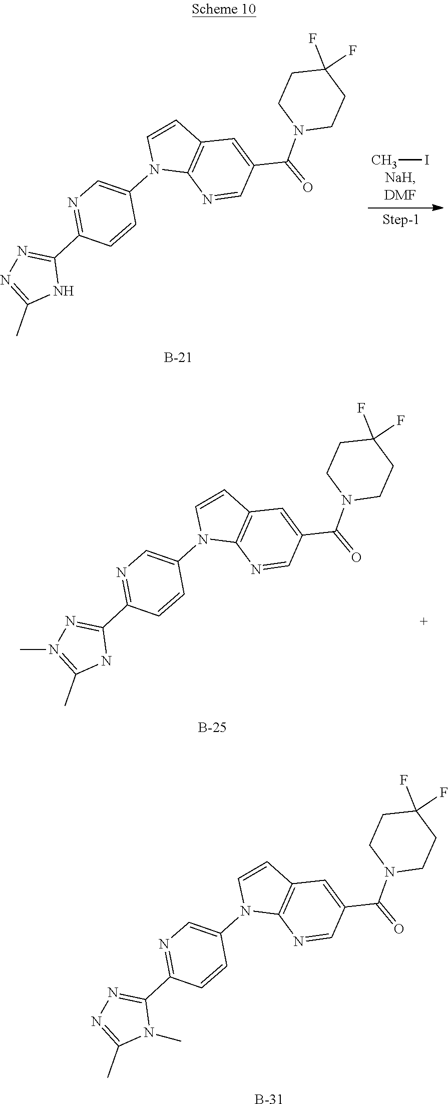

A solution of (4,4-difluoropiperidin-1-yl)(1-(6-(5-methyl-4H-1,2,4-triazol-3-yl)pyridin-3-yl)-1H-pyrrolo[2,3-b]pyridin-5-yl) methanone, B-21 (50 mg, 0.118 mmol, 1 eq) in DMF (10 mL) was cooled to 0° C. and NaH (60% in mineral oil) (163 mg, 0.200 mmol, 1.7 eq) added. After stirring at 0° C. for 20 min, methyl iodide (25 mg, 0.177 mmol, 1.5 eq) was added at 0° C. and allowed to warm to room temperature stirred for 6 h. The reaction was monitored by LCMS/TLC; after consumption of the starting material the reaction mixture was quenched with sat. NH₄C₁ solution (10 mL) and extracted with EtOAc (2×20 mL). The combined organic extracts were washed with brine (10 mL), dried over sodium sulfate, filtered and concentrated in vacuo to obtain the crude. The crude was purified by prep-HPLC to obtain (4,4-difluoropiperidin-1-yl)(1-(6-(1,5-dimethyl-1H-1,2,4-triazol-3-yl)pyridin-3-yl)-1H-pyrrolo [2,3-b]pyridin-5-yl)methanone, B-25 (23 mg, 46%) as an off white solid TLC: 10% MeOH:DCM (Rf: 0.43); MS: m/z=438.2 [M−H]⁺. and (4,4-difluoropiperidin-1-yl)(1-(6-(4,5-dimethyl-4H-1,2,4-triazol-3-yl)pyridin-3-yl)-1H-pyrrolo[2,3-b]pyridin-5-yl)methanone, B-31 (11 mg, 21.20%) as yellow solid. TLC: 10% MeOH:DCM (Rf: 0.43). MS: m/z=438.20 [M−H]⁺.

### Example 11. Synthesis of (1-(4-(1H-1,2,4-triazol-5-yl)phenyl-1H-pyrrolo[2,3-b]pyridin-5-yl) (4,4-difluoropiperidin-1-yl)methanone (B-26)

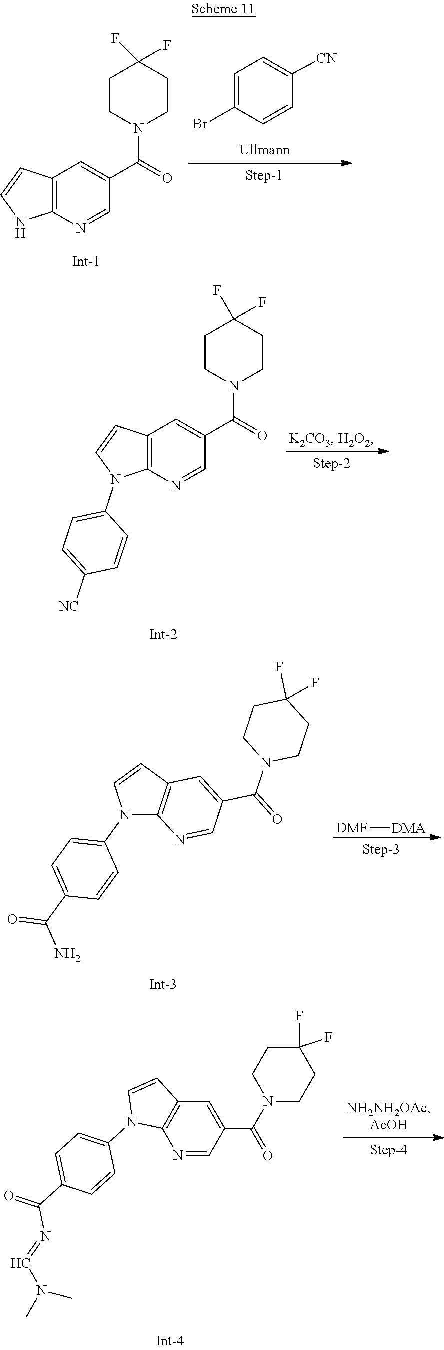

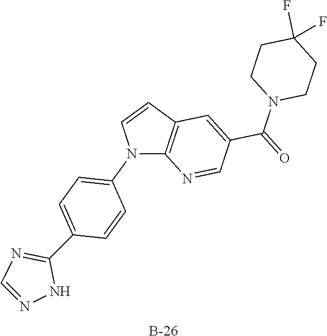

Int-1 is described above in the synthesis of B-12.

Step-1: Synthesis of 4-(5-(4,4-difluoropiperidine-1-carbonyl)-1H-pyrrolo[2,3-b]pyridin-1-yl) benzonitrile, Int-2: Using general procedure for Ullmann coupling, (4,4-difluoropiperidin-1-yl)(1H-pyrrolo[2,3-b]pyridin-5-yl)methanone, Int-1 (310 mg, 1.16 mmol, 1 eq) was converted to 4-(5-(4,4-difluoropiperidine-1-carbonyl)-1H-pyrrolo[2,3-b]pyridin-1-yl)benzonitrile, Int-2 (215 mg, 50.2%), TLC: 50% EtOAc/heptane(Rf 0.45), MS: m/z=367.1 [M+H]⁺.

Step-2: Synthesis of 4-(5-(4,4-difluoropiperidine-1-carbonyl)-1H-pyrrolo[2,3-b]pyridin-1-yl)benzamide (Int-3): To a stirred solution of 4-(5-(4,4-difluoropiperidine-1-carbonyl)-1H-pyrrolo[2,3-b]pyridin-1-yl)benzonitrile, Int-2 (185 mg, 0.50 mmol, 1 eq) in DMSO (3 mL) were added K₂CO₃ (70 mg, 0.50 mmol, 1.0 eq) followed by H₂O₂ (30%, 0.17 mL, 1.51 mmol, 3.0 eq) at 0° C. The reaction mixture was then allowed to warm up to room temperature and stirred at 60° C. for 2 h. The progress of the reaction was monitored with TLC. The solid obtained was filtered and dried and triturated with diethyl ether to afford 4-(5-(4,4-difluoropiperidine-1-carbonyl)-1H-pyrrolo[2,3-b]pyridin-1-yl)benzamide, Int-3 (160 mg, crude) as a yellow solid. The crude obtained was used in the next step without further purification. TLC: 80% EtOAc: heptane (Rf 0.35) MS: m/z=385.1 [M+H]⁺.

Step-3: Synthesis of (E)-4-(5-(4,4-difluoropiperidine-1-carbonyl)1H-pyrrolo[2,3-b]pyridin-1-yl)—N—((dimethylamino)methylene)benzamide (Int-4): A solution of 4-(5-(4,4-difluoropiperidine-1-carbonyl)-1H-pyrrolo [2,3-b]pyridin-1-yl)benzamide, Int-3 (151 mg, 0.392 mmol, 1 eq) and N,N-dimethylformamide dimethyl acetal (10 mL) was heated at 100° C. under nitrogen atmosphere for 1 h. The progress of the reaction was monitored with TLC. The reaction mixture was evaporated under reduced pressure and crude obtained was triturated with Et₂O to afford (E)-4-(5-(4,4-difluoropiperidine-1-carbonyl)-1H-pyrrolo[2,3-b]pyridin-1-yl)—N((dimethylamino)methylene)benzamide, Int-4 (180 mg, crude). as a pale yellow solid. The crude obtained was used in the next step without further purification. TLC: 100% EA/heptane (Rf 0.40) MS: m/z=440.2 [M+H]⁺.

Step-4: Synthesis of (1-(4-(1H-1,2,4-triazol-5-yl)phenyl)-1H-pyrrolo[2,3-b]pyridin-5-yl)(4,4-difluoropiperidin-1-yl)methanone, (B-26): To a stirred solution of (E)-4-(5-(4,4-difluoropiperidine-1-carbonyl)-1H-pyrrolo[2,3-b]pyridin-1-yl)—N((dimethylamino)methylene)benzamide (Int-4) (80 mg, 0.184 mmol, 1.0 eq.) in acetic acid (0.5 mL) was added hydrazine acetate (83 mg, 0.910 mmol, 5.0 eq.) at room temperature. The resultant reaction mixture was heated at 95° C. for 2 h. After completion of the reaction (monitored by TLC), reaction mixture was quenched with sat. NaHCO₃ solution and extracted with EtOAc. The combined organic layers were washed with water, dried over anhydrous Na₂SO₄, filtered and concentrated under reduced pressure. The crude compound was purified by combiflash chromatography (using a gradient method of 5% MeOH in DCM) to afford (1-(4-(1H-1,2,4-triazol-5-yl)phenyl)-1H-pyrrolo[2,3-b]pyridin-5-yl)(4,4-difluoropiperidin-1-yl)methanone, B-26 (15 mg, 20.23%) as an off white solid. TLC: 100% EA/heptane (Rf 0.40) MS: m/z=409.2 [M+H]⁺.

### Example 12. Synthesis of 4-(5-(4,4-difluoropiperidine-1-carbonyl)-1H-pyrrolo[2,3-b]pyridin-1-yl)-2-fluorobenzoic acid (B-27)

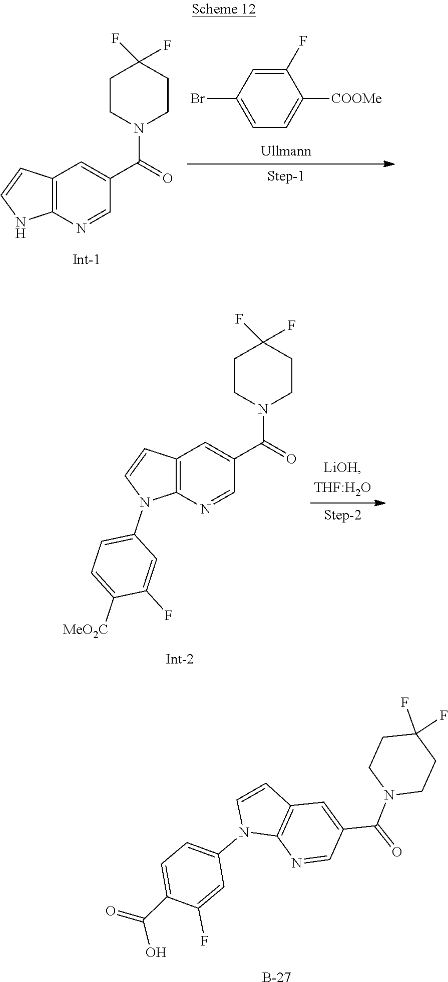

Int-1 is described above in the synthesis of B-12.

Step-1: Synthesis of methyl 4-(5-(4,4-difluoropiperidine-1-carbony9-1H-pyrrolo[2,3-b]pyridin-1-yl)-2-fluorobenzoate, Int-2: Using the general procedure for Ullmann coupling, (4,4-difluoropiperidin-1-yl)(1H-pyrrolo[2,3-b]pyridin-5-yl)methanone, Int-1 (500 mg, 1.89 mmol, 1 eq) was converted to methyl 4-(5-(4,4-difluoropiperidine-1-carbonyl)-1H-pyrrolo[2,3-b]pyridin-1-yl)-2-fluorobenzoate Int-2 (350 mg, 44%), TLC: 50% EtOAc/heptane(Rf, 0.35), MS: m/z=418.1 [M+H]⁺.

Step-2: Synthesis of 4-(5-(4,4-difluoropiperidine-1-carbonyl)-1H-pyrrolo[2,3-b]pyridin-1-yl)-2-fluorobenzoic acid, (B-27): Methyl 4-(5-(4,4-difluoropiperidine-1-carbonyl)-1H-pyrrolo[2,3-b]pyridin-1-yl)-2-fluorobenzoate Int-2 (200 mg, 0.740 mmol, 1 eq) was converted to 4-(5-(4,4-difluoropiperidine-1-carbonyl)-1H-pyrrolo[2,3-b]pyridin-1-yl)-2-fluorobenzoic acid using general procedure for ester hydrolysis with LiOH to afford 4-(5-(4,4-difluoropiperidine-1-carbonyl)-1H-pyrrolo[2,3-b]pyridin-1-yl)-2-fluorobenzoic acid, B-27 (80 mg, 41.4%) as an off white solid. MS: m/z=404.2 [M+H]⁺.

### Example 13. Synthesis of (1-(5-(5-amino-1,2,4-oxadiazol-3-yl)pyridin-3-yl)-1H-pyrrolo [2,3-b]pyridin-5-yl)(4,4-difluoropiperidin-1-yl)methanone (B-11) and (1-(5-(5-(tert-butylamino)-1.2.4-oxadiazol-3-yl)pyridin-3-yl)-1H-pyrrolo[2,3-b]pyridin-5-yl) (4,4-difluoropiperidin-1-yl) methanone (B-20)

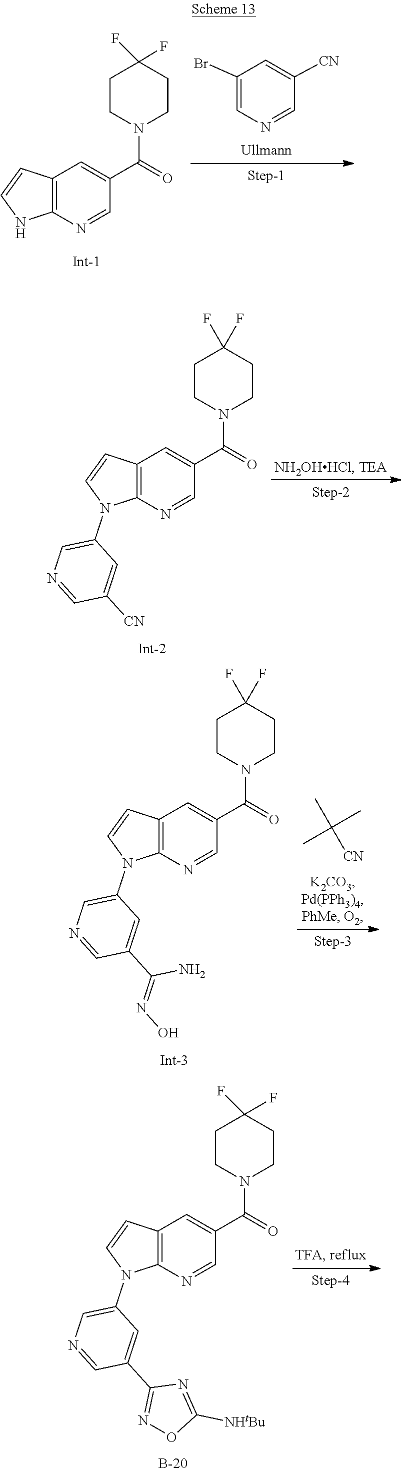

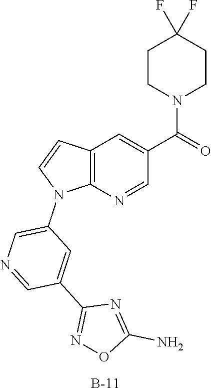

Int-1 is described above in the synthesis of B-12.

Step-1: Synthesis of 5-(5-(4,4-difluoropiperidine-1-carbonyl)-1H-pyrrolo[2,3-b]pyridine-1-yl) nicotinonitrile: Using the general procedure for Ullman coupling reaction Int-1 (1.5 g, 5.65 mmol, 1 eq) was converted to 5-(5-(4,4-difluoropiperidine-1-carbonyl)-1H-pyrrolo [2,3-b]pyridin-1-yl) nicotinonitrile, Int-2 (1.3 g, 62.50%), TLC: 70% EtOAc (Rf 0.45). MS: m/z=368.02[M+H]⁺.

Step-2: Synthesis of (Z)-5-(5-(4,4-difluoropiperidine-1-carbonyl)-1H-pyrrolo[2,3-b]pyridin-1-yl)—N′-hydroxynicotinimidamide (Int-3): To a stirred solution of 5-(5-(4,4-difluoropiperidine-1-carbonyl)-1H-pyrrolo [2,3-b]pyridin-1-yl) nicotinonitrile, Int-2 (500 mg, 1.36 mmol, 1 eq.) in EtOH (5 mL), NH₂OH·HCl (190 mg, 2.72 mmol, 2 eq) was added followed by addition of Et₃N (0.206 mL, 1.5 mmol, 1.1 eq.) at RT. The resultant mixture was heated to 80° C. for 2 h. The reaction was monitored by LCMS/TLC and, after complete consumption of the starting material, the reaction mixture was evaporated to dryness to remove ethanol and extracted with EtOAc (2×10 mL). Combined organic extracts were washed with brine (10 mL), dried over sodium sulfate, filtered and concentrated in vacuo to obtain the crude. The crude was triturated with Et₂O and dried in vacuo to afford (Z)-5-(5-(4,4-difluoropiperidine-1-carbonyl)-1H-pyrrolo [2,3-b]pyridin-1-yl)—N′-hydroxynicotinimidamide, Int-3 (450 mg) as off white solid TLC: 70% EtOAc (Rf 0.25). MS: m/z=401.01 [M+H]⁺.

Step-3: Synthesis of (1-(5-(5-(tert-butylamino)-1,2,4-oxadiazol-3-yl)pyridin-3-yl)-1H-pyrrolo[2,3-b]pyridin-5-yl)(4,4-difluoropiperidin-1-yl)methanone (B-20): To a stirred solution of (Z)-5-(5-(4,4-difluoropiperidine-1-carbonyl)-1H-pyrrolo [2,3-b]pyridin-1-yl)—N′-hydroxynicotinimidamide (500 mg, 1.25 mmol, 1 eq), tert-butyl isocyanide (0.212 mL, 1.88 mmol, 1.5 eq), Pd(PPh₃)₄ (72 mg, 0.063 mmol, 5.0 mol %), K₂CO₃ (518 mg, 3.75 mmol, 3.0 equiv), in 10 mL of toluene stirred in an air atmosphere for 8 h. The reaction was monitored by LCMS/TLC and, after completion of the starting material, the reaction mixture was diluted with water (10 mL) and extracted with ethyl acetate (3×10 mL). The combined organic layers were washed with water and brine, dried over Na₂SO⁴ and filtered. The solvent was removed in vacuo. The crude was purified by combiflash column chromatography using 10% MeOH: CH₂C₁₂ to afford (1-(5-(5-(tert-butylamino)-1,2,4-oxadiazol-3-yl)pyridin-3-yl)-1H-pyrrolo[2,3-b]pyridin-5-yl) (4,4-difluoropiperidin-1-yl)methanone B-20 (30 mg, 4.99%) as off white solid. TLC: 10% MeOH: CH₂C₁₂ (Rf 0.35) MS: m/z=482.2 [M+H]⁺.

Step-4: Synthesis of (1-(5-(5-amino-1,2,4-oxadiazol-3-yl)pyridin-3-yl)-1H-pyrrolo [2,3-b]pyridin-5-yl)(4,4-difluoropiperidin-1-yl)methanone (B-11): (1-(5-(5-(tert-butylamino)-1,2,4-oxadiazol-3-yl)pyridin-3-yl)-1H-pyrrolo[2,3-b]pyridin-5-yl) (4,4-difluoropiperidin-1-yl) methanone B-20 (15 mg, 0.031 mmol, eq) was dissolved in 2 mL neat trifluoroacetic acid and heated at reflux for 2 h. The reaction was monitored by LCMS/TLC and, after completion of the starting material, the reaction mixture was diluted with water (5 mL) and extracted with ethyl acetate (2×10 mL). The combined organic layers were washed with water and brine, dried over Na₂SO⁴ and filtered. The solvent was removed in vacuo. The crude was purified by combiflash column chromatography using 10% MeOH: CH₂Cl₂ to afford (1-(5-(5-amino-1,2,4-oxadiazol-3-yl)pyridin-3-yl)-1H-pyrrolo [2,3-b]pyridin-5-yl)(4,4-difluoropiperidin-1-yl) methanone, B-11 (5.13 mg, 38.70%) as off white solid. TLC: 10% MeOH: CH₂C₁₂ (Rf 0.35) MS: m/z=426.1 [M+H]⁺.

### Example 14. Synthesis of B-38. B-39. B-40. B-41. B-42. B-43 and B-44

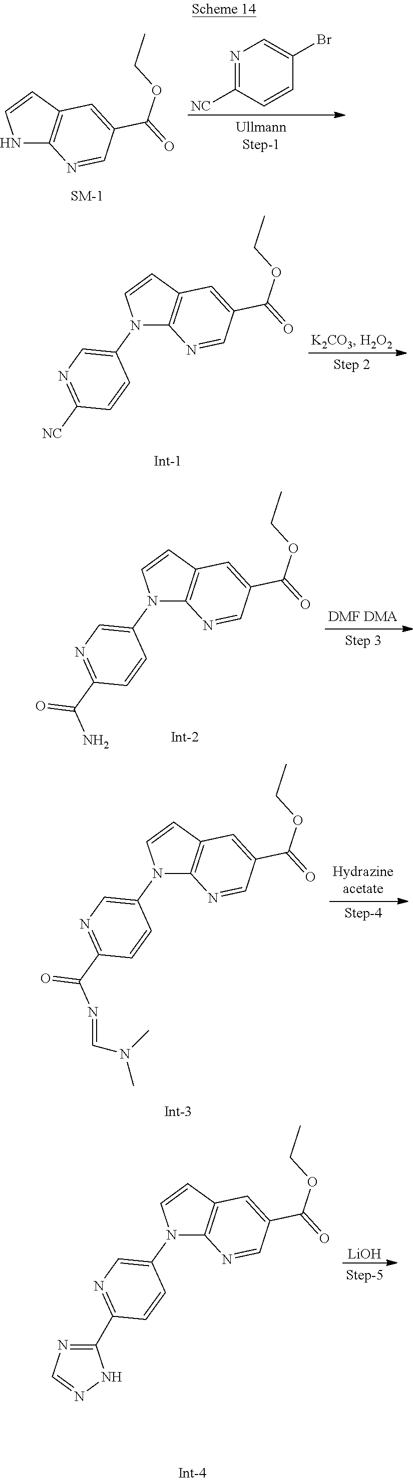

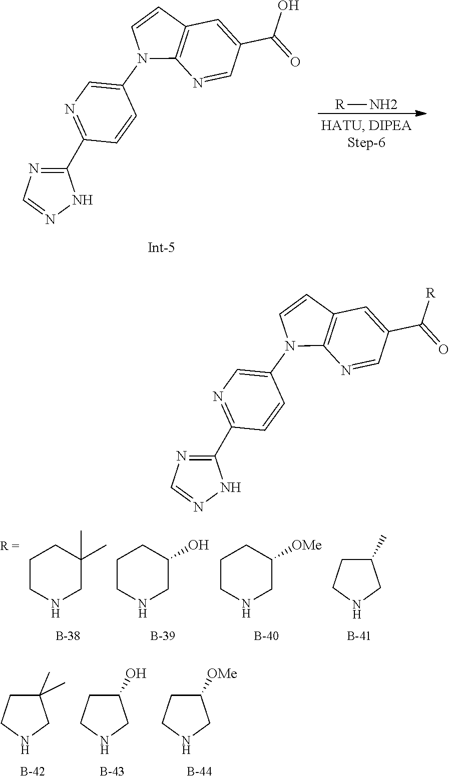

Step-1: Synthesis of ethyl 1-(6-cyanopyridin-3-yl)-1H-pyrrolo[2,3-b]pyridine-5-carboxylate (Int-1): Ethyl 1H-pyrrolo[2,3-b]pyridine-5-carboxylate, SM-1 (3.0 g, 15.7 mmol, 1.0 eq) was converted to Ethyl 1-(6-cyanopyridin-3-yl)-1H-pyrrolo[2,3-b]pyridine-5-carboxylate, Int-1 using general procedure for Ullmann coupling with 5-bromopicolinonitrile (3.4 g, 18.8 mmol, 1.2 eq) to obtain Int-1 (2.1 g, 46% yield) as an off white solid. MS: m/z=293.2 [M+1]⁺).

Step-2: Synthesis of ethyl 1-(6-carbamoylpyridin-3-yl)-1H-pyrrolo[2,3-b]pyridine-5-carboxylate Int-2: Ethyl 1-(6-carbamoylpyridin-3-yl)-1H-pyrrolo[2,3-b]pyridine-5-carboxylate Int-2 was synthesized from Int-1 (2.1 g, 7.19 mmol, 1.0 eq) by the general procedure for oxidation of nitriles using K₂CO₃ (1.48 g, 10.78 mmol, 1.5 eq) and H₂O₂ (0.73 g, 21.57 mmol, 3.0 eq) in DMSO (5 v) to obtain ethyl 1-(6-carbamoylpyridin-3-yl)-1H-pyrrolo[2,3-b]pyridine-5-carboxylate, Int-2 (2.0 g, 90% yield) as off white solid. MS: m/z=311.1 [M+1]).

Step-3: Synthesis of ethyl (E)-1-(6-(((dimethylamino)methylene)carbamoyl)pyridin-3-yl)-1H-pyrrolo[2,3-b]pyridine-5-carboxylate (Int-3): Ethyl 1-(6-carbamoylpyridin-3-yl)-1H-pyrrolo[2,3-b]pyridine-5-carboxylate, Int-2 (2.0 g, 6.45 mmol, 1.0 eq) was converted to (E)-1-(6-(((dimethylamino)methylene)carbamoyl)pyridin-3-yl)-1H-pyrrolo[2,3-b]pyridine-5-carboxylate using the general reaction procedure enaminone formation with DMF-DMA to obtain Int-3 (2.0 g, 92% yield). MS: m/z=366.2 [M+1]³⁰).

Step-4: Synthesis of ethyl 1-(6-(1H-1,2,4-triazol-5-yl)pyridin-3-yl 1H-pyrrolo[2,3-b]pyridine-5-carboxylate Int-4: Int-3 (2.0 g, 5.46 mmol, 1.0 eq) was converted to ethyl 1-(6-(1H-1,2,4-triazol-5-yl)pyridin-3-yl)-1H-pyrrolo[2,3-b]pyridine-5-carboxylate, Int-4 using the general procedure for triazole synthesis using Hydrazine acetate and acetic acid to obtain Int-4 (1.8 g, 98% yield). MS: m/z=335.2 [M+1]³⁰).

Step-5: Synthesis of 1-(6-(1H-1,2,4-triazol-5-yl)pyridin-3-yl)-1H-pyrrolo[2,3-b]pyridine-5-carboxylic acid (Int-5): Ethyl 1-(6-(1H-1,2,4-triazol-5-yl)pyridin-3-yl)-1H-pyrrolo[2,3-b]pyridine-5-carboxylate, Int-4 (1.8 g, 5.38 mmol) was converted to 1-(6-(1H-1,2,4-triazol-5-yl)pyridin-3-yl)-1H-pyrrolo[2,3-b]pyridine-5-carboxylic acid, Int-5 using general procedure for hydrolysis with LiOH (3.0 eq, 16.16 mmol) to afford Int-5 (1.3 g, 79.2% yield) as off white solid. MS: m/z=305.2 [M−1]).

Step-6: Synthesis of B-38, B-39, B-40, B-41, B-42, B-43 and B-44: 1-(6-(1H-1,2,4-triazol-5-yl)pyridin-3-yl)-1H-pyrrolo[2,3-b]pyridine-5-carboxylic acid, Int-5 was converted to B-38, B-39, B-40, B-41, B-42, B-43 and B-44 by using general procedure for acid-amine coupling using HATU, DIPEA to afford B-38 (25.5% MS: m/z=402.1 [M+1]), B-39 (7.8% yield, MS: m/z=390.1 [M+1]), B-40 (53.8% yield, MS: m/z=404.2 [M+1]³⁰), B-41 (4.96%, MS: m/z=374.1 [M+1]³⁰), B-42 (31.7% yield, MS: m/z=388.40 [M+1]), B-43 (1.75% yield, MS: m/z=375.1 [M+1]) and B-44 (35% yield, MS: m/z=389.2 [M+1]).

### Example 15. Synthesis of (S)-(1-(4-(1H-1,2,4-triazol-3-yl)phenyl)-1H-pyrrolo[2,3-b]pyridin-5-yl)(3-methylpiperidin-1-yl)methanone (B-33)/(1-(5-(1H-1,2,4-triazol-3-yl)pyridin-2-yl-1H-pyrrolo[2,3-b]pyridin-5-yl)(4,4-difluoropiperidin-1-yl)methanone (B-34)/(S)-(1-(5-(4H-1,2,4-triazol-3-yl)pyridin-2-yl-1H-pyrrolo[2,3-b]pyridin-5-yl)(3-methylpiperidin-1-yl)methanone (B-35)/(1-(3-(1H-1,2,4-triazol-3-yl)phenyl)-1H-pyrrolo[2,3-b]pyridin-5-yl)(4,4-difluoropiperidin-1-yl)methanone (B-36)/(S)-(1-(3-(1H-1,2,4-triazol-3-yl)phenyl)-1H-pyrrolo[2,3-b]pyridin-5-yl)1 (3-methylpiperidin-1-yl)methanone (B-37)

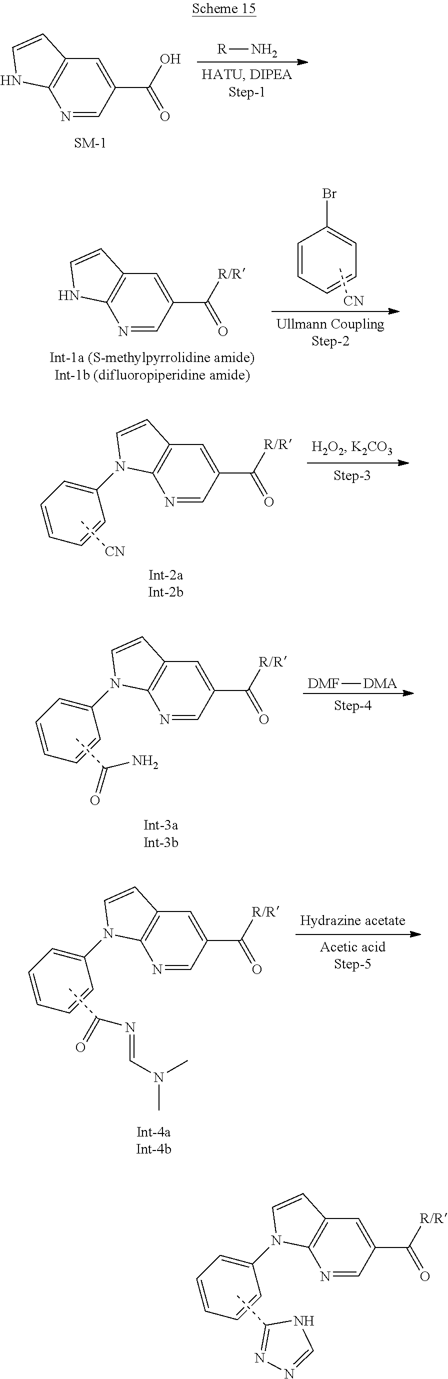

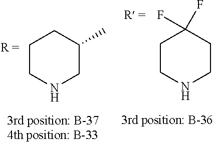

Int-1 is described above in the synthesis of B-12 and B-29.

Step-1: Synthesis (S)-(3-methylpiperidin-1-yl)(1H-pyrrolo[2,3-b]pyridin-5-yl)methanone (Int-1a)/(4,4-difluoropiperidin-1-yl)(1H-pyrrolo[2,3-b]pyridin-5-yl)methanone (Int-1b): pyrrolo[2,3-b]pyridine-5-carboxylic acid, SM-1 (1.0 eq) was converted to (S)-(3-methylpiperidin-1-yl)(1H-pyrrolo[2,3-b]pyridin-5-yl)methanone(Int-1a)/(4,4-difluoropiperidin-1-yl)(1H-pyrrolo[2,3-b]pyridin-5-yl)methanone (Int-b) using general procedure for acid-amine coupling with HATU and (S)-3-methylpiperidine (1.2 eq)/4,4-difluoropiperidine hydrochloride (1.2 eq.) to afford (S)-(3-methylpiperidin-l-yl)(1H-pyrrolo[2,3-b]pyridin-5-yl)methanone (Int-1a) (1.5 g, 66%)/(4,4-difluoropiperidin-1-yl)(1H-pyrrolo[2,3-b]pyridin-5-yl)methanone. (Int-1b) (3 g, 96%).

Step-2: Synthesis of (Int-2a)/(Int-2b): Int-1a/Int-1b (1.0 eq) were synthesized by using general Ullmann coupling of (S)-(3-methylpiperidin-1-yl)(1H-pyrrolo[2,3-b]pyridin-5-yl)methanone (Int-1a)/(4,4-difluoropiperidin-1-yl)(1H-pyrrolo[2,3-b]pyridin-5-yl)methanone (Int-1b) with respective Bromo benzo nitrile (1.2 eq) to afford Int-2a/Int-2b as an off white solid. (Int-2a) (4-position 32.8%, m/z=345.5 [M+1]³⁰) (3-position 40%, m/z=345.5 [M+1]³⁰)/(Int-2b) (3-position 87%, m/z=367.1 [M+1]³⁰).

Step-3: Synthesis of (Int-3a)/(Int-3b): Int-3a/Int3b were synthesized from Int-2a/Int-2b using general oxidation condition by using K₂CO₃ (2.0 eq) and H₂O₂ (5.0 eq) in DMSO (10 v) to afford Int-3a/Int-3b as an off-white solid. Int-3a (4-position 74%, m/z=363.25 [M+1]³⁰) (3-position 51%, m/z=363.25 [M+1]³⁰)/Int-3b (3-position 90%, m/z=385.2 [M+1]³⁰).

Step-4: Synthesis of (Int-4a)/(Int-4b): Int-3a/Int-3b (1.0 eq) were taken in DMF DMA (10 v) and heated to 90° C. for 1 h. The progress of the reaction was monitored with TLC. The solvent was evaporated under reduced pressure and triturated with ether to afford Int-4a/Int-4b as an off-white solid. Crude was used in the next step without further purification. Int-4a (4-position 62%, m/z=418.01 [M+1]³⁰) (3-position 66%, m/z=418.22 [M+1]³⁰)/Int-4b (3-position 78%, m/z+440.1 [M+1]³⁰).

### Example 16. Synthesis of B-34 and B-35

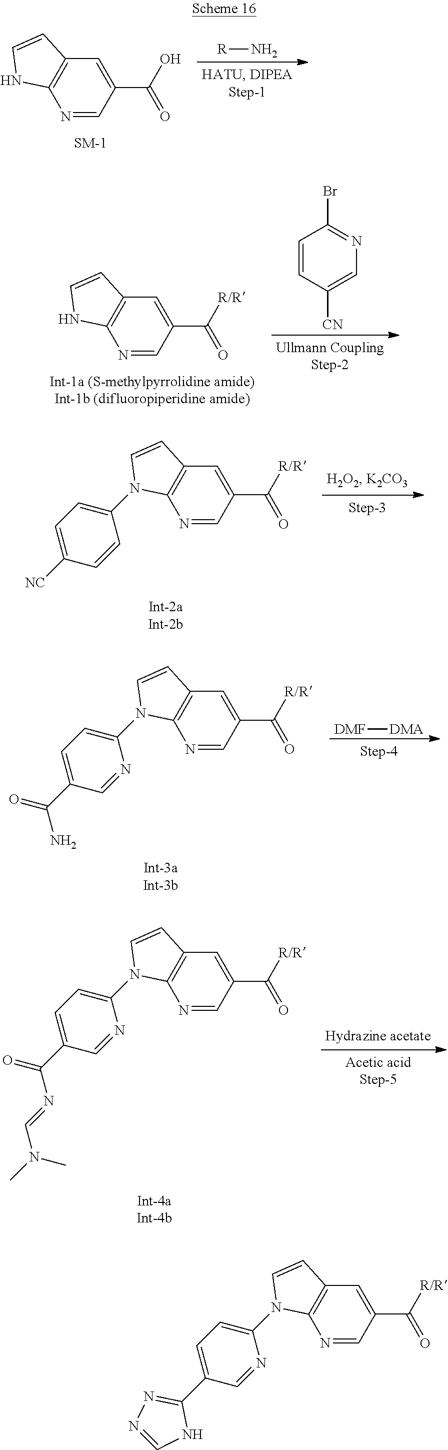

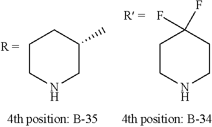

Int-1 is described above in the synthesis of B-12 and B-29.

Step-1: Synthesis (S)-(3-methylpiperidin-1-yl)(1H-pyrrolo[2,3-b]pyridin-5-yl)methanone (Int-1a)/(4,4-difluoropiperidin-1-yl)(1H-pyrrolo[2,3-b]pyridin-5-yl)methanone (Int-1b): pyrrolo[2,3-b]pyridine-5-carboxylic acid, SM-1 (1.0 eq) was converted to (S)-(3-methylpiperidin-1-yl)(1H-pyrrolo[2,3-b]pyridin-5-yl)methanone(Int-1a)/(4,4-difluoropiperidin-1-yl)(1H-pyrrolo[2,3-b]pyridin-5-yl)methanone (Int-b) using general procedure for acid-amine coupling with HATU and (S)-3-methylpiperidine (1.2 eq)/4,4-difluoropiperidine hydrochloride (1.2 eq.) to afford (S)-(3-methylpiperidin-1-yl)(1H-pyrrolo[2,3-b]pyridin-5-yl)methanone (Int-1a) (1.5 g, 66%)/(4,4-difluoropiperidin-1-yl)(1H-pyrrolo[2,3-b]pyridin-5-yl)methanone. (Int-1b) (3 g, 96%).

Step-2: Synthesis of (Int-2a)/(Int-2b): Int-1a/Int-1b (1.0 eq) were synthesized by using general Ullmann coupling of (Int-1a)/(Int-1b) with 4-Bromo benzo nitrile (1.2 eq) to afford Int-2a/Int-2b as an off white solid. (Int-2a) (4-position 41%, m/z=346.16 [M+1]³⁰)/(Int-2b) (4-position 73%, m/z=368.1 [M+1]³⁰).

Step-3: Synthesis of (Int-3a)/(Int-3b): Int-3a/Int3b were synthesized from Int-2a/Int-2b using general oxidation condition by using K₂CO₃ (2.0 eq) and H₂O₂ (5.0 eq) in DMSO (10 v) to afford Int-3a/Int-3b as an off-white solid. Int-3a (4-position 70%, m/z=364.2 [M+1]³⁰)/Int-3b (4-position 82%, m/z=386.2 [M+1]³⁰).

Step-4: Synthesis of (Int-4a)/(Int-4b): Int-3a/Int-3b (1.0 eq) were taken in DMF DMA (10 v) and heated to 90° C. for 1 h. The progress of the reaction was monitored with TLC. The solvent was evaporated under reduced pressure and triturated with ether to afford Int-4a/Int-4b as an off-white solid. Crude was used in the next step without further purification. Int-4a (4-position 58%, m/z=419.01 [M+1]³⁰)/Int-4b (4-position 72%, m/z=441.1 [M+1]³⁰).

Step-5: Synthesis of (S)-(1-(4-(1H-1,2,4-triazol-3-yl)phenyl)-1H-pyrrolo[2,3-b]pyridin-5-yl)(3-methylpiperidin-1-yl)methanone (B-33)/(1-(5-(1H-1,2,4-triazol-3-yl)pyridin-2-yl)-1H-pyrrolo[2,3-b]pyridin-5-yl)(4,4-difluoropiperidin-1-yl)methanone (B-34)/(S)-(1-(5-(4H-1,2,4-triazol-3-yl)pyridin-2-yl)-1H-pyrrolo[2,3-b]pyridin-5-yl)(3-methylpiperidin-1-yl)methanone (B-35)/(1-(3-(1H-1,2,4-triazol-3-yl)phenyl)-1H-pyrrolo[2,3-b]pyridin-5-yl)(4,4-difluoropiperidin-1-yl)methanone (B-36)/(S)-(1-(3-(1H-1,2,4-triazol-3-yl)phenyl)-1H-pyrrolo[2,3-b]pyridin-5-yl)(3-methylpiperidin-1-yl)methanone (B-37):To a stirred solution of Int-5 (1.0 eq) in acetic acid (10 v), was added hydrazine acetate (5.0 eq) and heated to 80° C., for 1 h. The progress of the reaction was monitored by TLC and LCMS. The acetic acid was evaporated, diluted with EtOAc and washed with NaHCO₃ solution, water and brine solution. The combined extracts were dried over sodium sulphate, filtered and concentrated.

### Example 17. Synthesis of (1-(2-(1H-1,2,4-triazol-5-yl)pyrimidin-5-yl)-1H-pyrrolo[2,3b]pyridin-5-yl)(4,4-difluoropiperidin-1-yl)methanone (B-45)

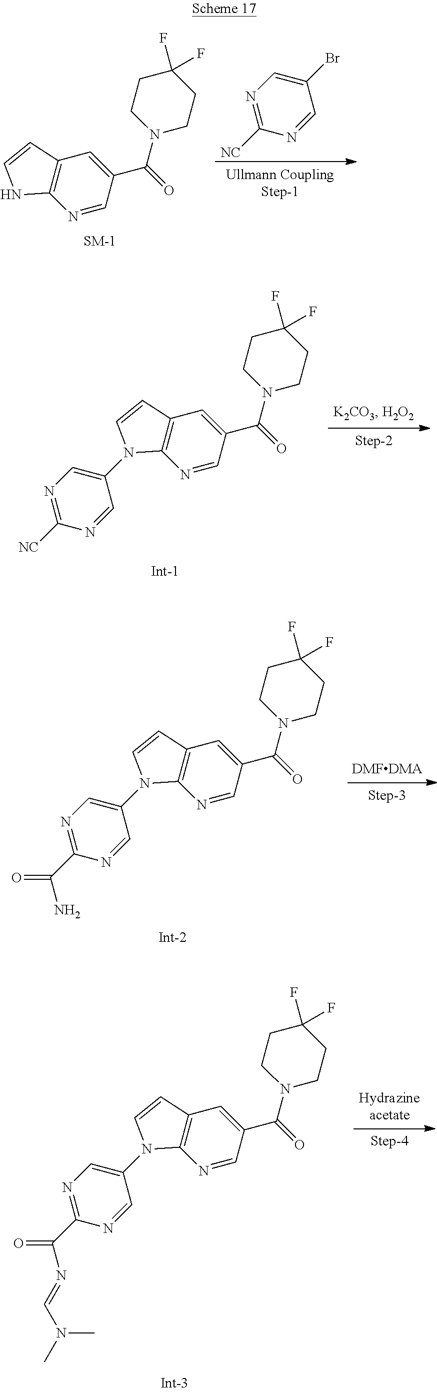 [SMILES: O=C(c1cnc2[nH]ccc2c1)N1CCC(F)(F)CC1 ; N#Cc1ncc(Br)cn1 ; N#Cc1ncc(-n2ccc3cc(C(=O)N4CCC(F)(F)CC4)cnc32)cn1 ; NC(=O)c1ncc(-n2ccc3cc(C(=O)N4CCC(F)(F)CC4)cnc32)cn1 ; CN(C)/C=N/C(=O)c1ncc(-n2ccc3cc(C(=O)N4CCC(F)(F)CC4)cnc32)cn1]

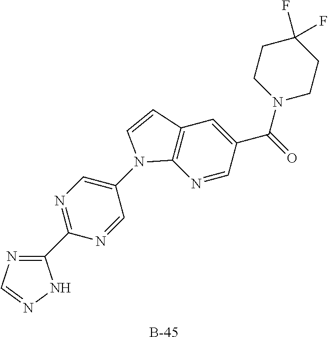

Int-1 is described above in the synthesis of B-12.

Step-1: Synthesis of 5-(5-(4,4-difluoropiperidine-1-carbonyl)-1H-pyrrolo[2,3-b]pyridin-1-yl)pyrimidine-2-carbonitrile (Int-1): Using the general procedure for Ullman coupling (4,4-difluoropiperidin-1-yl)(1H-pyrrolo[2,3-b]pyridin-5-yl)methanone (SM-1) (500 mg, 1.88 mmol, 1.0 eq.) was converted to 5-(5-(4,4-difluoropiperidine-1-carbonyl)-1H-pyrrolo[2,3-b]pyridin-1-yl)pyrimidine-2-carbonitrile (Int-1) (0.48 g, Yield=69.1%, MS: m/z=369.00 [M+H]⁺.)

Step-2: Synthesis of 5-(5-(4,4-difluoropiperidine-1-carbonyl)-1H-pyrrolo[2,3-b]pyridin-1-yl)pyrimidine-2-carbonitrile (Int-2): 4-(5-(4,4-difluoropiperidine-1-carbonyl)-1H-benzo[d][1,2,3]triazol-1-yl)benzonitrile (Int-1) (280 mg, 0.76 mmol, 1.0 eq.) was converted to 4-(5-(4,4-difluoropiperidine-1-carbonyl)-1H-benzo[d][1,2,3]triazol-1-yl)benzamide using general procedure for benzamide formation using H₂O₂ to afford 5-(5-(4,4-difluoropiperidine-1-carbonyl)-1H-pyrrolo[2,3-b]pyridin-1-yl)pyrimidine-2-carboxamide (Int-2) (200 mg, Yield=68.25%, Ms: m/z=387.1 [M+1]³⁰), as pale yellow solid.

Step-3: Synthesis of (E)-5-(5-(4,4-difluoropiperidine-1-carbonyl)1H-pyrrolo[2,3-b]pyridin-1-yl)—N—((dimethylamino)methylene)pyrimidine-2-carboxamide (Int-3): 5-(5-(4,4-difluoropiperidine-1-carbonyl)-1H-pyrrolo[2,3-b]pyridin-1-yl)pyrimidine-2-carboxamide (Int-2) (200 mg, 0.51 mmol, 1.0 eq.) was converted to (E)-5-(5-(4,4-difluoropiperidine-1-carbonyl)-1H-pyrrolo[2,3-b]pyridin-1-yl)—N—((dimethylamino)methylene)pyrimidine-2-carboxamide (Int-3) using general procedure for enamine formation. The crude obtained was triturated with Et₂O to afford (E)-5-(5-(4,4-difluoropiperidine-1-carbonyl)-1H-pyrrolo[2,3-b]pyridin-1-yl)—N—((dimethylamino)methylene)pyrimidine-2-carboxamide (Int-3) (180 mg, Yield=78.94%, Ms: m/z+442.00 [M+1]³⁰) as an off-white solid.

Step-4: Synthesis of (1-(2-(1H-1,2,4-triazol-5-yl)pyrimidin-5-yl)-1H-pyrrolo[2,3-b]pyridin-5-yl)(4,4-difluoropiperidin-1-yl)methanone (B-45): (E)-5-(5-(4,4-difluoropiperidine-1-carbonyl)-1H-pyrrolo[2,3-b]pyridin-1-yl)—N—((dimethylamino)methylene)pyrimidine-2-carboxamide (Int-3) (180 mg, 0.44 mmol, 1.0 eq.) was converted to (1-(2-(1H-1,2,4-triazol-5-yl)pyrimidin-5-yl)-1H-pyrrolo[2,3-b]pyridin-5-yl)(4,4-difluoropiperidin-1-yl)methanone B-45 using general procedure for triazole formation using hydrazine acetate. The crude was purified by combi-flash column chromatography using 5% MeOH: DCM to afford (1-(2-(1H-1,2,4-triazol-5-yl)pyrimidin-5-yl)-1H-pyrrolo[2,3-b]pyridin-5-yl)(4,4-difluoropiperidin-1-yl)methanone B-45 (110 mg, Yield=65.86%, Ms: m/z=411.2 [M+H]⁺) as off white solid. TLC: 5% MeOH/CH₂Cl₂ (Rf: 0.25).

### Example 18. Synthesis of ethyl 4-(5-(4,4-difluoropiperidine-1-carbonyl)-1H-pyrrolo[2,3-b]pyridin-1-yl)benzoate (B-32)

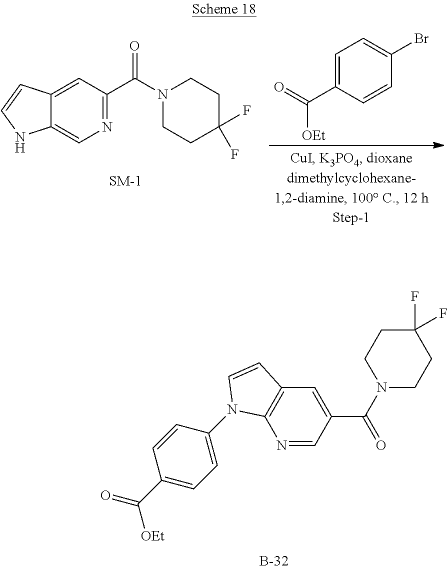

Int-1 is described above in the synthesis of B-12.

Using the general procedure for Ullman coupling (4,4-difluoropiperidin-1-yl)(1H-pyrrolo[2,3-b]pyridin-5-yl)methanone (SM-1) (75 mg, 0.19 mmol, 1.0 eq.) was converted to ethyl 4-(5-(4,4-difluoropiperidine-1-carbonyl)-1H-pyrrolo[2,3-b]pyridin-1-yl)benzoate B-32 (44.6 mg, Yield=55.4%, MS: m/z=414.20 [M+H]⁺.

### Eample 19. Synthesis of (1-(6 (1H-1,2,4-triazol-5-yl)pyridin-3-yl)-1H-pyrrolo[2,3-b]pyridin-5-yl)-1H-pyrrolo[2,3-b]pyridin-5-yl)(2,6-dimethylmorpholino)methanone (B-47)/5-(5-(4,4-difluoropiperidine-1-carbonyl)-1H-pyrrolo2,3-b1 pyridin-1-yl) picolinimidohydrazide (B-48) and (1-(6-(3-amino-1H-1,2A-triazol-5-yl) pyridin-3-yl)-1H-pyrrolo[2,3-b]pyridin-5-yl)1 (2-methylmorpholino)methanone (B-49)

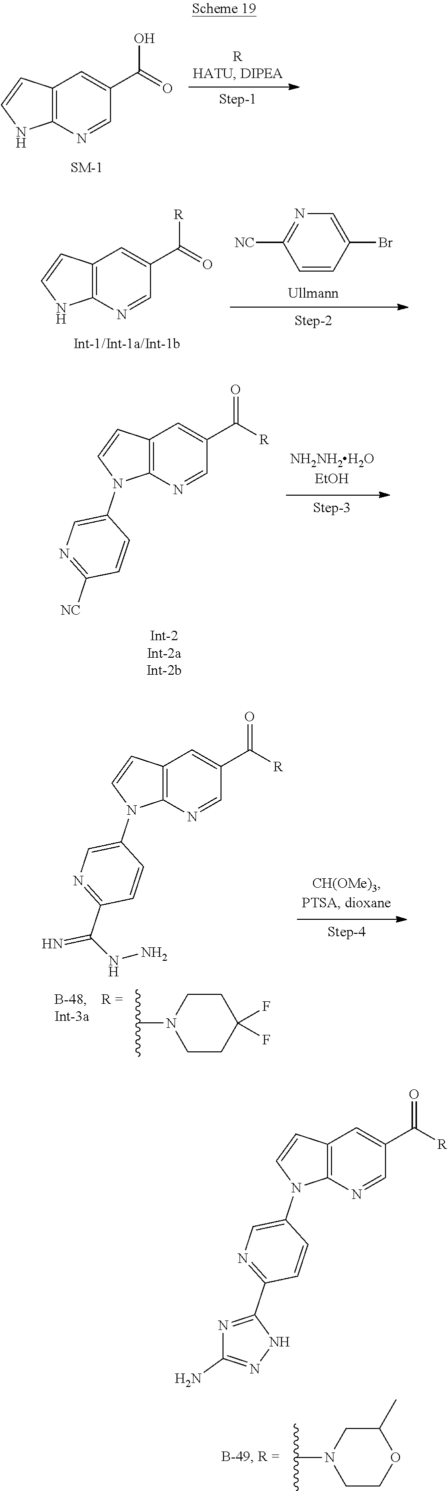

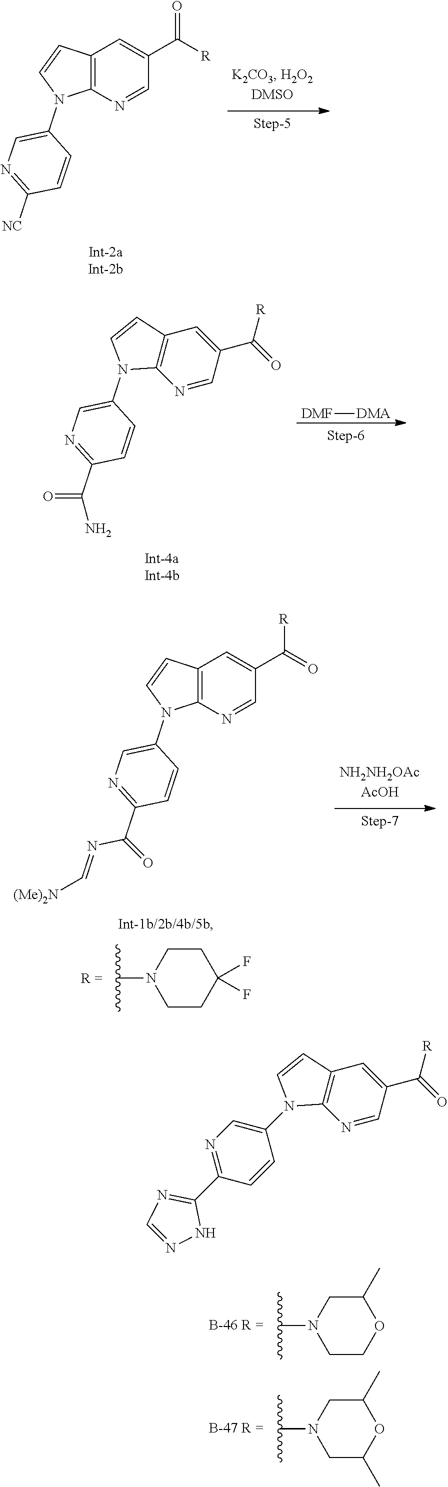

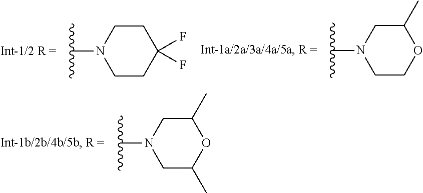

Step-1: Synthesis of (2-methylmorpholino)1H-pyrrolo[2,3-b]pyridin-5-yl) methanone, Int-1a and (2,6-dimethylmorpholino) (1H-pyrrolo[2,3-b]pyridin-5-yl) methanone Int-1b: 1H-pyrrolo[2,3-b]pyridine-5-carboxylic acid, SM-1 was converted to Int-1a and Int-1b using general procedure for HATU acid-amine coupling affording Int-1a (60% yield, m/z=246.1 [M+H]⁺) and Int-1b (68% yield, m/z=260.1 [M+H]) as an off-white solids. Int-1 was synthesized as previously described.

Step-2: Synthesis of 5-(5-(4,4-difluoropiperidine-1-carbonyl)-1H-pyrrolo[2,3-b]pyridin-1-yl) picolinonitrile, Int-2/5-(5-(2-methylmorpholine-4-carbonyl-1H-pyrrolo[2,3-b]pyridin-1-yl) picolinonitrile, Int-2a and 5-(5-(2,6-dimethylmorpholine-4-carbonyl)-1H-pyrrolo[2,3-b]pyridin-1-yl) picolinonitrile, Int-2b: Int-1, Int-1a and Int-1b was converted to Int-2, Int-2a and Int-2b using the general procedure for Ullmann coupling with 5-bromopicolinonitrile. Int-2 (43.50% yield, m/z=368.2 [M+H]⁺.), Int-2a (44.3% yield, m/z=348.1 [M+H]⁺) and Int-2b (32% yield, m/z=362.2 [M+H]⁺) were isolated as off-white solids.

Step-3: Synthesis of 5-(5-(4,4-difluoropiperidine-1-carbonyl)-1H-pyrrolo[2,3-b]pyridin-1-yl) picolinimidohydrazide and 5-(5-(2-methylmorpholine-4-carbonyl)-1H-pyrrolo[2,3-b]pyridin-1-yl)picolinimidohydrazide, Int-3a: Int-2/Int-2a was converted to B-48 (41% yield, m/z=400.1 [M+H]⁺) and Int-3a (100% crude, m/z=380.02 [M+H]⁺) respectively using general procedure for imidohydrazide formation with hydrazine.

Step-4: Synthesis of (1-(6-(3-amino-1H-1,2,4-triazol-5-yl)pyridin-3-yl)9-1H-pyrrolo [2,3-b]pyridin-5-yl)(2-methylmorpholino)methanone, B-49: To a stirred solution of (Int-3a) (1.0 eq) in 1,4-dioxane (10 vol.)), was added triethylorthoformate (5.0 eq) and p-toluenesulfonic acid monohydrate (0.2 eq). The resulting reaction mixture was stirred at 100° C. for 16 h. The progress of the reaction was monitored with TLC/LCMS, After completion, the reaction mixture was quenched with saturated NaHCO₃ solution and extracted with EtOAc. The combined organic layers were washed with water followed by brine, dried over Na₂SO₄, filtered and concentrated under reduced pressure. The crude was purified by combi-flash column chromatography using 5% MeOH: DCM to afford (1-(6-(3-amino-1H-1,2,4-triazol-5-yl)pyridin-3-yl)-1H-pyrrolo [2,3-b]pyridin-5-yl)(2-methylmorpholino)methanone, B-49 (15.33 mg, 7.1%) as an off white solid. MS: m/z=405.1 [M+H]⁺.

Step-5: Synthesis of 5-(5-(2-methylmorpholine-4-carbonyl)-1H-pyrrolo[2,3-b]pyridin-1-yl)picolinamide, Int-4a/5-(5-(2,6-dimethylmorpholine-4-carbonyl)-1H-pyrrolo[2,3-b]pyridin-1-yl)picolinamide, Int-4b: Int-2a/Int-2b was converted into Int-4a/Int-4b respectively using the general procedure for amide formation with K₂CO₃ and H₂O₂ to afford Int-4a (77% yield m/z=366.1 [M+H]⁺) and Int-4b (79% yield m/z=380.1 [M+H]⁺) as off-white solids.

Step-6: Synthesis of (E)-N—((dimethylamino)methylene)-5-(5-(2-methylmorpholine-4-carbonyl)-1H-pyrrolo[2,3-b]pyridin-1-yl)picolinamide, Int-5a/(E)-N-((dimethylamino)methylene)-5-(5-(2,6-dimethylmorpholine-4-carbonyl)-1H-pyrrolo[2,3-b]pyridin-1-yl)picolinamide, Int-5b: Int-4a/Int-4b (1 eq.) in DMF-DMA (10 V) was heated to 80° C., for 2 h. The progress of the reaction was monitored with TLC. The reaction mixture was concentrated under reduced pressure and washed with heptane to give an off-white solid Int-5a (77% yield, m/z=421.2 [M+H]⁺) and Int-5b (77% yield, m/z=435.2 [M+H]⁺.)

Step-7: Synthesis of (1-(6-(1H-1,2,4-triazol-5-yl)pyridin-3-yl)-1H-pyrrolo[2,3-b]pyridin-5-yl)(2-methylmorpholino)methanone B-46/(1-(6-(1H-1,2,4-triazol-5-yl)pyridin-3-yl)1H-pyrrolo[2,3-b]pyridin-5-yl)(2,6-dimethylmorpholino)methanone B-47: To Int-5a, Int-5b (1 eq) in acetic acid (10 v) was added hydrazine acetate (5 eq) and stirred at 90° C. for 1 h. The progress of the reaction was monitored with TLC. The reaction mixture was concentrated under reduced pressure, made basic with saturated NaHCO₃. The obtained solid was filtered and dried to give (1-(6-(1H-1,2,4-triazol-5-yl)pyridin-3-yl)-1H-pyrrolo[2,3-b]pyridin-5-yl)(2-methylmorpholino)methanone B-46 (55% yield, m/z=390.1 [M+H]⁺)/(1-(6-(1H-1,2,4-triazol-5-yl)pyridin-3-yl)-1H-pyrrolo[2,3-b]pyridin-5-yl)(2,6-dimethylmorpholino) methanone none B-47 (81% yield, m/z=390.1 [M+H]⁺.)

### Example 20. Synthesis of (R)-(1-(3-(4H-1,2,4-triazol-3-yl)phenyl)-1H-pyrrolo [2,3-b]pyridin-5-yl)(2-methylmorpholino)methanone (B-189)

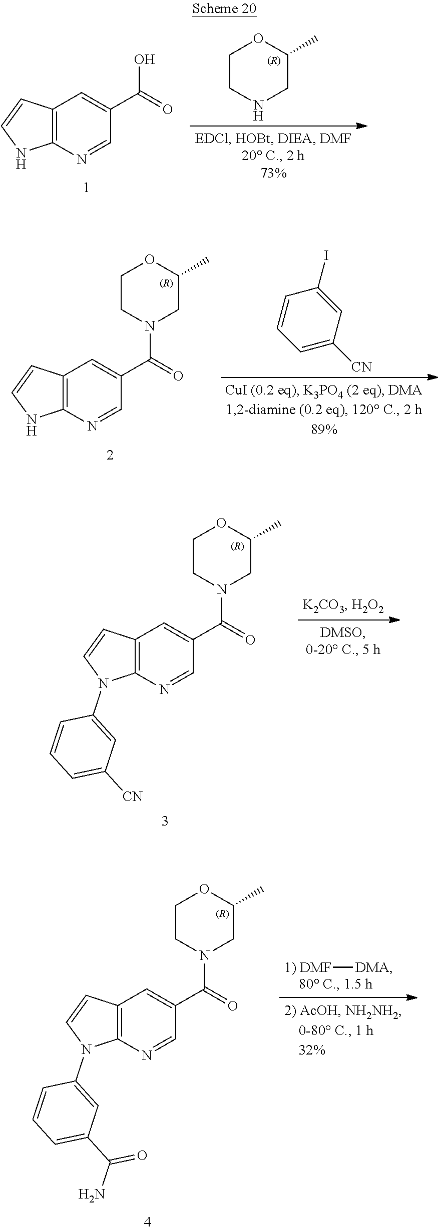

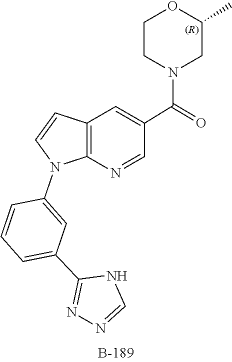

Step-1: Synthesis of Compound 2: To a mixture of 1H-pyrrolo[2,3-b]pyridine-5-carboxylic acid (500 mg, 3.08 mmol, 1.00 eq.), (R)-2-methylmorpholine (374 mg, 3.70 mmol, 1.20 eq.), EDCI (1.18 g, 6.17 mmol, 2.00 eq.), HOBt (833 mg, 6.17 mmol, 2.00 eq.) in DMF (5 mL) was added DIEA (1.20 g, 9.25 mmol, 1.61 mL, 3.00 eq.) and the mixture was stirred at 20° C. for 2 hours. The reaction mixture was diluted with H₂O (25 mL) and extracted with EtOAc (20 mL×3). The combined organic layers were washed with brine (20 mL×2), dried over Na₂SO₄, filtered and concentrated under reduced pressure to give a residue. The residue was purified by prep-HPLC (FA condition) to give (R)-(2-methylmorpholino) 1H-pyrrolo[2,3-b]pyridin-5-yl)methanone (590 mg, 2.26 mmol, 73% yield, 94% purity) as a yellow oil.

¹H NMR (400 MHz, chloroform-d) 8=10.46 (br s, 1H), 8.46 (d, J=1.2 Hz, 1H), 8.06 (d, J=1.6 Hz, 1H), 7.50-7.39 (m, 1H), 6.59 (dd, J=1.6, 3.2 Hz, 1H), 5.01-4.28 (m, 1H), 4.01-3.53 (m, 4H), 3.40-2.76 (m, 2H), 1.31-1.07 (m, 3H).

Step-2: Synthesis of Compound 3: To a mixture of (R)-(2-methylmorpholino) 1H-pyrrolo[2,3-b]pyridin-5-yl)methanone (300 mg, 1.22 mmol, 1.00 eq.), 3-iodobenzonitrile (336 mg, 1.47 mmol, 1.20 eq.), CuI (46.6 mg, 245 μmol, 0.20 eq.), K₃PO₄ (519 mg, 2.45 mmol, 2.00 eq.) and dimethylcyclohexane-1,2-diamine (34.8 mg, 245 μmol, 0.20 eq.) in DMA (3 mL) was degassed and purged with N₂ for 3 times, and then the mixture was stirred at 120° C. for 2 hours under N₂ atmosphere (15 psi). The reaction mixture was diluted with H₂O (30 mL) and EtOAc (30 mL), then filtered to give a filtrate and extracted with EtOAc (20 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=2/1 to ⅓) to give (R)-3-(5-(2-methylmorpholine-4-carbonyl)-1H-pyrrolo[2,3-b]pyridin-1-yl)benzonitrile (390 mg, 1.09 mmol, 89% yield, 97% purity) as a yellow oil.

¹H NMR (400 MHz, chloroform-d) S=8.46 (d, J=1.6 Hz, 1H), 8.21 (s, 1H), 8.12 (d, J=1.6 Hz, 1H), 8.08 (td, J=2.4, 6.8 Hz, 1H), 7.71-7.63 (m, 2H), 7.61 (d, J=4.0 Hz, 1H), 6.77 (d, J=3.6 Hz, 1H), 4.90-4.25 (m, 1H), 4.07-3.86 (m, 1H), 3.83-3.46 (m, 3H), 3.42-2.72 (m, 2H), 1.26-1.03 (m, 3H).

Step-3: Synthesis of Compound 4: To a solution of (R)-3-(5-(2-methylmorpholine-4-carbonyl)-1H-pyrrolo[2,3-b]pyridin-1-yl)benzonitrile (340 mg, 982 μmol, 1.00 eq.) in DMSO (3.5 mL) was added K₂CO₃ (203 mg, 1.47 mmol, 1.50 eq.) and the mixture was stirred at 0° C., then slowly added H₂O₂ (2.01 g, 17.7 mmol, 1.7 mL, 30% purity, 18.0 eq.) at 0° C. and stirred at 0° C. for 1 hour, then the mixture was stirred at 20° C. for another 4 hours. The reaction mixture was diluted with H₂O (30 mL) and extracted with EtOAc (30 mL×3). The combined organic layers were washed with brine (20 mL×2), dried over Na₂SO₄, filtered and concentrated under reduced pressure to give (R)-3-(5-(2-methylmorpholine-4-carbonyl)-1H-pyrrolo[2,3-b]pyridin-1-yl)benzamide (300 mg, crude) as a white solid.

LCMS (ESI, M+1): m/z=365.1

Step-4: (R)-(1-(3-(4H-1,2,4-triazol-3-yl)phenyl)-1H-pyrrolo[2,3-b]pyridin-5-yl)(2-methylmorpholino)methanone. To a solution of (R)-3-(5-(2-methylmorpholine-4-carbonyl)-1H-pyrrolo[2,3-b]pyridin-1-yl)benzamide (300 mg, 823 μmol, 1.00 eq.) in DMFDMA (3 mL) was stirred at 80° C. for 1.5 hours, then the reaction mixture was concentrated under reduced pressure to give a residue. The residue was added AcOH (6 mL), NH₂NH₂·H₂O (4.22 g, 84.3 mmol, 4.1 mL, 102 eq.) at 0° C. and the mixture was stirred at 0° C. for 0.25 hours. Then the mixture was stirred at 80° C. for 0.75 hours. The reaction mixture was diluted with H₂O (40 mL) and extracted with EtOAc (40 mL×3). The combined organic layers were washed with brine (30 mL×2), dried over Na₂SO₄, filtered and concentrated under reduced pressure to give a residue. The residue was purified by prep-HPLC (column: Phenomenex Synergi C18 150×25 mm×10 μm 10 um; mobile phase: [water(FA)-ACN]; B %: 21%-51%, 10 min) to give (R)-(1-(3-(4H-1,2,4-triazol-3-yl)phenyl)-1H-pyrrolo [2,3-b]pyridin-5-yl)(2-methylmorpholino)methanone (102 mg, 262 μmol, 32% yield, 99% purity) as a white solid. LCMS (ESI, M+1): m/z=389.1. ¹H NMR (400 MHz, DMSO-d₆δ8=14.65-14.01 (m, 1H), 8.55 (s, 2H), 8.41 (d, J=1.6 Hz, 1H), 8.19 (d, J=2.0 Hz, 1H), 8.11 (d, J=3.6 Hz, 1H), 8.02 (d, J=8.0 Hz, 1H), 7.92 (br d, J=7.6 Hz, 1H), 7.73-7.63 (m, 1H), 6.84 (d, J=3.6 Hz, 1H), 4.53-4.13 (m, 1H), 4.08-3.68 (m, 2H), 3.67-3.38 (m, 4H), 1.20-0.94 (m, 3H).

### Example 21. Synthesis of (R)-(5-(5-(3-ethylpiperidine-1-carbonyl)-1H-pyrrolo[2,3-b]pyridin-1-yl) pyridin-3-yl) carbamate (B-69) and methyl(S)-(5-(5-(3-ethylpiperidine-1-carbonyl)-1H-pyrrolo[23-b]pyridin-1-yl) pyridin-3-yl) carbamate (B-70)

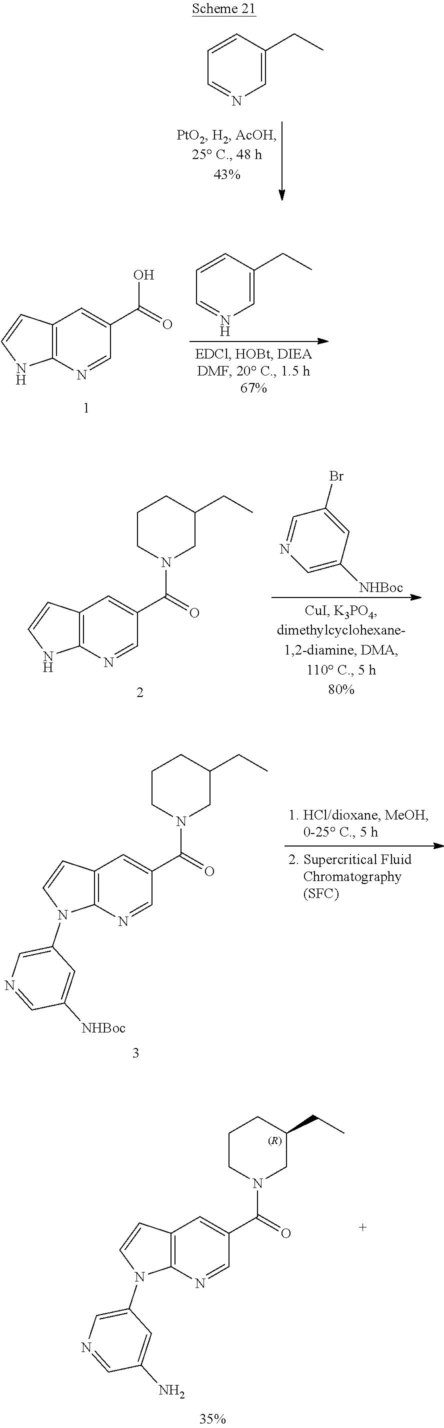

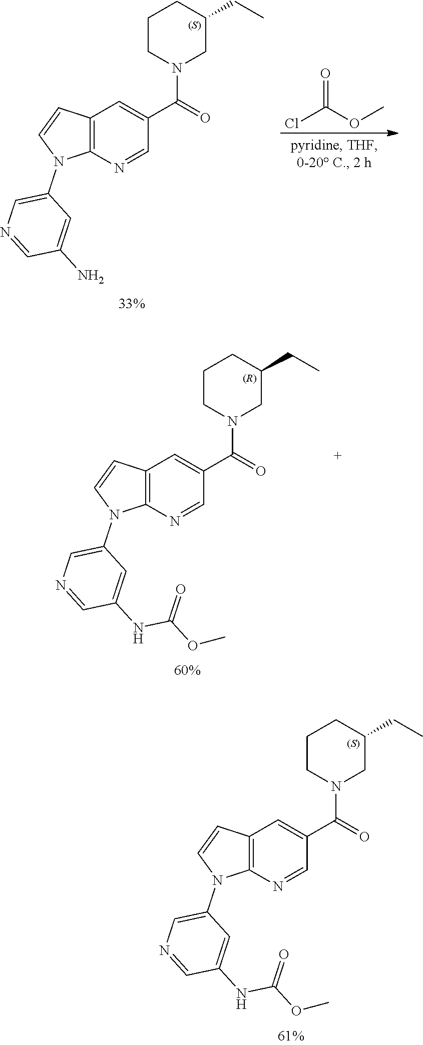

Step-1: Synthesis of Compound B: To a solution of 3-ethylpyridine (100 g, 933 mmol, 105 mL, 1.00 eq.) in AcOH (2000 mL) was added PtO₂ (20.0 g, 88.1 mmol, 9.44e⁻² eq). The mixture was degassed and purged with H₂ for 3 times, and then the mixture was stirred at 25° C. for 48 hours under H₂ (50 psi) atmosphere. The reaction mixture was filtered and added HCl (12 M, 100 mL), then concentrated under reduced pressure to give a residue. The crude product was triturated with MeCN (100 mL) and filtered to give 3-ethylpiperidine (60.0 g, 401 mmol, 43% yield, HCl) as a white solid.

¹H NMR (400 MHz, DMSO-d₆δ S=9.12 (br s, 1H), 3.20-3.08 (m, 2H), 2.70 (dt, J=3.2, 12.4 Hz, 1H), 2.44 (br t, J=12.0 Hz, 1H), 1.83-1.69 (m, 2H), 1.68-1.56 (m, 2H), 1.32-1.13 (m, 2H), 1.12-1.00 (m, 1H), 0.85 (t, J=7.6 Hz, 3H).

Step-2: Synthesis of Compound 2: To a mixture of 1H-pyrrolo[2,3-b]pyridine-5-carboxylic acid (27.0 g, 166 mmol, 1.00 eq.), 3-ethylpiperidine (27.4 g, 183 mmol, 1.10 eq., HCl), EDCI (63.8 g, 333 mmol, 2.00 eq.), HOBt (45.0 g, 333 mmol, 2.00 eq.) in DMF (300 mL) was added DIEA (108 g, 833 mmol, 145 mL, 5.00 eq.) and the mixture was stirred at 20° C. for 1.5 hours. The reaction mixture was diluted with H₂O (1500 mL) and extracted with EtOAc (500 mL×3). The combined organic layers were washed with brine (1000 mL×2), dried over Na₂SO₄, filtered and concentrated under reduced pressure to give a residue. The crude product was triturated with EtOAc (100 mL) and filtered to give (3-ethylpiperidin-1-yl)(1H-pyrrolo[2,3-b]pyridin-5-yl)methanone (31.0 g, 112 mmol, 67% yield, 93% purity) as a yellow solid. ¹H NMR (400 MHz, DMSO-d₆δ8=11.86 (br s, 1H), 8.23 (d, J=1.6 Hz, 1H), 7.98 (s, 1H), 7.55 (t, J=2.4 Hz, 1H), 6.51 (br d, J=1.6 Hz, 1H), 4.70-4.09 (m, 1H), 3.64 (br s, 1H), 3.18-2.83 (m, 1H), 2.81-2.55 (m, 1H), 1.92-1.76 (m, 1H), 1.63 (br s, 1H), 1.40 (br d, J=3.6 Hz, 2H), 1.30-1.01 (m, 3H), 0.83 (br d, J=1.2 Hz, 3H).

Step-3: Synthesis of Compound 3: To a mixture of (3-ethylpiperidin-1-yl)(1H-pyrrolo[2,3-b]pyridin-5-yl)methanone (31.0 g, 120 mmol, 1.00 eq.), tert-butyl (5-bromopyridin-3-yl)carbamate (49.3 g, 181 mmol, 1.50 eq.), K₃PO₄ (51.1 g, 241 mmol, 2.00 eq.), CuI (11.5 g, 60.2 mmol, 0.50 eq.) and dimethylcyclohexane-1,2-diamine (17.1 g, 120 mmol, 1.00 eq.) in DMA (300 mL) was degassed and purged with N₂ for 3 times, and then the mixture was stirred at 110° C. for 5 hours under N₂ atmosphere (15 psi). The reaction mixture was diluted with H₂O (2000 mL) and EtOAc (1000 mL), the mixture was added NH₃·H₂O (200 mL, 25% purity), then filtered to give a filtrate and extracted with EtOAc (1000 mL×3). The combined organic layers were washed with brine (2000 mL×2), dried over Na₂SO₄, filtered and concentrated under reduced pressure to give a residue. The residue was purified by column chromatography (SiO₂, PE/EA=3/1 to ½) to give tert-butyl (5-(5-(3-ethylpiperidine-1-carbonyl)-1H-pyrrolo[2,3-b]pyridin-1-yl)pyridin-3-yl)carbamate (47.0 g, 96.2 mmol, 80% yield, 92% purity) as a yellow solid.

¹H NMR (400 MHz, DMSO-d₆δ S=9.85 (s, 1H), 8.68 (d, J=2.4 Hz, 1H), 8.61 (d, J=1.6 Hz, 1H), 8.49 (t, J=2.0 Hz, 1H), 8.35 (d, J=2.0 Hz, 1H), 8.14 (d, J=2.0 Hz, 1H), 8.04 (d, J=3.6 Hz, 1H), 6.84 (d, J=3.6 Hz, 1H), 4.54-4.16 (m, 1H), 3.87-3.40 (m, 1H), 3.03-2.66 (m, 2H), 1.84 (br dd, J=4.4, 9.2 Hz, 1H), 1.77-1.55 (m, 1H), 1.50 (s, 9H), 1.45-1.35 (m, 2H), 1.32-1.20 (m, 1H), 1.17-1.03 (m, 2H), 0.91-0.74 (m, 3H).

Step-4: Synthesis of MF-642 (R)-(5-(5-(3-ethylpiperidine-1-carbonyl)-1H-pyrrolo[2,3-b]pyridin-1-yl)pyridin-3-yl)carbamate (B-69), and methyl(S)-(5-(5-(3-ethylpiperidine-1-carbonyl)-1H-pyrrolo[2,3-b]pyridin-1-yl)pyridin-3-yl)carbamate (B-70). To a solution of tert-butyl (5-(5-(3-ethylpiperidine-1-carbonyl)-1H-pyrrolo[2,3 b]pyridin-1-yl)pyridin-3-yl)carbamate (35.0 g, 77.9 mmol, 1.00 eq.) in MeOH (200 mL) was added HCl·dioxane (4 M, 200 mL, 10.3 eq.) at 0° C. and the mixture was stirred at 25° C. for 5 hours. The reaction mixture was concentrated under reduced pressure to give a residue. The residue was diluted with H₂O (300 mL) and EtOAc (300 mL), then added NaHCO₃ to adjust pH to 8, and then extracted with EtOAc (200 mL×3). The combined organic layers were washed with brine (300 mL×2), dried over Na₂SO₄, filtered and concentrated under reduced pressure to give a residue. The product was further separated by SFC (column: REGIS (s, s) WHELK-01 (250 mm×50 mm, 10 um); mobile phase: [MeOH-ACN]; B %: 32%-32%, 7.0 min) to give:

(R)-(1-(5-aminopyridin-3-yl)-1H-pyrrolo[2,3-b]pyridin-5-yl)(3-ethylpiperidin-1-yl)methanone (9.70 g, 27.2 mmol, 35% yield, 98% purity) as a yellow solid. LCMS (ESI, M+1): m/z=350.1

(S)-(1-(5-aminopyridin-3-yl)-1H-pyrrolo[2,3-b]pyridin-5-yl)(3-ethylpiperidin-1-yl)methanone (9.00 g, 25.6 mmol, 33% yield, 99% purity) as a yellow solid. LCMS (ESI, M+1): m/z=350.2. ¹H NMR (400 MHz, DMSO-d₆δ S=8.33 (d, J=2.0 Hz, 1H), 8.17 (d, J=2.0 Hz, 1H), 8.12 (d, J 2.0 Hz, 1H), 7.99 (d, J=3.6 Hz, 1H), 7.93 (d, J 2.4 Hz, 1H), 7.50 (t, J 2.4 Hz, 1H), 6.79 (d, J 3.6 Hz, 1H), 5.65 (s, 1H), 4.51-4.21 (m, 1H), 3.85-3.46 (m, 1H), 3.08-2.77 (m, 1H), 1.93-1.79 (m, 1H), 1.74-1.55 (m, 1H), 1.51-1.35 (m, 2H), 1.33-1.02 (m, 3H), 0.92-0.73 (m, 3H).

Compound B-69, (R)-(5-(5-(3-ethylpiperidine-1-carbonyl)-1H-pyrrolo[2,3-b]pyridin-1-yl)pyridin-3-yl)carbamate: To a mixture of (R)-(1-(5-aminopyridin-3-yl)-1H-pyrrolo[2,3-b]pyridin-5-yl)(3-ethylpiperidin-1-yl)methanone (5.00 g, 14.3 mmol, 1.00 eq.), pyridine (3.40 g, 42.9 mmol, 3.46 mL, 3.00 eq.) in THF (50 mL) was added methyl carbonochloridate (3.60 g, 38.1 mmol, 2.95 mL, 2.66 eq.) at 0° C. and stirred at 20° C. for 2 hours. The reaction mixture was diluted with EtOAc (200 mL) and quenched with saturated NaHCO₃ aqueous solution at 0° C. to adjust pH to neutral, and then extracted with EtOAc (300 mL×3). The combined organic layers were washed with brine (300 mL×2), dried over Na₂SO₄, filtered and concentrated under reduced pressure to give a residue. The residue was purified by column chromatography (SiO₂, Petroleum ether/Ethyl acetate=3/1 to 0/1). Then the crude product was triturated with MeCN (30 mL) to give methyl(R)-(5-(5-(3-ethylpiperidine-1-carbonyl)-1H-pyrrolo[2,3-b]pyridin-1-yl)pyridin-3-yl)carbamate (3.51 g, 8.57 mmol, 60% yield, 99% purity) as a white solid.

LCMS (ESI, M+1): m/z=408.2.

¹H NMR (400 MHz, DMSO-d₆δ8=10.15 (s, 1H), 8.71 (d, J=2.0 Hz, 1H), 8.63 (d, J=2.0 Hz, 1H), 8.53 (s, 1H), 8.35 (d, J=2.0 Hz, 1H), 8.15 (d, J=2.0 Hz, 1H), 8.07 (d, J=3.6 Hz, 1H), 6.85 (d, J=3.6 Hz, 1H), 4.54-4.14 (m, 1H), 3.72 (s, 3H), 3.68-3.45 (m, 1H), 3.15-2.60 (m, 2H), 1.92-1.79 (m, 1H), 1.77-1.54 (m, 1H), 1.42 (ddd, J=3.6, 6.8, 10.0 Hz, 2H), 1.34-1.01 (m, 3H), 1.00-0.73 (m, 3H).

Compound B-70, methyl(S)-(5-(5-(3-ethylpiperidine-1-carbonyl)-1H-pyrrolo[2,3-b]pyridin-1-yl)pyridin-3-yl)carbamate: To a mixture of (S)-(1-(5-aminopyridin-3-yl)-1H-pyrrolo[2,3-b]pyridin-5-yl)(3-ethylpiperidin-1-yl)methanone (5.00 g, 14.3 mmol, 1.00 eq.), pyridine (3.40 g, 42.9 mmol, 3.46 mL, 3.00 eq.) in THF (50 mL) was added methyl carbonochloridate (4.16 g, 44.0 mmol, 3.41 mL, 3.08 eq.) at 0° C. and stirred at 20° C. for 2 hours. The reaction mixture was diluted with EtOAc (200 mL) and quenched with saturated NaHCO₃ aqueous solution at 0° C. to adjust pH to neutral, and then extracted with EtOAc (300 mL×3). The combined organic layers were washed with brine (300 mL×2), dried over Na₂SO₄, filtered and concentrated under reduced pressure to give a residue. The residue was purified by column chromatography (SiO₂, Petroleum ether/Ethyl acetate=3/1 to 0/1). Then the crude product was triturated with MeCN (30 mL) to give methyl(S)-(5-(5-(3-ethylpiperidine-1-carbonyl)-1H-pyrrolo[2,3-b]pyridin-1-yl)pyridin-3-yl)carbamate (3.54 g, 8.66 mmol, 61% yield, 99% purity) as a white solid. LCMS (ESI, M+1): m/z=408.2. ¹H NMR (400 MHz, DMSO-d₆δ8=10.14 (br s, 1H), 8.72 (s, 1H), 8.63 (s, 1H), 8.53 (br s, 1H), 8.35 (s, 1H), 8.15 (s, 1H), 8.06 (br s, 1H), 6.85 (br s, 1H), 4.35 (br s, 1H), 3.72 (s, 3H), 3.60 (br s, 1H), 3.12-2.57 (m, 2H), 1.85 (br d, J=12.4 Hz, 1H), 1.77-1.54 (m, 1H), 1.43 (br s, 2H), 1.34-1.03 (m, 3H), 0.77-0.73 (m, 3H).

### Example 22. Synthesis of (4,4-difluoropiperidin-1-yl)(1-(2-morpholino [1,2,4]triazolo [1,5-a]pyridin-6-yl)-1H-pyrrolo [2,3-b]pyridin-5-yl)methanone (B-97)

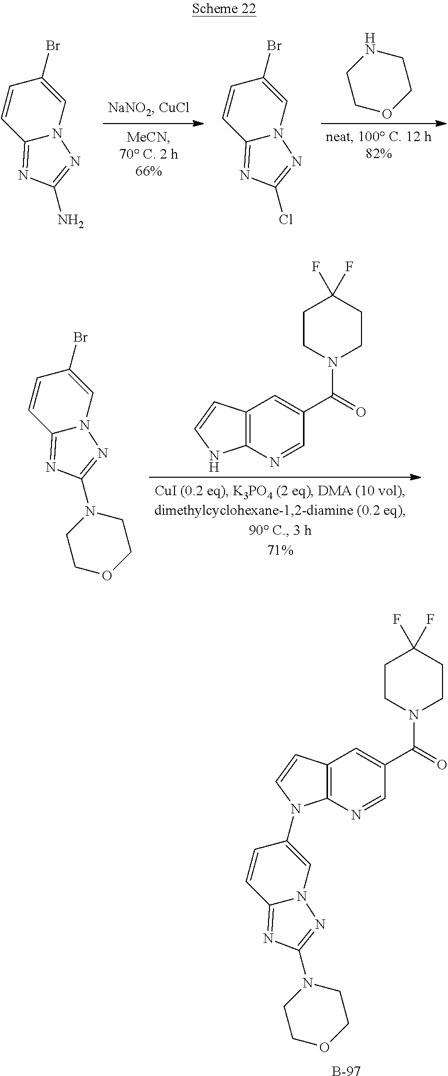

Step-1: To a solution of 6-bromo-[1,2,4]triazolo[1,5-a]pyridin-2-amine (5.00 g, 23.5 mmol, 1.00 eq.) in MeCN (100 mL) was added isopentyl nitrite (8.25 g, 70.4 mmol, 9.48 mL, 3.00 eq.) and CuCl₂ (9.47 g, 70.4 mmol, 3.00 eq). The mixture was stirred at 70° C. for 2 hours. The reaction mixture was filtered and concentrated under reduced pressure to give a residue. The residue was purified by column chromatography (SiO₂, Petroleum ether/Ethyl acetate=10/1 to 3/1). The desired fraction were concentrated to give compound 6-bromo-2-chloro-[1,2,4]triazolo[1,5-a]pyridine (3.6 g, 15.5 mmol, 66% yield) as a white solid. LCMS [ESI, M+1]: 233.8

Step-2: A solution of 6-bromo-2-chloro-[1,2,4]triazolo[1,5-a]pyridine (3.00 g, 12.9 mmol, 1.00 eq.) in morpholine (10.0 mL) was stirred at 100° C. for 12 hours. The reaction mixture was diluted with ethyl acetate (300 mL) and washed with water (300 mL×3). The organic layers were dried over anhydrous sodium sulfate, filtered and concentrated under reduced pressure to give a residue. The residue was purified by column chromatography (SiO₂, Petroleum ether/Ethyl acetate=5/1 to 2/1). The desired fractions were concentrated to give compound 4-(6-bromo-[1,2,4]triazolo[1,5-a]pyridin-2-yl)morpholine (3.0 g, 10.6 mmol, 82% yield) as a white solid. LCMS [ESI, M+1]: 283.0. ¹H NMR (400 MHz, DMSO-d₆δ8=9.06 (dd, J=0.8, 2.0 Hz, 1H), 7.64 (dd, J=2.0, 9.2 Hz, 1H), 7.45 (dd, J=0.8, 9.2 Hz, 1H), 3.73-3.66 (m, 4H), 3.48-3.42 (m, 4H).

Step-3: A mixture of (4,4-difluoro-1-piperidyl)-(1H-pyrrolo [2,3-b]pyridin-5-yl)methanone (2.20 g, 8.29 mmol, 1.00 eq.), 4-(6-bromo-[1,2,4]triazolo[1,5-a]pyridin-2-yl)morpholine (2.58 g, 9.12 mmol, 1.10 eq.), CuI (316 mg, 1.66 mmol, 0.20 eq.), K₃PO₄ (3.52 g, 16.6 mmol, 2.00 eq.) and N1,N2-dimethylcyclohexane-1,2-diamine (1.18 g, 8.29 mmol, 1.00 eq.) in DMAC (40 mL) was degassed and purged with N₂ for 3 times, and then the mixture was stirred at 90° C. for 3 hours under N₂ atmosphere (15 psi). The reaction mixture was diluted with water (200 mL) and extracted with ethyl acetate (150 mL×3). The combined organic layers were washed with brine (400 mL×2), dried over anhydrous sodium sulfate, filtered and concentrated under reduced pressure to give a residue. The residue was purified by column chromatography (SiO₂, Petroleum ether/Ethyl acetate=1/1 to ¼). The desired fraction were concentrated to give compound (4,4-difluoro-1-piperidyl)-[1-(2-morpholino-[1,2,4]triazolo[1,5a]pyridin-6-yl)pyrrolo[2,3-b]pyridin-5-yl]methanone (2.78 g, 5.90 mmol, 71% yield, 99.5% purity) as a white solid. LCMS [ESI, M+1]: 468.1.

¹H NMR (400 MHz, DMSO-d₆δ S=9.33 (s, 1H), 8.44 (s, 1H), 8.24 (s, 1H), 8.08 (d, J=3.6 Hz, 1H), 8.06-8.01 (m, 1H), 7.66 (d, J=9.2 Hz, 1H), 6.84 (d, J=3.6 Hz, 1H), 3.82-3.71 (m, 4H), 3.66 (br d, J=4.0 Hz, 4H), 3.52-3.45 (m, 4H), 2.08 (br s, 4H).

### Example 23. Synthesis of (4,4-difluoropiperidin-1-yl)1 (1-(2-(pyrrolidin-1-ylmethyl)pyridin-4-1H-pyrrolo[2,3-b]pyridin-5-methanone (B-152

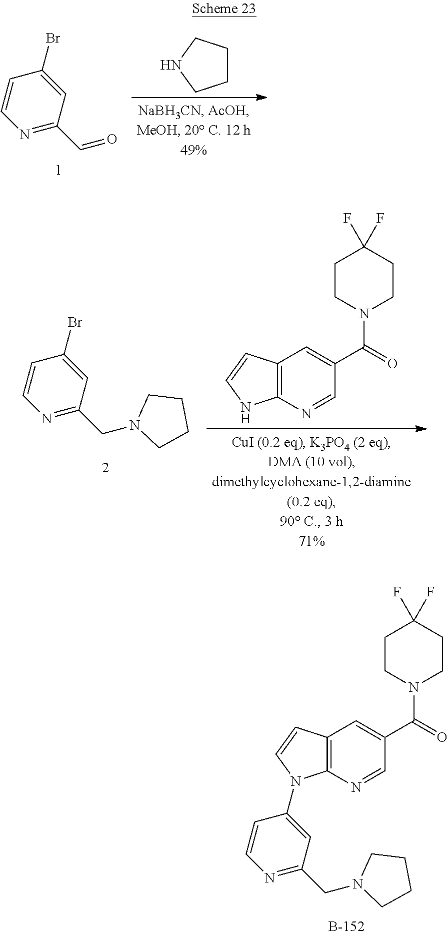 [SMILES: O=Cc1cc(Br)ccn1 ; C1CCNC1 ; Brc1ccnc(CN2CCCC2)c1 ; O=C(c1cnc2[nH]ccc2c1)N1CCC(F)(F)CC1 ; O=C(c1cnc2c(ccn2-c2ccnc(CN3CCCC3)c2)c1)N1CCC(F)(F)CC1]

Step 1: Synthesis of Compound 2: To a solution of 4-bromopicolinaldehyde (1.50 g, 8.06 mmol, 1.00 eq), pyrrolidine (1.15 g, 16.1 mmol, 1.35 mL, 2.0 q) in MeOH (20.0 mL) was added AcOH (242 mg, 4.03 mmol, 231 μL, 0.50 eq), then NaBH₃CN (1.01 g, 16.1 mmol, 2.00 eq). The mixture was stirred at 20° C. for 4 hours. The reaction mixture was quenched with water (10.0 mL) at 0° C. and extracted with EtOAc (20 mL×3). The combined organic layers were washed with brine (20 mL×2), dried over Na₂SO₄, filtered and concentrated under reduced pressure to give a residue. The crude product was purified by reversed-phase HPLC (0.1% NH₃·H₂O) and the mixture was lyophilized to obtain 4-bromo-2-(pyrrolidin-1-ylmethyl)pyridine (980 mg, 3.98 mmol, 49% yield, 98% purity) as a yellow oil. LCMS [ESI, M+1]: 243.1.

Step-2: A mixture of 4-bromo-2-(pyrrolidin-1-ylmethyl)pyridine (200 mg, 829 μmol, 1.00 eq), (4,4-difluoropiperidin-1-yl)(1H-pyrrolo[2,3-b]pyridin-5-yl)methanone (264 mg, 995 μmol, 1.20 eq), K₃PO₄ (352 mg, 1.66 mmol, 2.00 eq), CuI (31.6 mg, 166 mol, 0.20 eq) and dimethylcyclohexane-1,2-diamine (23.6 mg, 166 μmol, 0.20 eq) in DMAC (2 mL) was degassed and purged with N₂ for 3 times, and then the mixture was stirred at 90° C. for 3 hours under N₂ atmosphere (15 psi). The mixture was diluted with H₂O (40 mL) and extracted with EA (30 mL×3), the organic layers were washed with saturated salt solution (30 mL×3), dried over Na₂SO₄, filtered and concentrated under reduced pressure to give a reside and it was purified by column chromatography (SiO₂, Ethyl acetate/Methanol=10/1 to 8/1) to give (4,4-difluoropiperidin-1-yl(1-(2-(pyrrolidin-1-ylmethyl)pyridin-4-yl)-1H-pyrrolo [2,3-b]pyridin-5-yl)methanone (189 mg, 440 μmol, 53% yield, 99.3% purity) as a light yellow solid. LCMS (ESI, M+1): m/z=426.2. ¹H NMR (400 MHz, DMSO-d₆δ8=8.60 (br d, J=5.2 Hz, 1H), 8.49 (d, J=2.0 Hz, 1H), 8.27-8.26 (m, 1H), 8.25-8.24 (m, 1H), 8.20 (s, 1H), 8.02 (br d, J=3.6 Hz, 1H), 6.89 (d, J=4.0 Hz, 1H), 3.80 (s, 2H), 3.78-3.44 (m, 4H), 2.56 (br s, 4H), 2.09 (br d, J=5.2 Hz, 4H), 1.73 (br s, 4H).

### Example 24. Synthesis of (1-(2-(1H-pyrazol-4-yl)pyridin-4-yl)-1H-pyrrolo[2,3-b]pyridin-5-yl)(4,4-difluoropiperidin-1-yl)methanone (B-194)

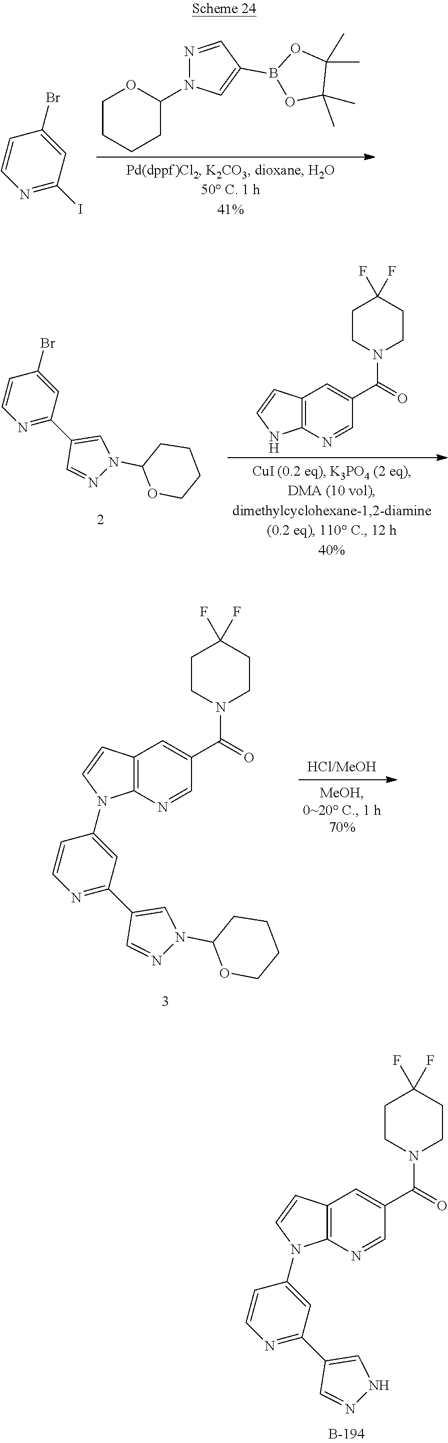

Step-1: Synthesis of Compound 2: To a mixture of-bromo-2-iodo-pyridine (450 mg, 1.59 mmol, 1.00 eq.), 1-tetrahydropyran-2-yl-4-(4,4,5,5-tetramethyl-1,3,2-dioxaborolan-2-yl)pyrazole (485 mg, 1.74 mmol, 1.10 eq.), Pd(dppf)C₁₂ (115 mg, 158 μmol, 0.10 eq.), K₂CO₃ (262 mg, 1.90 mmol, 1.20 eq.) in dioxane (10.0 mL) and H₂O (2.00 mL) was degassed and purged with N₂ for 3 times, and then the mixture was stirred at 50° C. for 1 hour under N₂ atmosphere (15 psi). The reaction mixture was diluted with H₂O (30 mL) and EtOAc (30 mL), then filtered to give a filtrate and extracted with EtOAc (20 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=30/1 to 3/1) to give 4-bromo-2-(1-tetrahydropyran-2-ylpyrazol-4-yl)pyridine (225 mg, 657 μmol, 41% yield, 90% purity) as a white oil. LCMS (ESI, M+3): m/z=310.1.

Step-2: To a mixture of 4-bromo-2-(1-tetrahydropyran-2-ylpyrazol-4-yl)pyridine (225 mg, 730 μmol, 1.20 eq.), (4,4-difluoro-1-piperidyl)-(1H-pyrrolo[2,3-b]pyridin-5-yl)methanone (161 mg, 608 μmol, 1.00 eq.), CuI (23.1 mg, 121 μmol, 0.20 eq.), K₃PO₄ (258 mg, 1.22 mmol, 2.00 eq.) and (1 R,2R)—N1,N2-dimethylcyclohexane-1,2-diamine (17.3 mg, 121 μmol, 0.20 eq) in DMAC (5.00 mL) was degassed and purged with N₂ for 3 times, and then the mixture was stirred at 110° C. for 12 hours under N₂ atmosphere. The reaction mixture was diluted with H₂O (30 mL) and EtOAc (30 mL), then filtered to give a filtrate and extracted with EtOAc (20 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=3/1 to 0/1) to give (4,4-difluoro-1-piperidyl)-[1-[2-(1-tetrahydropyran-2-ylpyrazol-4-yl)-4-pyridyl]pyrrolo[2,3-b]pyridin-5-yl]methanone (120 mg, 241 μmol, 40% yield, 99% purity) as a yellow oil. LCMS (ESI, M+1): m/z=493.3.

Step-3: To a solution of (4,4-difluoro-1-piperidyl)-[1-[2-(1-tetrahydropyran-2-ylpyrazol-4-yl)-4-pyridyl]pyrrolo[2,3-b]pyridin-5-yl]methanone (120 mg, 243 μmol, 1.00 eq.) in MeOH (0.50 mL) was added HCl/MeOH (4.00 M, 2.00 mL,) at 0° C. The mixture was stirred at 20° C. for 1 hour. The reaction mixture was concentrated under reduced pressure to give a residue and purified by prep-HPLC (column: Phenomenex Synergi C18 150×25 mm×10 μm 10 um; mobile phase: [water (FA)-ACN]; B %: 11%-41%, 10 min) to give (4,4-difluoro-1-piperidyl)[1-[2-(1H-pyrazol-4-yl)-4-pyridyl]pyrrolo[2,3-b]pyridin-5-yl]methanone (70.0 mg, 169 μmol, 70% yield, 99% purity) as a white solid. LCMS (ESI, M+1): m/z=409.3. ¹H NMR (400 MHz, DMSO-d₆δ8=13.50-12.74 (m, 1H), 8.63 (d, J=5.6 Hz, 1H), 8.52 (d, J=2.0 Hz, 1H), 8.37 (d, J=3.6 Hz, 1H), 8.29-8.26 (m, 4H), 8.15 (dd, J=2.0, 5.6 Hz, 1H), 6.93 (d, J=3.6 Hz, 1H), 3.90-3.48 (m, 4H), 2.09 (br s, 4H).

### Example 25. Synthesis of (1-(4-(5-amino-4H-1,2,4-triazol-3-yl)phenyl)-1H-pyrrolo[2,3-b]pyridin-5-yl)(4,4-difluoropiperidin-1-yl)methanone (B-78)

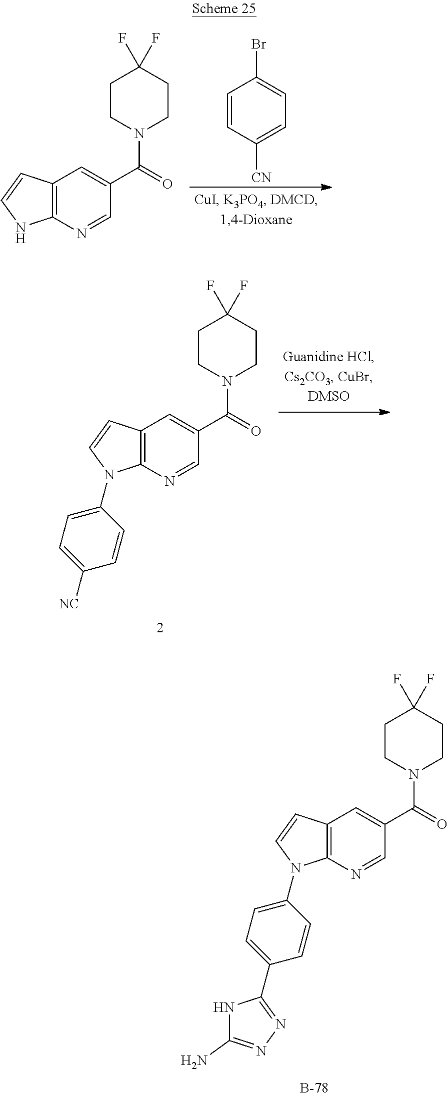

To a stirred solution of (4,4-difluoropiperidin-1-yl)(1H-pyrrolo[2,3-b]pyridin-5-yl)methanone (2 g, 7.57 mmol, 1.0 eq.) and 4-bromobenzonitrile (2.1 g, 11.36 mmol, 1.5 eq.) in 1,4-dioxane (10 mL) were added copper iodide (0.28 g, 1.5 mmol, 0.2 eq.), DMCD (0.2 g, 1.51 mmol, 0.2 eq.) and potassium phosphate (3.9 g, 18.7 mmol, 2.5 eq.) at room temperature in presence of argon gas. The reaction mixture was stirred at 80° C. for 16 hr under atmosphere of argon. After completion of reaction, the mixture was poured in water and extracted with ethyl acetate. Organic layer was washed with saturated brine and dried over anhydrous sodium sulfate. The solvent was evaporated in vacuo to get crude compound 4-(5-(4,4-difluoropiperidine-1-carbonyl)-1H-pyrrolo[2,3-b]pyridin-1-yl)benzonitrile 2. It was then purified by column chromatography (100-200 silica mesh) in 20% ethyl acetate-hexane to get the desired compound 2, (1.3 g, 47.3%) as white solid. ESI-MS (adz) C₈ lculated for C₂₀H₁₆F₂N₄O: 366.37. Found 367.2 (M+H)³⁰.

To a stirred solution of 2 (200 mg, 0.54 mmol, 1.0 eq.) in dimethyl sulfoxide were added guanidine hydrochloride (62.2 mg, 0.65 mmol, 1.2 eq.), cesium carbonate (266 mg, 0.65 mmol, 1.2 eq.) and copper bromide (3.9 mg, 0.027 mmol, 0.05 eq.) at room temperature. The reaction mixture was stirred at 120° C. for 16 hours. After completion of reaction, it was quenched with water. It was then extracted with ethyl acetate. Organic layer was washed with saturated brine and dried over anhydrous sodium sulfate. The solvent was evaporated in vacuo to obtain crude product, which was then purified by column chromatography (100-200 silica mesh) in 60% ethyl acetate -hexane to get the desired compound (1-(4-(5-amino-4H-1,2,4-triazol-3-yl)phenyl)-1H-pyrrolo[2,3-b]pyridin-5-yl)(4,4-difluoropiperidin-1-yl)methanone (12 mg, 2.8%) as white solid. ESI-MS (adz) C₈ lculated for C₂₁H₁₉F₂N₇O: 423.43 Found 424.1 (M+H)³⁰; HPLC Purity: 97.23%; ¹H NMR (300 MHz, DMSO-d₆): δ/ppm 12.11 (s, 1H), 8.44-8.43 (d, J=1.8 Hz, 1H), 8.23-8.22 (d, J=3.6 Hz, 1H), 8.11-8.10 (d, J 3.6 Hz, 1H), 8.06-7.95 (m, 4H), 6.84-6.82 (d, J=3.9 Hz, 1H), 6.13 (s, 2H), 3.65 (s, 4H), 2.08 (s, 4H).

### Example 26. Synthesis of (4,4-difluoropiperidin-1-yl) (1-(4-(5-morpholino-4H-1,2,4-triazol-3-yl)phenyl)-1H-pyrrolo [2,3-b]pyridine-5-yl) methanone (B-67)

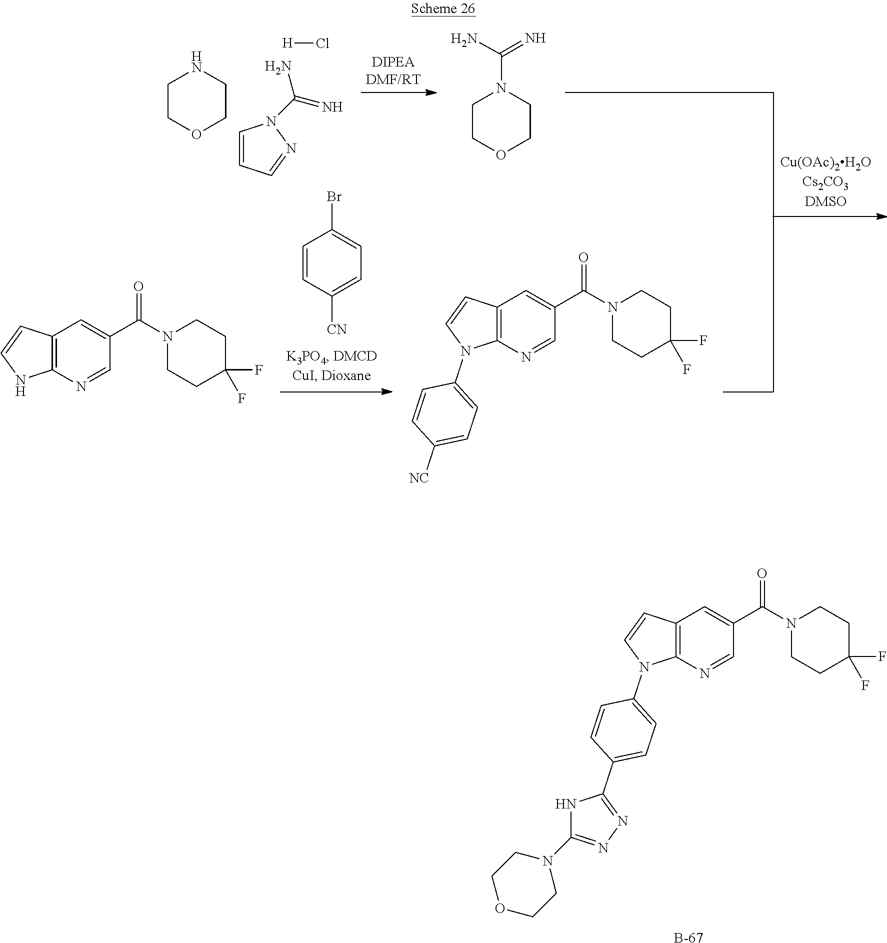

Morpholine (0.875 g, 10 mmol, 1 eq.) was added to stirred solution of 1H-pyrazole-1-carboxamidine hydrochloride (1.46 g, 10 mmol; 1 eq.) and DIPEA (1.7 mL; 11 mmol; 1.1 eq.) in DMF (5 mL). Stir the reaction mixture at ambient temperature for 3 h. The precipitated obtained were filtered, washed Et₂O (10 mL×2) and dried on open air to afford morpholine-1-carboximidamide (0.7 g, 54%, white solid). ESI-MS (m/z) C₈ lculated for C₅H₁₁N30: 129.16; Found 130.1. [M+H]NMR (300 MHz, DMSO-d₆): δ(ppm 7.64 (s, 1H), 3.64-3.63 (m, 1H), 3.42-3.36 (m, 1H).

To a stirred solution of 4-bromobenzonitrile (0.76 g, 4.15 mmol, 1.1 eq.) and (4,4-difluoropiperidin-1-yl) 1H-pyrrolo[2,3-b]pyridin-5-yl)methanone (1.0 g, 3.7 mmol, 1.0 eq.) in 1,4-dioxane (25 mL) were added copper iodide (145 mg, 0.75 mmol, 0.2 eq.), DMCD (108 mg, 0.75 mmol, 0.2 eq.) and potassium phosphate (1.6 g, 7.54 mmol, 2 eq.) at room temperature in presence of Argon gas. The reaction mixture was stirred at 110° C. for 16 hours under atmosphere of Argon. After completion of reaction, the mixture was poured in water and extracted with ethyl acetate. Organic layer was washed with saturated brine and dried over anhydrous sodium sulfate. The solvent was evaporated in vacuo to get crude intermediate, which was then purified by column chromatography (100-200 mesh) in 5% ethyl acetate -hexane to get the desired compound 4-(5-(4,4-difluoropiperidine-1-carbonyl)-1H-pyrrolo[2,3-b]pyridin-1-yl)benzonitrile (915 mg, 60% yield, White solid). ESI-MS (m/z) C₈ lculated for C₂₀H₁₆F₂N₄₀: 366.37; Found 367.0. [M+H]⁺. NMR (300 MHz, DMSO-d₆): δ(ppm 8.47 (s, 1H), 8.27 (m, 2H), 8.24 (m, 2H), 8.06-8.03 (m, 2H), 6.90-6.89 (m, 1H), 3.64 (b s, 4H), 2.12-2.03 (m, 4H).

Morpholine-1-carboximidamide (250 mg, 1.93 mmol, 1.1 eq.), was added to a stirred solution of 4-(5-(4,4-difluoropiperidine-1-carbonyl)-1H-pyrrolo[2,3-b]pyridin-1-yl)benzonitrile (644 mg, 1.76 mmol, 1 eq.), Cesium carbonate (2,3 g, 7.0 mmol, 3.6 eq.) and Copper (I)bromide (250 mg, 1.76 mmol, 1 eq.) in DMSO (10 mL) and refluxed in air for 24 h. After completion of reactions, the reaction mixture was cooled to room temperature, and the content were poured over crushed ice (100 g). The solid obtained were filtered, washed well with cold water and dried in air. The crude thus obtained was purified by column chromatography on a silica gel (100-200 mesh) using ethyl acetate:hexane (12:88) as the eluent to afford desired compound (4,4-difluoropiperidin-1-yl) (1-(4-(5-morpholino-4H-1,2,4-triazol-3-yl)phenyl-1H-pyrrolo[2,3-b]pyridine-5-yl) methanone. The compound obtained was purified by Prep-HPLC. (Yield: 45.97 mg, 6%) as white solid. ESI-MS (adz) C₈ lculated for C₂₅H₂₅F₂N₇O₂: 493.52. Found 492.1 [M−H]. HPLC Purity: 99.63%; ¹H NMR (300 MHz, DMSO-d₆): δ/ppm 12.81 (s, 1H), 8.44 (s, 1H), 8.23 (s, 1H), 8.15-8.14 (m, 1H), 8.09-8.07 (m, 3H,), 7.98-7.95 (m, 2H), 6.84-6.82 (m, 1H), 3.73-3.71 (m, 8H), 3.39 (m, 4H), 2.07-2.03 (m, 4H).

### Example 27. Synthesis of (1-(3-(4H-1,2,4-triazol-3-yl)phenyl)-1H-pyrrolo[2,3-b]pyridin-5-yl)(4,4-difluoropiperidin-1-yl)methanone (B-36)

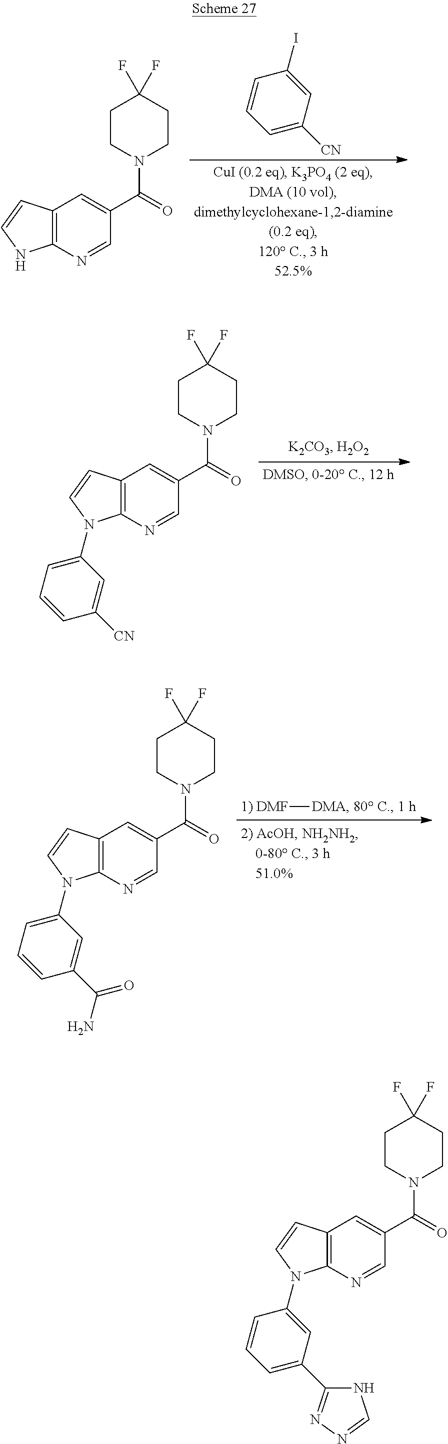

To a solution of (4,4-difluoropiperidin-1-yl)(1H-pyrrolo[2,3-b]pyridin-5-yl)methanone (282 mg, 1.06 mmol, 1.00 eq.), 3-iodobenzonitrile (292 mg, 1.28 mmol, 1.20 eq.) in DMA (3 mL) was added Cu (40.5 mg, 213 μmol, 0.20 eq.), dimethylcyclohexane-1,2-diamine (30.2 mg, 213 μmol, 0.20 eq.) and K₃PO₄ (451 mg, 2.13 mmol, 2.00 eq). The mixture was stirred at 120° C. for 3 hr. The mixture was filtered and concentrated under reduced pressure to give a residue. The crude product was purified by reversed-phase HPLC (0.1% FA condition) to give 3-(5-(4,4-difluoropiperidine-1-carbonyl)-1H-pyrrolo[2,3-b]pyridin-1-yl)benzonitrile (220 mg, 558 μmol, 52.5% yield, 93.5% purity) as a yellow solid. LCMS (ESI, M+1): m/z=367.2.

To a solution of 3-(5-(4,4-difluoropiperidine-1-carbonyl)-1H-pyrrolo[2,3-b]pyridin-1-yl)benzonitrile (200 mg, 546 μmol, 1.00 eq.) in DMSO (2 mL) was added K₂CO₃ (113 mg, 819 μcool, 1.50 eq.) and H₂O₂ (9.63 g, 85.0 mmol, 8.16 mL, 30% purity, 156 eq). The mixture was stirred at 0° C. for 2 hrs. The reaction mixture was diluted with saturated Na₂SO³ and extracted with EA (5 mL×3). The combined organic layers were washed with brine (20 mL×1), dried over Na₂SO₄, filtered and concentrated under reduced pressure to give 3-(5-(4,4-difluoropiperidine-1-carbonyl)-1H-pyrrolo[2,3-b]pyridin-1-yl)benzamide (200 mg, crude) as a yellow solid. LCMS (ESI, M+1): m/z=385.1.

A solution of 3-(5-(4,4-difluoropiperidine-1-carbonyl)-1H-pyrrolo[2,3-b]pyridin-1-yl)benzamide (200 mg, 520 μmol, 1.00 eq.) was stirred in DMF-DMA (1.79 g, 15.1 mmol, 2 mL, 28.9 eq.) was stirred at 80° C. for 1.5 hours. The volatiles were removed, and AcOH was added (4.20 g, 69.9 mmol, 4 mL, 134 eq.) to the reaction mixture, then NH₂NH₂·H₂O (0.65 g, 13.0 mmol, 631 μL, 25.0 eq.) was added dropwise. The resulting mixture was stirred at 80° C. for 1 hour. The reaction mixture was diluted with saturated Na₂SO³ and extracted with EA (50 mL×3). The combined organic layers were washed with brine (50 mL×1), dried over Na₂SO₄, filtered and concentrated under reduced pressure to give a residue. The crude product was purified by reversed-phase HPLC (0.1% FA condition) to give (1-(3-(4H-1,2,4-triazol-3-yl)phenyl)-1H-pyrrolo[2,3-b]pyridin-5-yl)(4,4-difluoropiperidin-1-yl)methanone (104 mg, 250 μmol, 50.9% yield, 98% purity) as a white solid. LCMS (ESI, M+1): m/z=409.2. ¹H NMR (400 MHz, DMSO-d₆δ8=14.39-14.13 (m, 1H), 8.73-8.48 (m, 2H), 8.45 (d, J=2.0 Hz, 1H), 8.24 (d, J=2.0 Hz, 1H), 8.12 (d, J=3.6 Hz, 1H), 8.03 (d, J=7.6 Hz, 1H), 7.96-7.87 (m, 1H), 7.68 (br t, J=8.0 Hz, 1H), 6.85 (d, J=3.6 Hz, 1H), 3.79-3.48 (m, 4H), 2.15-2.01 (m, 4H)

### Representative Procedure for Synthesis of Sulfides

### Example 28. Synthesis of 5-(cyclopropylmethylsulfinyl)-1-[4 (4H-1,2,4-triazol-3-yl)phenyl]pyrrolo2,3-b1 pyridine (B-259)

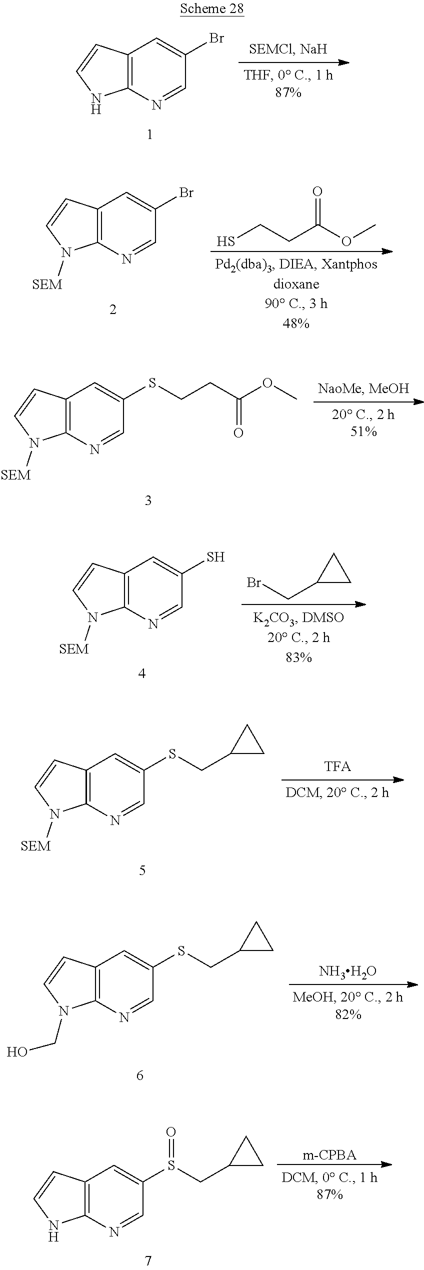

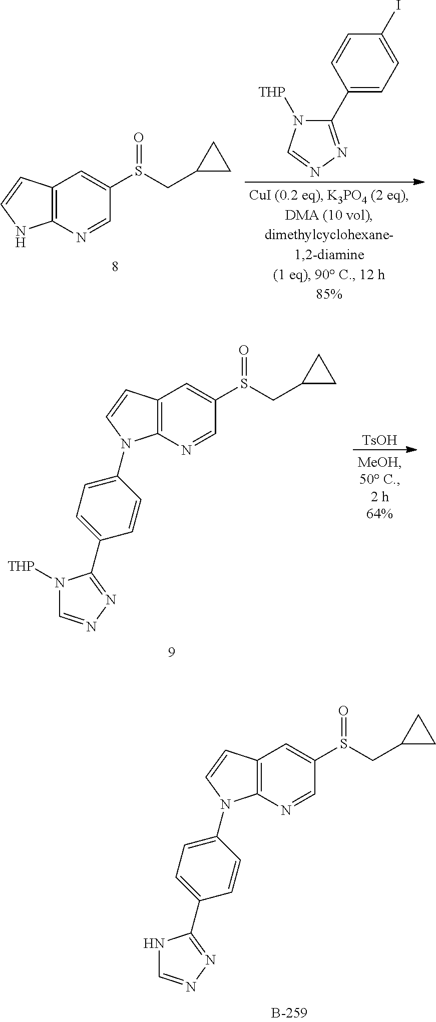

Step-1: Synthesis of Compound 2: To a solution of 5-bromo-1H-pyrrolo[2,3-b]pyridine (6.00 g, 30.4 mmol, 1.00 eq.) in THE (100 mL) was added NaH (2.44 g, 60.9 mmol, 60% purity, 2.00 eq.) and SEM-C₁ (10.1 g, 60.9 mmol, 10.7 mL, 2.00 eq). The mixture was stirred at 0° C. for 1 hr. The mixture was poured into NH₄Cl (100 mL) and extracted with EA (200 ml×3). The combined organic layer was washed with brine (100 mL), dried over Na₂SO₄, then the mixture was filtered and the filtrate was concentrated. The residue was purified by column chromatography (SiO₂, PE/EA=50/1 to 10/1) to give compound 2-[(5-bromopyrrolo[2,3-b]pyridin-1-yl)methoxy]ethyl-trimethyl-silane (8.80 g, 26.6 mmol, 87% yield, 99% purity) as an off-white oil.

LCMS (ESI, M+3): m/z=329.1.

Step-2: Synthesis of Compound 3: A mixture of 2-[(5-bromopyrrolo[2,3-b]pyridin-1-yl)methoxy]ethyl-trimethyl-silane (8.80 g, 26.8 mmol, 1.00 eq.), methyl 3-sulfanylpropanoate (4.85 g, 40.3 mmol, 4.37 mL, 1.50 eq.), Pd₂(dba)₃ (1.55 g, 2.69 mmol, 0.10 eq.), Xantphos (3.11 g, 5.38 mmol, 0.20 eq.) and DIEA (6.95 g, 53.7 mmol, 9.37 mL, 2.00 eq.) in dioxane (140 mL) was degassed and purged with N₂ for 3 times, and then the mixture was stirred at 90° C. for 3 hours under N₂ atmosphere (15 psi). The reaction was diluted with EA (200 mL) and quenched with H₂O (100 mL). The mixture was extracted with EtOAc (3×200 mL). The combined organic extracts were washed with saturated H₂O (3×100 mL) and brine (200 mL) and then dried over Na₂SO₄. The residue was purified by column chromatography (SiO₂, Petroleum ether/Ethyl acetate=30/1 to 3/1) to give compound methyl 3-[1-(2-trimethylsilylethoxymethyl)pyrrolo[2,3-b]pyridin-5-yl]sulfanylpropanoate (4.80 g, 12.9 mmol, 48% yield, 99% purity) as an off-white oil.

LCMS (ESI, M+1): m/z=367.2.

Step-3: Synthesis of Compound 4: To a solution of methyl 3-[1-(2-trimethylsilylethoxymethyl)pyrrolo[2,3-b]pyridin-5-yl]sulfanylpropanoate (4.75 g, 12.9 mmol, 1.00 eq.) in MeOH (50.0 mL) was added NaOMe (2.10 g, 38.8 mmol, 3.00 eq). The mixture was stirred at 20° C. for 2 hours. The mixture was poured into NaHCO₃ (60 mL) and extracted with EA (100 ml×3). The combined organic layer was washed with brine (100 mL), dried over Na₂SO₄, then the mixture was filtered and the filtrate was concentrated. The residue was purified by column chromatography (SiO₂, PE/EA=50/1 to 5/1) to give compound 1-(2-trimethylsilylethoxymethyl)pyrrolo[2,3-b]pyridine-5-thiol (2.00 g, 6.63 mmol, 51% yield, 93% purity) as a white oil.

LCMS (ESI, M+1): m/z=281.1.

¹H NMR (400 MHz, DMSO-d₆δ8=8.21 (d, J=2.4 Hz, 1H), 7.97 (d, J=2.0 Hz, 1H), 7.63 (d, J=3.6 Hz, 1H), 6.46 (d, J=3.2 Hz, 1H), 5.58 (s, 2H), 5.31 (s, 1H), 3.51-3.46 (m, 2H), 0.82-0.77 (m, 2H),-0.11--0.13 (m, 9H).

Step-4: Synthesis of Compound 5: To a solution of 1-(2-trimethylsilylethoxymethyl)pyrrolo[2,3-b]pyridine-5-thiol (500 mg, 1.78 mmol, 1.00 eq.) and bromomethylcyclopropane (481 mg, 3.57 mmol, 341 μL, 2.00 eq.) in DMSO (10.0 mL) was added K₂CO₃ (492 mg, 3.57 mmol, 2.00 eq). The mixture was stirred at 20° C. for 2 hours. The mixture was poured into H₂O (40 mL) and extracted with EA (50 mL×3). The combined organic layer was washed with brine (50 mL), dried over Na₂SO₄, then the mixture was filtered and the filtrate was concentrated. The residue was purified by column chromatography (SiO₂, PE/EA=50/1 to 5/1) to give 2-[[5-(cyclopropylmethylsulfanyl)pyrrolo[2,3-b]pyridin-1-yl]methoxy]ethyl-trimethyl-silane (500 mg, 1.48 mmol, 83% yield, 99% purity) as a yellow oil.

LCMS (ESI, M+1): m/z=335.2.

¹H NMR (400 MHz, DMSO-d₆δ8=8.33 (d, J=2.0 Hz, 1H), 8.12 (d, J=2.4 Hz, 1H), 7.66 (d, J=3.6 Hz, 1H), 6.51 (dd, J=1.6, 3.6 Hz, 1H), 5.61 (s, 2H), 3.49 (t, J=7.6 Hz, 2H), 2.84 (d, J=6.8 Hz, 2H), 0.98-0.88 (m, 1H), 0.80 (t, J=8.4 Hz, 2H), 0.50-0.39 (m, 2H), 0.13 (br d, J=4.8 Hz, 2H),-0.12 (d, J=1.6 Hz, 9H).

Step-5: Synthesis of Compound 6: To a solution of 2-[[5-(cyclopropylmethylsulfanyl)pyrrolo[2,3-b]pyridin-1-yl]methoxy]ethyl-trimethyl-silane (500 mg, 1.49 mmol, 1.00 eq.) in DCM (1.00 mL) was added TFA (1.28 g, 11.2 mmol, 833 μL, 7.53 eq). The mixture was stirred at 0° C. for 1 hour. The reaction mixture was concentrated under reduced pressure to give [5-(cyclopropylmethylsulfanyl)pyrrolo[2,3-b]pyridin-1-yl]methanol (350 mg, crude) as a yellow oil.

LCMS (ESI, M+1): m/z=235.0.

Step-6: Synthesis of Compound 7: To a solution of [5-(cyclopropylmethylsulfanyl)pyrrolo[2,3-b]pyridin-1-yl]methanol (350 mg, crude) in MeOH (3.00 mL) was added NH₃·H₂O (3.98 g, 28.4 mmol, 4.37 mL, 25% purity, 19.0 eq). The mixture was stirred at 20° C. for 2 hours. After being cooled to room temperature, the mixture was concentrated. The residue was diluted with EtOAc (10 mL) and water (10 mL). The layers were separated and the aqueous phase was extracted with EtOAc (3×10 mL). The organic layer was dried over Na₂SO₄, filtered and concentrated. The residue was purified by column chromatography (SiO₂, PE/EA=20/1 to 2/1) to give 5-(cyclopropylmethylsulfanyl)-1H-pyrrolo[2,3-b]pyridine (280 mg, 1.23 mmol, 82% yield, 90% purity) as a white solid.

$$LCMS ( ESI , M + 1 ) : m / z = 205. .$$

Step-7: Synthesis of Compound 8: To a solution of 5-(cyclopropylmethylsulfanyl)-1H-pyrrolo[2,3-b]pyridine (280 mg, 1.37 mmol, 1.00 eq.) in DCM (10.0 mL) was added m-CPBA (278 mg, 1.37 mmol, 85% purity, 1.00 eq). The mixture was stirred at 0° C. for 1 hour. The reaction mixture was diluted with DCM (30 mL) and saturated Na₂SO³ (10 mL) and extracted with DCM (20 mL×3). The combined organic layers were washed with brine (10 mL×1), dried over Na₂SO₄, filtered and concentrated under reduced pressure to give a residue. The residue was purified by column chromatography (SiO₂, PE/EA=10/1 to 1/1) to give 5-(cyclopropylmethylsulfinyl)-1H-pyrrolo[2,3-b]pyridine (280 mg, 1.19 mmol, 87% yield, 94% purity) as a white solid.

$$LCMS ( ESI , M + 1 ) : m / z = 221. .$$

Step-8: Synthesis of Compound 9: A mixture of 5-(cyclopropylmethylsulfinyl)-1H-pyrrolo[2,3-b]pyridine (140 mg, 635 μmol, 1.00 eq.), 3-(4-iodophenyl)-4-tetrahydropyran-2-yl-1,2,4-triazole (270 mg, 762 μmol, 1.20 eq.), CuI (24.2 mg, 127 μmol, 0.20 eq.), K₃PO₄ (134 mg, 635 μmol, 1.00 eq.) and (1R,2R) N₁,N₁-dimethylcyclohexane-1,2-diamine (90.4 mg, 635 μmol, 1.00 eq.) in DMAC (5.00 mL) was degassed and purged with nitrogen for 3 times and then the mixture was stirred at 90° C. for 12 hours (15 psi). The mixture was poured into H₂O (20 mL) and extracted with EA (40 mL×3). The combined organic layer was washed with brine (30 mL), dried over Na₂SO₄, then the mixture was filtered and the filtrate was concentrated. The residue was purified by column chromatography (SiO₂, PE/EA=10/1 to 0/1) to give 5-(cyclopropylmethylsulfinyl)-1-[4-(4-tetrahydropyran-2-yl -1,2,4-triazol-3-yl)phenyl]pyrrolo[2,3-b]pyridine (260 mg, 540 μmol, 85% yield, 93% purity) as a white solid.

$$LCMS ( ESI , M + 1 ) : m / z = 448.2 .$$

Step 9: Synthesis of 5-(cyclopropylmethylsulfinyl)-1-[4-(4H-1,2,4-triazol-3-yl)phenyl]pyrrolo[2,3-b]pyridine (B-259): To a solution of 5-(cyclopropylmethylsulfinyl)-1-[4-(4-tetrahydropyran-2-yl-1,2,4-triazol-3-yl)phenyl]pyrrolo[2,3-b]pyridine (210 mg, 469 μmol, 1.00 eq.) in MeOH (21.0 mL) was added TsOH (121 mg, 703 μmol, 1.50 eq). The mixture was stirred at 50° C. for 2 hours. After being cooled to room temperature, the mixture was concentrated. The residue was diluted with EtOAc (10 mL) and water (10 mL). The layers were separated and the aqueous phase was extracted with EtOAc (3×10 mL). The organic layer was dried over Na₂SO₄, filtered and concentrated. The residue was purified by prep-HPLC (column: Phenomenex luna C18 150×40 mm×15 um; mobile phase: [water (FA)-ACN]; B %: 24%-54%, 10 min) to give 5-(cyclopropylmethylsulfinyl)-1-[4-(4H-1,2,4-triazol-3-yl)phenyl]pyrrolo[2,3-b]pyridine (110 mg, 299 μmol, 64% yield, 99% purity) as a white gum.

$$LCMS ( ESI , M + 1 ) : m / z = 364. .$$

¹H NMR (400 MHz, DMSO-d₆δ8=14.71-13.65 (m, 1H), 8.59 (d, J=2.0 Hz, 2H), 8.45 (d, J=2.0 Hz, 1H), 8.23-8.16 (m, 3H), 8.07 (br s, 2H), 6.91 (d, J=3.6 Hz, 1H), 3.04-2.89 (m, 2H), 0.97-0.87 (m, 1H), 0.54 (br dd, J=1.6, 8.0 Hz, 2H), 0.29 (br t, J=6.0 Hz, 2H).

Additional representative compounds of the disclosure where made by the routes and schemes of Examples 1-28.

Analytical data for compounds described herein can be seen in Table 3.

TABLE 3
Analytical data.

| | | Mass Spec. | |
|---|---|---|---|
| Compound | Yield/ | Calculated/Mass | |
| No. | Purity | Spec. Found (m/z) | ¹H NMR (DMSO-d₆, 400 MHZ) |
| B-1 | 8.19%/ | 449.4 for | δ 13.92 (s, .1H), 9.11-9.09 (m, 2H), 8.83 (t, J = 2 |
| | 96.91% | C23H21F2N7O/ | Hz, 1H), 8.47 (d, J = 2 Hz, 1H), 8.26-8.22 (m, 2H), |
| | | 450.1 | 6.88 (d, J = 4 Hz, 1H), 3.65 (brs, 4H), 2.11-2.04 |
| | | (M + 1) | (m, 6H) |
| B-2 | 33%/ | 426.16 for | δ 8.74-8.64 (m, 2H), 8.49 (d, J = 1.9 Hz, 1H), 8.44- |
| | 97.98% | C21H20F2N6O2/ | 8.34 (m, 1H), 8.28-8.22 (m, 2H), 7.20-7.14 (m, |
| | | 427.2 | 1H), 6.84-6.81 (m, 1H), 3.98-3.92 (m, 2H), 3.81- |
| | | (M + 1) | 3.56 (m, 4H), 3.51-3.44 (m, 2H), 2.16-2.01 (m, |
| | | | 4H). |
| B-3 | 45.2%/ | 363.16 for | δ 13.10-12.92 (m, 1H), 8.38 (d, J = 1.6 Hz, 1H), |
| | 97.3% | C21H21N3O3/ | 8.20-8.07 (m, 6H), 6.86 (d, J = 3.8 Hz, 1H), 4.46- |
| | | 364.1 | 4.19 (m, 1H), 3.74-3.48 (m, 1H), 3.12-0 (m, 2H), |
| | | (M + 1) | 1.84-1.75 (m, 1H), 1.72-1.55 (m, 2H), 1.53-1.41 |
| | | | (m, 1H), 1.25-1.11 (m, 1H), 0.99-0.67 (m, 3H). |
| B-4 | 61.3%/ | 363.16 for | δ 13.18-12.81 (m, 1H), 8.38 (d, J = 2.0 Hz, 1H), |
| | 97.4% | C21H21N3O3/ | 8.19-8.09 (m, 6H), 6.86 (d, J = 3.8 Hz, 1H), 4.42- |
| | | 364.2 | 4.20 (m, 1H), 3.70-3.51 (m, 1H), 3.09-2.69 (m, |
| | | (M + 1) | 2H), 1.84-1.76 (m, 1H), 1.70-1.40 (m, 3H), 1.36- |
| | | | 1.01 (m, 2H), 0.98-0.64 (m, 3H). |
| B-5 | 78%/ | 349.14 for | δ 13.14-12.94 (m, 1H), 8.55-8.52 (m, 1H), 8.32- |
| | 99.6% | C20H19N3O3/ | 8.28 (m, 1H), 8.17-8.10 (m, 5H), 6.86 (br s, 1H), |
| | | 350.2 | 3.73-3.50 (m, 3H), 3.19-3.01 (m, 1H), 2.35-2.17 |
| | | (M + 1) | (m, 1H), 2.08-1.93 (m, 1H), 1.59-1.43 (m, 1H), |
| | | | 1.10-0.96 (m, 3H). |
| B-6 | 80%/ | 353.12 for | δ 13.14-12.88 (m, 1H), 8.56 (br d, J = 9.5 Hz, 1H), |
| | 99.9% | C19H16FN3O3/ | 8.34 (br d, J = 15.4 Hz, 1H), 8.19-8.09 (m, 5H), |
| | | 354.1 | 6.87 (d, J = 3.7 Hz, 1H), 5.49-5.23 (m, 1H), 4.00- |
| | | (M + 1) | 3.58 (m, 4H), 2.25-2.02 (m, 2H). |
| B-7 | 84.5%/ | 349.14 for | δ 13.26-12 (m, 1H), 8.53 (s, 1H), 8.30 (br d, J = 2.9 |
| | 99.1% | C20H19N3O3/ | Hz, 1H), 8.20-8.08 (m, 5H), 6.89-6.83 (m, 1H), |
| | | 350.2 | 3.73-3.50 (m, 3H), 3.19-3.03 (m, 1H), 2.34-2.16 |
| | | (M + 1) | (m, 1H), 2.08-1.92 (m, 1H), 1.58-1.44 (m, 1H), |
| | | | 1.10-0.94 (m, 3H). |
| B-8 | 88.5%/ | 353.12 for | δ 13.16-12.92 (m, 1H), 8.57 (br s, 1H), 8.36 (br s, |
| | 99.73% | C19H16FN3O3/ | 1H), 8.21-8.08 (m, 5H), 6.87 (d, J = 3.7 Hz, 1H), |
| | | 354.1 | 5.50-5.23 (m, 1H), 3.99-3.59 (m, 4H), 2.25-2.01 |
| | | (M + 1) | (m, 2H). |
| B-9 | 23.23%/ | 344.16 for | δ 8.39 (d, J = 2.0 Hz, 1H), 8.30 (d, J = 8.9 Hz, 2H), |
| | 99.13% | C21H20N4O/345.2 | 8.22-8.20 (m, 1H), 8.16-8.14 (m, 1H), 8.05 (d, J = |
| | | (M + 1) | 8.9 Hz, 2H), 6.89 (d, J = 3.9 Hz, 1H), 4.40-4.24 (m, |
| | | | 1H), 3.74-3.40 (m, 1H), 3.11-2.87 (m, 1H), 1.84- |
| | | | 1.39 (m, 5H), 1.25-1.15 (m, 1H), 1.01-0.63 (m, |
| | | | 3H). |
| B-10 | 35.5%/ | 344.16 for | δ 8.39 (d, J = 2.0 Hz, 1H), 8.33-8.26 (m, 2H), 8.23- |
| | 99.97% | C21H20N4O/ | 8.19 (m, 1H), 8.17-8.14 (m, 1H), 8.07-8.02 (m, |
| | | 345.2 | 2H), 6.88 (d, J = 3.9 Hz, 1H), 4.43-4.22 (m, 1H), |
| | | (M + 1) | 3.73-3.45 (m, 1H), 3.15-2.68 (m, 2H), 1.84-1.41 |
| | | | (m, 4H), 1.24-1.13 (m, 1H), 0.97-0.66 (m, 3H). |
| B-11 | 2.7%/ | 425.14 for | δ 9.28-9.25 (m, 1H), 9.02-8.99 (m, 1H), 8.92- |
| | 85.32% | C20H17F2N7O2/ | 8.88 (m, 1H), 8.49-8.45 (m, 1H), 8.29-8.24 (m, |
| | | 426.1 | 2H), 8.17-8.13 (m, 2H), 6.92-6.89 (m, 1H), 3.79- |
| | | (M + 1) | 3.59 (m, 4H), 2.13-2.06 (m, 4H). |
| B-12 | 26.70%/ | 397.15 for | δ 9.18 (s, 1H), 8.42-8.40 (m, 1H), 8.24-8.21 (m, |
| | 98.76% | C19H17F2N7O/ | 1H), 8.05-8.03 (m, 1H), 7.96-7.89 (m, 1H), 7.57- |
| | | 398.1 | 7.51 (m, 1H), 6.84-6.82 (m, 1H), 6.16-6.08 (m, |
| | | (M + 1) | 2H), 3.81-3.51 (m, 4H), 2.19-2.01 (m, 4H). |
| B-13 | 15.3%/ | 766.76 for | δ 8.84 (d, J=2.8 Hz, 2H), 8.82-8.79 (m, 2H), 8.51 (d, |
| | 95.34 | C39H34F4N10O3/ | J =2 Hz, 2H), 8.44 (d, J =4 Hz, 2H), 8.38-8.35 (m, |
| | | 767.2 | 2H), 8.25 (d, J=2. Hz, 2H), 6.85 (d, J =3.6 Hz, 2H), |
| | | (M + 1) | 4.17 (s, 3H), 2.09-2.07 (m, 2H). |
| B-14 | 23.5%/ | 422.17 for | δ 12.60-12.53 (m, 1H), 9.06 (br d, J = 4.4 Hz, 2H), |
| | 95.33% | C22H20F2N6O/ | 8.76-8.72 (m, 1H), 8.48 (d, J = 1.8 Hz, 1H), 8.27- |
| | | 423.1 | 8.16 (m, 2H), 7.11-6.81 (m, 2H), 3.79-3.50 (m, |
| | | (M + 1) | 4H), 2.30-2.18 (m, 3H), 2.15-2.02 (m, 4H). |
| B-15 | 4.7%/ | 422.17 for | δ 12.12-11.97 (m, 1H), 8.92 (br d, J = 17.4 Hz, 1H), |
| | 95.91% | C22H20F2N6O/ | 8.54 (br s, 1H), 8.47-8.45 (m, 1H), 8.35-8.30 (m, |
| | | 421.45 | 1H), 8.26-8.23 (m, 1H), 8.18-8.13 (m, 1H), 7.76- |
| | | (M + 1) | 7.70 (m, 1H), 6.89-6.85 (m, 1H), 3.74-3.58 (m, |
| | | | 4H), 2.36-2.34 (m, 3H), 2.13-2.05 (m, 4H). |
| B-16 | 3.86%/ | 422.17 for | δ 12.12-11.86 (m, 1H), 8.89-8.87 (m, 1H), 8.78 (d, |
| | 99.87% | C22H20F2N6O/ | J = 8.5 Hz, 1H), 8.51 (d, J = 2.1 Hz, 1H), 8.47 (d, |
| | | 423.15 | J = 3.8 Hz, 1H), 8.30 (dd, J = 8.6, 2.2 Hz, 1H), 8.25 |
| | | (M + 1) | (d, J = 2.1 Hz, 1H), 7.64-7.58 (m, 1H), 6.86-6.83 |
| | | | (m, 1H), 3.76-3.62 (m, 4H), 2.35 (s, 3H), 2.13-2.04 |
| | | | (m, 4H). |
| B-17 | 13.2%/ | 422.17 for | δ 12.74-12.54 (m, 1H), 9.15-9.08 (m, 1H), 8.51- |
| | 97.62% | C22H20F2N6O/ | 8.39 (m, 2H), 8.25 (d, J = 2.0 Hz, 1H), 8.21-8.13 |
| | | 423.2 | (m, 2H), 6.88 (d, J = 3.8 Hz, 2H), 3.79-3.47 (m, |
| | | (M + 1) | 4H), 2.23 (s, 3H), 2.14-2.03 (m, 4H). |
| B-18 | 18.5%/ | 423.16 for | δ 15.26-14.86 (m, 1H), 9.16-9.10 (m, 1H), 8.93- |
| | 94% | C21H19F2N7O/ | 8.91 (m, 1H), 8.75-8.72 (m, 1H), 8.47 (d, J = 2.0 |
| | | 424.5 | Hz, 1H), 8.27-8.23 (m, 2H), 6.90 (d, J = 3.7 Hz, |
| | | (M + 1) | 1H), 3.77-3.56 (m, 4H), 2.59-2.54 (m, 3H), 2.13- |
| | | | 2.04 (m, 4H). |
| B-19 | 21.9%/ | 423.16 for | δ 9.22 (d, J = 2.5 Hz, 1H), 8.50-8.43 (m, 2H), 8.27- |
| | 99.6% | C21H19F2N7O/ | 8.17 (m, 3H), 6.89 (d, J = 3.8 Hz, 1H), 3.75-3.53 |
| | | 424.1 | (m, 4H), 2.65 (s, 3H), 2.14-2.03 (m, 4H). |
| | | (M + 1) | |
| B-20 | 4.99%/ | 481.20 for | δ 9.29-9.27 (m, 1H), 9.05-9.02 (m, 1H), 8.91-8.87 |
| | 99.88% | C24H25F2N7O2/ | (m, 1H), 8.56-8.54 (m, 1H), 8.48-8.45 (m, 1H), |
| | | 482.2 | 8.29-8.24 (m, 2H), 6.92-6.89 (m, 1H), 3.74-3.54 |
| | | (M + 1) | (m, 4H), 2.14-2.04 (m, 4H), 1.42 (s, 9H). |
| B-21 | 30%/ | 423.16 for | δ 14.57-13.57 (m, 1H), 9.28-9.20 (m, 1H), 8.61- |
| | 99.18% | C21H19F2N7O/ | 8.46 (m, 2H), 8.28-8.20 (m, 3H), 6.90 (d, J = 3.7 |
| | | 424.2 | Hz, 1H), 3.80-3.56 (m, 4H), 2.43-2.37 (m, 3H), |
| | | (M + 1) | 2.14-2.04 (m, 4H). |
| B-22 | 12%/ | 423.16 for | δ 14.05-13.87 (m, 1H), 9.13 (br s, 2H), 8.90 (t, J = |
| | 99.61% | C21H19F2N7O/ | 2.0 Hz, 1H), 8.48 (d, J = 2.0 Hz, 1H), 8.29-8.22 (m, |
| | | 424.2 | 2H), 6.89 (d, J = 3.7 Hz, 1H), 3.77-3.50 (m, 4H), |
| | | (M + 1) | 2.45 (s, 3H), 2.08 (br t, J = 12.9 Hz, 4H). |
| B-23 | 5.6%/ | 542.22 for | δ 9.53-9.49 (m, 1H), 8.81-8.76 (m, 2H), 8.48-8.45 |
| | 98.06% | C30H28F2N6O2/ | (m, 1H), 8.12-8.08 (m, 1H), 7.93-7.89 (m, 1H), |
| | | 543.2 | 7.32-7.29 (m, 1H), 7.21-7.17 (m, 2H), 7.01-6.96 |
| | | (M + 1) | (m, 2H), 6.82-6.79 (m, 1H), 5.16-5.13 (m, 2H), |
| | | | 3.86-3.83 (m, 4H), 3.82-3.71 (m, 3H), 2.84-2.82 |
| | | | (m, 3H), 2.14-2.03 (m, 4H). |
| B-24 | 19.29%/ | 542.22 for | δ 9.10-9.05 (m, 1H), 8.76-8.72 (m, 1H), 8.51-8.45 |
| | 98.42% | C30H28F2N6O2/ | (m, 2H), 8.31-8.25 (m, 1H), 8.09-8.05 (m, 1H), |
| | | 543.2 | 7.22-7.17 (m, 2H), 7.15-7.12 (m, 1H), 7.02-6.96 |
| | | (M + 1) | (m, 2H), 6.73-6.70 (m, 1H), 5.14 (s, 2H), 3.85 (s, |
| | | | 3H), 3.83-3.64 (m, 4H), 2.81 (s, 3H), 2.14-2.03 |
| | | | (m, 4H). |
| B-25 | 46%/ | 437.18 for | δ 9.20-9.18 (m, 1H), 8.52-8.48 (m, 1H), 8.48-8.46 |
| | 99.63% | C22H21F2N7O/ | (m, 1H), 8.25 (d, J = 1.9 Hz, 1H), 8.21 (d, J = 3.8 |
| | | 438.25 | Hz, 3H), 8.19-8.16 (m, 1H), 6.89 (d, J = 3.8 Hz, |
| | | (M + 1) | 1H), 3.89-3.87 (m, 3H), 3.75-3.54 (m, 4H), 2.48- |
| | | | 2.48 (m, 3H), 2.13-2.04 (m, 4H). |
| B-26 | 20.23%/ | 408.15 for | δ 14.31-14.09 (m, 1H), 8.72-8.49 (m, 1H), 8.46 (d, |
| | 98.09% | C21H18F2N6O/ | J = 2.0 Hz, 1H), 8.24 (d, J = 2.1 Hz, 1H), 8.22-8.17 |
| | | 409.2 | (m, 2H), 8.13 (d, J = 3.7 Hz, 1H), 8.10-8.05 (m, |
| | | (M + 1) | 2H), 6.85 (d, J = 3.7 Hz, 1H), 3.78-3.52 (m, 4H), |
| | | | 2.12-2.03 (m, 4H). |
| B-27 | 41.4%/ | 403.11 for | δ 13.33-13.17 (m, 1H), 8.50 (d, J = 2.0 Hz, 1H), |
| | 99.71% | C20H16F3N3O3/ | 8.26-8.16 (m, 3H), 8.11-8.02 (m, 2H), 6.89 (d, |
| | | 402.3 | J = 3.9 Hz, 1H), 3.82-3.48 (m, 4H), 2.16-2.01 (m, |
| | | (M − 1) | 4H). |
| B-28 | 12.20%/ | 387.18 for | δ 14.74-14.42 (m, 1H), 9.29 (d, J = 2.3 Hz, 1H), |
| | 98.34% | C21H21N7O/ | 8.63-8.59 (m, 1H), 8.40 (d, J = 2.0 Hz, 1H), 8.35- |
| | | 386.25 | 8.27 (m, 1H), 8.27-8.25 (m, 1H), 8.30-8.21 (m, |
| | | (M + 1) | 1H), 8.25-8.20 (m, 1H), 8.17 (d, J = 2.0 Hz, 1H), |
| | | | 6.90 (d, J = 3.7 Hz, 1H), 4.41-4.26 (m, 1H), 3.71- |
| | | | 3.52 (m, 1H), 3.12-5 (m, 2H), 1.83-1.48 (m, 4H), |
| | | | 1.24-1.12 (m, 1H), 0.98-0.72 (m, 3H). |
| B-29 | 5.7%/ | 375.18 for | δ 9.18-9.15 (m, 1H), 8.36-8.33 (m, 1H), 8.15-8.13 |
| | 94.4% | C20H21N7O/ | (m, 1H), 8.05-8.02 (m, 1H), 7.96-7.90 (m, 1H), |
| | | 376.2 | 7.54-7.50 (m, 1H), 6.83-6.80 (m, 1H), 6.15-6.11 |
| | | (M + 1) | (m, 2H), 4.42-4.20 (m, 1H), 3.07-2.88 (m, 2H), |
| | | | 1.84-1.50 (m, 4H), 1.33-1.09 (m, 2H), 1.02-0.76 |
| | | | (m, 3H). |
| B-30 | 88%/ | 404.20 for | δ 8.75 (dd, J = 8.8, 2.2 Hz, 2H), 8.53 (t, J = 2.3 Hz, |
| | 98.99% | C22H24N6O2/ | 1H), 8.35 (d, J = 2.0 Hz, 1H), 8.15 (d, J = 2.0 Hz, |
| | | 405.2 | 1H), 8.08 (d, J = 3.8 Hz, 1H), 7.28 (s, 1H), 6.85 (d, |
| | | (M + 1) | J = 3.8 Hz, 1H), 4.44-4.23 (m, 1H), 3.98 (dd, J = |
| | | | 8.9, 6.9 Hz, 2H), 3.73-3.56 (m, 1H), 3.53-3.46 (m, |
| | | | 2H), 3.20-3 (m, 2H), 1.80 (br d, J = 12.8 Hz, 1H), |
| | | | 1.71-1.56 (m, 2H), 1.52-1.41 (m, 1H), 1.25-1.11 |
| | | | (m, 1H), 0.98-0.69 (m, 3H). |
| B-31 | %/ | 437.18 for | δ 9.39-9.36 (m, 1H), 8.66-8.62 (m, 1H), 8.50-8.47 |
| | 97.66% | C22H21F2N7O/ | (m, 1H), 8.31-8.25 (m, 3H), 6.94-6.91 (m, 1H), |
| | | 438.25 | 4.27-4.23 (m, 3H), 3.77-3.50 (m, 4H), 2.33-2.31 |
| | | (M + 1) | (m, 3H), 2.15-2.02 (m, 4H). |
| B-32 | 55.44%/ | 413.16 for | δ 8.47 (d, J = 2.0 Hz, 1H), 8.24 (d, J = 2.0 Hz, 1H), |
| | 99.75% | C₂₂H₂₁F₂N₃O₃/ | 8.20-8.16 (m, 3H), 8.16-8.12 (m, 2H), 6.88 (d, J = |
| | | 414.20 | 3.7 Hz, 1H), 4.38-4.33 (m, 2H), 3.78-3.50 (m, 4H), |
| | | (M + 1) | 2.12-2.03 (m, 4H), 1.35 (t, J = 7.1 Hz, 3H). |
| B-33 | 57%/ | 386.19 for | δ 14.55-14.06 (m, 1H), 8.69-8.62 (m, 1H), 8.37 (d, |
| | 92.93% | C22H22N6O/ | J = 1.8 Hz, 1H), 8.21-8.02 (m, 6H), 6.87-6.79 (m, |
| | | 387.2 | 1H), 4.45-4.19 (m, 1H), 3.79-3.37 (m, 1H), 3.19- |
| | | (M + 1) | 3 (m, 2H), 1.85-1.76 (m, 1H), 1.71-1.57 (m, 2H), |
| | | | 1.53-1.42 (m, 1H), 1.28-1.19 (m, 1H), 0.97-0.70 |
| | | | (m, 3H). |
| B-34 | 58%/ | 409.15 for | δ 14.32-14.21 (m, 1H), 9.14-9.12 (m, 1H), 9.08- |
| | 97.61% | C20H17F2N7O/ | 8.96 (m, 1H), 8.75-8.72 (m, 1H), 8.59 (s, 1H), 8.55- |
| | | 410.05 | 8.53 (m, 1H), 8.22-8.18 (m, 1H), 6.89-8.87 (m, |
| | | (M + 1) | 1H), 3.84-3.52 (m, 4H), 2.18-2.02 (m, 4H). |
| B-35 | 69%/ | 387.18 for | δ 14.85-13.50 (m, 1H), 9.13 (br s, 1H), 9.02-8.96 |
| | 95.07% | C21H21N7O/ | (m, 1H), 8.60 (br s, 2H), 8.54-8.42 (m, 2H), 8.17 |
| | | 388.10 | (br s, 1H), 6.88 (br s, 1H), 4.47-4.26 (m, 1H), 3.73- |
| | | (M + 1) | 3.55 (m, 1H), 3.11-2.79 (m, 2H), 1.90-1.77 (m, |
| | | | 1H), 1.71-1.54 (m, 2H), 1.52-1.41 (m, 1H), 1.25- |
| | | | 1.12 (m, 1H), 1.01-0.71 (m, 3H) |
| B-36 | 47%/ | 408.15 for | δ 14.35-14.07 (m, 1H), 8.75-8.60 (m, 1H), 8.57- |
| | 95.58% | C21H18F2N6O/ | 8.52 (m, 1H), 8.47-8.43 (m, 1H), 8.25-8.22 (m, |
| | | 409.2 | 1H), 8.14-8.10 (m, 1H), 8.06-8.00 (m, 1H), 7.93- |
| | | (M + 1) | 7.83 (m, 1H), 7.73-7.63 (m, 1H), 6.88-6.82 (m, |
| | | | 1H), 3.72-3.56 (m, 4H), 2.14-2.05 (m, 4H). |
| B-37 | 50%/ | 386.19 for | δ 14.18 (s, 1H), 8.67 (s, 1H), 8.55 (s, 1H), 8.36 (d, |
| | 96.21% | C22H22N6O/ | J = 1.6 Hz, 1H), 8.14-7.87 (m, 4H), 7.67-7.63 (m, 1H), |
| | | 387.2 | 6.83 (d, J = 2.8 Hz, 1H), 4.33 (brs, 1H), 3.58 (brs, |
| | | (M + 1) | 1H), 2.96 (brs, 1H), 1.98-1.78 (m, 1H), 1.64-1.62 (m, |
| | | | 2H), 1.49-1.46 (m, 1H), 0.89-0.85 (m, 3H), |
| B-38 | 25.5%/ | 401.20 for | δ 14.8 (s, 1H), 9.32 (s, 1H), 8.64-8.11 (m, 6H), 6.91 |
| | 97.31% | C22H23N7O/ | (d, J = 3.2 Hz, 1H), 3.59-3.16 (m, 3H), 1.58 (brs, |
| | | 402.05 | 2H), 1.43 (d, J = 5.6 Hz, 2H), 0.96-0.80 (m, 7H). |
| | | (M + 1) | |
| B-39 | 7.8%/ | 389.16 for | δ 14.7 (brs, 1H), 9.29 (s, 1H), 8.62 (d, J = 8.4 Hz, |
| | 99.05% | C20H19N7O/ | 1H), 8.42 (s, 1H), 8.26-8.19 (m, 3H), 6.91 (d, J = |
| | | 390.05 | 3.6 Hz, 1H), 5.40-4.80 (m, 1H), 3.56 (brs, 2H), |
| | | (M + 1) | 3.16-3.12 (m, 3H), 1.85 (brs, 2H), 1.46 (brs, 2H). |
| B-40 | 53.8%/ | 403.18 for | δ 14.8 (brs, 1H), 9.29 (s, 1H), 8.62 (s, 1H), 8.40 (s, |
| | 92.90% | C₂₁H₂₁N₇O₂/ | 1H), 8.28-8.18 (m, 5H), 6.93-6.90 (m, 1H), 3.99- |
| | | 404.15 | 3.75 (m, 1H), 3.43-3.11 (m, 3H), 1.87-1.46 (m, 3H). |
| | | (M + 1) | |
| B-41 | 4.96%/ | 373.17 for | δ 14.61 (brs, 1H), 9.29 (s, 1H), 8.62-8.52 (m, 2H), |
| | 99.71% | C20H19N7O/ | 8.33-8.23 (m, 4H), 6.91 (d, J = 4 Hz, 1H), 3.73-3.47 |
| | | 374.00 | (m, 3H), 3.18-3.04 (m, 1H), 2.25-2.20 (m, 1H), 2.06- |
| | | (M + 1) | 1.96 (m, 1H), 1.58-1.46 (m, 1H), 1.10-0.97 (m, 3H). |
| B-42 | 31.7%/ | 387.18 for | δ 14.8 (s, 1H), 9.32 (s, 1H), 8.70-8.53 (m, 2H), 8.36- |
| | 96.07% | C21H21N7O/ | 8.11 (m, 4H), 6.90 (s, 1H), 3.63-3.59 (m, 2H), 3.30- |
| | | 388.40 | 3.28 (m, 2H), 1.74-1.66 (m, 2H), 1.13 (s, 2H), 0.99 |
| | | (M + 1) | (s, 3H) |
| B-43 | 1.75%/ | 375.14 for | δ 14.22 (brs, 1H), 9.30 (d, J = 4 Hz, 1H), 8.62-8.55 |
| | 99.92% | C19H17N7O2/ | (m, 2H), 8.34-8.23 (m, 4H), 6.91 (d, J = 4 Hz, 1H), |
| | | 376.00 | 5.03-4.95 (m, 1H), 4.36-4.26 (m, 1H), 3.72-3.57 (m, |
| | | (M + 1) | 4H), 2.0-1.79 (m, 2H). |
| B-44 | 35%/ | 389.16 for | δ 14.22 (brs, 1H), 9.30 (s, 1H), 9.29-8.32 (m, 2H), |
| | 94.13% | C20H19N7O2/ | 8.28-8.23 (m, 4H), 6.91 (s, 1H), 5.03 (d, J = 4 Hz, |
| | | 390.05 | 1H), 4.36-4.26 (m, 1H), 3.69-3.57 (m, 4H), 2.49- |
| | | (M + 1) | 1.97 (m, 2H). |
| B-45 | 65.86%/ | 410.1 for | δ 15.04 (brs, 1 H), 9.62 (s, 2 H), 8.51 (d, J = 2.00 Hz, |
| | 95.58% | C₁₉H₁₆F₂N₈O/ | 1 H), 8.32 (d, J = 3.75 Hz, 1 H), 8.29 (d, J = 2.00 Hz, 1 |
| | | 411.2 | H), 6.96 (d, J = 3.75 Hz, 1 H), 3.58-3.76 (m, 4 H), |
| | | (M + 1) | 2.02-2.16 (m, 6 H). |
| B-46 | 44.30%/ | 389.16 for | δ 9.29-9.22 (m, 1H), 8.56 (dd, J = 8.5, 2.2 Hz, 1H), |
| | 96.89% | C20H19N7O2/ | 8.50-8.38 (m, 1H), 8.30-8.18 (m, 4H), 6.90 (d, J = |
| | | 390.2 | 3.5 Hz, 1H), 4.46-4.21 (m, 1H), 3.95-3.68 (m, 2H), |
| | | (M + 1) | 3.64-3.41 (m, 4H), 1.09 (br s, 3H) |
| B-47 | 81%/ | 403.45 for | δ 14.64 (brs, 1H), 9.28 (d, J = 2 Hz, 1H), 8.61-8.58 |
| | 97.20% | C21H21N7O2/ | (m, 1H), 8.43 (s, 1H), 8.30-8.21 (m, 4H), 6.91 (d, |
| | | 404.2 | J = 2.8 Hz, 1H), 4.42 (brs, 1H), 3.60-3.57 (m, 3H), |
| | | (M + 1) | 2.99 (s, 1H), 1.14-1.00 (m, 6H). |
| B-48 | 41%/ | 399.41 for | δ 11.29 (s, 1H), 9.60-9.47 (m, 1H), 8.80-8.51 (m, |
| | 47.55% | C19H19F2N7O/ | 1H), 8.49 (s, 1H), 8.43-8.27 (m, 3H), 6.97-6.94 (m, |
| | | 400.00 | 1H), 5.41-5.32 (m, 1H), 2.08-2.0 (m, 4H) |
| | | (M + 1) | |
| B-49 | 7.1%/ | 404.17 for | δ 9.39 (d, J = 2.4 Hz, 1H), 8.65-8.61 (m, 2H), 8.45 |
| | 96.21% | C₂₀H₂₀N₈O₂/ | (d, J = 1.8 Hz, 1H), 8.32 (d, J = 8.8 Hz, 1H), 8.27- |
| | | 405.2 | 8.22 (m, 2H), 6.94-6.91 (m, 1H), 6.76 (s, 2H), 3.89- |
| | | (M + 1) | 3.79 (m, 2H), 3.58-3.49 (m, 3H), 3.26-3.19 (m, |
| | | | 2H), 1.12-1.04 (m, 3H) |
| B-50 | 76%/ | 476.41 for | δ = 8.48 (d, J = 2.0 Hz, 1H), 8.25 (d, J = 2.0 Hz, 1H), |
| | 99.0% | C22H17F5N6O/ | 8.22 (s, 4H), 8.19 (d, J = 3.6 Hz, 1H), 6.88 (d, J = 3.6 |
| | | 475.0 | Hz, 1H), 3.77-3.49 (m, 4H), 2.16-2.00 (m, 4H). |
| | | (M − 1) | |
| B-51 | 36%/ | 435.46 for | δ: 10.8 (s, 1H), 8.69 (s, 1H), 8.62 (s, 1H), 8.53-8.52 |
| | 99.9% | C22H23F2N5O3/ | (d, J =2.1 Hz, 1H), 8.36-8.35 (d, J = 2.1 Hz, 1H), |
| | | 440.0 | 8.18-8.17 (d, J = 1.8 Hz, 1H), 8.09-8.08 (d, J = |
| | | (M + 1) | 3.6 Hz, 1H), 6.87-6.86 (d, J = 3.3 Hz, 1H), 4.97- |
| | | | 4.89 (m, 1H), 3.89 (bs, 2H), 3.56 (bs, 2H), 2.17- |
| | | | 2.08 (m, 2H), 1.73 (bs, 2H), 1.29-1.27 (d, J = 6.6 |
| | | | Hz, 6H). |
| B-52 | 71%/ | 398.47 for | δ = 8.72-8.41 (m, 1H), 8.40 (d, J = 2.0 Hz, 1H), 8.24- |
| | 99.4% | C23H22N6O/ | 8.13 (m, 3H), 8.13-7.99 (m, 3H), 6.82 (d, J = 3.6 |
| | | 399.1 | Hz, 1H), 3.75-3.41 (m, 4H), 1.37 (br s, 4H), 0.33 (s, |
| | | (M + 1) | 4H). |
| B-53 | 3.2%/ | 407.43 for | δ: 12.61 (s, 1H), 8.61 (s, 1H), 8.45-8.44 (d, J = 1.5 |
| | 94.2% | C22H19F2N5O/ | Hz, 1H), 8.23-8.22 (d, J = 2.1 Hz, 1H), 8.11-8.09 |
| | | 408.1 | (d, J = 6.9 Hz, 3H), 8.02-7.99 (d, J = 8.4 Hz, 2H), |
| | | (M + 1) | 6.84-6.83 (d, J = 3.6 Hz, 1H), 6.54 (s, 2H), 3.65 |
| | | | (brs, 4H), 2.08 (brs, 4H). |
| B-54 | 6.6%/ | 423.43 for | δ/ppm 12.11 (s, 1H), 8.38-8.37 (d, J = 1.2 Hz, 1H), |
| | 95.1% | C21H19F2N7O/ | 8.17-8.16 (d, J = 1.2 Hz, 1H), 8.11-8.09 (d, J = |
| | | 424.1 | 3.6 Hz, 1H), 8.05-8.03 (m, 2H), 7.96-7.93 (m, |
| | | (M + 1) | 2H), 6.84-6.82 (d, J = 3.6 Hz, 1H), 6.12 (s, 2H), |
| | | | 3.89 (bs, 2H), 3.58 (bs, 2H), 2.17-2.08 (m, 2H), |
| | | | 1.73 (bs, 2H). |
| B-55 | 27.2%/ | 429.43 for | δ: 10.13 (s, 1H), 8.64 (s, 1H), 8.52 (s, 1H), 8.36 (s, |
| | 98.8% | C21H21F2N5O3/ | 1H), 8.18 (s, 1H), 8.08 (s, 1H), 7.37 (s, 1H), 6.87- |
| | | 430.1 | 6.86 (d, J = 3.3 Hz, 1H), 4.24-4.14 (q, 2H), 3.39 |
| | | (M + 1) | (bs, 2H), 3.33 (bs, 2H), 2.15 (bs, 2H), 1.73 (bs, 2H), |
| | | | 1.29-1.24 (t, 3H). |
| B-56 | 57.8%/ | 399.46 for | δ: 9.17 (s, 1H), 8.55-8.25 (m, 3H), 8.13 (d, J = 1.8 |
| | 92.7% | C22H21N7O/ | Hz, 2H), 7.66 (d, J = 3.3 Hz, 1H), 6.80 (d, J = 3.6 Hz, |
| | | 400.3 | 1H), 3.84 (brs, 2H), 8.70-8.40 (m, 2H), 0.85 (brs, |
| | | (M + 1) | 4H), 0.40 (brs, 4H). |
| B-57 | 23%/ | 413.35 for | δ/ppm 8.67 (s, 1H), 8.39-8.38 (d, J = 2.1 Hz, 1H), |
| | 99.6% | C18H16F5N5O/ | 8.25 (s, 1H), 8.15-8.14 (d, J = 1.2 Hz, 1H), 8.09- |
| | | 414.1 | 8.08 (d, J = 3.6 Hz, 1H), 6.81-6.79 (d, J = 3.6 Hz, |
| | | (M + 1) | 1H), 5.31-5.22 (m, 2H), 3.88 (b s, 2H), 3.57 (b s, |
| | | | 2H), 2.16-2.08 (m, 2H), 1.73 (bs, 2H). |
| B-58 | 15%/ | 450.49 for | δ: 13.71 (s, 1H), 8.45 (d, J = 1.8 Hz, 1H), 8.23 (d, |
| | 98.3% | C24H24F2N6O/ | J = 1.8 Hz, 1H), 8.16 (s, 1H), 8.13-8.11 (t, 2H), 8.02 |
| | | 451.1 | (d, J = 8.4 Hz, 2H), 6.84 (d, J = 6 Hz, 1H), 3.63 (br |
| | | (M + 1) | s, 4H), 2.72 (s, 1H), 2.08 (br s, 4H), 1.33 (d, J = 9 |
| | | | Hz, 6H). |
| B-59 | 54%/ | 445.37 for | δ: 8.67 (s, 1H), 8.42 (s, 1H), 8.25 (s, 1H), 8.15 (s, |
| | 90.2% | C19H17F6N5O/ | 1H), 8.07 (d, J = 3 Hz, 1H), 6.79 (d, J = 3.6 Hz, 1H), |
| | | 446.1 | 5.26 (m, 2H), 4.54 (s, 1H), 3.68 (s, 1H), 3.07 (s, 2H), |
| | | (M + 1) | 2.67 (s, 1H), 1.98 (s, 1H), 1.72-1.66 (m, 1H), 1.62- |
| | | | 1.55 (m, 2H). |
| B-60 | 10%/ | 467.48 for | δ/ppm 8.96 (s, 1H), 8.45 (s, 1H), 8.13 (s, 1H), 7.77- |
| | 98.6% | C23H23F2N7O2/ | 7.74 (dd, 1H,), 7.60-7.54 (m, 2H), 6.76-6.75 (m, |
| | | 468.2 | 1H), 3.86-3.83 (m, 4H), 3.66-3.62 (m, 4H), 2.13- |
| | | (M + 1) | 2.09 (m, 2H), 1.88 (bs, 4H), 0.84-0.82 (m, 2H). |
| B-61 | 12.2%/ | 408.41 for | δ: 14.16 (s, 1H), 8.66 (s, 1H), 8.38 (d, J = 1.2 Hz, |
| | 97.8% | C21H18F2N6O/ | 1H), 8.21-8.02 (m, 6H), 6.85 (d, J = 3.0 Hz, 1H), |
| | | 409.2 | 3.90 (d, 2H), 3.57 (s, 3H), 2.17-2.05 (m, 2H), 1.74 |
| | | (M + 1) | (s, 2H). |
| B-62 | 66%/ | 389.41 for | δ: 8.44 (d, J = 2.7 Hz, 2H), 8.18 (d, J = 1.8 Hz, 1H), |
| | 95.5% | C19H21F2N5O2/ | 8.07 (s, 1H), 7.99 (d, J = 3 Hz, 1H), 6.75 (d, J = 3.6 |
| | | 390.1 | Hz, 1H), 4.34 (t, J = 12 Hz, 2H), 3.73 (t, J = 9 Hz, |
| | | (M + 1) | 2H), 3.63 (br s, 4H), 3.25 (s, 3H), 2.07 (br s, 4H). |
| B-63 | 68%/ | n.a. | δ: 8.48 (s, 1H), 8.44 (s, 1H), 8.18 (s, 1H), 8.05 (s, |
| | 97.7% | | 1H), 7.99 (d, J = 3.6 Hz, 1H), 6.76 (d, J = 3.3 Hz, |
| | | | 1H), 4.05 (d, J = 7.2 Hz, 2H), 3.63 (br s, 4H), 2.07 |
| | | | (br s, 4H). 1.29-1.22 (m, 2H), 0.55 (d, J = 7.2 Hz, |
| | | | 2H), 0.41 (d, J = 6 Hz, 2H). |
| B-64 | 15%/ | 436.47 for | δ: 8.45 (d, J = 2.1 Hz, 1H), 8.23 (d, J = 1.8 Hz, 1H), |
| | 99.2% | C23H22F2N6O/ | 8.17-8.12 (m, 3H), 8.02 (d, J = 8.7 Hz, 2H), 6.84 |
| | | 437.2 | (d, J = 3.6 Hz, 1H), 2.81-2.74 (m, 3H), 2.07 (s, 4H), |
| | | (M + 1) | 1.35-1.27 (m, 4H), 1.17 (t, J = 6 Hz, 2H) |
| B-65 | 26%/ | 447.42 for | δ = 8.75 (br s, 1H), 8.59 (br s, 1H), 8.51 (br s, 1H), |
| | 98.0% | C21H20F3N5O3/ | 8.46 (d, J = 1.6 Hz, 1H), 8.10 (d, J = 1.6 Hz, 1H), |
| | | 448.2 | 7.63 (d, J = 3.6 Hz, 1H), 7.04 (br s, 1H), 6.75 (d, J = |
| | | (M + 1) | 3.6 Hz, 1H), 3.84 (s, 3H), 3.20-2.85 (m, 2H), 2.50- |
| | | | 2.26 (m, 1H), 2.20-2.11 (m, 1H), 1.89-1.80 (m, |
| | | | 1H), 1.75-1.63 (m, 4H) |
| B-66 | n.a./ | 447.42 for | δ: 10.16 (s, 1H), 8.71-8.70 (d, J = 2.1 Hz, 1H), 8.63- |
| | 100% | C21H20F3N5O3/ | 8.62 (d, J = 2.4 Hz, 1H), 8.54-8.53 (d, J = 2.1 Hz, |
| | | 448.1 | 1H), 8.39 (s, 1H), 8.19 (s, 1H), 8.09-8.08 (d, J = 2.4 |
| | | (M + 1) | Hz, 1H), 6.87-6.86 (d, J = 3.9 Hz, 1H), 4.54 (br s, |
| | | | 1H), 3.72 (s, 3H), 3.08 (br s, 2H), 2.01 (m, 1H), 1.61 |
| | | | (m, 2H), 1.29 (m, 3H). |
| B-67 | 6%/ | 493.52 for | δ ppm 12.81 (s, 1H), 8.44 (s, 1H), 8.23 (s, 1H), 8.15- |
| | 99.6% | C25H25F2N7O2/ | 8.14 (m, 1H), 8.09-8.07 (m, 3H,), 7.98-7.95 (m, |
| | | 492.1 | 2H), 6.84-6.82 (m, 1H), 3.73-3.71 (m, 8H), 3.39 |
| | | (M − 1) | (m, 4H), 2.07-2.03 (m, 4H). |
| B-69 | 60%/ | 407.47 for | δ = 10.15 (s, 1H), 8.71 (d, J = 2.0 Hz, 1H), 8.63 (d, |
| | 99.0% | C22H25N5O3/ | J = 2.0 Hz, 1H), 8.53 (s, 1H), 8.35 (d, J = 2.0 Hz, 1H), |
| | | 408.2 | 8.15 (d, J = 2.0 Hz, 1H), 8.07 (d, J = 3.6 Hz, 1H), |
| | | (M + 1) | 6.85 (d, J = 3.6 Hz, 1H), 4.54-4.14 (m, 1H), 3.72 (s, |
| | | | 3H), 3.68-3.45 (m, 1H), 3.15-2.60 (m, 2H), 1.92- |
| | | | 1.79 (m, 1H), 1.77-1.54 (m, 1H), 1.42 (ddd, J = 3.6, |
| | | | 6.8, 10.0 Hz, 2H), 1.34-1.01 (m, 3H), 1.00-0.73 |
| | | | (m, 3H). |
| B-70 | 61%/ | 407.47 for | δ = 10.14 (br s, 1H), 8.72 (s, 1H), 8.63 (s, 1H), 8.53 |
| | 99.0% | C22H25N5O3/ | (br s, 1H), 8.35 (s, 1H), 8.15 (s, 1H), 8.06 (br s, 1H), |
| | | 408.2 | 6.85 (br s, 1H), 4.35 (br s, 1H), 3.72 (s, 3H), 3.60 (br |
| | | (M + 1) | s, 1H), 3.12-2.57 (m, 2H), 1.85 (br d, J = 12.4 Hz, |
| | | | 1H), 1.77-1.54 (m, 1H), 1.43 (br s, 2H), 1.34-1.03 |
| | | | (m, 3H), 0.77-0.73 (m, 3H). |
| B-71 | 17%/ | 423.43 for | δ/ppm 12.06 (s, 1H), 8.43-8.39 (m, 2H), 8.23-8.22 |
| | 97.6% | C21H19F2N7O/ | (d, J = 1.8 Hz, 1H), 8.08-8.07 (d, J = 3.6 Hz, 1H), |
| | | 424.2 | 7.90-7.59 (m, 3H), 6.83-6.82 (d, J = 3.6 Hz, 1H), |
| | | (M + 1) | 6.14 (s, 2H), 3.64 (s, 4H), 2.08-2.06 (d, J = 5.4 Hz, |
| | | | 4H). |
| B-72 | 10.6%/ | 448.48 for | 1H-NMR (300 MHz, DMSO-d6) δ: 13.7 (s, 1H), |
| | 98.3% | C24H22F2N6O/ | 8.45 (s, 1H), 8.23 (s, 1H), 8.09 (d, J = 11.4 Hz, 3H), |
| | | 449.2 | 7.99 (d, J = 8.4 Hz, 2H), 6.84 (d, J = 3 Hz, 1H), 3.65 |
| | | (M + 1) | (brs, 4H), 2.08 (s, 4H), 1.24 (d, J = 6 Hz, 2H), 1.07 |
| | | | (d, J = 6 Hz, 3H). |
| B-73 | 15.6%/ | 451.48 for | δ/ppm 8.43 (s, 1H), 8.36 (s, 1H), 8.24-8.23 (d, J = |
| | 92.0% | C23H23F2N7O/ | 1.5 Hz, 1H), 8.12-8.07 (m, 1H), 7.94-7.78 (m, 2H), |
| | | 452.2 | 7.67-7.54 (m, 1H), 6.88-6.82 (m, 1H), 3.65 (s, 4H), |
| | | (M + 1) | 2.99 (s, 6H), 2.07-2.06 (d, J = 4.5 Hz, 4H). |
| B-74 | 15.6/ | 422.44 for | δ: 13.7 (s, 1H), 8.45 (s, 1H), 8.23 (s, 1H), 8.15 (d, |
| | 91.6% | C22H20F2N6O/ | J = 8.1 Hz, 3H), 8.00 (d, J = 7.8 Hz, 2H), 6.84 (s, 1H), |
| | | 423.1 | 3.65 (brs, 4H), 2.43 (s, 3H), 2.08 (brs, 4H). |
| | | (M + 1) | |
| B-75 | n.a./ | 441.44 for | δ: 8.90 (s, 1H), 8.42-8.36 (m, 2H), 8.27 (s, 1H), 8.24- |
| | 99.6% | C22H21F2N5O3/ | 8.23 (m, 1H), 8.09 (d, J = 3.6 Hz, 1H), 6.86 (d, J = |
| | | 442.2 | 3.6 Hz, 1H), 4.50 (t, J = 7.5 Hz, 2H), 4.24 (t, J = 8.4 |
| | | (M + 1) | Hz, 2H), 3.06 (brs, 2H), 2.13 (brs, 2H), 0.94 (brs, |
| | | | 3H); |
| B-76 | n.a./ | 441.44 for | δ: 8.90 (s, 1H), 8.42-8.36 (m, 2H), 8.27 (s, 1H), 8.24- |
| | 99.9% | C22H21F2N5O3/ | 8.23 (m, 1H), 8.09 (d, J = 3.6 Hz, 1H), 6.86 (d, J = |
| | | 442.2 | 3.6 Hz, 1H), 4.50 (t, J = 7.5 Hz, 2H), 4.24 (t, J = 8.4 |
| | | (M + 1) | Hz, 2H), 3.06 (brs, 2H), 2.13 (brs, 2H), 0.94 (brs, |
| | | | 3H); |
| B-77 | 5.7%/ | 451.48 for | δ: ppm 12.56 (s, 1H), 8.46-7.54 (m, 7H), 6.86-6.82 |
| | 96.0% | C23H23F2N7O/ | (m, 1H), 3.64 (s, 4H), 2.99 (s, 6H), 2.07-2.06 (d, |
| | | 452.2 | J = 4.5 Hz, 4H). |
| | | (M + 1) | |
| B-78 | 2.8%/ | 423.43 for | d: ppm 12.11 (s, 1H), 8.44-8.43 (d, J = 1.8 Hz, 1H), |
| | 97.2% | C21H19F2N7O/ | 8.23-8.22 (d, J = 3.6 Hz, 1H), 8.11-8.10 (d, J = 3.6 |
| | | 422.1 | Hz, 1H), 8.06-7.95 (m, 4H), 6.84-6.82 (d, J = 3.9 |
| | | (M − 1) | Hz, 1H), 6.13 (s, 2H), 3.65 (s, 4H), 2.08 (s, 4H). |
| B-79 | 23.9%/ | 457.48 for | d: ppm 9.87 (s, 1H), 8.67 (s, 1H), 8.61 (s, 1H), 8.49 |
| | 94.6% | C23H25F2N5O3/ | (s, 1H), 8.43 (s, 1H), 8.24 (s, 1H), 8.07-8.05 (d, J = |
| | | 458.1 | 3.9 Hz, 1H), 6.86-6.84 (d, J = 3.6 Hz, 1H), 3.63 (s, |
| | | (M + 1) | 4H), 2.07 (s, 4H), 1.49 (s, 9H). |
| B-80 | n.a./ | 459.43 for | δ: 8.89 (s, 1H), 8.39-8.35 (m, 2H), 8.27-8.24 (m, |
| | 99.8% | C22H20F3N5O3/ | 1H), 8.19 (s, 1H), 8.09 (d, J = Hz, 1H), 6.86 (d, J = |
| | | 460.1 | Hz, 1H), 4.50 (t, J = Hz, 2H), 4.24 (t, J = Hz, 2H), |
| | | (M + 1) | 3.09 (brs, 2H), 2.01-1.98 (m, 1H), 1.72-1.52 (m, |
| | | | 3H), 1.29-1.25 (m, 3H); |
| B-81 | n.a./ | 459.43 for | δ: 8.89 (s, 1H), 8.39-8.35 (m, 2H), 8.27-8.24 (m, |
| | 99.2% | C22H20F3N5O3/ | 1H), 8.19 (s, 1H), 8.09 (d, J = Hz, 1H), 6.86 (d, J = |
| | | 460.2 | Hz, 1H), 4.50 (J = Hz, 2H), 4.24 (t, J = Hz, 2H), 3.09 |
| | | (M + 1) | (brs, 2H), 2.01-1.98 (1H), 1.72-1.52 (m, 3H), 1.29- |
| | | | 1.25 (m, 3H |
| B-82 | 55.2%/ | 415.4 for | δ: 10.16 (s, 1H), 8.71 (s, 1H), 8.64 (s, 1H), 8.52 (s, |
| | 98.6% | C20H19F2N5O3/ | 1H), 8.36 (s, 1H), 8.18 (s, 1H), 8.10 (d, J = 3.9 Hz, |
| | | 416.2 | 1H), 6.88 (d, J = 3.9 Hz, 1H), 3.89 (brs, 2H), 3.72 (s, |
| | | (M + 2) | 3H), 3.57 (brs, 2H), 2.17-2.08 (m, 2H), 1.73 (brs, |
| | | | 2H); |
| B-83 | 47%/ | 407.47 for | δ: 10.16 (s, 1H), 8.71 (bd, 1H), 5.53 (s, 1H), 8.39 (s, |
| | 95.2% | C22H25N5O3/ | 1H), 8.19 (d, J = 3.0 Hz, 1H), 8.09 (s, 1H), 6.87 (d, |
| | | 408.2 | J = 3.3 Hz, 1H), 4.57 (br s, 1H), 3.68 (s, 3H), 3.09 (br |
| | | (M + 1) | s, 2H), 2.67 (bs, 1H), 2.01 (m, 1H), 1.66 (m, 2H), |
| | | | 1.29 (m, 3H). ESI-MS (m/z) Calcd for |
| | | | C22H25N5O3: 407.47 |
| B-84 | 48%/ | 447.42 for | δ: 10.16 (s, 1H), 8.71 (bd, 1H), 5.53 (s, 1H), 8.39 (s, |
| | 97.5% | C21H20F3N5O3/ | 1H), 8.19 (d, J = 3.0 Hz, 1H), 8.09 (s, 1H), 6.87 (d, |
| | | 448.2 | J = 3.3 Hz, 1H), 4.57 (br s, 1H), 3.68 (s, 3H), 3.09 (br |
| | | (M + 1) | s, 2H), 2.67 (bs, 1H), 2.01 (m, 1H), 1.66 (m, 2H), |
| | | | 1.29 (m, 3H). |
| B-85 | 31.5%/ | 443.46 for | δ/ppm 9.01-9.00 (d, J = 2.1 Hz, 1H), 8.60-8.59 (d, |
| | 98.9% | C22H23F2N5O3/ | J = 1.8 Hz, 1H), 8.45-8.38 (m, 2H), 8.25-8.24 (d, |
| | | 441.0 | J = 1.8 Hz, 1H), 8.16-8.14 (d, J = 3.6 Hz, 1H), 6.88- |
| | | (M + 1) | 6.87 (d, J = 3.6 Hz, 1H), 4.17-4.11 (m, 2H), 3.64 |
| | | | (s, 4H), 3.34-3.33 (d, J = 2.1 Hz, 3H), 2.07 (s, 4H), |
| | | | 1.24-1.19 (m, 3H). |
| B-86 | 50.8%/ | 412.40 for | δ (ppm): 8.50-8.49 (d, J = 2.1 Hz, 1H), 8.25-8.24 |
| | 97.6% | C20H18F2N6O2/ | (d, J = 2.1 Hz, 2H), 8.08-8.06 (dd, 1H), 7.96 (s, |
| | | 413.2 | 1H), 7.25-7.55 (m, 1H), 6.90-6.89 (d, J = 3.9 Hz, |
| | | (M + 1) | 1H), 3.65 (s, 4H), 3.56 (s, 3H), 2.07 (s, 4H). |
| B-87 | 35.7%/ | 395.41 for | δ: 8.62 (d, J = 7.5 Hz, 1H), 8.48 (d, J = 1.8 Hz, 1H), |
| | 98.4% | C21H19F2N5O/ | 8.23 (d, J = 1.8 Hz, 1H), 8.21 (d, J = 3.6 Hz, 1H), |
| | | 396.1 | 8.11 (s, 1H), 7.73 (s, 1H), 7.61 (dd, J = 2.1 Hz, 1H), |
| | | (M + 1) | 6.86 (d, J = 3.9 Hz, 1H), 3.64 (brs, 4H), 2.36 (s, 3H), |
| | | | 2.20-2.00 (m, 4H). |
| B-88 | 30%/ | 443.46 for | δ: 10.07 (s, 1H), 8.69 (d, J = 2.1 Hz, 1H), 8.61 (d, J = |
| | 97.6% | C22H23F2N5O3/ | 2.1 Hz, 1H), 8.53 (d, J = 2.1 Hz, 1H), 8.42 (d, J = |
| | | 444.1 | 1.8 Hz, 1H), 8.24 (d, J = 2.1 Hz, 1H), 8.07 (d, J = 3.9 |
| | | (M + 1) | Hz, 1H), 6.85 (d, J = 3.6 Hz, 1H), 4.97-4.89 (m, 1H), |
| | | | 3.65 (br s, 4H), 2.07 (br s, 4H), 1.28 (d, J = 6.3 Hz, |
| | | | 6H). |
| B-89 | 28.8%/ | 411.42 for | δ: 9.24 (d, J = 1.5 Hz, 1H), 8.43 (d, J = 1.8 Hz 1H), |
| | 99.4% | C20H19F2N7O/ | 8.23 (d, J = 3.6 Hz, 1H), 7.96 (d, J = 2.1 Hz, 1H), |
| | | 412.3 | 7.93 (d, J = 2.1 Hz, 1H), 7.54 (d, J = 9.3 Hz, 1H), |
| | | (M + 1) | 6.83 (d, J = 3.9 Hz, 1H), 6.6 (brs, 1H), 3.65 (brs, 4H), |
| | | | 2.85 (d, 3H). 2.07 (brs, 4H) |
| B-90 | 48.2%/ | 399.45 for | δ: 8.44 (t, J = 10.8, 0.6 Hz, 2H), 8.18 (d, J = 1.8 Hz |
| | 96.0 | C21H23F2N5O/ | 1H), 8.05 (s, J = 3.6 Hz, 1H), 7.97 (d, J = 3.6 Hz, |
| | | 400.2 | 1H), 6.75 (d, J = 3.6 Hz, 1H), 4.828-4.735 (m, 4H), |
| | | (M + 1) | 2.15-1.92 (m, 9H), 1.87-1.76 (m, 2H), 1.72-1.63 (m, |
| | | | 2H). |
| B-91 | 30.6%/ | 440.46 for | δ: 8.76 (d, J = 2.4 Hz, 1H), 8.41 (d, J = 1.8 Hz 1H), |
| | 96.4% | C22H22F2N6O2/ | 8.36 (m, 1H), 8.2 (m, 2H), 8.13 (d, J = 3.6 Hz, 1H), |
| | | 441.1 | 6.82 (d, J = 3.6 Hz, 1H), 4.0 (t, J = 7.5, 2H), 3.5 (t, |
| | | (M + 1) | J = 8.1, 2H), 2.83 (s, 3H), 2.1 (m, 4 H). |
| B-92 | 38%/ | 465.47 for | δ/ppm 10.29 (s, 1H), 8.92-8.91 (d, J = 1.5 Hz, 1H), |
| | 90.8% | C23H21F2N7O2/ | 8.78 (s, 2H), 8.46-8.45(d, J = 1.8 Hz, 1H), 8.38 (s, |
| | | 466.1 | 1H), 8.26-8.25 (d, J = 2.1 Hz, 1H), 8.13-8.07 (t, |
| | | (M + 1) | 2H), 6.88-6.87 (d, J = 3.6 Hz, 1H), 3.92 (s, 3H), 3.65 |
| | | | (s, 4H), 2.12-2.08 (m, 4H). |
| B-93 | 23%/ | 425.44 for | δ/ppm 10.70 (s, 1H), 8.76-8.73 (q, 2H), 8.67 (s, 1H), |
| | 94.7% | C22H21F2N5O2/ | 8.44-8.43 (d, J = 1.5 Hz, 1H), 8.24-8.23 (d, J = 1.5 |
| | | 426.1 | Hz, 1H), 8.10-8.09 (d, J = 3.6 Hz, 1H), 6.86-6.85 |
| | | (M + 1) | (d, J = 3.6 Hz, 1 H), 3.64 (s, 4H), 2.07 (s, 4H), |
| | | | 1.89-1.81 (m, 1H), 0.87-0.81 (m, 4H). |
| B-94 | 66.3%/ | 415.46 for | δ/ppm 8.49 (s, 1H), 8.44-8.43 (d, J = 1.8 Hz, 1H), |
| | 99.9% | C21H23F2N5O2/ | 8.19-8.18 (d, J = 2.1 Hz, 1H), 8.09 (s, 1H), 7.98- |
| | | 416.2 | 7.97 (d, J = 3.6 Hz, 1H), 6.76-6.75 (d, J = 3.6 Hz, |
| | | (M + 1) | 1H), 4.55-4.45 (m, 1H), 4.01-3.97 (m, 2H), 3.64 |
| | | | (s, 4H), 3.53-3.44 (m, 2H), 2.07-1.92 (m, 8H). |
| B-95 | 57.6%/ | 359.38 for | δ: 8.44 (d, J = 6 Hz, 1H), 8.18 (s, 1H), 8.04 (s, 1H), |
| | 99.3% | C18H19F2N5O/ | 7.97 (d, J = 3.3 Hz, 1H), 6.75 (d, J = 3.6 Hz, 1H), |
| | | 360.1 | 4.21 (q, J = 7.2 Hz, 2H), 3.63 (brs, 4H), 2.07 (brs, |
| | | (M + 1) | 4H), 1.42 (t, J = 7.2 Hz, 3H). |
| B-96 | 83.8%/ | 443.46 for | δ: 9.32 (s, 1H), 8.73 (d, J = 1.8 Hz, 1H), 8.42 (d, J = |
| | 99.1% | C22H23F2N5O3/ | 7.8 Hz, 2H), 8.24 (s, 1H), 8.08 (d, J = 3.6 Hz, 1H), |
| | | 444.2 | 6.84 (d, J = 3.6 Hz, 1H), 4.16 (q, J = 7.2 Hz, 2H), |
| | | (M + 1) | 3.64 (brs, 4H), 2.07 (brs, 4H), 1.27 (t, J = 7.2 Hz, |
| | | | 3H). |
| B-97 | 12.3%/ | 467.48 for | δ: 9.34 (d, J = 1.8, 1H), 8.44 (d, J = 1.8 Hz, 1H), 8.24 |
| | 99.7% | C23H23F2N7O2/ | (d, J = 2.1 Hz, 1H), 8.06 (m, 2H), 7.67 (d, J = 9.6 Hz, |
| | | 468.2 | 1H), 6.84 (d, J = 3.9 Hz, 1H), 3.73 (m, 4H), 3.64 (brs, |
| | | (M + 1) | 4H), 3.5 (m, 4H). 2.07 (m, 4H) |
| B-98 | 3.8%/ | 408.41 for | δ: 9.41 (s, 1H), 8.46-8.45 (d, J = 1.0 Hz, 1H), 8.30 |
| | 98.6% | C21H18F2N6O/ | (s, 1H), 8.25-8.24 (d, J = 3.0 Hz, 1H), 8.16-8.13 |
| | | 409.2 | (m, 2H), 8.07-8.04 (m, 2H), 6.86-6.85 (d, J = 3.0 Hz, |
| | | (M + 1) | 1H), 3.64 (s, 4H), 2.08 (s, 4H). |
| B-99 | 59%/ | 397.39 for | δ/ppm 8.72-8.70 (d, J = 6 Hz, 1H), 8.50-8.49 (d, |
| | 96.5% | C19H17F2N7O/ | J = 3 Hz, 1H), 8.27-8.24 (m, 2H), 8.07 (s, 1H), 7.33- |
| | | 398.2 | 7.31 (d, J = 6 Hz, 1H), 6.88-6.87 (d, J = 3 Hz, 1H), |
| | | (M + 1) | 6.11 (s, 2H), 3.64 (s, 4H), 2.08 (s, 4H). |
| B-100 | 41.7%/ | 391.40 for | δ: 8.67 (s, 1H), 8.37 (d, J = 2.1Hz 1H), 8.25 (s, 1H), |
| | 95.7% | C19H20F3N5O/ | 8.11 (d, J = 0.9 Hz, 1H), 8.06 (d, J = 3.6 Hz, 1H), |
| | | 392.2 | 6.78 (d, J = 3.6 Hz, 1H), 5.27 (d, J = 9.1 Hz, 1H), |
| | | (M + 1) | 4.33 (s, 1H), 3.58 (s, 1H), 2.97 (s, 1H). 1.79 (d, J = |
| | | | 1.5 Hz, 1H), 1.62 (s, 1H), 1.46 (d, J = 12.3 Hz, 1H), |
| | | | 1.16 (dd, J = 13.8, 12.4 Hz, 1H), 0.85 (d, J = 20.1 Hz, |
| | | | 3H). |
| B-101 | 52.1%/ | 407.43 for | δ: 8.58(d, J = 2.4 Hz, 1H), 8.44 (d, J = 1.8 Hz, 1H), |
| | 99.3% | C22H19F2N5O/ | 8.23 (d, J = 2.1Hz, 1H), 8.12 (d, J = 3.9 Hz, 1H), 8.04 |
| | | 408.2 | (s, 4H), 7.79 (d, J = 1.5 Hz, 1H), 6.84 (d, J = 3.6 Hz, |
| | | (M + 1) | 1H), 6.59 (t, J = 2.1 Hz, 1H), 3.65 (brs, 4H), 2.08 |
| | | | (brs, 4H). |
| B-102 | 23.5%/ | 425.44 for | δ: 9.3 (d, J = 1.5 Hz, 1H), 8.44 (d, J = 1.8 Hz, 1H), |
| | 97.5% | C21H21F2N7O/ | 8.24 (d, J = 2.1 Hz, 1H), 8.08 (d, J = 3.6 Hz, 1H), 8.0 |
| | | 426.1 | (dd, J = 9.3, 2.1 Hz, 1H), 7.61 (d, J = 9.3 Hz, 1H), |
| | | (M + 1) | 6.84 (d, J = 3.6, 1H), 3.65 (brs, 4H), 3.33 (s, 6H). |
| | | | 2.05 (m, 4H) |
| B-103 | 27.9%/ | 395.41 for | δ: 9.05 (s, 1H), 8.43 (d, J = 1.5 Hz 1H), 8.23 (d, J = |
| | 94.9% | C21H19F2N5O/ | 1.5 Hz, 1H), 8.02 (d, J = 3.3 Hz, 1H), 7.81 (s, 1H), |
| | | 396.2 | 7.62 (s, 2H), 6.83 (d, J = 3.3 Hz, 1H), 3.64 (brs, 4H), |
| | | (M + 1) | 2.38 (s, 3H), 2.08 (m, 4H). |
| B-104 | 91.2%/ | 385.38 for | δ: 9.27 (d, J = 2.4 Hz, 1H), 8.62 (dd, J = 8.4, 2.4 Hz, |
| | 98.2% | C19H17F2N5O2/ | 1H), 8.48 (d, J = 1.8 Hz, 1H), 8.22 (m, 4H), 7.72 (s, |
| | | 386.2 | 1H), 6.92 (d, J = 3.6 Hz, 1H), 3.64 (brs, 4H), 2.08 |
| | | (M + 1) | (m, 4H). |
| B-105 | 63.6%/ | 367.36 for | δ: 9.50 (d, J = 2.4 Hz, 1H), 8.79 (dd, J = 8.7, 2.7 Hz, |
| | 96.9% | C19H15F2N5O/ | 1H), 8.5 (d, J = 1.8 Hz, 1H), 8.32 (d, J = 3.6 Hz, 1H), |
| | | 368.2 | 8.27 (m, 2H), 6.95 (d, J = 3.6 Hz, 1H), 3.64 (brs, 4H), |
| | | (M + 1) | 2.08 (m, 4H). |
| B-106 | n.a./ | 438.44 for | δ: 8.43 (d, J = 1.8 Hz, 1H), 8.23 (d, J = 1.5, 1H), |
| | 98.1% | C23H20F2N4O3/43 | 8.106 (d, J = 3.3 Hz, 1H), 8.02 (d, J = 8.7 Hz, 2H), |
| | | 9.2 (M + 1) | 7.46 (d, J = 3.6 Hz, 2H), 6.84 (d, J = 3.6 Hz, 1H), |
| | | | 3.64 (brs, 4H), 2.82 (s, 4H), 2.08 (brs, 4H). |
| B-107 | 19.5%/ | 408.41 for | δ: 9.31 (bs, 2H), 8.45 (s, 1H), 8.24 (s, 1H), 8.15- |
| | 97.4% | C21H18F2N6O/ | 8.13 (m, 3H), 9.72 (d, J = 8.4 Hz, 2H), 6.86 (d, J = |
| | | 409.1 | 3.3 Hz, 1H), 3.65 (br s, 4H), 2.08 (br s, 4H). |
| | | (M + 1) | |
| B-108 | 39.3%/ | 403.39 for | δ: 9.3 (d, J = 1.5 Hz, 1H), 8.36 (d, J = 1.8 Hz 1H), |
| | 94.4% | C22H25N7O/ | 8.15 (d, J = 1.8 Hz, 1H), 8.03 (m, 2H), 7.61 (d, J = |
| | | 404.2 | 9.3 Hz, 1H), 6.83 (d, J = 3.6 Hz, 1H), 4.34 (brs, 1H), |
| | | (M + 1) | 3.62 (brs, 1H), 3.07 (s, 6H). 1.63 (m, 5H), 1.15 (m, |
| | | | 2H), 0.86 (m, 3H) |
| B-109 | 69.1%/ | 391.39 for | δ: 13.47 (brs, 1H), 8.51 (m, 2H), 8.38 (d, J = 1.5 Hz, |
| | 99.4% | C22H21F2N5O3/ | 1H), 8.22 (m, 2H), 6.81 (d, J = 3.6 Hz, 1H), 3.64 (brs, |
| | | 392.1 | 4H), 2.06 (m, 4H). |
| | | (M + 1) | |
| B-110 | 33.6%/ | 441.44 for | δ: 8.89 (d, J = 2.4 Hz, 1H), 8.9-8.36 (m, 2H), 8.27- |
| | 99.6% | C22H21F2N5O3/ | 8.23 (m, 2H), 8.09 (d, J = 3.6 Hz, 1H), 8.1 (d, J = 3.6 |
| | | 442.1 | Hz, 1H), 6.85 (d, J = 3.6 Hz, 1H), 4.52 (t, J = 7.8 Hz, |
| | | (M + 1) | 2H), 4.24 (t, J = 8.1, 2H), 3.03 (brs, 1H), 2.6-2.12 |
| | | | (m, 6H), 0.95(brs, 3H). |
| B-112 | 30.5%/ | 414.43 for | δ: 14.16 (brs, 1H), 8.51 (d, J = 2.1, 2H), 8.26 (m, 2H), |
| | 99.9% | C19H16F2N6OS/ | 7.94 (d, J = 1.5 Hz, 1H), 7.84 (s, 1H), 6.87 (d, J = 3.6 |
| | | 415.0 | Hz, 1H), 3.65 (brs, 4H), 2.08 (m, 4H). |
| | | (M + 1) | |
| B-113 | 27.9%/ | 419.49 for | δ: 8.9 (d, J = 2.4 Hz, 1H), 8.4-8.34 (m, 2H), 8.25 (d, |
| | 99.5% | C23H25N5O3/ | J = 9.0 Hz, 1H), 8.14 (d, J = 1.5 Hz, 1H), 8.07 (d, |
| | | 420.4 | J = 3.6 Hz, 1H), 6.84 (d, J = 3.6 Hz, 1H), 4.50 (t, J = |
| | | (M + 1) | 7.8 Hz, 2H), 4.24 (t, J = 8.1, 2H), 3.64 (brs, 1H), 3.05 |
| | | | (brs, 1H), 1.84-1.83 (m, 1 H), 1.64 (s, 1H), 1.42-1.35 |
| | | | (m, 3H), 1.23-1.064 (m, 4H), 0.91-0.74 (m, 3H). |
| B-114 | 54.4%/ | 459.43 for | δ: 8.89 (d, J = 2.7 Hz, 1H), 8.4-8.36 (m, 2H), 8.23 (d, |
| | 99.5% | C22H20F3N5O3/ | J = 9.3 Hz, 1H), 8.19 (s, 1H), 8.08 (d, J = 3.6 Hz, |
| | | 460.2 | 1H), 6.86 (d, J = 3.6 Hz, 1H), 4.51 (t, J = 7.8 Hz, |
| | | (M + 1) | 2H), 4.25 (t, J = 8.1, 2H), 3.6 (brs, 1H), 3.08 (brs, |
| | | | 2H), 2.70-2.67 (m, 2H), 2.01-1.98 (m, 1H), 1.73- |
| | | | 1.56 (m, 3H). |
| B-115 | 62.6%/ | 427.41 for | δ: 8.89 (d, J = 2.7 Hz, 1H), 8.4-8.36 (m, 2H), 8.21 (d, |
| | 92.4% | C21H19F2N5O3/ | J = 9.0 Hz, 1H), 8.18 (d, J = 2.1 Hz, 1H), 8.1 (d, J = |
| | | 428.1 | 3.6 Hz, 1H), 6.86 (d, J = 3.6 Hz, 1H), 4.51 (t, J = 7.8 |
| | | (M + 1) | Hz, 2H), 4.24 (t, J = 8.1, 2H), 3.85 (brs, 2H), 3.71 |
| | | | (brs, 2H), 2.17-208 (m, 2H), 1.73(brs, 2H). |
| B-116 | 55.2%/ | 390.41 for | δ: 8.5 (d, J = 1.8 Hz, 1H), 8.24 (d, J = 1.8 Hz, 1H), |
| | 97.7 | C18H16F2N4O2S/ | 8.17 (d, J = 3.6 Hz, 1H), 7.97 (d, J = 0.9 Hz, 1H), |
| | | 391.1 | 7.85 (brs, 1H), 7.8 (d, J = 1.2, 1H), 7.33 (s, 1H), 6.86 |
| | | (M + 1) | (d, J = 3.6, 1H), 3.64 (brs, 4H), 2.06 (m, 4H). |
| B-117 | 84.5%/ | 391.39 for | δ: 12.9 (brs, 1H), 8.5 (d, J = 1.8 Hz, 1H), 8.28 (d, J = |
| | 95.9% | C18H15F2N3O3S/ | 3.6 Hz, 1H), 8.23 (d, J = 2.1, 1H), 8.08 (d, J = 1.5 |
| | | 392.1 | Hz, 1H), 7.79 (d, J = 1.5 Hz, 1 H), 6.86 (d, J = 3.6 |
| | | (M + 1) | Hz, 1H), 3.63 (brs, 4H), 2.05 (m, 4H). |
| B-118 | 84%/ | 391.39 for | δ: 13.07 (s, 1H), 8.54 (d, J = 1.8 Hz, 1H), 8.34 (d, |
| | 91.1% | C18H15F2N3O3S/ | J = 3.9 Hz, 1H), 8.25 (d, J = 2.1 Hz, 1H), 7.71 (d, J = |
| | | 392.1 | 4.2 Hz, 1H), 7.58 (d, J = 4.2 Hz, 1H), 6.91 (d, J = 3.9 |
| | | (M + 1) | Hz, 1H), 3.63 (br s, 4H), 2.08 (br s, 4H). |
| B-119 | 60%/ | 414.43 for | δ: 14.21 (s, 1H), 8.53 (d, J = 1.8 Hz, 2H), 8.28-8.24 |
| | 98.4% | C19H16F2N6OS/ | (m, 2H), 7.53 (t, J = 9.9 Hz, 2H), 6.89 (d, J = 3.6 Hz, |
| | | 415.1 | 1H), 3.65 (br s, 4H), 2.1-2.03 (br m, 4H). |
| | | (M + 1) | |
| B-120 | 30.9% | 396.40 for | δ: 9.52 (d, J = 1.5 Hz, 1H), 8.46 (d, J = 1.8 Hz 1H), |
| | 99.7% | C20H18F2N6O/ | 8.26 (d, J = 1.8 Hz, 1H), 8.20 (dd, J = 9.6, 2.1 Hz, |
| | | 398.1 | 1H), 8.14 (d, J = 3.6 Hz, 1H), 7.91 (d, J = 9.6 Hz, |
| | | (M + 1) | 1H), 6.87 (d, J = 3.9 Hz, 1H), 3.65 (brs, 4H), 2.1 (brs, |
| | | | 4H). |
| B-121 | 81.8%/ | 390.41 for | δ: 8.52 (s, 1H), 8.24 (t, J = 6.9 Hz, 2H), 8.00 (s, 1H), |
| | 94.2% | C18H16F2N4O2S/ | 7.71 (d, J = 4.2 Hz, 1H), 7.51 (d, J = 4.2 Hz, 1H), |
| | | 391.1 | 7.40 (s, 1H), 6.89 (d, J = 3.6 Hz, 1H), 3.63 (br s, 4H), |
| | | (M + 1) | 2.08 (br s, 4H), |
| B-122 | 70%/ | 372.39 for | δ: 8.56 (d, J = 1.8 Hz, 1H), 8.44 (d, J = 3.9 Hz, 1H), |
| | 97.1% | C18H14F2N4OS/ | 8.28 (d, J = 1.8 Hz, 1H), 7.99 (d, J = 4.2 Hz, 1H), |
| | | 373.1 | 7.68 (d, J = 4.2 Hz, 1H), 6.96 (d, J = 3.9 Hz, 1H), |
| | | (M + 1) | 3.64 (br s, 4H), 2.08 (br s, 4H). |
| B-123 | 48.3%/ | 414.43 for | δ: 14.24 (brs, 1H), 8.67 (s, 1H), 8.50 (s, 1H), 8.41 (s, |
| | 98.0% | C19H16F2N6OS/ | 1H), 8.23 (s, 2H), 8.1 (s, 1H), 6.81 (s, 1H), 3.64 (brs, |
| | | 415.3 | 4H), 2.08 (m, 4H). |
| | | (M + 1) | |
| B-124 | 85.4%/ | 390.41 for | δ: 8.47 (d, J = 2.1, 1H), 8.4 (d, J = 1.5 Hz, 1H), 8.22 |
| | 98.5% | C18H16F2N4O2S/ | (m, 2H), 8.11 (brs, 1H), 8.01 (d, J = 3.6, 1H), 7.59 |
| | | 391.2 | (brs, 1H), 6.82 (d, J = 3.6, 1 H), 3.64 (brs, 4 H), 2.07 |
| | | (M + 1) | (m, 4H). |
| B-125 | 58.0%/ | 372.39 for | δ: 8.69 (d, J = 1.5 Hz, 1H), 8.64 (d, J = 1.5 Hz 1H), |
| | 99.5% | C18H14F2N4OS/ | 8.49 (d, J = 2.1 Hz, 1H), 8.24 (dd, J = 2.1 Hz, 1H), |
| | | 373.1 | 8.18 (d, J = 3.6 Hz, 1H), 6.84 (d, J = 3.6 Hz, 1H), |
| | | (M + 1) | 3.64 (brs, 4H), 2.1 (m, 4H). |
| B-128 | n.a./ | 435.53 for | δ = 10.06 (s, 1H), 8.70 (d, J = 2.4 Hz, 1H), 8.62 (s, |
| | 95.9% | C24H29N5O3/ | 1H), 8.52 (s, 1H), 8.35 (d, J = 2.0 Hz, 1H), 8.15 (d, |
| | | 436.3 | J = 1.6 Hz, 1H), 8.06 (d, J = 4.0 Hz, 1H), 6.85 (d, J = |
| | | (M + 1) | 3.6 Hz, 1H), 4.93 (td, J = 6.4, 12.4 Hz, 1H), 4.55- |
| | | | 4.15 (m, 1H), 3.77-3.52 (m, 1H), 1.94-1.76 (m, |
| | | | 2H), 1.72-1.57 (m, 1H), 1.53-1.38 (m, 2H), 1.28 |
| | | | (d, J = 6.4 Hz, 6H), 1.23-1.08 (m, 3H), 1.00-0.57 |
| | | | (m, 3H). |
| B-129 | 39%/ | 435.53 for | δ = 10.06 (s, 1H), 8.70 (d, J = 2.4 Hz, 1H), 8.62 (d, |
| | 99.9% | C24H29N5O3/ | J = 2.0 Hz, 1H), 8.52 (t, J = 2.4 Hz, 1H), 8.35 (d, J = |
| | | 436.2 | 2.0 Hz, 1H), 8.15 (d, J = 2.0 Hz, 1H), 8.06 (d, J = 3.6 |
| | | (M + 1) | Hz, 1H), 6.85 (d, J = 3.6 Hz, 1H), 5.09-4.80 (m, |
| | | | 1H), 4.55-4.10 (m, 1H), 3.98-3.43 (m, 1H), 3.13- |
| | | | 2.74 (m, 1H), 1.86 (br dd, J = 4.0, 9.6 Hz, 1H), 1.74- |
| | | | 1.56 (m, 1H), 1.52-1.36 (m, 2H), 1.28 (d, J = 6.4 |
| | | | Hz, 6H), 1.25-1.09 (m, 3H), 1.08-0.48 (m, 3H). |
| B-130 | 74%/ | 389.38 for | δ = 8.37 (d, J = 1.6 Hz, 1H), 8.16 (d, J = 2.0 Hz, 2H), |
| | 99% | C19H18F3N5O/ | 8.00 (d, J = 3.6 Hz, 1H), 7.94 (d, J = 2.4 Hz, 1H), |
| | | 390.1 | 7.49 (t, J = 2.4 Hz, 1H), 6.81 (d, J = 3.6 Hz, 1H), 5.65 |
| | | (M + 1) | (s, 2H), 4.67-4.36 (m, 1H), 3.77-3.57 (m, 1H), 3.19- |
| | | | 2.91 (m, 2H), 2.67 (br dd, J = 1.8, 3.6 Hz, 1H), 2.05- |
| | | | 1.95 (m, 1H), 1.78-1.48 (m, 3H). |
| B-131 | 32%/ | 349.44 for | δ = 8.33 (d, J = 2.0 Hz, 1H), 8.16 (s, 1H), 8.13 (br s, |
| | 99% | C20H23N5O/ | 1H), 8.11 (d, J = 2.0 Hz, 1H), 7.98 (d, J = 3.6 Hz, |
| | | 350.1 | 1H), 7.93 (br s, 1H), 7.50 (t, J = 2.4 Hz, 1H), 6.79 (d, |
| | | (M + 1) | J = 3.6 Hz, 1H), 5.63 (br s, 1H), 4.65-3.95 (m, 1H), |
| | | | 3.92-3.41 (m, 1H), 3.12-2.69 (m, 1H), 1.85 (br dd, |
| | | | J = 3.2, 10.8 Hz, 1H), 1.79-1.56 (m, 1H), 1.55-1.39 |
| | | | (m, 2H), 1.32-1.02 (m, 3H), 1.01-0.62 (m, 3H). |
| B-132 | 43%/ | 421.50 for | δ = 10.13 (s, 1H), 8.71 (d, J = 2.0 Hz, 1H), 8.62 (d, |
| | 99% | C23H27N5O3/ | J = 2.0 Hz, 1H), 8.57-8.50 (m, 1H), 8.35 (d, J = 2.0 |
| | | 422.2 | Hz, 1H), 8.15 (d, J = 2.0 Hz, 1H), 8.06 (d, J = 3.6 Hz, |
| | | (M + 1) | 1H), 6.85 (d, J = 3.6 Hz, 1H), 4.51-4.25 (m, 1H), |
| | | | 4.18 (q, J = 7.2 Hz, 2H), 3.79-3.51 (m, 1H), 3.16- |
| | | | 2.70 (m, 2H), 1.94-1.78 (m, 1H), 1.76-1.55 (m, |
| | | | 1H), 1.51-1.38 (m, 2H), 1.36-1.07 (m, 6H), 1.04- |
| | | | 0.63 (m, 3H). |
| B-133 | 51%/ | 421.50 for | δ = 10.12 (s, 1H), 8.71 (d, J = 2.4 Hz, 1H), 8.63 (d, J = |
| | 99% | C23H27N5O3/ | 2.0 Hz, 1H), 8.53 (s, 1H), 8.35 (d, J = 2.0 Hz, 1H), |
| | | 422.1 | 8.15 (d, J = 2.0 Hz, 1H), 8.06 (d, J = 3.6 Hz, 1H), |
| | | (M + 1) | 6.85 (d, J = 3.6 Hz, 1H), 4.51-4.24 (m, 1H), 4.18 (q, |
| | | | J = 7.2 Hz, 2H), 3.75-3.40 (m, 1H), 3.14-2.57 (m, |
| | | | 2H), 1.96-1.82 (m, 1H), 1.76-1.56 (m, 1H), 1.51- |
| | | | 1.36 (m, 2H), 1.27 (t, J = 7.2 Hz, 3H), 1.24-0.99 (m, |
| | | | 3H), 0.98-0.56 (m, 3H). |
| B-134 | 29%/ | 417.51 for | δ = 10.67 (br s, 1H), 8.75 (dd, J = 2.0, 10.4 Hz, 2H), |
| | 99% | C24H27N5O2/ | 8.65 (br d, J = 1.6 Hz, 1H), 8.39-8.29 (m, 1H), 8.19- |
| | | 418.2 | 8.11 (m, 1H), 8.09-8.04 (m, 1H), 7.02-6.68 (m, |
| | | (M + 1) | 1H), 4.54-4.14 (m, 1H), 3.74-3.51 (m, 1H), 3.09- |
| | | | 2.75 (m, 2H), 1.93-1.77 (m, 2H), 1.71-1.55 (m, |
| | | | 1H), 1.50-1.39 (m, 2H), 1.27-1.01 (m, 3H), 0.89- |
| | | | 0.73 (m, 7H). |
| B-135 | | 417.51 for | δ = 10.68 (s, 1H), 8.76 (dd, J = 2.0, 10.4 Hz, 2H), |
| | | C24H27N5O2/ | 8.68-8.63 (m, 1H), 8.35 (d, J = 2.0 Hz, 1H), 8.15 (d, |
| | | 418.1 | J = 2.0 Hz, 1H), 8.08 (d, J = 3.6 Hz, 1H), 6.85 (d, |
| | | (M + 1) | J = 3.6 Hz, 1H), 4.66-4.05 (m, 1H), 3.89-3.48 (m, |
| | | | 1H), 3.14-2.65 (m, 2H), 1.92-1.76 (m, 2H), 1.75- |
| | | | 1.55 (m, 1H), 1.50-1.39 (m, 2H), 1.34-1.06 (m, |
| | | | 3H), 0.83 (br s, 7H). |
| B-136 | 38%/ | 349.44 for | δ = 8.33 (d, J = 2.0 Hz, 1H), 8.18 (s, 1H), 8.12 (d, |
| | 98% | C20H23N5O/ | J = 2.0 Hz, 1H), 7.99 (d, J = 3.6 Hz, 1H), 7.93 (s, 1H), |
| | | 350.1 | 7.52 (t, J = 2.4 Hz, 1H), 6.80 (d, J = 3.6 Hz, 1H), |
| | | (M + 1) | 5.79-5.58 (m, 1H), 4.55-4.03 (m, 1H), 3.61 (br s, 1H), |
| | | | 3.08-2.61 (m, 2H), 1.96-1.77 (m, 1H), 1.75-1.53 |
| | | | (m, 1H), 1.41 (td, J = 3.6, 6.8 Hz, 2H), 1.32-1.03 |
| | | | (m, 3H), 1.00-0.73 (m, 3H). |
| B-137 | 38%/ | 402.46 for | δ = 14.26 (br s, 1H), 8.52 (br s, 1H), 8.41 (br s, 1H), |
| | 99% | C22H22N6O2/ | 8.26-8.16 (m, 3H), 8.15-8.01 (m, 3H), 6.84 (d, J = |
| | | 403.1 | 3.6 Hz, 1H), 4.69-3.94 (m, 1H), 3.70-3.43 (m, 1H), |
| | | (M + 1) | 3.26-2.99 (m, 2H), 1.88-1.69 (m, 1H), 1.67-1.40 |
| | | | (m, 3H), 1.30-0.88 (m, 3H). |
| B-138 | 92%/ | 402.46 for | δ = 14.73-13.64 (m, 1H), 8.81-8.36 (m, 2H), 8.31 |
| | 99.6% | C22H22N6O2/ | (br d, J = 2.8 Hz, 1H), 8.19 (d, J = 8.4 Hz, 2H), 8.15- |
| | | 403.1 | 7.99 (m, 3H), 6.84 (br d, J = 2.8 Hz, 1H), 4.19- |
| | | | 3.98 (m, 1H), 3.78-3.34 (m, 6H), 2.08-1.85 (m, |
| | | | 2H), 1.21-0.96 (m, 3H). |
| B-139 | 64%/ | 448.48 for | δ = 8.46-8.38 (m, 2H), 8.23 (d, J = 2.0 Hz, 1H), 8.09 |
| | 99.1% | C24H22F2N6O/ | (d, J = 3.6 Hz, 1H), 7.95 (d, J = 7.6 Hz, 1H), 7.88 (br |
| | | 449.1 | d, J = 7.6 Hz, 1H), 7.64 (t, J = 7.6 Hz, 1H), 6.84 (d, |
| | | (M + 1) | J = 3.6 Hz, 1H), 4.11-3.99 (m, 2H), 2.20-1.96 (m, |
| | | | 5H), 1.09-1.01 (m, 2H), 1.00-0.90 (m, 2H). |
| B-140 | 58%/ | 445.53 for | δ = 8.76 (d, J = 7.6 Hz, 1H), 8.41 (d, J = 1.6 Hz, 1H), |
| | 99% | C24H27N7O2/ | 8.27-8.04 (m, 3H), 7.80 (dd, J = 2.4, 7.2 Hz, 1H), |
| | | 446.2 | 6.88 (d, J = 3.6 Hz, 1H), 3.95 (br dd, J = 3.6, 8.4 Hz, |
| | | (M + 1) | 2H), 3.74 (t, J = 4.8 Hz, 4H), 3.52 (t, J = 4.8 Hz, 4H), |
| | | | 3.04-2.95 (m, 1H), 2.72 (br d, J = 11.2 Hz, 1H), 1.86- |
| | | | 1.78 (m, 1H), 1.74-1.59 (m, 2H), 1.55-1.44 (m, |
| | | | 1H), 1.27-1.18 (m, 1H), 0.87 (br d, J = 6.4 Hz, 3H). |
| B-141 | 30%/ | 402.46 for | δ = 14.25 (br s, 1H), 8.53 (d, J = 2.0 Hz, 2H), 8.31 |
| | 99% | C22H22N6O2/ | (br d, J = 2.8 Hz, 1H), 8.19 (d, J = 8.8 Hz, 2H), 8.12 |
| | | 403.2 | (d, J = 4.0 Hz, 1H), 8.08 (br d, J = 8.4 Hz, 2H), 6.84 |
| | | (M + 1) | (d, J = 2.4 Hz, 1H), 4.24-3.96 (m, 1H), 3.81-3.35 |
| | | | (m, 6H), 2.10-1.86 (m, 2H), 1.25-1.03 (m, 3H). |
| B-142 | 46%/ | 423.47 for | δ = 10.12 (br s, 1H), 8.78-8.58 (m, 2H), 8.51 (br d, |
| | 99% | C22H25N5O4/ | J = 10.0 Hz, 2H), 8.31 (br d, J = 2.4 Hz, 1H), 8.06 |
| | | 424.1 | (br d, J = 2.4 Hz, 1H), 6.85 (br d, J = 1.6 Hz, 1H), |
| | | (M + 1) | 4.18 (q, J = 7.2 Hz, 2H), 4.14-4.01 (m, 1H), 3.79- |
| | | | 3.34 (m, 6H), 2.09-1.87 (m, 2H), 1.27 (t, J = 7.2 Hz, |
| | | | 3H), 1.19-0.99 (m, 3H). |
| B-143 | 41%/ | 423.47 for | δ = 10.12 (s, 1H), 8.80-8.55 (m, 2H), 8.55-8.44 |
| | 99% | C22H25N5O4/ | (m, 2H), 8.31 (br d, J = 2.4 Hz, 1H), 8.06 (br d, J = |
| | | 424.1 | 3.2 Hz, 1H), 6.85 (br d, J = 2.0 Hz, 1H), 4.18 (q, J = |
| | | (M + 1) | 7.2 Hz, 2H), 4.14-4.00 (m, 1H), 3.79-3.35 (m, 6H), |
| | | | 2.09-1.88 (m, 2H), 1.27 (t, J = 6.8 Hz, 3H), 1.18- |
| | | | 1.00 (m, 3H). |
| B-144 | 37%/ | 402.46 for | δ = 8.38 (d, J = 2.0 Hz, 1H), 8.27 (s, 1H), 8.25-8.20 |
| | 99.2% | C22H22N6O2/ | (m, 2H), 8.07 (d, J = 2.0 Hz, 1H), 8.00-7.94 (m, |
| | | 403.1 | 2H), 7.81 (d, J = 3.6 Hz, 1H), 6.77 (d, J = 4.0 Hz, |
| | | (M + 1) | 1H), 4.45-3.88 (m, 1H), 3.76-3.11 (m, 3H), 2.66- |
| | | | 2.44 (m, 1H), 1.59 (br s, 4H), 1.22 (s, 3H). |
| B-145 | 58%/ | 413.35 for | δ = 8.67 (s, 1H), 8.46 (d, J = 2.0 Hz, 1H), 8.26 (s, |
| | 100% | C18H16F5N5O/ | 1H), 8.20 (d, J = 2.0 Hz, 1H), 8.07 (d, J = 3.7 Hz, |
| | | 414.2 | 1H), 6.79 (d, J = 3.6 Hz, 1H), 5.27 (q, J = 9.2 Hz, |
| | | (M + 1) | 2H), 3.64 (br s, 4H), 2.13-2.01 (m, 4H) |
| B-146 | 54%/ | 467.48 for | δ = 8.85 (d, J = 7.2 Hz, 1H), 8.44 (d, J = 2.0 Hz, 1H), |
| | 99% | C23H23F2N7O2/ | 8.30 (d, J = 4.0 Hz, 1H), 8.24 (d, J = 2.0 Hz, 1H), |
| | | 468.1 | 8.20 (d, J = 2.0 Hz, 1H), 7.85 (dd, J = 2.4, 7.2 Hz, |
| | | (M + 1) | 1H), 6.92 (d, J = 3.6 Hz, 1H), 3.99-3.53 (m, 8H), |
| | | | 3.52-3.48 (m, 4H), 2.21-2.05 (m, 2H), 1.75 (br s, |
| | | | 2H). |
| B-147 | 71%/ | 467.48 for | δ = 8.84 (d, J = 7.2 Hz, 1H), 8.50 (d, J = 2.0 Hz, 1H), |
| | 97% | C23H23F2N7O2/ | 8.28 (d, J = 4.0 Hz, 1H), 8.25 (d, J = 2.0 Hz, 1H), |
| | | 468.1 | 8.24 (d, J = 2.0 Hz, 1H), 7.85 (dd, J = 2.4, 7.2 Hz, |
| | | (M + 1) | 1H), 6.90 (d, J = 4.0 Hz, 1H), 3.86-3.54 (m, 8H), |
| | | | 3.52-3.47 (m, 4H), 2.14-2.03 (m, 4H). |
| B-148 | 20%/ | 428.44 for | δ = 8.76 (s, 1H), 8.68 (d, J = 2.0 Hz, 1H), 8.63 (d, J = |
| | 96% | C21H22F2N6O2/ | 2.0 Hz, 1H), 8.50 (br d, J = 2.0 Hz, 1H), 8.43 (d, J = |
| | | 429.2 | 1.6 Hz, 1H), 8.24 (d, J = 2.0 Hz, 1H), 8.05 (d, J = |
| | | (M + 1) | 3.6 Hz, 1H), 6.85 (d, J = 3.6 Hz, 1H), 3.65 (br d, J = |
| | | | 7.2 Hz, 4H), 2.97 (s, 6H), 2.14-2.00 (m, 4H). |
| B-149 | 27%/ | 454.48 for | δ = 8.72 (d, J = 2.0 Hz, 1H), 8.66-8.58 (m, 2H), 8.54 |
| | 96% | C23H24F2N6O2/ | (t, J = 2.0 Hz, 1H), 8.43 (d, J = 2.0 Hz, 1H), 8.24 (d, |
| | | 455.2 | J = 2.0 Hz, 1H), 8.05 (d, J = 3.6 Hz, 1H), 6.85 (d, J = |
| | | (M + 1) | 3.6 Hz, 1H), 3.73-3.56 (m, 4H), 3.41 (br t, J = 6.4 |
| | | | Hz, 4H), 2.08 (br d, J = 5.6 Hz, 4H), 1.88 (br s, 4H). |
| B-150 | 17%/ | 481.51 for | δ = 9.32 (d, J = 2.0 Hz, 1H), 8.44 (d, J = 2.0 Hz, 1H), |
| | 99.3% | C24H25F2N7O2/ | 8.24 (d, J = 2.0 Hz, 1H), 8.08 (d, J = 3.6 Hz, 1H), |
| | | 482.2 | 8.03 (dd, J = 2.0, 9.2 Hz, 1H), 7.65 (d, J = 9.2 Hz, |
| | | (M + 1) | 1H), 6.84 (d, J = 3.6 Hz, 1H), 4.26-4.08 (m, 1H), |
| | | | 3.92 (dd, J = 3.2, 11.6 Hz, 1H), 3.74 (br dd, J = 2.0, |
| | | | 13.2 Hz, 2H), 3.71-3.59 (m, 4H), 3.55-3.55 (m, |
| | | | 1H), 3.31-3.24 (m, 2H), 2.14-2.02 (m, 4H), 1.23 |
| | | | (d, J = 6.8 Hz, 3H). |
| B-151 | 17%/ | 481.51 for | δ = 9.32 (d, J = 1.6 Hz, 1H), 8.44 (d, J = 2.0 Hz, 1H), |
| | 99.8% | C24H25F2N7O2/ | 8.24 (d, J = 2.0 Hz, 1H), 8.08 (d, J = 3.6 Hz, 1H), |
| | | 482.1 | 8.06-8.00 (m, 1H), 7.65 (d, J = 9.2 Hz, 1H), 6.84 (d, |
| | | (M + 1) | J = 3.6 Hz, 1H), 4.18 (q, J = 6.8 Hz, 1H), 3.92 (dd, J = |
| | | | 3.2, 11.2 Hz, 1H), 3.72 (br d, J = 2.4 Hz, 2H), 3.71- |
| | | | 3.59 (m, 4H), 3.58-3.51 (m, 1H), 3.31-3.21 (m, |
| | | | 2H), 2.13-2.02 (m, 4H), 1.23 (d, J = 6.8 Hz, 3H). |
| B-152 | 53%/ | 425.48 for | δ = 8.60 (br d, J = 5.2 Hz, 1H), 8.49 (d, J = 2.0 Hz, |
| | 99.3% | C23H25F2N5O/ | 1H), 8.27-8.26 (m, 1H), 8.25-8.24 (m, 1H), 8.20 |
| | | 426.2 | (s, 1H), 8.02 (br d, J = 3.6 Hz, 1H), 6.89 (d, J = 4.0 |
| | | (M + 1) | Hz, 1H), 3.80 (s, 2H), 3.78-3.44 (m, 4H), 2.56 (br |
| | | | s, 4H), 2.09 (br d, J = 5.2 Hz, 4H), 1.73 (br s, 4H). |
| B-153 | 24%/ | 493.52 for | δ = 9.31 (d, J = 1.6 Hz, 1H), 8.42-8.32 (m, 1H), 8.17 |
| | 99.4% | C25H25F2N7O2/ | (d, J = 2.0 Hz, 1H), 8.08 (d, J = 3.6 Hz, 1H), 8.03 |
| | | 494.2 | (dd, J = 2.0, 9.6 Hz, 1H), 7.63 (d, J = 9.6 Hz, 1H), |
| | | (M + 1) | 6.85 (d, J = 3.6 Hz, 1H), 4.43 (br s, 2H), 4.20-3.78 |
| | | | (m, 2H), 3.73 (d, J = 12.0 Hz, 2H), 3.69-3.41 (m, |
| | | | 2H), 3.16 (dd, J = 2.4, 12.4 Hz, 2H), 2.21-2.05 (m, |
| | | | 2H), 1.94-1.79 (m, 4H), 1.78-1.67 (m, 2H). |
| B-154 | 29%/ | 493.52 for | δ = 9.30 (d, J = 2.0 Hz, 1H), 8.44 (d, J = 1.6 Hz, 1H), |
| | 99.8% | C25H25F2N7O2/ | 8.24 (d, J = 1.6 Hz, 1H), 8.07 (d, J = 3.6 Hz, 1H), |
| | | 494.2 | 8.03 (dd, J = 2.0, 9.6 Hz, 1H), 7.63 (d, J = 9.6 Hz, |
| | | (M + 1) | 1H), 6.84 (d, J = 3.6 Hz, 1H), 4.43 (br s, 2H), 3.75 |
| | | | (s, 2H), 3.72-3.43 (m, 4H), 3.16 (dd, J = 2.0, 12.4 |
| | | | Hz, 2H), 2.14-2.01 (m, 4H), 1.90-1.76 (m, 4H). |
| B-155 | 44%/ | 400.49 for | δ = 14.25 (br s, 1H), 8.52 (br s, 1H), 8.38 (d, J = 2.0 |
| | 99% | C23H24N6O/ | Hz, 1H), 8.19 (d, J = 8.8 Hz, 2H), 8.15 (d, J = 2.0 Hz, |
| | | 401.1 | 1H), 8.11 (d, J = 3.6 Hz, 1H), 8.08 (br d, J = 8.4 Hz, |
| | | (M + 1) | 2H), 6.83 (d, J = 3.6 Hz, 1H), 3.86-3.36 (m, 4H), |
| | | | 1.35 (br s, 4H), 0.97 (s, 6H). |
| B-156 | 29%/ | 481.51 for | δ = 9.29 (d, J = 0.8 Hz, 1H), 8.44 (d, J = 1.2 Hz, 1H), |
| | 99.3% | C24H25F2N7O2/ | 8.24 (d, J = 1.2 Hz, 1H), 8.07 (d, J = 3.6 Hz, 1H), |
| | | 482.2 | 7.99 (dd, J = 1.6, 9.6 Hz, 1H), 7.60 (d, J = 9.6 Hz, |
| | | (M + 1) | 1H), 6.83 (d, J = 3.6 Hz, 1H), 3.76 (br s, 8H), 3.67- |
| | | | 3.49 (m, 4H), 2.14-2.01 (m, 4H), 1.93 (quin, J = 5.6 |
| | | | Hz, 2H). |
| B-157 | 26%/ | 370.42 for | δ = 14.72-13.87 (m, 1H), 8.84-8.36 (m, 2H), 8.23 |
| | 99.0% | C21H18N6O/ | (d, J = 2.0 Hz, 1H), 8.21-8.16 (m, 2H), 8.12 (d, J = |
| | | 371.2 | 4.0 Hz, 1H), 8.08 (br d, J = 8.0 Hz, 2H), 6.83 (d, J = |
| | | (M + 1) | 3.6 Hz, 1H), 4.18-3.94 (m, 1H), 3.89-3.68 (m, 1H), |
| | | | 3.43 (br d, J = 10.8 Hz, 2H), 1.79-1.49 (m, 2H), 0.75- |
| | | | 0.58 (m, 1H), 0.14 (q, J = 4.4 Hz, 1H). |
| B-158 | 25%/ | 398.47 for | δ = 14.52-14.00 (m, 1H), 8.78-8.41 (m, 2H), 8.26 |
| | 99% | C23H22N6O/ | (d, J = 2.0 Hz, 1H), 8.19 (d, J = 8.8 Hz, 2H), 8.12 (d, |
| | | 399.2 | J = 3.6 Hz, 1H), 8.08 (br d, J = 8.4 Hz, 2H), 6.84 (d, |
| | | (M + 1) | J = 3.6 Hz, 1H), 3.94-3.63 (m, 2H), 3.50-3.36 (m, |
| | | | 1H), 3.25 (brs, 1H), 2.66 (br dd, J = 2.8, 4.8 Hz, 2H), |
| | | | 1.88-1.65 (m, 3H), 1.60-1.31 (m, 3H). |
| B-159 | 47%/ | 509.56 for | δ = 9.29 (d, J = 1.6 Hz, 1H), 8.44 (d, J = 2.0 Hz, 1H), |
| | 98.6% | C26H29F2N7O2/ | 8.24 (d, J = 2.0 Hz, 1H), 8.07 (d, J = 3.6 Hz, 1H), |
| | | 510.2 | 8.00 (dd, J = 2.0, 9.6 Hz, 1H), 7.61 (d, J = 9.6 Hz, |
| | | (M + 1) | 1H), 6.83 (d, J = 3.6 Hz, 1H), 3.80 (td, J = 4.0, 12.8 |
| | | | Hz, 2H), 3.76-3.36 (m, 4H), 3.32-3.24 (m, 2H), |
| | | | 3.14 (s, 3H), 2.15-2.00 (m, 4H), 1.74 (br d, J = 13.2 |
| | | | Hz, 2H), 1.58-1.46 (m, 2H), 1.13 (s, 3H). |
| B-160 | 77%/ | 498.92 for | δ = 10.18 (s, 1H), 8.46 (d, J = 2.0 Hz, 1H), 8.36 (s, |
| | 99% | C24H21ClF2N6O2/ | 1H), 8.24 (d, J = 2.0 Hz, 2H), 8.09-8.01 (m, 2H), |
| | | 499.3 | 7.94 (d, J = 1.6 Hz, 1H), 7.71 (t, J = 1.8 Hz, 1H), 6.85 |
| | | (M + 1) | (d, J = 3.6 Hz, 1H), 3.91 (s, 3H), 3.77-3.56 (m, 4H), |
| | | | 2.08 (br d, J = 2.9 Hz, 4H). |
| B-161 | 40%/ | 464.48 for | δ = 10.04 (s, 1H), 8.42 (d, J = 2.0 Hz, 1H), 8.34 (s, |
| | 99% | C24H22F2N6O2/ | 1H), 8.26 (s, 1H), 8.23 (d, J = 2.0 Hz, 1H), 8.05 (s, |
| | | 465.3 | 1H), 7.99 (d, J = 3.6 Hz, 1H), 7.75 (td, J = 2.4, 4.5 |
| | | (M + 1) | Hz, 1H), 7.56-7.49 (m, 2H), 6.82 (d, J = 3.6 Hz, |
| | | | 1H), 3.90 (s, 3H), 3.84-3.57 (m, 4H), 2.15-2.07 |
| | | | (m, 4H). |
| B-162 | 71%/ | 357.37 for | δ = 8.35 (d, J = 2.0 Hz, 1H), 8.16 (dd, J = 2.0, 5.0 |
| | 99% | C18H17F2N5O/ | Hz, 2H), 8.01 (d, J = 3.6 Hz, 1H), 7.94 (d, J = 2.4 Hz, |
| | | 358.2 | 1H), 7.49 (t, J = 2.4 Hz, 1H), 6.82 (d, J = 3.6 Hz, 1H), |
| | | (M + 1) | 5.65 (m, 2H), 4.06-3.76 (m, 2H), 3.70-3.46 (m, |
| | | | 2H), 2.21-2.06 (m, 2H), 1.73 (br s, 2H) |
| B-163 | 41%/ | 481.51 for | δ = 9.33 (d, J = 1.2 Hz, 1H), 8.44 (d, J = 1.6 Hz, 1H), |
| | 99.7% | C24H25F2N7O2/ | 8.24 (d, J = 1.6 Hz, 1H), 8.08 (d, J = 3.6 Hz, 1H), |
| | | 482.2 | 8.04 (dd, J = 2.0, 9.6 Hz, 1H), 7.65 (d, J = 9.6 Hz, |
| | | (M + 1) | 1H), 6.84 (d, J = 3.6 Hz, 1H), 4.00 (br d, J = 12.4 Hz, |
| | | | 1H), 3.95-3.84 (m, 2H), 3.81-3.40 (m, 6H), 3.02 |
| | | | (dt, J = 3.2, 12.0 Hz, 1H), 2.70 (dd, J = 10.4, 12.4 |
| | | | Hz, 1H), 2.16-1.99 (m, 4H), 1.16 (d, J = 6.0 Hz, |
| | | | 3H) |
| B-164 | 30.5%/ | 479.49 for | δ = 8.74 (t, J = 5.8 Hz, 1H), 8.63 (br d, J = 5.6 Hz, |
| | 99.2% | C24H23F2N7O2/ | 1H), 8.39 (d, J = 2.0 Hz, 1H), 8.26-8.21 (m, 2H), |
| | | 480.1 | 8.19 (d, J = 1.8 Hz, 1H), 8.17 (s, 1H), 8.03 (dd, J = |
| | | (M + 1) | 2.0, 5.6 Hz, 1H), 7.91 (s, 1H), 6.89 (d, J = 3.6 Hz, |
| | | | 1H), 4.58 (d, J = 5.6 Hz, 2H), 3.86 (s, 3H), 3.82- |
| | | | 3.41 (m, 4H), 2.20-1.96 (m, 4H). |
| B-165 | 40.4%/ | 413.43 for | δ = 8.62 (d, J = 5.6 Hz, 1H), 8.54-8.44 (m, 2H), 8.28- |
| | 98.6% | C21H21F2N5O2/ | 8.23 (m, 2H), 8.12 (d, J = 1.8 Hz, 1H), 8.06 (dd, J = |
| | | 414.1 | 2.0, 5.6 Hz, 1H), 6.91 (d, J = 3.6 Hz, 1H), 4.43 (d, |
| | | (M + 1) | J = 5.6 Hz, 2H), 3.86-3.43 (m, 4H), 2.17-1.99 (m, |
| | | | 4H), 1.93 (s, 3H). |
| B-166 | 28%/ | 495.54 for | δ = 9.29 (d, J = 1.6 Hz, 1H), 8.44 (d, J = 2.0 Hz, 1H), |
| | 99.9% | C25H27F2N7O2/ | 8.24 (d, J = 2.0 Hz, 1H), 8.07 (d, J = 3.6 Hz, 1H), |
| | | 496.2 | 8.01 (dd, J = 2.0, 9.6 Hz, 1H), 7.62 (d, J = 9.6 Hz, |
| | | (M + 1) | 1H), 6.83 (d, J = 3.6 Hz, 1H), 3.94-3.84 (m, 2H), |
| | | | 3.65 (br d, J = 1.2 Hz, 4H), 3.43 (tt, J = 4.0, 8.4 Hz, |
| | | | 1H), 3.28 (s, 3H), 3.28-3.19 (m, 2H), 2.14-2.02 |
| | | | (m, 4H), 1.93 (td, J = 4.4, 8.5 Hz, 2H), 1.54-1.43 |
| | | | (m, 2H). |
| B-167 | 27%/ | 495.54 for | δ = 9.31 (d, J = 2.0 Hz, 1H), 8.44 (d, J = 2.0 Hz, 1H), |
| | 99.9% | C25H27F2N7O2/ | 8.24 (d, J = 2.0 Hz, 1H), 8.08 (d, J = 3.6 Hz, 1H), |
| | | 496.3 | 8.03 (dd, J = 2.0, 9.6 Hz, 1H), 7.63 (d, J = 9.6 Hz, |
| | | (M + 1) | 1H), 6.84 (d, J = 3.6 Hz, 1H), 3.81-3.74 (m, 2H), |
| | | | 3.74-3.51 (m, 4H), 3.51-3.45 (m, 2H), 3.36 (s, 2H), |
| | | | 2.17-1.98 (m, 4H), 1.22 (s, 6H). |
| B-168 | 63%/ | 476.14 for | δ = 8.53 (d, J = 1.6 Hz, 1H), 8.46 (d, J = 2.0 Hz, 1H), |
| | 99% | C22H17F5N6O/ | 8.25 (d, J = 2.0 Hz, 1H), 8.16-8.11 (m, 2H), 8.04 (d, |
| | | 477.1 | J = 7.6 Hz, 1H), 7.79 (t, J = 8.0 Hz, 1H), 6.88 (d, J = |
| | | (M + 1) | 4.0 Hz, 1H), 3.81-3.50 (m, 4H), 2.20-1.96 (m, 4H). |
| B-169 | 31%/ | 438.48 for | δ = 9.97 (s, 1H), 8.41 (d, J = 2.1 Hz, 1H), 8.22 (d, J = |
| | 99% | C24H24F2N4O2/ | 2.0 Hz, 1H), 8.16 (s, 1H), 7.96 (d, J = 3.6 Hz, 1H), |
| | | 439.3 | 7.63 (br dd, J = 2.0, 5.0 Hz, 1H), 7.47 (d, J = 4.8 Hz, |
| | | (M + 1) | 2H), 6.80 (d, J = 3.6 Hz, 1H), 3.65 (br d, J = 4.0 Hz, |
| | | | 4H), 3.27 (t, J = 8.4 Hz, 1H), 2.28-2.19 (m, 2H), |
| | | | 2.17-2.03 (m, 6H), 2.01-1.91 (m, 1H), 1.87-1.76 |
| | | | (m, 1H) |
| B-170 | 30%/ | 482.47 for | δ = 10.21 (s, 1H), 8.46 (s, 1H), 8.36 (s, 1H), 8.24 (s, |
| | 95% | C24H21F3N6O2/ | 1H), 8.11 (s, 1H), 8.08-8.01 (m, 2H), 7.74 (br d, J = |
| | | 483.3 | 11.6 Hz, 1H), 7.51 (br d, J = 9.2 Hz, 1H), 6.85 (d, |
| | | (M + 1) | J = 3.6 Hz, 1H), 3.91 (s, 3H), 3.75-3.56 (m, 4H), |
| | | | 2.15-2.03 (m, 4H). |
| B-171 | 46%/ | 468.51 for | δ = 10.98 (s, 1H), 9.15-9.02 (m, 1H), 8.98-8.86 |
| | 99% | C24H26F2N6O2/ | (m, 2H), 8.84-8.69 (m, 1H), 8.46 (s, 1H), 8.26 (s, |
| | | 469.1 | 1H), 8.18-8.04 (m, 1H), 6.98-6.84 (m, 1H), 3.77- |
| | | (M + 1) | 3.49 (m, 4H), 3.33 (br d, J = 11.2 Hz, 2H), 3.00-2.86 |
| | | | (m, 2H), 2.83-2.74 (m, 1H), 2.20-1.98 (m, 6H), |
| | | | 1.92-1.85 (m, 2H). |
| B-172 | 66%/ | 399.40 for | δ = 8.47 (d, J = 2.0 Hz, 1H), 8.24 (d, J = 2.0 Hz, 1H), |
| | 99% | C21H19F2N3O3/ | 8.22-8.17 (m, 3H), 8.16-8.11 (m, 2H), 6.88 (d, J = |
| | | 400.2 | 3.6 Hz, 1H), 3.89 (s, 3H), 3.80-3.49 (m, 4H), 2.17- |
| | | (M + 1) | 2.02 (m, 4H) |
| B-173 | 24%/ | 481.51 for | δ = 9.32 (d, J = 2.0 Hz, 1H), 8.44 (d, J = 1.6 Hz, 1H), |
| | 97.5% | C24H25F2N7O2/ | 8.24 (d, J = 2.0 Hz, 1H), 8.08 (d, J = 3.6 Hz, 1H), |
| | | 482.2 | 8.04 (dd, J = 2.0, 9.2 Hz, 1H), 7.65 (d, J = 9.2 Hz, |
| | | (M + 1) | 1H), 6.84 (d, J = 3.6 Hz, 1H), 3.99 (br d, J = 12.0 Hz, |
| | | | 1H), 3.94-3.87 (m, 2H), 3.80-3.50 (m, 6H), 3.02 |
| | | | (dt, J = 3.6, 12.0 Hz, 1H), 2.69 (dd, J = 10.4, 12.6 |
| | | | Hz, 1H), 2.17-2.00 (m, 4H), 1.16 (d, J = 6.4 Hz, |
| | | | 3H). |
| B-174 | 21%/ | 495.54 for | δ = 9.28 (d, J = 1.6 Hz, 1H), 8.44 (d, J = 2.0 Hz, 1H), |
| | 99.9% | C25H27F2N7O2/ | 8.24 (d, J = 2.0 Hz, 1H), 8.06 (d, J = 3.7 Hz, 1H), |
| | | 496.2 | 7.99 (dd, J = 2.0, 9.6 Hz, 1H), 7.60 (d, J = 9.6 Hz, |
| | | (M + 1) | 1H), 6.83 (d, J = 3.6 Hz, 1H), 4.37 (s, 1H), 3.77 (td, |
| | | | J = 4.1, 12.8 Hz, 2H), 3.65 (br d, J = 1.6 Hz, 4H), |
| | | | 3.45-3.36 (m, 2H), 2.17-2.00 (m, 4H), 1.54 (br d, |
| | | | J = 4.4 Hz, 4H), 1.16 (s, 3H). |
| B-175 | 48%/ | 467.52 for | δ = 10.30 (s, 1H), 8.77 (d, J = 2.0 Hz, 1H), 8.73 (d, |
| | 99% | C25H27F2N5O2/ | J = 2.4 Hz, 1H), 8.71-8.67 (m, 1H), 8.44 (d, J = 2.0 |
| | | 468.3 | Hz, 1H), 8.24 (d, J = 2.0 Hz, 1H), 8.09 (d, J = 3.6 Hz, |
| | | (M + 1) | 1H), 6.86 (d, J = 3.6 Hz, 1H), 3.98-3.41 (m, 4H), |
| | | | 2.45-2.34 (m, 1H), 2.22-1.96 (m, 4H), 1.92-1.71 |
| | | | (m, 4H), 1.66 (br d, J = 11.6 Hz, 1H), 1.52-1.35 (m, |
| | | | 2H), 1.35-1.07 (m, 3H). |
| B-176 | 58%/ | 388.43 for | δ = 14.28 (br s, 1H), 8.60-8.35 (m, 2H), 8.23-8.16 |
| | 99% | C21H20N6O2/ | (m, 3H), 8.13 (d, J = 3.6 Hz, 1H), 8.07 (br d, J = 8.4 |
| | | 389.1 | Hz, 2H), 6.85 (d, J = 3.6 Hz, 1H), 4.61-4.04 (m, |
| | | (M + 1) | 1H), 3.97-3.71 (m, 1H), 3.70-3.37 (m, 3H), 3.24- |
| | | | 2.69 (m, 2H), 1.08 (br s, 3H). |
| B-177 | 66.8%/ | 425.48 for | δ = 9.06 (br s, 1H), 8.54 (br s, 1H), 8.45 (d, J = 2.0 |
| | 99% | C23H25F2N5O/ | Hz, 1H), 8.29 (s, 1H), 8.24 (d, J = 2.0 Hz, 1H), 8.15 |
| | | 426.3 | (d, J = 3.6 Hz, 1H), 6.86 (d, J = 3.6 Hz, 1H), 3.78 (s, |
| | | (M + 1) | 2H), 3.76-3.19 (m, 4H), 2.55 (br s, 4H), 2.07 (br s, |
| | | | 4H), 1.73 (br t, J = 3.2 Hz, 4H). |
| B-178 | 67%/ | 479.49 for | δ = 9.01 (br s, 1H), 8.74 (br t, J = 6.0 Hz, 1H), 8.53 |
| | 99% | C24H23F2N7O2/ | (br s, 1H), 8.41 (d, J = 1.6 Hz, 1H), 8.28 (s, 1H), 8.24 |
| | | 480.2 | (d, J = 2.0 Hz, 1H), 8.14 (s, 1H), 8.10 (d, J = 3.6 Hz, |
| | | (M + 1) | 1H), 7.86 (s, 1H), 6.86 (d, J = 3.6 Hz, 1H), 4.55 (d, |
| | | | J = 5.6 Hz, 2H), 3.84 (s, 3H), 3.78-3.41 (m, 4H), 2.22- |
| | | | 1.91 (m, 4H). |
| B-179 | 63%/ | 423.47 for | δ = 9.03 (br s, 1H), 8.61 (brs, 1H), 8.38 (br d, J = 1.6 |
| | 99% | C22H25N5O4/ | Hz, 2H), 8.17 (s, 1H), 8.14 (d, J = 3.6 Hz, 1H), 6.87 |
| | | 424.2 | (d, J = 4.0 Hz, 1H), 5.14-4.76 (m, 1H), 4.15 (q, J = |
| | | (M + 1) | 7.2 Hz, 2H), 3.90-3.63 (m, 1H), 3.62-3.43 (m, 2H), |
| | | | 3.35 (s, 3H), 3.18-2.83 (m, 1H), 1.93-1.59 (m, 2H), |
| | | | 1.55-1.33 (m, 2H), 1.22 (t, J = 7.2 Hz, 3H). |
| B-180 | 20%/ | 423.47 for | δ = 9.02 (br s, 1H), 8.59 (br s, 1H), 8.42-8.35 (m, |
| | 99% | C22H25N5O4/ | 2H), 8.17 (s, 1H), 8.14 (d, J = 3.6 Hz, 1H), 6.87 (d, |
| | | 424.2 | J = 3.6 Hz, 1H), 5.17-4.71 (m, 1H), 4.15 (q, J = 7.2 |
| | | (M + 1) | Hz, 2H), 3.85-3.65 (m, 1H), 3.62-3.47 (m, 2H), |
| | | | 3.33-3.26 (m, 3H), 3.13-2.83 (m, 1H), 2.00-1.57 |
| | | | (m, 2H), 1.55-1.33 (m, 2H), 1.21 (t, J = 7.2 Hz, 3H). |
| B-181 | 62%/ | 388.43 for | δ = 14.28 (br s, 1H), 8.60-8.47 (m, 2H), 8.41 (d, J = |
| | 99% | C21H20N6O2/ | 2.0 Hz, 1H), 8.19 (d, J = 2.0 Hz, 1H), 8.11 (d, J = 4.0 |
| | | 389.1 | Hz, 1H), 8.03 (d, J = 7.6 Hz, 1H), 7.92 (br d, J = 8.0 |
| | | (M + 1) | Hz, 1H), 7.68 (t, J = 7.6 Hz, 1H), 6.84 (d, J = 3.6 Hz, |
| | | | 1H), 4.62-4.00 (m, 1H), 3.93-3.72 (m, 1H), 3.69- |
| | | | 3.39 (m, 3H), 3.24-2.72 (m, 2H), 1.08 (br s, 3H). |
| B-182 | 47%/ | 388.43 for | δ = 14.49-14.00 (m, 1H), 8.67-8.44 (m, 1H), 8.42 |
| | 98% | C21H20N6O2/ | (d, J = 2.0 Hz, 1H), 8.25-8.16 (m, 3H), 8.13 (d, J = |
| | | 389.1 | 3.6 Hz, 1H), 8.07 (br d, J = 8.4 Hz, 2H), 6.84 (d, J = |
| | | (M + 1) | 3.6 Hz, 1H), 4.75-4.05 (m, 1H), 4.02-3.44 (m, 4H), |
| | | | 3.29-2.68 (m, 2H), 1.08 (br d, J = 3.2 Hz, 3H). |
| B-183 | 23%/ | 482.54 for | δ = 10.37 (s, 1H), 8.89-8.65 (m, 3H), 8.44 (d, J = |
| | 98% | C25H28F2N6O2/ | 2.0 Hz, 1H), 8.25 (d, J = 2.0 Hz, 1H), 8.09 (d, J = 3.6 |
| | | 483.3 | Hz, 1H), 6.86 (d, J = 3.6 Hz, 1H), 3.79-3.54 (m, |
| | | (M + 1) | 4H), 2.86 (br d, J = 11.6 Hz, 2H), 2.42-2.35 (m, |
| | | | 1H), 2.19 (s, 3H), 2.08 (br s, 4H), 1.92 (br t, J = 10.8 |
| | | | Hz, 2H), 1.81 (br d, J = 12.0 Hz, 2H), 1.69 (m, 2H). |
| B-184 | 32%/ | 456.47 for | δ = 10.16 (s, 1H), 8.45 (d, J = 1.8 Hz, 1H), 8.23 (d, |
| | 99% | C24H23F3N4O2/ | J = 1.6 Hz, 1H), 8.02 (d, J = 3.6 Hz, 1H), 7.97 (s, 1H), |
| | | 457.3 | 7.64 (br d, J = 11.6 Hz, 1H), 7.46 (dd, J = 1.6, 10.0 |
| | | (M + 1) | Hz, 1H), 6.83 (d, J = 3.2 Hz, 1H), 3.87-3.45 (m, |
| | | | 4H), 3.30-3.21 (m, 1H), 2.30-2.20 (m, 2H), 2.20- |
| | | | 2.01 (m, 6H), 1.96 (br d, J = 9.2 Hz, 1H), 1.89-1.77 |
| | | | (m, 1H). |
| B-185 | 21%/ | 442.44 for | δ = 10.65 (s, 1H), 8.45 (d, J = 2.0 Hz, 1H), 8.23 (d, J = |
| | 99% | C23H21F3N4O2/ | 2.0 Hz, 1H), 8.03 (d, J = 3.6 Hz, 1H), 7.97 (s, 1H), |
| | | 443.2 | 7.61 (br d, J = 11.2 Hz, 1H), 7.47 (br dd, J = 2.0, 10.1 |
| | | (M + 1) | Hz, 1H), 6.83 (d, J = 3.6 Hz, 1H), 3.80-3.53 (m, |
| | | | 4H), 2.08 (br d, J = 5.2 Hz, 4H), 1.90-1.75 (m, 1H), |
| | | | 0.84 (d, J = 6.0 Hz, 4H). |
| B-186 | 34%/ | 439.47 for | δ = 9.32-8.89 (m, 1H), 8.73 (br t, J = 5.6 Hz, 1H), |
| | 99% | C23H23F2N5O2/ | 8.66-8.48 (m, 1H), 8.44 (d, J = 2.0 Hz, 1H), 8.31- |
| | | 440.3 | 8.21 (m, 2H), 8.12 (d, J = 3.6 Hz, 1H), 6.87 (d, J = |
| | | (M + 1) | 3.6 Hz, 1H), 4.43 (d, J = 5.6 Hz, 2H), 3.64-3.72 (m, |
| | | | 4H), 2.07 (br s, 4H), 1.62-1.59 (m, 1H), 0.71-0.68 |
| | | | (m, 4H) |
| B-187 | 54%/ | 439.47 for | δ = 8.90-8.77 (m, 1H), 8.73 (br d, J = 5.2 Hz, 1H), |
| | 100% | C23H23F2N5O2/ | 8.51 (d, J = 2.0 Hz, 1H), 8.48 (br s, 1H), 8.34 (d, J = |
| | | 440.2 | 3.6 Hz, 1H), 8.32-8.21 (m, 2H), 6.98 (d, J = 3.6 Hz, |
| | | (M + 1) | 1H), 4.53 (br d, J = 4.4 Hz, 2H), 3.63 (br s, 4H), 2.08 |
| | | | (br d, J = 5.2 Hz, 4H), 1.77-1.64 (m, 1H), 0.73 (d, J = |
| | | | 6.0 Hz, 4H) |
| B-188 | 24%/ | 475.45 for | δ = 10.60 (s, 1H), 8.79 (br d, J = 3.2 Hz, 2H), 8.74- |
| | 97% | C23H21F4N5O2/ | 8.70 (m, 1H), 8.45 (d, J = 2.0 Hz, 1H), 8.25 (d, J = |
| | | 476.2 | 2.0 Hz, 1H), 8.12 (d, J = 3.6 Hz, 1H), 6.88 (d, J = 3.6 |
| | | (M + 1) | Hz, 1H), 3.81-3.51 (m, 4H), 3.19 (m, 1H), 2.95- |
| | | | 2.76 (m, 4H), 2.15-2.02 (m, 4H). |
| B-189 | 32%/ | 388.43 for | δ = 14.65-14.01 (m, 1H), 8.55 (s, 2H), 8.41 (d, J = |
| | 99% | C21H20N6O2/ | 1.6 Hz, 1H), 8.19 (d, J = 2.0 Hz, 1H), 8.11 (d, J = 3.6 |
| | | 389.1 | Hz, 1H), 8.02 (d, J = 8.0 Hz, 1H), 7.92 (br d, J = 7.6 |
| | | (M + 1) | Hz, 1H), 7.73-7.63 (m, 1H), 6.84 (d, J = 3.6 Hz, |
| | | | 1H), 4.53-4.13 (m, 1H), 4.08-3.68 (m, 2H), 3.67- |
| | | | 3.38 (m, 4H), 1.20-0.94 (m, 3H). |
| B-190 | 25%/ | 472.92 for | δ = 10.14 (s, 1H), 8.45 (d, J = 1.6 Hz, 1H), 8.23 (d, |
| | 98% | C24H23ClF2N4O2/ | J = 2.0 Hz, 1H), 8.09 (s, 1H), 8.03 (d, J = 3.6 Hz, 1H), |
| | | 473.2 | 7.84 (s, 1H), 7.69-7.60 (m, 1H), 6.83 (d, J = 3.6 Hz, |
| | | (M + 1) | 1H), 3.83-3.47 (m, 4H), 3.26 (t, J = 8.4 Hz, 1H), |
| | | | 2.31-2.20 (m, 2H), 2.18-2.02 (m, 6H), 1.95 (br d, |
| | | | J = 9.2 Hz, 1H), 1.82 (br dd, J = 5.2, 9.2 Hz, 1H). |
| B-191 | 39%/ | 458.89 for | δ = 10.62 (s, 1H), 8.45 (d, J = 1.6 Hz, 1H), 8.22 (d, |
| | 98% | C23H21ClF2N4O2/ | J = 1.6 Hz, 1H), 8.08 (s, 1H), 8.03 (d, J = 3.6 Hz, 1H), |
| | | 459.2 | 7.80 (s, 1H), 7.66 (s, 1H), 6.83 (d, J = 3.6 Hz, 1H), |
| | | (M + 1) | 3.79-3.51 (m, 5H), 2.07 (br s, 4H), 1.86-1.73 (m, |
| | | | 1H), 0.84 (br d, J = 5.9 Hz, 4H). |
| B-192 | 31%/ | 442.85 for | δ = 14.69-14.09 (m, 1H), 8.64 (br s, 1H), 8.60 (t, J = |
| | 99.6% | C21H17ClF2N6O/ | 1.6 Hz, 1H), 8.49 (d, J = 2.0 Hz, 1H), 8.25 (d, J = |
| | | 443.2 | 2.0 Hz, 1H), 8.23-8.18 (m, 1H), 8.13 (s, 1H), 8.00 |
| | | (M + 1) | (t, J = 1.6 Hz, 1H), 6.87 (d, J = 3.6 Hz, 1H), 3.65 (br |
| | | | s, 4H), 2.20-1.97 (m, 4H). |
| B-193 | 31%/ | 426.40 for | δ = 8.62 (br s, 1H), 8.53 (d, J = 1.6 Hz, 1H), 8.49 (d, |
| | 99.6% | C21H17F3N6O/ | J = 2.0 Hz, 1H), 8.25 (d, J = 2.0 Hz, 1H), 8.20 (d, J = |
| | | 427.2 | 3.6 Hz, 1H), 7.94 (td, J = 2.0, 10.3 Hz, 1H), 7.80- |
| | | (M + 1) | 7.70 (m, 1H), 6.87 (d, J = 3.6 Hz, 1H), 3.66 (br s, |
| | | | 4H), 2.13-2.03 (m, 4H). |
| B-194 | 70%/ | 408.41 for | δ = 13.50-12.74 (m, 1H), 8.63 (d, J = 5.6 Hz, 1H), |
| | 99% | C21H18F2N6O/ | 8.52 (d, J = 2.0 Hz, 1H), 8.37 (d, J = 3.6 Hz, 1H), |
| | | 409.3 | 8.29-8.26 (m, 4H), 8.15 (dd, J = 2.0, 5.6 Hz, 1H), |
| | | (M + 1) | 6.93 (d, J = 3.6 Hz, 1H), 3.90-3.48 (m, 4H), 2.09 |
| | | | (br s, 4H). |
| B-195 | 66%/ | 402.46 for | δ = 14.59-13.99 (m, 1H), 8.71-8.44 (m, 1H), 8.38 |
| | 99% | C22H22N6O2/ | (br s, 1H), 8.19 (br d, J = 8.8 Hz, 2H), 8.15 (br s, 1H), |
| | | 403.1 | 8.12 (br d, J = 3.6 Hz, 1H), 8.08 (br d, J = 8.4 Hz, |
| | | (M + 1) | 2H), 6.84 (d, J = 3.6 Hz, 1H), 4.15-3.70 (m, 1H), |
| | | | 3.40 (br dd, J = 6.4, 13.2 Hz, 5H), 3.09 (br d, J = 2.0 |
| | | | Hz, 2H), 1.93-1.42 (m, 4H). |
| B-196 | 27.3%/ | 356.38 for | δ = 8.56 (d, J = 5.6 Hz, 1H), 8.50 (d, J = 2.0 Hz, 1H), |
| | 99% | C19H18F2N4O/ | 8.30-8.19 (m, 2H), 8.07-7.97 (m, 2H), 6.89 (d, J = |
| | | 357.2 | 4.0 Hz, 1H), 4.08-3.43 (m, 4H), 2.56 (s, 3H), 2.18- |
| | | (M + 1) | 1.96 (m, 4H). |
| B-197 | 69%/ | 402.46 for | δ = 14.62-13.89 (m, 1H), 8.49 (br s, 1H), 8.38 (br s, |
| | 99% | C22H22N6O2/ | 1H), 8.19 (d, J = 8.8 Hz, 2H), 8.15 (br s, 1H), 8.12 |
| | | 403.2 | (d, J = 4.0 Hz, 1H), 8.08 (d, J = 8.8 Hz, 2H), 6.84 (d, |
| | | (M + 1) | J = 3.6 Hz, 1H), 4.17-3.70 (m, 1H), 3.55-3.34 (m, |
| | | | 5H), 3.21-2.98 (m, 2H), 1.94-1.42 (m, 4H). |
| B-198 | 16%/ | 413.43 for | δ = 9.12-8.93 (m, 1H), 8.63-8.46 (m, 2H), 8.44 (d, |
| | 98% | C21H21F2N5O2/ | J = 2.0 Hz, 1H), 8.25 (d, J = 2.0 Hz, 1H), 8.23 (s, |
| | | 414.3 | 1H), 8.11 (d, J = 4.0 Hz, 1H), 6.87 (d, J = 3.6 Hz, |
| | | (M + 1) | 1H), 4.40 (d, J = 6.0 Hz, 2H), 3.84-3.47 (m, 4H), |
| | | | 2.15-2.00 (m, 4H), 1.89 (s, 3H). |
| B-199 | 93%/ | 424.45 for | δ = 10.43 (s, 1H), 8.41 (d, J = 2.0 Hz, 1H), 8.21 (d, |
| | 98% | C23H22F2N4O2/ | J = 2.0 Hz, 1H), 8.15 (s, 1H), 7.96 (d, J = 3.6 Hz, 1H), |
| | | 425.3 | 7.63-7.57 (m, 1H), 7.51-7.42 (m, 2H), 6.80 (d, J = |
| | | (M + 1) | 3.6 Hz, 1H), 3.65 (m, 4H), 2.07 (brt, J = 5.6 Hz, 4H), |
| | | | 1.88-1.75 (m, 1H), 0.90-0.70 (m, 4H). |
| B-200 | 36%/ | 426.40 for | δ = 14.54-14.08 (m, 1H), 8.53 (br d, J = 1.6 Hz, |
| | 98% | C21H17F3N6O/ | 1H), 8.50 (d, J = 2.0 Hz, 1H), 8.25 (d, J = 2.0 Hz, |
| | | 427.3 | 1H), 8.23 (d, J = 4.0 Hz, 1H), 8.21-8.14 (m, 2H), |
| | | (M + 1) | 8.04 (dd, J = 2.0, 8.6 Hz, 1H), 6.88 (d, J = 4.0 Hz, |
| | | | 1H), 3.86-3.48 (m, 4H), 2.16-2.00 (m, 4H). |
| B-201 | 46%/ | 476.91 for | δ = 8.46 (d, J = 1.6 Hz, 1H), 8.23 (d, J = 2.0 Hz, 1H), |
| | 98% | C23H23ClF2N4O3/ | 8.13 (d, J = 4.0 Hz, 1H), 8.00 (s, 1H), 7.92 (s, 1H), |
| | | 477.2 | 7.46 (s, 1H), 6.84 (d, J = 4.0 Hz, 1H), 4.13 (q, J = 7.2 |
| | | (M + 1) | Hz, 2H), 3.77-3.52 (m, 4H), 3.31 (s, 3H), 2.07 (br |
| | | | s, 4H), 1.21 (t, J = 7.2 Hz, 3H). |
| B-202 | 38%/ | 442.85 for | δ = 14.29 (br s, 1H), 8.57 (br s, 1H), 8.50 (d, J = 2.0 |
| | 97% | C21H17ClF2N6O/ | Hz, 1H), 8.35 (d, J = 1.6 Hz, 1H), 8.28-8.18 (m, |
| | | 443.1 | 2H), 8.15-8.07 (m, 1H), 8.07-7.99 (m, 1H), 6.88 |
| | | (M + 1) | (d, J = 4.0 Hz, 1H), 3.65 (br d, J = 2.8 Hz, 4H), 2.15- |
| | | | 2.00 (m, 4H). |
| B-203 | 45%/ | 462.88 for | δ = 9.24 (s, 1H), 8.36 (d, J = 2.0 Hz, 1H), 8.21-8.14 |
| | 98% | C22H21ClF2N4O3/ | (m, 2H), 8.04 (d, J = 3.6 Hz, 1H), 7.78-7.71 (m, |
| | | 463.1 | 1H), 7.70-7.63 (m, 1H), 6.84 (d, J = 3.6 Hz, 1H), |
| | | (M + 1) | 4.15 (q, J = 7.2 Hz, 2H), 4.01-3.74 (m, 2H), 3.70- |
| | | | 3.44 (m, 2H), 2.21-2.05 (m, 2H), 1.73 (br s, 2H), |
| | | | 1.25 (t, J = 7.1 Hz, 3H). |
| B-204 | 27%/ | 462.88 for | δ = 9.24 (s, 1H), 8.43 (d, J = 2.0 Hz, 1H), 8.23 (d, |
| | 99% | C22H21ClF2N4O3/ | J = 2.0 Hz, 1H), 8.21-8.18 (m, 1H), 8.04 (d, J = 3.6 |
| | | 463.2 | Hz, 1H), 7.76-7.71 (m, 1H), 7.69-7.64 (m, 1H), |
| | | (M + 1) | 6.83 (d, J = 4.0 Hz, 1H), 4.15 (q, J = 7.2 Hz, 2H), |
| | | | 3.82-3.48 (m, 4H), 2.13-2.01 (m, 4H), 1.25 (t, J = |
| | | | 7.2 Hz, 3H). |
| B-205 | 41%/ | 462.88 for | δ = 10.09 (s, 1H), 8.38 (d, J = 2.0 Hz, 1H), 8.17 (d, |
| | 99% | C22H21ClF2N4O3/ | J = 2.0 Hz, 1H), 8.03-7.98 (m, 2H), 7.63 (t, J = 1.6 |
| | | 463.3 | Hz, 1H), 7.59 (s, 1H), 6.85 (d, J = 4.0 Hz, 1H), 4.17 |
| | | (M + 1) | (q, J = 7.2 Hz, 2H), 4.07-3.75 (m, 2H), 3.74-3.45 |
| | | | (m, 2H), 2.20-2.06 (m, 2H), 1.74 (br s, 2H), 1.27 (t, |
| | | | J = 7.2 Hz, 3H). |
| B-206 | 30%/ | 462.88 for | δ = 10.08 (s, 1H), 8.44 (d, J = 2.0 Hz, 1H), 8.22 (d, |
| | 99% | C22H21ClF2N4O3/ | J = 2.0 Hz, 1H), 8.00 (d, J = 3.2 Hz, 2H), 7.63 (t, J = |
| | | 463.3 | 2.0 Hz, 1H), 7.57 (s, 1H), 6.83 (d, J = 3.6 Hz, 1H), |
| | | (M + 1) | 4.16 (q, J = 7.2 Hz, 2H), 3.85-3.47 (m, 4H), 2.07 |
| | | | (br t, J = 12.8 Hz, 4H), 1.26 (t, J = 7.2 Hz, 3H). |
| B-207 | 23%/ | 460.46 for | δ = 8.46 (d, J = 2.0 Hz, 1H), 8.23 (d, J = 2.0 Hz, 1H), |
| | 99% | C23H23F3N4O3/ | 8.13 (d, J = 3.6 Hz, 1H), 7.84 (d, J = 1.6 Hz, 1H), |
| | | 461.1 | 7.80 (td, J = 2.0, 10.4 Hz, 1H), 7.27 (td, J = 2.0, 10.4 |
| | | (M + 1) | Hz, 1H), 6.84 (d, J = 3.6 Hz, 1H), 4.13 (q, J = 7.2 Hz, |
| | | | 2H), 3.64 (br s, 4H), 3.31 (s, 3H), 2.19-1.96 (m, |
| | | | 4H), 1.21 (t, J = 6.8 Hz, 3H). |
| B-208 | 35%/ | 446.43 for | δ = 10.09 (s, 1H), 8.37 (d, J = 2.0 Hz, 1H), 8.16 (d, |
| | 98% | C22H21F3N4O3/ | J = 2.0 Hz, 1H), 7.99 (d, J = 4.0 Hz, 1H), 7.87 (s, 1H), |
| | | 447.1 | 7.43 (td, J = 2.0, 10.0 Hz, 1H), 7.38 (br d, J = 11.2 |
| | | (M + 1) | Hz, 1H), 6.84 (d, J = 4.4 Hz, 1H), 4.16 (q, J = 7.2 Hz, |
| | | | 2H), 4.04-3.72 (m, 2H), 3.70-3.40 (m, 2H), 2.19- |
| | | | 2.06 (m, 2H), 1.74 (br s, 2H), 1.26 (t, J = 7.2 Hz, 3H). |
| B-209 | 33%/ | 446.43 for | δ = 10.09 (s, 1H), 8.44 (d, J = 2.0 Hz, 1H), 8.22 (d, |
| | 99% | C22H21F3N4O3/ | J = 2.0 Hz, 1H), 7.98 (d, J = 3.6 Hz, 1H), 7.88 (s, 1H), |
| | | 447.1 | 7.48-7.40 (m, 1H), 7.37 (br d, J = 11.2 Hz, 1H), 6.83 |
| | | (M + 1) | (d, J = 4.0 Hz, 1H), 4.16 (q, J = 7.2 Hz, 2H), 3.65 (br |
| | | | d, J = 1.2 Hz, 4H), 2.07 (br t, J = 12.4 Hz, 4H), 1.26 |
| | | | (t, J = 7.2 Hz, 3H). |
| B-212 | 36%/ | 406.46 for | δ/ppm 14.59-14.21 (s, 1H), 8.68 (s, 1H), 8.55 (s, |
| | 99.2% | C20H18N6O2S/ | 1H), 8.45 (d, J = 1.8 Hz, 1H), 8.23 (d, J = 1.8 Hz, |
| | | 407.2 | 1H), 8.12 (d, J = 3.6 Hz, 1H), 8.03 (d, J = 6.9 Hz, |
| | | (M + 1) | 1H), 7.87 (s, 1H), 7.67 (s,1H), 6.85 (d, J = 3.3 Hz, |
| | | | 1H), 4.35 (brs, 1H), 3.82 (brs, 3H), 3.01 (t, J = 13.2 |
| | | | Hz, J = 27.6 Hz, 2H), 2.81 (brs, 2H). |
| B-213 | 9%/ | 406.46 for | δ/ppm 14.18 (s, 1H), 8.65 (s, 1H), 8.45 (d, J = 1.8 |
| | 99.0% | C20H18N6O2S/ | Hz, 1H), 8.23 (d, J = 1.8 Hz, 1H), 8.19 (d, J = 8.4 Hz, |
| | | 407.1 | 2H), 8.14 (d, J = 3.3 Hz, 1H), 8.07 (s, 2H), 6.85 (d, |
| | | (M + 1) | J = 3.9 Hz, 1H), 4.34 (brs, 1H), 3.81 (brs, 3H), 3.01 (t, |
| | | | J = 13.5 Hz, J = 24.6 Hz, 2H), 2.81 (d, J = 6 Hz, 2H). |
| B-214 | 21.4%/ | 440.52 for | δ: 9.33 (s, 1H), 8.77 (d, J = 2.1 Hz, 1H), 8.64 (d, J = |
| | 93.9% | C21H24N6O3S/ | 2.1 Hz, 1H), 8.2 (d, J = 3.9 Hz, 1H), 8.02 (dd, J = |
| | | 441.2 | 9.3, 1.8 Hz, 1H), 7.69 (d, J = 9.3, 1H), 7.0 (q, J = 3.6 |
| | | (M + 1) | Hz, 1H), 3.75-3.72 (m, 4H), 3.52-3.49 (m, 4H), |
| | | | 3.37-3.42 (m, 2H), 1.58-1.55 (m, 2H), 1.37-1.26 |
| | | | (m, 2H), 0.83 (t, J = 7.2, 3H). |
| B-215 | 7%/ | 422.46 for | δ/ppm 14.56-14.18 (s, 1H), 8.66 (s, 1H), 8.48 (d, J = |
| | 94.8% | C20H18N6O3S/ | 2.1 Hz, 1H), 8.27 (d, J = 1.8 Hz, 1H), 8.19 (d, J = |
| | | 423.2 | 8.4 Hz, 2H), 8.14 (d, J = 3.3 Hz, 1H), 8.07 (s, 2H), |
| | | (M + 1) | 6.86 (d, J = 3.6 Hz, 1H), 3.93 (s, 4H), 3.30 (s, 4H). |
| B-216 | 12.9%/ | 426.49 for | δ: 9.87 (s, 1H), 8.40 (d, J = 1.8 Hz, 1H), 8.21 (d, J = |
| | 97.5% | C21H22N4O4S/ | 1.8 Hz, 1H), 8.00 (s, 1H), 7.95 (d, J = 3.6 Hz, 1H), |
| | | 427.2 | 7.44 (s, 3H), 6.81 (d, J = 3.6 Hz, 1H) 4.14 (q, J = |
| | | (M + 1) | 7.2, 6.9 Hz, 2H), 3.81 (brs, 3H), 3.43 (brs, 1H), 3.00 |
| | | | (brs, 2H), 2.82 (brs, 2H), 1.25 (t, J = 7.2 Hz, 3H); |
| B-217 | 19.3%/ | 427.48 for | δ: 10.14 (s, 1H), 8.70 (d, J = 2.1 Hz, 1H), 8.62 (s, |
| | 99.5% | C20H21N5O4S/ | 1H), 8.54 (s, 1H), 8.43 (s, 1H), 8.23 (s, 1H), 8.09 (d, |
| | | 428.1 | J = 3.9 Hz, 1H), 6.87 (d, J = 3.6 Hz, 1H) 4.17 (q, J = |
| | | (M + 1) | 7.2, 6.9 Hz, 2H), 3.80 (brs, 3H), 3.43 (brs, 2H), |
| | | | 3.01 (brs, 3H), 2.84 (brs, 2H), 1.27 (t, J = 7.2 Hz, 3H) |
| B-218 | 38.5%/ | 443.48 for | δ: 10.14 (s, 1H), 8.69 (s, 1H), 8.62 (s, 1H), 8.54 (s, |
| | 94.5% | C20H21N5O5S/ | 1H), 8.46 (s, 1H), 8.28 (d, J = 2.1 Hz, 1H), 8.09 (d, |
| | | 444.3 | J = 3.9 Hz, 1H), 6.87 (d, J = 3.6 Hz, 1H) 4.17 (q, J = |
| | | (M + 1) | 7.2, 6.9 Hz, 2H), 3.93 (brs, 4H), 3.28 (brs, 4H), 1.27 |
| | | | (t, J = 7.2 Hz, 3H) |
| B-219 | 34%/ | 422.46 for | 1H NMR (300 MHz, DMSO): δ/ppm 14.57-14.20 (s, |
| | 99.6% | C20H18N6O3S/ | 1H), 8.68 (s, 1H), 8.55 (s, 1H), 8.47 (d, J = 1.5 Hz, |
| | | 423.2 | 1H), 8.28 (s, 1H), 8.12 (d, J = 3.6 Hz, 1H), 8.03 (d, |
| | | (M + 1) | J = 6.6 Hz, 1H), 7.92-7.85 (m, 1H), 7.76-7.63 (m, |
| | | | 1H), 6.85 (d, J = 3.0 Hz, 1H), 3.94 (s, 4H), 3.29 (s, |
| | | | 4H). |
| B-220 | 61.1%/ | 405.46 for | δ: 8.74 (s, 1H), 8.58 (s, 1H), 8.46 (d, J = 1.8 Hz, 1H), |
| | 90.4% | C22H23N5O3/ | 8.46 (s, 1H), 8.09 (d, J = 1.2 Hz, 1H), 7.60 (d, J = 3.6 |
| | | 406.2 | Hz, 1H), 6.90-6.88 (m, 1H), 6.73 (d, J = 3.6 Hz, |
| | | (M + 1) | 1H), 3.54 (bs, 7H), 1.43 (bs, 4H), 0.39 (s, 4H). |
| B-221 | 13%/ | 388.43 for | δ/ppm 14.23 (s, 1H), 8.61 (s, 1H), 8.39 (s, 1H), 8.20- |
| | 98.6% | C21H20N6O2/ | 8.06 (m, 6H), 6.84 (d, J = 3.6 Hz, 1H), 5.01-4.84 |
| | | 389.2 | (m, 1H), 4.22 (brs, 1H), 3.78 (brs, 1H), 3.55 (s, 2H), |
| | | (M + 1) | 3.11 (brs, 1H), 2.86 (brs, 1H), 1.86 (brs, 2H), 1.45 |
| | | | (s, 1H). |
| B-222 | 64.3%/ | 442.49 for | δ: 9.88 (s, 1H), 8.43 (d, J = 1.8 Hz, 1H), 8.25 (d, J = |
| | 93.3% | C21H22N4O5S/ | 2.1 Hz, 1H), 8.00 (s, 1H), 7.95 (d, J = 3.6 Hz, 1H), |
| | | 443.2 | 7.46-7.41 (m, 3H), 6.81 (d, J = 3.6 Hz, 1H), 4.14 (q, |
| | | (M + 1) | J = 7.2, 6.9 Hz, 2H), 3.93 (brs, 3H), 3.43 (brs, 1H), |
| | | | 3.28 (brs, 4H), 1.25 (t, J = 7.2 Hz, 3H) |
| B-223 | 60%/ | 390.47 for | δ: 14.21 (s, 1H), 8.68 (s, 1H), 8.55 (s, 1H), 8.39 (d, |
| | 99.3% | C20H18N6OS/ | J = 1.8 Hz, 1H), 8.18(d, J = 1.8 Hz, 1H), 8.11(d, J = |
| | | 391.1 | 3.6 Hz, 1H), 8.03(m, 1H), 7.87(m, 1H), 7.63- |
| | | (M + 1) | 7.68(m, 1H), 6.83(m, 1H), 3.78(brs, 4H), 2.68(brs, |
| | | | 4H). |
| B-224 | 13%/ | 388.43 for | δ/ppm 11.73 (s, 1H), 8.53 (d, J = 1.5 Hz, 1H), 8.23- |
| | 99.8% | C21H20N6O2/ | 8.18 (m, 3H), 8.11 (d, J = 1.8 Hz, 1H), 7.86 (d, J = |
| | | 389.1 | 8.7 Hz, 2H), 7.62 (d, J = 3.6 Hz, 1H), 6.70 (d, J = 3.9 |
| | | (M + 1) | Hz, 1H), 3.93 (brs, 2H), 3.63 (brs, 3H), 2.17 (s, 1H), |
| | | | 1.89 (brs, 2H), 1.76 (brs, 2H). |
| B-225 | 55.8%/ | 410.49 for | δ: 9.87 (s, 1H), 8.35 (d, J = 1.8 Hz, 1H), 8.16 (d, J = |
| | 99.6% | C21H22N4O3S/ | 1.8 Hz, 1H), 7.99 (s, 1H), 7.94 (d, J = 3.6 Hz, 1H), |
| | | 411.2 | 7.44 (s, 3H), 6.79 (d, J = 3.6 Hz, 1H), 4.14 (q, J = |
| | | (M + 1) | 7.2, 6.9 Hz, 2H), 3.79 (brs, 4H), 2.68 (brs, 4H), 1.25 |
| | | | (t, J = 7.2 Hz, 3H) |
| B-226 | 7.0%/ | 407.43 for | δ: 13.00 (s, 1H), 8.42 (d, J = 1.5 Hz, 1H), 8.27 (s, |
| | 91.8% | C22H19F2N5O/ | 1H), 8.22 (d, J = 1.8 Hz, 1H), 8.07 (d, J = 3.6 Hz, |
| | | 408.2 | 1H), 7.99 (s, 1H), 7.88-7.77 (m, 4H), 6.81 (d, J = |
| | | (M + 1) | 3.3 Hz, 1H), 3.65 (brs, 4H), 2.08 (brs, 4H) |
| B-227 | 23.1%/ | 390.47 for | δ: 14.27(brs, 1H), 8.44(brs, 1H), 8.4(d, J = 1.8 Hz, |
| | 99.7% | C20H18N6OS/ | 1H), 8.06-8.2(m, 7H), 6.84(s, 1H), 3.18 (brs, 4H), |
| | | 389.2 | 2.69(brs, 4H). |
| | | (M − 1) | |
| B-228 | 29%/ | 440.43 for | δ/ppm 14.17 (s, 1H), 8.66 (s, 1H), 8.41 (s, 1H), 8.21- |
| | 99.6% | C22H19F3N6O/ | 8.14 (m, 4H), 8.05 (d, J = 6.6 Hz, 2H), 6.86 (d, J = |
| | | 441.1 | 3.6 Hz, 1H), 4.58 (brs, 1H), 3.70 (brs, 1H), 3.08 (brs, |
| | | (M + 1) | 3H), 1.99 (brs, 1H), 1.62-1.56 (m, 2H). |
| B-229 | 22%/ | 440.43 for | δ/ppm 14.55-14.16 (s, 1H), 8.66 (s, 1H), 8.41 (s, 1H), |
| | 99.3% | C22H19F3N6O/ | 8.21-8.12 (m, 4H), 8.07-8.02 (m, 2H), 6.85 (t, J = |
| | | 441.3 | 8.4 Hz, 1H), 4.56 (brs, 1H), 3.73 (brs, 1H), 3.04 (brs, |
| | | (M + 1) | 2H), 1.99 (brs, 1H), 1.73-1.55 (m, 1H), 1.23 (brs, |
| | | | 2H). |
| B-230 | 64.6%/ | 411.48 for | δ: 10.14 (s, 1H), 8.70 (s, 1H), 8.62 (s, 1H), 8.52 (s, |
| | 95.9% | C20H21N5O3S/ | 1H), 8.38 (s, 1H), 8.19 (s, 1H), 8.08 (d, J = 3.6 Hz, |
| | | 412.1 | 1H), 6.85 (d, J = 3.6 Hz, 1H), 4.17 (q, J = 7.2 Hz, 6.9 |
| | | (M + 1) | Hz, 2H), 3.76 (brs, 4H), 2.68 (brs, 4H), 1.26 (t, J = |
| | | | 7.2 Hz, 3H) |
| B-231 | 22.2%/ | 418.50 for | δ: 8.76 (d, J = 2.4, 1H), 8.32-8.37 (m, 2H), 8.21 (dd, |
| | 97.3% | C23H26N6O2/ | J = 9.3, 2.7 Hz, 1H), 8.13 (d, J = 1.8 Hz, 1H), 8.02 |
| | | 419.3 | (d, J = 3.6 Hz, 1H), 6.81 (d, J = 3.6 Hz, 1H), 4.34 |
| | | (M + 1) | (brs, 1H), 3.99 (t, J = 7.5 Hz, 2H), 3.5 (t, J = 6.3 Hz, |
| | | | 3H), 2.82 (s, 3H), 1.77-1.81 (m, 1H), 1.62-1.63 (m, |
| | | | 2H), 1.42-1.48 (m, 1H), 1.14-1.23 (m, 2H), 0.8-0.93 |
| | | | (m, 4H). |
| B-232 | 20%/ | 469.49 for | δ/ppm 10.40 (s, 1H), 8.78 (d, J = 1.8 Hz 1H), 8.74 |
| | 99.3% | C24H25F2N5O3/ | (d, J = 2.1 Hz, 1H), 8.71 (d, J = 2.1 Hz, 1H), 8.44 (d, |
| | | 470.3 | J = 1.8 Hz, 1H), 8.24 (d, J = 1.8 Hz, 1H), 8.10 (d, J = |
| | | (M + 1) | 3.6 Hz, 1H), 6.86 (d, J = 3.6 Hz, 1H), 3.92 (d, J = |
| | | | 11.1 Hz, 2H), 3.64 (brs, 4H), 3.40 (s, 2H), 2.07 (brs, |
| | | | 4H), 1.77-1.64 (m, 5H). |
| B-233 | 9.6%/ | 407.43 for | δ: 13.00 (s, 1H), 8.42 (s, 1H), 8.27 (s, 1H), 8.22 (d, |
| | 98.6% | C22H19F2N5O/ | J = 1.8 Hz, 1H), 8.07 (d, J = 3.6 Hz, 1H), 7.99 (s, 1H), |
| | | 408.3 | 7.88-7.77 (m, 4H), 6.81 (d, J = 3.3 Hz, 1H), 3.65 |
| | | (M + 1) | (brs, 4H), 2.08 (brs, 4H). |
| B-234 | 41%/ | 453.49 for | δ/ppm 10.37 (s, 1H), 8.78 (d, J = 2.1 Hz 1H), 8.74 |
| | 99.6% | C24H25F2N5O2/ | (d, J = 2.1 Hz, 1H), 8.69 (t, J = 4.5 Hz, 1H), 8.44 (d, |
| | | 454.2 | J = 2.1 Hz, 1H), 8.24 (d, J = 2.1 Hz, 1H), 8.09 (d, |
| | | (M + 1) | J = 3.6 Hz, 1H), 6.86 (d, J = 3.6 Hz, 1H), 3.64 (brs, |
| | | | 4H), 2.88-2.83 (m, 1H), 2.07 (brs, 4H), 1.90-1.59 (m, |
| | | | 8H). |
| B-235 | 16%/ | 395.36 for | δ: 8.59 (s, J = 2.1 Hz, 1H), 8.45 (d, J = 1.8 Hz, 1H), |
| | 90.7% | C18H17F4N5O/ | 8.19-8.20 (m, 2H), 8.05 (d, J = 3.6 Hz, 1H), 6.78 (d, |
| | | 396.1 | J = 3.6 Hz, 1H), 3.64 (brs, 4H), 2.07 (brs, 4H) |
| | | (M + 1) | |
| B-236 | 66%/ | 439.47 for | δ/ppm 10.22 (s, 1H), 8.77 (s, 1H), 8.73 (d, J = 2.4 |
| | 98.9% | C23H23F2N5O2/ | Hz, 1H), 8.69 (s, 1H), 8.44 (d, J = 1.8 Hz, 1H), 8.24 |
| | | 440.1 | (d, J = 1.8 Hz, 1H), 8.09 (d, J = 3.9 Hz, 1H), 6.86 (d, |
| | | (M + 1) | J = 3.6 Hz, 1H), 3.65 (brs, 4H), 2.16-2.07 (m, 9H), |
| | | | 1.83 (brs, 2H). |
| B-237 | 57%/ | 503.50 for | δ/ppm 10.46 (s, 1H), 8.78 (d, J = 2.1 Hz 1H), 8.75 |
| | 99.4% | C25H25F4N5O2/ | (d, J = 2.1 Hz, 1H), 8.7 (d, J = 2.4 Hz, 1H), 8.44 (d, |
| | | 504.2 | J = 2.1 Hz, 1H), 8.24 (d, J = 1.8 Hz, 1H), 8.10 (d, |
| | | (M + 1) | J = 3.3 Hz, 1H), 6.86 (d, J = 3.9 Hz, 1H), 3.64 (brs, |
| | | | 4H), 2.50 (m, 6H), 2.12-1.95 (m, 4H), 1.84-1.68 |
| | | | (m, 3H). |
| B-238 | 38.7%/ | 461.45 for | δ: 10.134 (s, 1H), 8.70 (d, J = 1.8 Hz, 1H), 8.626 (d, |
| | 99.9% | C22H22F3N5O3/ | J = 1.8 Hz, 1H), 8.54 (d, J = 1.8 Hz, 1H), 8.08 (d, |
| | | 462.2 | J = 3.6 Hz, 1H), 6.86 (d, J = 3.6 Hz, 1H), 4.55 (brs, |
| | | (M + 1) | 1H), 4.18 (q, J = 7.2 Hz, 2H), 3.69 (brs, 1H), 3.08 |
| | | | (brs, 1H), 2.68 (brs, 1H), 1.99 (brs, 1H), 1.66 (brs, |
| | | | 1H), 1.62 (brs, 2H), 1.27 (t, J = 6.9 Hz, 3H). |

### Biological Examples

### Example B-1. hPGDH Inhibitor Screening Biochemical Assay

A hydroxyprostaglandin dehydrogenase inhibition screening biochemical assay can be performed to assess the synthesized inhibitors provided herein. Provided herein is an exemplary biochemical assay for hPGDH inhibitor screening.

The in vitro biochemical assay can be performed in white, 384 plates in total 20 μl reaction volume consisting of 10 nM of 15-PGDH/HPGD (R&D System #5660-DH), 15 μM Prostaglandin E2 (Sigma, C₈ t #P5640-10 MG) and 0.25 mM β-Nicotinamide adenine dinucleotide sodium salt (Sigma, C₈ t #N₀₆₃₂-5G) made in reaction buffer (50 mM Tris-HCl, pH 7.5, 0.01% Tween 20) at 10-point dose response curve for test/tool compounds. Briefly, 5 μl (4×) of compounds solution and 5 μl (final concentration, 10 nM) of enzyme solution is added to white 384 well plates and incubated for 10 mins at 37° C. 5 μl (4×) of Prostaglandin β2 and 5 μl (4×) of β-Nicotinamide adenine dinucleotide sodium salt is added to the wells and incubated for 10 mins at room temperature. Fluorescence is recorded at ex/em=340 nm/485 nm. The percentage (%) inhibition of enzyme activity was determined relative to positive control (1% DMSO) and IC50 was calculated using GraphPad prism software (four parameter-variable slope equation). Exemplary data are shown in Table 4.

TABLE 4
hPGDH inhibition potency.

| Compound | hPGDH: Average | Compound | hPGDH: Average |
|---|---|---|---|
| No | IC₅₀ (μM) | No | IC₅₀ (μM) |
| B-42 | A | B-24 | A |
| B-38 | A | B-23 | A |
| B-43 | B | B-19 | A |
| B-41 | A | B-11 | A |
| B-39 | A | B-22 | A |
| B-44 | A | B-21 | A |
| B-40 | A | B-20 | A |
| B-37 | A | B-18 | A |
| B-35 | A | B-17 | A |
| B-33 | A | B-14 | A |
| B-45 | A | B-12 | A |
| B-36 | A | B-7 | A |
| B-34 | A | B-5 | A |
| B-28 | A | B-13 | B |
| B-26 | A | B-2 | A |
| B-15 | A | B-10 | A |
| B-30 | A | B-9 | A |
| B-29 | A | B-8 | A |
| B-31 | A | B-6 | A |
| B-27 | A | B-3 | A |
| B-16 | A | B-4 | A |
| B-25 | A | B-1 | A |
| B-48 | A | B-47 | A |
| B-49 | A | B-54 | A |
| B-55 | A | B-79 | A |
| B-56 | A | B-80 | A |
| B-57 | A | B-81 | B |
| B-58 | A | B-82 | A |
| B-59 | B | B-83 | A |
| B-60 | A | B-84 | A |
| B-61 | A | B-85 | A |
| B-62 | B | B-88 | A |
| B-65 | A | B-89 | A |
| B-66 | A | B-91 | A |
| B-69 | A | B-92 | A |
| B-70 | A | B-93 | A |
| B-71 | A | B-94 | A |
| B-72 | A | B-95 | A |
| B-73 | A | B-96 | A |
| B-74 | A | B-102 | B |
| B-75 | A | B-104 | A |
| B-76 | A | B-110 | A |
| B-77 | A | B-111 | A |
| B-78 | A | B-113 | A |
| B-114 | A | B-127 | A |
| B-126 | A | B-315 | A |
| B-226 | A | B-316 | A |
| B-227 | A | B-239 | A |
| B-228 | A | B-240 | A |
| B-229 | A | B-241 | A |
| B-230 | A | B-242 | A |
| B-231 | A | B-243 | A |
| B-232 | A | B-244 | A |
| B-233 | A | B-245 | A |
| B-234 | A | B-246 | A |
| B-235 | B | B-247 | A |
| B-236 | A | B-248 | A |
| B-237 | A | B-249 | A |
| B-238 | A | B-250 | A |
| B-251 | A | B-252 | A |
| B-253 | A | B-254 | A |
| B-255 | A | B-256 | A |
| B-257 | A | B-258 | A |
| B-259 | A | B-260 | A |
| B-261 | B | B-272 | A |
| B-263 | A | B-265 | A |
| B-266 | A | B-267 | A |
| B-268 | A | B-269 | A |
| B-270 | A | B-271 | A |
| B-272 | A | B-273 | A |
| B-274 | A | | |

A < 0.1 μM;
0.1 μM ≤ B < 1 μM;
1 μM ≤ C

### Example B-2. Additional Biochemical Assays

Cell Based Assay: 15-PGDH is highly expressed in resting human lung adenocarcinoma cells (A549) (Tong et al., 2006), and this cell line was used to assess 15-PGDH inhibition by MF-300Na in vitro.

In this assay, A549 cells are treated with interleukin (IL) 1β, which induces the expression of cycloxygenase-2 and the synthesis of PGE2 (Tong et al., 2006). In the studies evaluating test articles, thirty thousand A549 cells were seeded in 100 μL F12K completed media and incubated for 24 hours at 37° C. with 5% CO₂ before being serum-starved for 24 hours. On the day of the experiment, buffer was changed to complete medium, and cells were incubated for 30 minutes with compounds prior to the addition of IL-1β (final concentration of 0.1-0.25 ng/mL) overnight at 37° C. with 5% CO₂. Each concentration was run in triplicate. In this assay, tool compounds increased PGE2 in the supernatant, and a half maximal effective concentration (EC50) was calculated for each compound. The PGE2 in the supernatant was detected and quantified using a Cisbio HTRF technology (Homogeneous Time-Resolved Fluorescence) kit (62P2APEG-62P2APEH) according to the manufacturer's recommendations, quantifying the fold induction of PGE2 of cells treated with IL-1β plus test article, versus treatment with IL-1β only.

Representative data can be seen in Table 5.

TABLE 5
PGE2 - HTFR over IL-β

| | PGE2 - | PGE2 - | PGE2 - | PGE2 - |
|---|---|---|---|---|
| | HTRF | HTRF | HTRF | HTRF |
| | over IL-β | over IL-β | over IL-β | over IL-β |
| Cmpd | induction at | induction at | induction at | induction at |
| No. | 1 μM | 0.1 μM | 0.01 μM | 0.001 μM |
| B-36 | 4.33 | 3.31 | 4.16 | 2.93 |
| B-70 | 4.34 | 4.8 | 4.44 | 3.59 |
| B-72 | 3.96 | 3.96 | 3.03 | 2.17 |
| B-79 | 2.39 | 2.99 | 1.83 | 2.34 |
| B-80 | 2.4 | 2.86 | 2.83 | 2.36 |
| B-81 | 1.92 | 2.21 | 1.08 | 0.51 |
| B-82 | 4.19 | 5.36 | 5.52 | 2.7 |
| B-83 | 4.39 | 2.84 | 2.64 | 1.4 |
| B-84 | 5.76 | 4.02 | 3.38 | 1.94 |
| B-85 | 7.01 | 6.28 | 4.35 | 1.54 |
| B-88 | 4.67 | 3.25 | 3.77 | 2.14 |
| B-89 | 5.13 | 3 | 3.31 | 1.57 |
| B-92 | 3.56 | 4.01 | 2.91 | 2.67 |
| B-92 | 3.92 | 3.89 | 2.33 | 0.97 |
| B-96 | 5.19 | 3.22 | 1.54 | 0.77 |
| B-97 | 5.37 | 4.98 | 2.42 | 1.08 |
| B-100 | 5.26 | 4.49 | 1.78 | 0.79 |
| B-102 | 5.02 | 3.09 | 1.08 | 0.95 |
| B-103 | 6.3 | 5.1 | 1.34 | 1.15 |
| B-106 | 2.8 | 0.86 | 0.81 | 0.90 |
| B-107 | 3.51 | 2.12 | 1.31 | 1.73 |
| B-108 | 3.26 | 3.46 | 2.98 | 1.57 |
| B-110 | 4.03 | 3.7 | 3.35 | 3.31 |
| B-113 | 10.7 | 8.74 | 3.06 | 6.05 |
| B-114 | 11.1 | 7.25 | 5.78 | 2.23 |
| B-115 | 5.24 | 4.92 | 3.07 | 4.36 |
| B-118 | 1.02 | 1 | 1.55 | 1.48 |
| B-119 | 3.06 | 2.87 | 1.2 | 1.01 |
| B-120 | 6.26 | 4.15 | 1.28 | 1.34 |
| B-231 | 4.03 | 9.67 | 7.31 | 4.9 |
| B-232 | 4.46 | 2.68 | 1.02 | 0.34 |
| B-234 | 3.42 | 4.06 | 4.01 | 0.70 |
| B-236 | 8.32 | 6.46 | 3.57 | 1.63 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of Formula (N), or a pharmaceutically acceptable salt or solvate thereof: 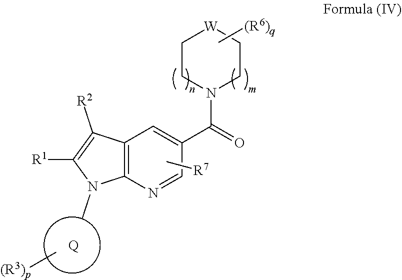
wherein,
ring Q is C₆ aryl or 5-to 10-membered heteroaryl;
W is —CR⁶R⁶—, —O—,-5-, —NR⁵—, —S(O)₂—, or —C(O)—;
R¹ and R² are each independently H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —OC(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁵ is H, C₁-C₆ alkyl, or —C(O)R¹⁰;
each R⁶ is independently H, halogen, CN, —NR⁸R⁹, —OR¹⁰, —C(O)R¹⁰, —C(O)OR¹⁰, —C(O)NR⁸R⁹, —SOR¹¹, —SO₂R¹¹, substituted or unsubstituted C₁-C₆ alkyl;
or two R⁶ can join together with the atom(s) to which they are attached to form a C₃-C₆ cycloalkyl or C₃-C₈ heterocycloalkyl ring;
R⁷ is H, halogen, —CN, —NR¹⁰R¹⁰, —OR¹⁰, —C(O)R¹⁰, —C(O)OR¹⁰, or substituted or unsubstituted C₁-C₆ alkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
n and m are each independently 0, 1, 2, or 3;
q is 0, 1, 2, or 3; and
p is 1, 2, 3, or 4.

2. The compound of claim 1, or a pharmaceutically acceptable salt or solvate thereof, wherein ring Q is a bicyclic or monocyclic heteroaryl optionally comprising 1, 2, or 3 heteroatoms selected from O, S, or N.

3. The compound of claim 1, or a pharmaceutically acceptable salt or solvate thereof, wherein ring Q is a phenyl, pyrimidinyl, or pyridinyl.

4. The compound of any one of claims 1-3, wherein the compound has the structure of Formula (V), or a pharmaceutically acceptable salt or solvate thereof: 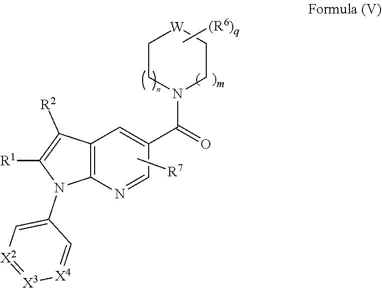
wherein,
X² is N, NR^{3A}, or CR^{3A};
X³ is N or CR^{3B};
X⁴ is N, NR^{3C}, or CR^{3C}; and
R^{3A}, R^{3B}, and R^{3C} are each independently H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R^{3A} and R^{3B} together with the atoms to which they are attached form a substituted or unsubstituted 5 to 6-membered heteroaryl; or
R^{3B} and R^{3C} together with the atoms to which they are attached form a substituted or unsubstituted 5 to 6-membered heteroaryl;
wherein R^{3A}, R^{3B}, and R are not all H at the same time.

5. The compound of claim 4, or a pharmaceutically acceptable salt or solvate thereof, wherein: X² is N or CR^{3A};
X³ is N or CR^{3B}; and
X⁴ is N or CR^{3C}.

6. The compound of claim 4 or 5, or a pharmaceutically acceptable salt or solvate thereof, wherein one of one of X², X³, or X⁴ is N.

7. The compound of any one of claims 4-6, or a pharmaceutically acceptable salt or solvate thereof, wherein X² is N; X³ is CR^{3B}; and X⁴ is CR^{3C}.

8. The compound of any one of claims 4-6, or a pharmaceutically acceptable salt or solvate thereof, wherein X² is CR^{3A}; X⁴ is N; X³ is CR^{3B}.

9. The compound of any one of claims 4-6, or a pharmaceutically acceptable salt or solvate thereof, wherein X² is CR^{3A}; X³ is CR^{3B}; and X⁴ is N.

10. The compound of any one of claims 4-9, or a pharmaceutically acceptable salt or solvate thereof, wherein R^{3A}, R^{3B}, and R^{3C} are each independently H, halogen, —CN, —C(O)R¹⁰, —C(O)OR¹⁰, —C(O)NR⁸R⁹, —NR¹²C(O)R¹⁰, —NR¹²C(O)OR¹⁰, —NR¹²C(O)NR⁸R⁹, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl.

11. The compound of any one of claims 4-9, or a pharmaceutically acceptable salt or solvate thereof, wherein R^{3A}, R^{3B}, and R^{3C} are each independently H, halogen, —C(O)NR⁸R⁹, —C(O)R¹⁰, —NR¹²C(O)OR¹⁰, substituted or unsubstituted C₃-C₈ heterocycloalkyl, or substituted or unsubstituted 5-membered heteroaryl.

12. The compound of claim 4, or a pharmaceutically acceptable salt or solvate thereof, wherein R^{3A} and R^{3B} together with the atoms to which they are attached form a substituted or unsubstituted 5 to 6-membered aryl or heteroaryl optionally comprising 1, 2, or 3 heteroatoms selected from 0, S, and N.

13. The compound of claim 4, or a pharmaceutically acceptable salt or solvate thereof, wherein R^{3B} and R^{3C} together with the atoms to which they are attached form a substituted or unsubstituted 5 to 6-membered aryl or heteroaryl optionally comprising 1, 2, or 3 heteroatoms selected from 0, S, and N.

14. The compound of claim 4, wherein the compound has the structure of Formula (Va), or a pharmaceutically acceptable salt or solvate thereof: 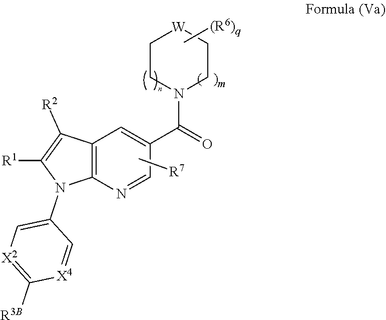
wherein,
X² is N or CR^{3A}; and
X⁴ is N or CR^{3C}; and
R^{3A}, R^{3B}, and R^{3C} are each independently H, halogen, —CN, —NR⁸R⁹, —OR¹⁰, CN, —C(O)R¹⁰, —C(O)OR¹⁰, —C(O)NR⁸R⁹, —NR¹²C(O)R¹⁰, —NR¹²C(O)OR¹⁰, —NR¹²C(O)NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
wherein R^{3A}, R^{3B}, and R^{3C} are not each H.

15. The compound of claim 14, or a pharmaceutically acceptable salt or solvate thereof, wherein X² is N and X⁴ is CR^{3C}.

16. The compound f claim 15, or a pharmaceutically acceptable salt or solvate thereof, wherein R^{3B} is H, and R^{3C} is —C(O)R¹⁰, —C(O)OR¹⁰, —C(O)NR⁸R⁹, —NR¹²C(O)OR¹⁰, substituted or unsubstituted C₃-C₈ heterocycloalkyl, or substituted or unsubstituted 5-membered heteroaryl.

17. The compound f claim 15, or a pharmaceutically acceptable salt or solvate thereof, wherein R^{3C} is H, and R^{3B} is —C(O)R¹⁰, —C(O)OR¹⁰, —C(O)NR⁸R⁹, —NR¹²C(O)OR¹⁰, substituted or unsubstituted C₃-C₈ heterocycloalkyl, or substituted or unsubstituted 5-membered heteroaryl.

18. The compound of claim 14, or a pharmaceutically acceptable salt or solvate thereof, wherein X² is C^{3A} and X⁴ is N.

19. The compound of claim 18, or a pharmaceutically acceptable salt or solvate thereof, wherein R^{3A} is H, and R^{3B} is —C(O)R¹⁰, —NR¹²C(O)OR¹⁰, substituted or unsubstituted C₃-C₈ heterocycloalkyl, or substituted or unsubstituted 5-membered heteroaryl.

20. The compound of claim 18, or a pharmaceutically acceptable salt or solvate thereof, wherein R^{3B} is H, and R^{3A} is —C(O)R¹⁰, —C(O)OR¹⁰, —C(O)NR⁸R⁹, —NR¹²C(O)OR¹⁰, substituted or unsubstituted C₃-C₈ heterocycloalkyl, or substituted or unsubstituted 5-membered heteroaryl.

21. The compound of claim 14, or a pharmaceutically acceptable salt or solvate thereof, wherein X² is CR^{3A} and X⁴ is CRI.

22. The compound of claim 21, or a pharmaceutically acceptable salt or solvate thereof, wherein R^{3A} and R^{3B} are each H; and R is —C(O)R¹⁰, —NR¹²C(O)OR¹⁰, substituted or unsubstituted C₃-C₈ heterocycloalkyl, or substituted or unsubstituted 5-membered heteroaryl.

23. The compound of claim 21, or a pharmaceutically acceptable salt or solvate thereof, wherein R^{3A} and R^{3C} are each H; and R^{3B} is —C(O)R¹⁰, —NR¹²C(O)OR¹⁰, substituted or unsubstituted C₃-C₈ heterocycloalkyl, or substituted or unsubstituted 5-membered heteroaryl.

24. The compound of claim 21, or a pharmaceutically acceptable salt or solvate thereof, wherein R^{3B} and R^{3C} are each H; and R^{3A} is —C(O)R¹⁰, —NR¹²C(O)OR¹⁰, substituted or unsubstituted C₃-C₈ heterocycloalkyl, or substituted or unsubstituted 5-membered heteroaryl.

25. The compound of any one of claims 14-24, wherein one of R^{3A}, R^{3B}, or RIC is a substituted or unsubstituted 5-membered heteroaryl.

26. The compound of claim 25, wherein the 5-membered heteroaryl is triazinyl, pyrrolyl, furanyl,
imidazolyl, pyrazolyl, oxazolyl, isoxazolyl, thiophenyl, thiazolyl, isothiazolyl, triazolyl,
oxadiazolyl, thiadiazolyl, or tetrazolyl.

27. The compounds of any one of claims 14-26, wherein one of R^{3A}, R^{3B}, or R^{C} is represented by moiety: 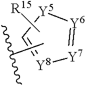
wherein,
Y⁵ is NR^{15A}, S, or O;
Y⁶, Y⁷, and Y⁸ are each independently N or CR¹⁵;
R¹⁵ is H, halogen, —NR⁸R⁹, —C₁-C₆ alkyl, C₁-C₆ haloalkyl, substituted or unsubstituted C₃-C₈ cycloalkyl, or substituted or unsubstituted C₃-C₈ heterocycloalkyl; and
R^{15A} is H or C₁-C₆ alkyl.

28. The compound of claim 27, or a pharmaceutically acceptable salt or solvate thereof, wherein one of R^{3A}, R^{3B}, or R^{3C} is represented by moiety: 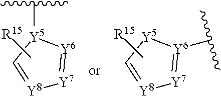
wherein 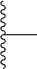
represents the connection point to R^{3A}, R^{3B}, or R^{3C}.

29. The compound of claim 1 or 2, or a pharmaceutically acceptable salt or solvate thereof, wherein ring Q is a 5-membered heteroaryl.

30. The compound of claim 1 or 2, or a pharmaceutically acceptable salt or solvate thereof, wherein ring Q is triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl.

31. The compound of claim 1, or a pharmaceutically acceptable salt or solvate thereof, wherein ring Q is: 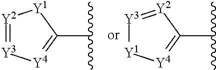
wherein,
Y¹ is O, S, or NR^{3D};
Y² is N or CR^{3A};
Y³ and Y³ are each independently N or CR^{3B};
R^{3A} and R^{3B} are each independently selected from H, halogen, —NR⁸R⁹, —OR¹⁰, —C(O)R¹⁰, —C(O)OR¹⁰, —C(O)NR⁸R⁹,-substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, or substituted or unsubstituted 5-membered heteroaryl; and
R^{3D} is H or C₁-C₆ alkyl.

32. The compound of claim 31, wherein the compound has the structure of Formula (VIIa) or (VIIb), or a pharmaceutically acceptable salt or solvate thereof:

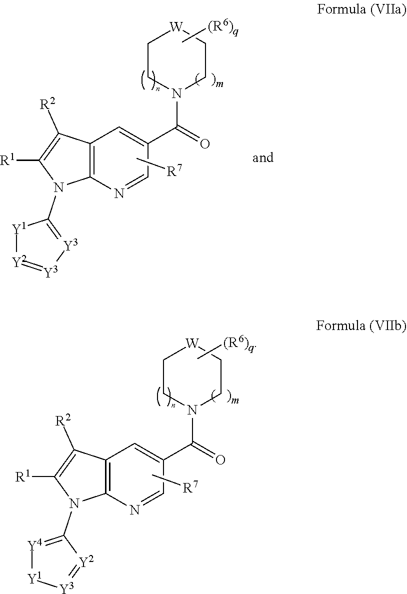

33. The compound of claim 31 or 32, wherein:
Y¹ is O or S;
Y² is CR^{3A}; and
Y³ and Y⁴ are each independently N or CR^{3B}.

34. The compound of claim 31 or 32, wherein:
Y¹ is O or S;
Y² is N; and
Y³ and Y³ are each independently N or CR^{3B}.

35. The compound of claim 1 or 2, or a pharmaceutically acceptable salt or solvate thereof, wherein ring Q is a bicyclic heteroaryl comprising 1-3 heteroatoms selected from N, O, and S.

36. The compound of claim 1, or a pharmaceutically acceptable salt or solvate thereof, wherein ring Q is: 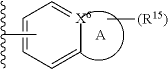
wherein,
ring A is a 5-membered heteroaryl optionally comprising 1 or 2 N atoms;
X⁶ is C or N; and
R¹⁵ is H, halogen, —NR⁸R⁹,-substituted or unsubstituted C₁-C₆ alkyl, substituted or unsubstituted C₁-C₆ haloalkyl, substituted or unsubstituted C₃-C₈ cycloalkyl, or substituted or unsubstituted C₃-C₈ heterocycloalkyl.

37. The compound of claims 1 or 36, wherein the compound has the structure of Formula (VIII), or a pharmaceutically acceptable salt or solvate thereof:

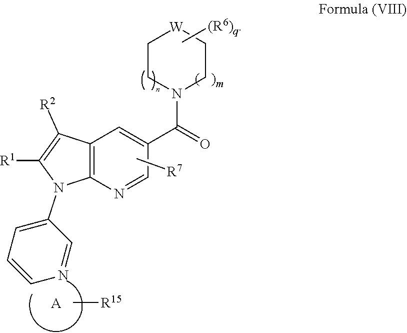

38. A compound of Formula (Ia), or a pharmaceutically acceptable salt or solvate thereof: 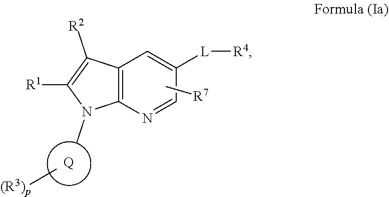
wherein,
ring Q is C₆ aryl or 5-to 10-membered heteroaryl;
L is —CR^{13A}, R^{13B}—C(O)—, —S—, —S(O)—, or —S(O)₂—;
R¹ and R² are each independently H, halogen, —CN, —OR¹⁰, —C(O)R¹⁰, —C(O)OR¹⁰, —NR⁸R⁹, —C(O)NR⁸R⁹, —NR¹⁰C(O)R¹⁰, substituted or unsubstituted C₁-C₆ alkyl, or substituted or unsubstituted C₃-C₈ cycloalkyl, substituted or unsubstituted alkenyl, or substituted or unsubstituted alkynyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —OC(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ alkenyl, substituted or unsubstituted C₁-C₆ alkynyl substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁴ is substituted or unsubstituted C₁, —C₈ alkyl, substituted or unsubstituted C₂-C₈ alkenyl, substituted or unsubstituted C₁, —C₈ heteroalkyl; 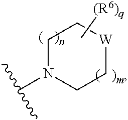
or R⁴ is
wherein
W is —CR⁶R⁶—, —O—, —S—, —NR⁵—, —S(O)₂—, or —C(O)—;
R⁵ is H or substituted or unsubstituted C₁-C₆ alkyl, substituted or unsubstituted cycloalkyl; or substituted or unsubstituted heterocycloalkyl;
each R⁶ is independently H, halogen, CN, —NR⁸R⁹, —OR¹⁰, —C(O)R¹⁰, —C(O)OR¹⁰, —C(O)NR⁸R⁹, —NR⁸C(O)R¹⁰—SOR¹¹, —SO₂R¹¹, —SR¹¹, substituted or unsubstituted C₁-C₆ alkyl or C₃-C₈ cycloalkyl;
or two R⁶ can join together with the atom(s) to which they are attached to form a C₃-C₆ cycloalkyl or C₃-C₈ heterocycloalkyl ring;
n and m are each independently 0, 1, 2, or 3; and
q is 0, 1, 2, 3, 4, 5, or 6;
R⁷ is H, halogen, —CN, —NR¹⁰R¹⁰, —OR¹⁰, —C(O)R¹⁰, —C(O)OR¹⁰, or substituted or unsubstituted C₁-C₆ alkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆haloalkyl, and C₃-C₁₀cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
R^{13A} and R^{13B} are each independently H, CF₃, halogen, or C₁-C₆ alkyl; and p is 1, 2, 3, or 4.

39. The compound of claim 38, or a pharmaceutically acceptable salt or solvate thereof, wherein ring Q is phenyl or a 6-membered heteroaryl.

40. The compound of claim 38 or 39, or a pharmaceutically acceptable salt or solvate thereof, wherein ring Q is pyridinyl, pyrazinyl, pyrimidinyl, or pyridazinyl.

41. The compound of any one of claims 1-3 or 38-39, or a pharmaceutically acceptable salt or solvate thereof, wherein each R³ is independently selected from H, halogen, —C(O)R¹⁰, —C(O)OR¹⁰, —C(O)NR⁸R⁹, —NR¹²C(O)OR¹⁰, substituted or unsubstituted C₁-C₆ haloalkyl, substituted or unsubstituted C₃-C₈ heterocycloalkyl, or substituted or unsubstituted 5-membered heteroaryl.

42. The compound of claim 41, or a pharmaceutically acceptable salt or solvate thereof, wherein each R³ is independently selected from H, halogen, —C(O)R¹⁰, —C(O)NR⁸R⁹, —NR¹²C(O)OR¹⁰, substituted or unsubstituted C₃-C₈ heterocycloalkyl, or substituted or unsubstituted 5-membered heteroaryl.

43. The compound of claim 38, wherein the compound has the structure of Formula (II), or a pharmaceutically acceptable salt or solvate thereof: 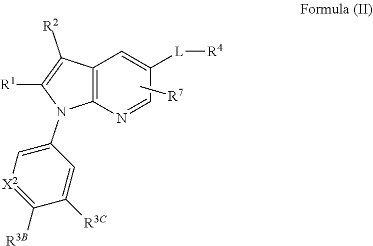
wherein,
X² is N or CR^{3A}; and
R^{3A}, R^{3B}, and R^{a}C are each independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
provided that R^{3A}, R^{3B}, and R^{3C} are not all H at the same time.

44. The compound of claim 43, or a pharmaceutically acceptable salt or solvate thereof, wherein X² is N.

45. The compound of claim 43, or a pharmaceutically acceptable salt or solvate thereof, wherein X² is CR^{3A}.

46. The compound of claim 38, or a pharmaceutically acceptable salt or solvate thereof, wherein ring Q is a 5-membered heteroaryl.

47. The compound of claim 38 or 46, or a pharmaceutically acceptable salt or solvate thereof, wherein ring Q is triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl.

48. The compound of claim 38, wherein the compound has the structure of Formula (IIIa) or (IIIb), or a pharmaceutically salt or solvate thereof: 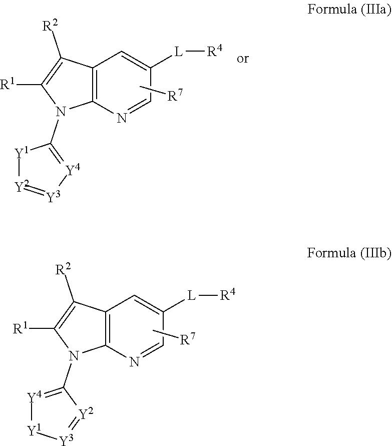
wherein,
Y¹ is O, S, or NR^{3D};
Y² is N or CR^{3A};
Y³ and Y⁴ are each independently N or CR^{3B};
R^{3A} and R^{3B} are each independently selected from H, halogen, —NR⁸R⁹, —OR¹⁰, —C(O)R¹⁰, —C(O)OR¹⁰, —C(O)NR⁸R⁹,-substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, or substituted or unsubstituted 5-membered heteroaryl; and
R^{3D} is H or C₁-C₆ alkyl.

49. The compound of any one of claims 38-48, or a pharmaceutically acceptable salt or solvate thereof, wherein:
L is —S—, —S(O)—, or —S(O)₂—; and
R⁷ is substituted or unsubstituted C₁-C₆ alkyl.

50. The compound of any one of claims 38-48, or a pharmaceutically acceptable salt or solvate thereof, wherein:
L is C(O); and
R⁷ is

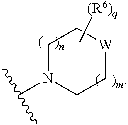

51. The compound of any one of claims 1-48 or 50, or a pharmaceutically acceptable salt or solvate thereof, wherein W is —O—, —S(O)₂—.

52. The compound of any one of claims 1-48 or 50, of a pharmaceutically acceptable salt or solvate thereof, wherein W is —NR⁵—.

53. The compound of any one of claims 1-48 or 50, of a pharmaceutically acceptable salt or solvate thereof, wherein W is —CR⁶R⁶—.

54. The compound of any one of claims 1-48, 50, or 53, or a pharmaceutically acceptable salt or solvate thereof, wherein each R⁶ is independently H, halogen, —NR⁸R⁹, —OR¹⁰, —C(O)R¹⁰, —C(O)OR¹⁰, —C(O)NR⁸R⁹, or substituted or unsubstituted C₁-C₆ alkyl.

55. The compound of claim 54, or a pharmaceutically acceptable salt or solvate thereof, wherein each R⁶ is independently F, —NH₂, —OH, —OCH₃, or —CH₃.

56. The compound of any one of claims 1-55, or a pharmaceutically acceptable salt or solvate thereof, wherein q is 1.

57. The compound of any one of claims 1-55, or a pharmaceutically acceptable salt or solvate thereof, wherein q is 2.

58. The compound of any one of claims 1-57, or a pharmaceutically acceptable salt or solvate thereof, wherein n is 0, 1, or 2; and m is 0 or 1.

59. The compound of any one of claims 1-48 or 50-58, or a pharmaceutically acceptable salt or solvate thereof, wherein:

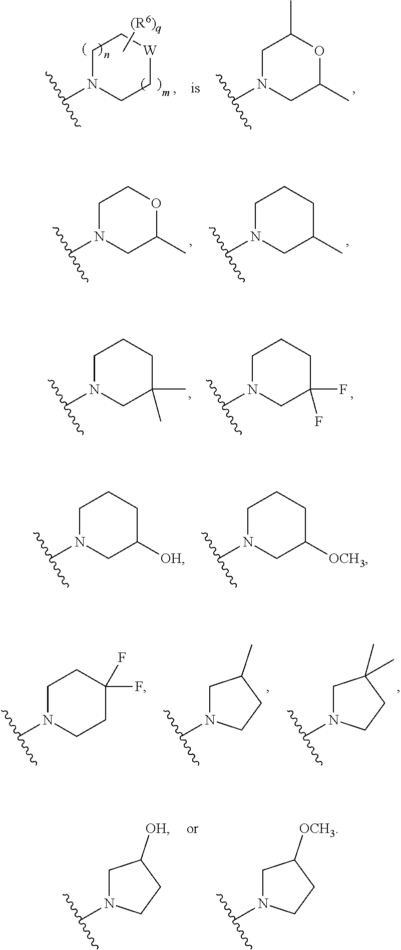

60. The compound of claim 59, or a pharmaceutically acceptable salt or solvate thereof, wherein

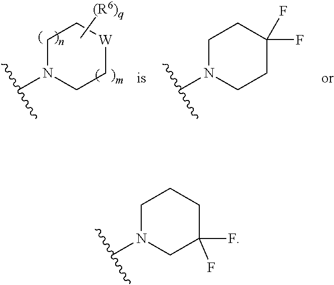

61. The compound of any one of claims 1-60, or a pharmaceutically acceptable salt or solvate thereof, wherein:
R⁷ is H or substituted or unsubstituted C₁-C₆ alkyl; and
R² is H or substituted or unsubstituted C₁-C₆ alkyl.

62. The compound of claim 61, or a pharmaceutically acceptable salt of solvate thereof, wherein R¹ and R² are each H.

63. The compound of any one of claims 1-62, or a pharmaceutically acceptable salt or solvate thereof, wherein R⁷ is H.

64. The compound of any one of claims 1-63, or a pharmaceutically acceptable salt or solvate thereof, wherein the compound is selected from Table 1 or Table 2.

65. A pharmaceutical composition comprising a compound of any one of claims 1-64, or a pharmaceutically acceptable salt or solvate thereof; and a pharmaceutically acceptable excipient.

66. A method of promoting and/or stimulation skin pigmentation, comprising administering one or more of said compositions of any of the preceding claims to a subject in need thereof.

67. A method of inhibiting hair loss, comprising administering one or more of said compositions of any of the preceding claims to a subject in need thereof.

68. A method of preventing and/or treating skin inflammation and/or damage, comprising administering one or more of said compositions of any of the preceding claims to a subject in need thereof.

69. A method of preventing and/or treating vascular insufficiency, comprising administering one or more of said compositions of any of the preceding claims to a subject in need thereof.

70. A method of preventing, treating, minimizing and/or reversing congestive heart failure, cardiomyopathy, comprising administering one or more of said compositions of any of the preceding claims to a subject in need thereof.

71. A method of reducing cardiac ejection fraction, comprising administering one or more of said compositions of any of the preceding claims to a subject in need thereof.

72. A method of preventing and/or treating a gastrointestinal disease, comprising administering one or more of said compositions of any of the preceding claims to a subject in need thereof.

73. A method of preventing and/or treating renal dysfunction, comprising administering one or more of said compositions of any of the preceding claims to a subject in need thereof.

74. A method of stimulation bone resorption and bone formation, comprising administering one or more of said compositions of any of the preceding claims to a subject in need thereof.

75. A method of stimulating tissue regeneration by stimulating, comprising administering one or more of said compositions of any of the preceding claims to a subject in need thereof.

76. A method of modulating cervical ripening, comprising administering one or more of said compositions of any of the preceding claims to a subject in need thereof.

77. A method of promoting neuroprotection and/or stimulating neuronal regeneration, comprising administering one or more of said compositions of any of the preceding claims to a subject in need thereof.

78. A method of treating and/or preventing a neurological disorder, a neuropsychiatric disorder, a neural injury, a neural toxicity disorder, a neuropathic pain, or a neural degenerative disorder, comprising administering one or more of said compositions of any of the preceding claims to a subject in need thereof.

79. A method of treating and/or preventing fibrotic or adhesion disease, disorder or condition, comprising administering one or more of said compositions of any of the preceding claims to a subject in need thereof.

80. A method of reducing and/or preventing scar formation, comprising administering one or more of said compositions of any of the preceding claims to a subject in need thereof.

81. A method of treating and/or preventing muscle disorder, muscle injury and/or muscle atrophy, comprising administering one or more of said compositions of any of the preceding claims to a subject in need thereof.

82. A method of treating and/or preventing fibrosis, comprising administering one or more of said compositions of any of the preceding claims to a subject in need thereof.

83. A method of treating and/or preventing idiopathic pulmonary fibrosis, comprising administering one or more of said compositions of any of the preceding claims to a subject in need thereof.

84. A method of treating and/or preventing kidney fibrosis, comprising administering one or more of said compositions of any of the preceding claims to a subject in need thereof.

85. A method of stimulating muscle regeneration, comprising administering one or more of said compositions of any of the preceding claims to a subject in need thereof.

86. A method of promoting organ fitness, comprising administering one or more of said compositions of any of the preceding claims to a subject in need thereof.

87. A method of promoting wound healing, comprising administering one or more of said compositions of any of the preceding claims to a subject in need thereof.

88. A method of treating acute kidney injury, comprising administering one or more of said compositions of any of the preceding claims to a subject in need thereof.

89. A method of treating sarcopenia, comprising administering one or more of said compositions of any of the preceding claims to a subject in need thereof.

90. A method of treating a neuromuscular disease, comprising administering one or more of said compositions of any of the preceding claims to a subject in need thereof.
